SEQUENCE LISTING (6 sequences)

SEQ ID NO: 1 - DNA, 15 bp, Homo sapiens
tggccggtacctgag

SEQ ID NO: 2 - DNA, 15 bp, Homo sapiens
atcaagatctggcct

SEQ ID NO: 3 - DNA, 23 bp, Homo sapiens
tgtgtggagcaccatacctacca

SEQ ID NO: 4 - DNA, 29 bp, Homo sapiens
ccacacttgaacaaaactctattgtcaac

SEQ ID NO: 5 - DNA, 25 bp, Homo sapiens
ggtaggacacaagtctccacaaagc

SEQ ID NO: 6 - DNA, 24 bp, Homo sapiens
ggcagagttcatcagattgtagcg

(19) United States
(12) Patent Application Publication
(10) Pub. No.: US 2021/0074378 A1
(43) Pub. Date: Mar. 11, 2021

(54) Methods for Analyzing Genetic Data to Classify Multifactorial Traits Including Complex Medical Disorders

(71) Applicants: The Trustees of Princeton University, Princeton, NJ (US); The Simons Foundation, Inc., New York, NY (US); The Rockefeller University, New York, NY (US)

(72) Inventors: Jian Zhou, Jersey City, NJ (US); Christopher Y. Park, North Bergen, NJ (US); Chandra Theesfeld, Plainsboro, NJ (US); Robert B. Darnell, New York, NY (US); Olga G. Troyanskaya, Princeton, NJ (US)

(73) Assignees: The Trustees of Princeton University, Princeton, NJ (US); The Simons Foundation, Inc., New York, NY (US); The Rockefeller University, New York, NY (US)

(21) Appl. No.: 16/965,292

(22) PCT Filed: Jan. 28, 2019
(86) PCT No.: PCT/US2019/015484

§ 371 Date: Jul. 27, 2020

Related U.S. Application Data
(60) Provisional application No. 62/622,556, filed on Jan. 26, 2018.
(60) Provisional application No. 62/622,655, filed on Jan. 26, 2018.
(60) Provisional application No. 62/797,926, filed on Jan. 28, 2019.

(51) Int. Cl. G16B 20/00; C12Q 1/6869; G16B 40/20

(52) U.S. Cl. CPC G16B 20/00; C12Q 2522/10; G16B 40/20; C12Q 1/6869

(57) ABSTRACT

Processes to identify variants that affect biochemical regulation are described. Generally, models are used to identify variants that affect biochemical regulation, which can be used in several downstream applications. A pathogenicity of identified variants is also determined in some instances, which can also be used in several. Various methods further develop research tools, perform diagnostics, and treat individuals based on identified variants.

### CROSS REFERENCE TO RELATED APPLICATIONS

This application claims priority to U.S. Provisional Application Ser. No. 62/622,556 entitled “Methods of Identifying Non-coding Genomic RNA Regulatory Sequences and Sequence Variants and Correlating Them with Phenotypic Variations,” filed Jan. 26, 2018, U.S. Provisional Application Ser. No. 62/622,655 entitled “Methods of Identifying Non-coding Regulatory Genomic Sequences and Sequence Variants and Correlating Them with Phenotypic Variations,” filed Jan. 26, 2018, and U.S. Provisional Application Ser. No. 62/797,926 entitled “Methods for Analyzing Genetic Data to Classify Multifactorial Traits Including Complex Medical Disorders,” filed Jan. 28, 2019, each of which is herein incorporated by reference in its entirety.

### STATEMENT REGARDING FEDERALLY SPONSORED RESEARCH OR DEVELOPMENT

This invention was made with Government support under Grants No. HHSN272201000054C, No. HG008901, No. GM071966, No. HL117798, No. HG005998, No. NS034389, and No. NS081706, awarded by the National Institutes of Health. The Government has certain rights in the invention.

### REFERENCE TO A SEQUENCE LISTING SUBMITTED ELECTRONICALLY VIA EFS-WEB

The instant application contains a Sequence Listing which has been filed electronically in ASCII format and is hereby incorporated by reference in its entirety. Said ASCII copy, created on Jan. 24, 2019, is named 05934_ST25.txt and is 1100 bytes in size.

### REFERENCE TO DATA TABLES SUBMITTED ELECTRONICALLY VIA EFS-WEB

The instant application contains four data tables which have been filed electronically and each table is hereby incorporated by reference in its entirety. The four data tables were created on Jan. 28, 2019, and are named as follows (with size in parentheticals): E_Data_Table_1.txt (70 KB), E_Data_Table_2. txt (16 KB), E_Data_Table_3. txt (13 MB), and E_Data_Table_4. txt (1 MB).

### FIELD OF THE INVENTION

The invention is generally directed to methods and processes for genetic data evaluation, and more specifically to methods and systems utilizing genetic data involving multifactorial traits and/or disorders and applications thereof.

### BACKGROUND

Within a typical mammalian genome, the coding DNA (i.e., DNA gene sequences that encode proteins) makes up a very small portion. For example, approximately 2% of the human genome contains sequence that encodes protein. The rest of the genome is noncoding DNA.

Noncoding DNA has long thought to be nonfunctional and often referred to as “junk” DNA. It is now understood, however, that noncoding DNA does in fact have several functions. These functions include encoding various noncoding RNA (e.g., transfer RNA, ribosomal RNA, snoRNA) and regulating gene function. Noncoding DNA can regulate gene transcription and translation by recruiting various transcriptional and posttranscriptional regulatory factors to a gene via various sequence elements. Various transcriptional sequence elements includes transcription factor binding sites, operators, enhancers, silencers, promoters, transcriptional start sites, and insulators. Various posttranscriptional sequence elements include RNA binding protein (RBP) sites, splice acceptors, splice donors, and cis-acting sequence elements.

### SUMMARY OF THE INVENTION

Several embodiments are directed to methods and processes to evaluate variants that affect biochemical regulation.

In an embodiment to treat an individual for a medical disorder, genetic material of an individual that includes a set of genomic loci is sequenced. Each locus of the set of genomic loci contains sequence that has been determined to harbor a pathogenic variant that affects at least one biochemical regulatory process. The effect of harboring a pathogenic variant within each genomic loci has been associated with the pathogenicity of a medical disorder as determined by the effects of the variant on the at least one biochemical regulatory process. A set of variants that reside within the set of genomic loci sequenced is identified. A trained computational model to determine pathogenicity of each variant of the set of variants identified is obtained. The pathogenicity of each variant is based upon an aggregation of the variant's effects upon the at least one biochemical regulatory process. The computational model is trained utilizing a set of known pathogenic variants and a set of null variants. Utilizing the trained computational model, a diagnosis of the individual is determined based upon a cumulative pathogenicity score of the individual. The diagnosis indicates a propensity for the medical disorder. The cumulative pathogenicity score is determined by aggregating pathogenicity of the individual's variants within the set of genomic loci. When the individual is determined to have a diagnosis indicating a propensity for the medical disorder, the individual is treated for the medical disorder.

In another embodiment, the effects of the variant on at least one biochemical regulatory process is determined by a second computational model that has been trained utilizing a set of features of a regulatory effect profile and the regulatory effect profile is one of: a chromatin regulatory effect profile and a RNA binding protein (RBP) and RNA element profile.

In yet another embodiment, the second computational model is a deep neural network.

In a further embodiment, the second computational model is a convolutional neural network.

In still yet another embodiment, the regulatory profile is the chromatin regulatory effect profile, and wherein the set of features are cell-type specific.

In yet a further embodiment, the regulatory profile is the chromatin regulatory effect profile, and wherein the set of features include at least one of: sites of chromatin accessibility, chromatin marks, and transcription factor binding sites.

In an even further embodiment, the chromatin regulatory effect profile is determined utilizing at least one epigenetic assay selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and a methyl array.

In yet an even further embodiment, the regulatory profile is the RBP and RNA element profile, and wherein the set of features are cell-type specific.

In still yet an even further embodiment, the regulatory profile is the RBP and RNA element profile, and wherein the set of features include RBP binding sites.

In still yet an even further embodiment, the RBP and RNA element profile is determined utilizing at least one RNA-binding assays selected from a group consisting of: cross-linking immunoprecipitation sequencing (CLIP-seq) and RNA immunoprecipitation sequencing (RIP-seq).

In still yet an even further embodiment, the genetic material is one of: a whole genome or a partial genome.

In still yet an even further embodiment, the genetic material is obtained from a biopsy of the individual.

In still yet an even further embodiment, the sequencing performed is one of: whole genome sequencing or capture sequencing.

In still yet an even further embodiment, the biochemical regulatory process is selected from a group consisting of: transcriptional regulation, posttranscriptional regulation, and translational regulation.

In still yet an even further embodiment, the identified set of variants include at least one de novo variant.

In still yet an even further embodiment, the identified set of variants include at least one inherited variant.

In still yet an even further embodiment, at least one locus the set of genomic loci is determined based upon the pathogenicity results of applying the trained computational model to a set a variants that have been identified for a collection of individuals having been diagnosed for the medical disorder.

In still yet an even further embodiment, at least one locus the set of genomic loci is identified experimentally to be associated with the medical disorder.

In still yet an even further embodiment, the computational model is a linear regression.

In still yet an even further embodiment, the linear regression model is L2 regularized.

In still yet an even further embodiment, the diagnosis is determined based upon a threshold, and wherein when the individual's cumulative pathogenicity score is above a threshold, the individual is determined to have a propensity for the medical disorder is determined.

In still yet an even further embodiment, the medical disorder is a complex medical disorder.

In still yet an even further embodiment, the medical disorder is selected from a group consisting of: autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis, psoriasis, multiple sclerosis, schizophrenia, sleep apnea, spina bifida, and stroke.

In still yet an even further embodiment, the medical disorder is autism spectrum disorder and treating the individual comprises administering at least one of: behavioral therapy, communication therapy, educational therapy, and risperidone.

In still yet an even further embodiment, the set of set of known pathogenic variants is derived from the Human Gene Mutation Database.

In still yet an even further embodiment, the set of null variants is derived from at least one of: the International Genome Sample Resource (IGSR) 1000 Genomes project, a set of common variants with no expected pathogenicity, a set of variants randomly generated by in silico methods.

In an embodiment to treat an individual for a medical disorder, genetic material of an individual that includes a set of genomic loci is sequenced. Each locus of the set of genomic loci contains sequence that has been determined to harbor a pathogenic variant that affects at least one biochemical regulatory process. The effect of harboring a pathogenic variant within each genomic loci has been associated with the pathogenicity of a medical disorder as determined by the effects of the variant on the at least one biochemical regulatory process. A set of variants that reside within the set of genomic loci sequenced is identified. A first trained computational model to determine a biochemical regulatory effects of the identified variants is obtained. The biochemical regulatory effects are one of: effects on transcriptional regulation or effects on posttranscriptional regulation. The first computational model is trained utilizing a set of features of a regulatory effect profile. The regulatory effect profile is one of: a chromatin regulatory effect profile and a RNA binding protein (RBP) and RNA element profile. The biochemical regulatory effect of each identified variant is determined. A second trained computational model to determine pathogenicity of each variant of the set of variants identified is obtained. The pathogenicity of each variant is based upon an aggregation of the variant's effects upon the at least one biochemical regulatory process. The second computational model is trained utilizing a set of known pathogenic variants and a set of null variants. Utilizing the trained computational model, a diagnosis of the individual is determined based upon a cumulative pathogenicity score of the individual. The diagnosis indicates a propensity for the medical disorder. The cumulative pathogenicity score is determined by aggregating pathogenicity of the individual's variants within the set of genomic loci. When the individual is determined to have a diagnosis indicating a propensity for the medical disorder, the individual is treated for the medical disorder

In another embodiment, the first computational model is a deep neural network.

In yet another embodiment, the first computational model is a convolutional neural network.

In a further embodiment, the regulatory profile is the chromatin regulatory effect profile, and wherein the set of features are cell-type specific.

In still yet another embodiment, the regulatory profile is the chromatin regulatory effect profile, and wherein the set of features include at least one of: sites of chromatin accessibility, chromatin marks, and transcription factor binding sites.

In yet a further embodiment, the chromatin regulatory effect profile is determined utilizing at least one epigenetic assay selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and a methyl array.

In an even further embodiment,

In yet an even further embodiment, the regulatory profile is the RBP and RNA element profile, and wherein the set of features are cell-type specific.

In still yet an even further embodiment, the regulatory profile is the RBP and RNA element profile, and wherein the set of features include RBP binding sites.

In still yet an even further embodiment, the RBP and RNA element profile is determined utilizing at least one RNA-binding assays selected from a group consisting of: cross-linking immunoprecipitation sequencing (CLIP-seq) and RNA immunoprecipitation sequencing (RIP-seq).

In still yet an even further embodiment, the genetic material is one of: a whole genome or a partial genome.

In still yet an even further embodiment, the genetic material is obtained from a biopsy of the individual.

In still yet an even further embodiment, the sequencing performed is one of: whole genome sequencing or capture sequencing.

In still yet an even further embodiment, the biochemical regulatory process is selected from a group consisting of: transcriptional regulation, posttranscriptional regulation, and translational regulation.

In still yet an even further embodiment, the identified set of variants include at least one de novo variant.

In still yet an even further embodiment, the identified set of variants include at least one inherited variant.

In still yet an even further embodiment, at least one locus the set of genomic loci is determined based upon the pathogenicity results of applying the second trained computational model to a set a variants that have been identified for a collection of individuals having been diagnosed for the medical disorder.

In still yet an even further embodiment, at least one locus the set of genomic loci is identified experimentally to be associated with the medical disorder.

In still yet an even further embodiment, the second computational model is a linear regression.

In still yet an even further embodiment, the linear regression model is L2 regularized.

In still yet an even further embodiment, the diagnosis is determined based upon a threshold, and wherein when the individual's cumulative pathogenicity score is above a threshold, the individual is determined to have a propensity for the medical disorder is determined.

In still yet an even further embodiment, the medical disorder is a complex medical disorder.

In still yet an even further embodiment, the medical disorder is selected from a group consisting of: autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis, psoriasis, multiple sclerosis, schizophrenia, sleep apnea, spina bifida, and stroke.

In still yet an even further embodiment, the medical disorder is autism spectrum disorder and treating the individual comprises administering at least one of: behavioral therapy, communication therapy, educational therapy, and risperidone.

In still yet an even further embodiment, the set of set of known pathogenic variants is derived from the Human Gene Mutation Database.

In still yet an even further embodiment, the set of null variants is derived from at least one of: the International Genome Sample Resource (IGSR) 1000 Genomes project, a set of common variants with no expected pathogenicity, a set of variants randomly generated by in silico methods.

In an embodiment of treating autism spectrum disorder, genetic material of an individual that includes a set of genomic loci is sequenced. Each locus of the set of genomic loci contains sequence that has been determined to harbor a pathogenic variant that affects at least one biochemical regulatory process. The effect of harboring a pathogenic variant within each genomic loci has been associated with the pathogenicity of autism spectrum disorder as determined by the effects of the variant on the at least one biochemical regulatory process. A set of variants that reside within the set of genomic loci sequenced is identified. A trained computational model to determine pathogenicity of each variant of the set of variants identified is obtained. The pathogenicity of each variant is based upon an aggregation of the variant's effects upon the at least one biochemical regulatory process. The computational model is trained utilizing a set of known pathogenic variants and a set of null variants. Utilizing the trained computational model, a diagnosis of the individual is determined based upon a cumulative pathogenicity score of the individual. The diagnosis indicates a propensity for autism spectrum disorder. The cumulative pathogenicity score is determined by aggregating pathogenicity of the individual's variants within the set of genomic loci. When the individual is determined to have a diagnosis indicating a propensity for autism spectrum disorder, the individual is treated for autism spectrum disorder.

In another embodiment, the effects of the variant on at least one biochemical regulatory process is determined by a second computational model that has been trained utilizing a set of features of a regulatory effect profile and the regulatory effect profile is one of: a chromatin regulatory effect profile and a RNA binding protein (RBP) and RNA element profile.

In yet another embodiment, the second computational model is a deep neural network.

In a further embodiment, the second computational model is a convolutional neural network.

In still yet another embodiment, the regulatory profile is the chromatin regulatory effect profile, and wherein the set of features are cell-type specific.

In yet a further embodiment, the regulatory profile is the chromatin regulatory effect profile, and wherein the set of features include at least one of: sites of chromatin accessibility, chromatin marks, and transcription factor binding sites.

In an even further embodiment, the chromatin regulatory effect profile is determined utilizing at least one epigenetic assay selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and a methyl array.

In yet an even further embodiment, the regulatory profile is the RBP and RNA element profile, and wherein the set of features are cell-type specific.

In still yet an even further embodiment, the regulatory profile is the RBP and RNA element profile, and wherein the set of features include RBP binding sites.

In still yet an even further embodiment, the RBP and RNA element profile is determined utilizing at least one RNA-binding assays selected from a group consisting of: cross-linking immunoprecipitation sequencing (CLIP-seq) and RNA immunoprecipitation sequencing (RIP-seq).

In still yet an even further embodiment, the genetic material is one of: a whole genome or a partial genome

In still yet an even further embodiment, the genetic material is obtained from a biopsy of the individual.

In still yet an even further embodiment, the sequencing performed is one of: whole genome sequencing or capture sequencing.

In still yet an even further embodiment, the biochemical regulatory process is selected from a group consisting of: transcriptional regulation, posttranscriptional regulation, and translational regulation.

In still yet an even further embodiment, the identified set of variants include at least one de novo variant.

In still yet an even further embodiment, the identified set of variants include at least one inherited variant.

In still yet an even further embodiment, at least one locus the set of genomic loci is determined based upon the pathogenicity results of applying the trained computational model to a set a variants that have been identified for a collection of individuals having been diagnosed for autism spectrum disorder.

In still yet an even further embodiment, at least one locus the set of genomic loci is identified experimentally to be associated with autism spectrum disorder.

In still yet an even further embodiment, the computational model is a linear regression.

In still yet an even further embodiment, the linear regression model is L2 regularized.

In still yet an even further embodiment, the diagnosis is determined based upon a threshold, and wherein when the individual's cumulative pathogenicity score is above a threshold, the individual is determined to have a propensity for autism spectrum disorder is determined.

In still yet an even further embodiment, treating the individual comprises administering at least one of: behavioral therapy, communication therapy, educational therapy, and risperidone.

In still yet an even further embodiment, behavioral therapy is administered and includes teaching the individual behavioral skills across different settings and reinforcing desirable characteristics.

In still yet an even further embodiment, communication therapy is administered and includes performing speech and language pathology to improve development of language and communication skills.

In still yet an even further embodiment, educational therapy is administered and includes enrolling the subject in special education classes.

In still yet an even further embodiment, the set of set of known pathogenic variants is derived from the Human Gene Mutation Database.

In still yet an even further embodiment, the set of null variants is derived from at least one of: the International Genome Sample Resource (IGSR) 1000 Genomes project, a set of common variants with no expected pathogenicity, a set of variants randomly generated by in silico methods.

In an embodiment for evaluating genetic data to determine biochemical regulatory effects of variants, using computer systems, a neural network computational model is trained to yield a composite of biochemical regulatory effects. The biochemical regulatory effects are one of: effects on transcriptional regulation or effects on posttranscriptional regulation. The deep neural network computational model is trained utilizing a set of features of a regulatory effect profile. The regulatory effect profile is one of: a chromatin regulatory effect profile and a RNA binding protein (RBP) and RNA element profile. Using computer systems, genetic data of a collection of individuals is obtained. Using computer systems, a set of variants is identified within the genetic data of the collection of individuals. Using computer systems and the trained neural network computational model, he biochemical regulatory effects of each variant of the set variants is determined.

In another embodiment, the collection of individuals share a complex trait and each individual has been diagnosed as having the complex trait.

In yet another embodiment, the collection of individuals are unaffected and each individual has not been diagnosed as having the complex trait.

In a further embodiment, the neural network is a deep neural network.

In still yet another embodiment, the neural network is a convolutional neural network.

In yet a further embodiment, the regulatory profile is the chromatin regulatory effect profile, and wherein the set of features are cell-type specific.

In an even further embodiment, the regulatory profile is the chromatin regulatory effect profile, and wherein the set of features include at least one of: sites of chromatin accessibility, chromatin marks, and transcription factor binding sites.

In yet an even further embodiment, the chromatin regulatory effect profile is determined utilizing at least one epigenetic assay selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and a methyl array.

In still yet an even further embodiment, the regulatory profile is the RBP and RNA element profile, and wherein the set of features are cell-type specific.

In still yet an even further embodiment, the regulatory profile is the RBP and RNA element profile, and wherein the set of features include RBP binding sites.

In still yet an even further embodiment, the RBP and RNA element profile is determined utilizing at least one RNA-binding assays selected from a group consisting of: cross-linking immunoprecipitation sequencing (CLIP-seq) and RNA immunoprecipitation sequencing (RIP-seq).

In still yet an even further embodiment, the genetic material is one of: a whole genome or a partial genome

In still yet an even further embodiment, the genetic material is obtained from a biopsy of each individual of the collection of individuals.

In still yet an even further embodiment, the identified set of variants includes at least one de novo variant.

In still yet an even further embodiment, the identified set of variants includes at least one inherited variant.

In still yet an even further embodiment, a biochemical assay is performed to further assess at least one variant of the set variants, wherein the biochemical assay assesses one of: transcription, RNA processing, translation, or cell function.

In still yet an even further embodiment, the biochemical assay is selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis, qPCR, RNA hybridization, cross-linking immunoprecipitation sequencing (CLIP-seq), RNA immunoprecipitation sequencing (RIP-seq), RNA-seq, western blot, immunodetection, flow cytometry, enzyme-linked immunosorbent assay (ELISA), and mass spectrometry.

In an embodiment for evaluating pathogenicity of variants, using computer systems, a linear regression model is trained to yield a pathogenicity of a variant based on the variant's effect on biochemical regulation. The pathogenicity of the variant is based upon an aggregation of the effects upon the at least one biochemical regulatory process. The computational model is trained utilizing a set of known pathogenic variants and a set of null variants. The effects on biochemical regulation has been determined for each variant of the set of pathogenic variants and of the set of null variants. Using the computer systems, a set of variants to determine pathogenicity is obtained. The effects on biochemical regulation has been determined for each variant of the set of variants to determine pathogenicity. Using the computer systems and the trained linear regression model, the pathogenicity of each variant of the set of variants is determined.

In another embodiment, the effects of biochemical regulation have been determined by a neural network computational model, wherein the biochemical regulatory effects are one of: effects on transcriptional regulation or effects on posttranscriptional regulation, wherein the deep neural network computational model is trained utilizing a set of features of a regulatory effect profile, and wherein the regulatory effect profile is one of: a chromatin regulatory effect profile and a RNA binding protein (RBP) and RNA element profile.

In yet another embodiment, the neural network is a deep convolutional neural network.

In a further embodiment, the linear regression model is L2 regularized

In still yet another embodiment, the biochemical regulatory process is selected from a group consisting of: transcriptional regulation, posttranscriptional regulation, and translational regulation.

In yet a further embodiment, the set of known pathogenic variants is retrieved from the Human Gene Mutation Database.

In an even further embodiment, the set of null variants is derived from at least one of: the International Genome Sample Resource (IGSR) 1000 Genomes project, a set of common variants with no expected pathogenicity, a set of variants randomly generated by in silico methods.

In yet an even further embodiment, each variant of the obtained set of variants is associated with a complex trait.

In still yet an even further embodiment, the complex trait is a medical disorder.

In still yet an even further embodiment, the obtained set of variants is derived from a collection of individuals, and wherein each individual of the collection of individuals share the complex trait.

In still yet an even further embodiment, each obtained variant's pathogenicity is aggregated to achieve a cumulative pathogenicity score for the set of obtained variants.

In still yet an even further embodiment, the obtained set of variants includes at least one de novo variant.

In still yet an even further embodiment, the obtained set of variants includes at least one inherited variant.

In still yet an even further embodiment, a biochemical assay is performed to further assess at least one variant of the set variants, wherein the biochemical assay assesses one of: transcription, RNA processing, translation, or cell function.

In still yet an even further embodiment, the biochemical assay is selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis, qPCR, RNA hybridization, cross-linking immunoprecipitation sequencing (CLIP-seq), RNA immunoprecipitation sequencing (RIP-seq), RNA-seq, western blot, immunodetection, flow cytometry, enzyme-linked immunosorbent assay (ELISA), and mass spectrometry.

In an embodiment to develop a molecular assay to detect the presence of variants in pathogenic loci, using computer systems and a computational model, the pathogenicity of each variant of a first set of variants is determined. The pathogenicity is determined by the computational model and is based upon the variant's cumulative effects on a set of biochemical regulations. The computational model is trained utilizing a set of known pathogenic variants and a set of null variants. A set of genomic loci is identified. Each genetic locus spans across at least one variant of a second set of variants. The second set of variants is at least a subset of the first set of variants.

In another embodiment, the second set of variants are selected based on their pathogenicity. A set of nucleic acid oligomers is synthesized such that the set of nucleic acid oligomers can be utilized in a molecular assay to detect the presence of variants within the set of identified genomic loci.

In yet another embodiment, the computational model is a linear regression model.

In a further embodiment, the linear regression model is L2 regularized.

In still yet another embodiment, the effects of biochemical regulation have been determined by a neural network computational model, wherein the biochemical regulatory effects are one of: effects on transcriptional regulation or effects on posttranscriptional regulation, wherein the deep neural network computational model is trained utilizing a set of features of a regulatory effect profile, and wherein the regulatory effect profile is one of: a chromatin regulatory effect profile and a RNA binding protein (RBP) and RNA element profile.

In yet a further embodiment, the neural network is a deep convolutional neural network.

In an even further embodiment, the biochemical regulatory process is selected from a group consisting of: transcriptional regulation, posttranscriptional regulation, and translational regulation.

In yet an even further embodiment, the set of null variants is derived from at least one of: the International Genome Sample Resource (IGSR) 1000 Genomes project, a set of common variants with no expected pathogenicity, a set of variants randomly generated by in silico methods.

In still yet an even further embodiment, each variant of the first set of variants is associated with a complex trait.

In still yet an even further embodiment, the complex trait is a medical disorder.

In still yet an even further embodiment, the obtained set of variants is derived from a collection of individuals, and wherein each individual of the collection of individuals share the complex trait.

In still yet an even further embodiment, the second set of variants includes at least one de novo variant.

In still yet an even further embodiment, the second set of variants includes at least one inherited variant.

In still yet an even further embodiment, the pathogenicity of each variant of the second set of variants is greater than a threshold.

In still yet an even further embodiment, the molecular assay is capture sequencing and the set of nucleic acid oligomers is capable of hybridizing to the set of identified genomic loci.

In still yet an even further embodiment, the molecular assay is a single nucleotide polymorphism (SNP) array and the set of nucleic acid oligomers is capable of hybridizing to the set of identified genomic loci.

In still yet an even further embodiment, the molecular assay is a sequencing assay and the set of nucleic acid oligomers is capable of amplifying the set of identified genomic loci by polymerase chain reaction (PCR).

In an embodiment, a kit to detect the presence of variants within pathogenic loci includes a set of nucleic acid oligomers to detect the presence of variants within a set of genomic loci. The set of genomic loci have been identified to have harbored a pathogenic variant. The pathogenicity of each pathogenic variant is determined by a computational model and is based upon cumulative effects on a set of biochemical regulations. The computational model is trained utilizing a set of known pathogenic variants and a set of null variants. Each locus the set of genomic loci is selected based upon the pathogenicity of the pathogenic variant it has been identified to have harbored.

In another embodiment, the computational model is a linear regression model.

In yet another embodiment, the linear regression model is L2 regularized.

In a further embodiment, the effects of biochemical regulation have been determined by a neural network computational model, wherein the biochemical regulatory effects are one of: effects on transcriptional regulation or effects on posttranscriptional regulation, wherein the deep neural network computational model is trained utilizing a set of features of a regulatory effect profile, and wherein the regulatory effect profile is one of: a chromatin regulatory effect profile and a RNA binding protein (RBP) and RNA element profile.

In still yet another embodiment, the neural network is a deep convolutional neural network.

In yet a further embodiment, the biochemical regulatory process is selected from a group consisting of: transcriptional regulation, posttranscriptional regulation, and translational regulation.

In an even further embodiment, the set of known pathogenic variants is retrieved from the Human Gene Mutation Database.

In yet an even further embodiment, the set of null variants is derived from at least one of: the International Genome Sample Resource (IGSR) 1000 Genomes project, a set of common variants with no expected pathogenicity, a set of variants randomly generated by in silico methods.

In still yet an even further embodiment, each pathogenic variants is associated with a complex trait.

In still yet an even further embodiment, the complex trait is a medical disorder.

In still yet an even further embodiment, at least one pathogenic variant is a de novo variant.

In still yet an even further embodiment, at least one pathogenic variant is inherited.

In still yet an even further embodiment, the pathogenicity of each pathogenic variant is greater than a threshold.

In still yet an even further embodiment, the set of nucleic acid oligomers is capable of hybridizing to the set of genomic loci for use in a capture sequencing assay.

In still yet an even further embodiment, the set of nucleic acid oligomers is capable of hybridizing to the set of genomic loci for use in a single nucleotide polymorphism (SNP) array.

In still yet an even further embodiment, the set of nucleic acid oligomers is capable of amplifying the set of genomic loci for use in a sequencing assay.

In an embodiment to treat an individual with a medication, genetic material of an individual that includes a set of genomic loci is sequenced. Each locus of the set of genomic loci contains sequence that has been determined to harbor a pathogenic variant that affects at least one biochemical regulatory process. The effect of harboring a pathogenic variant within each genomic loci has been associated with the ability to metabolize a medication as determined by the effects of the variant on the at least one biochemical regulatory process. A set of variants that reside within the set of genomic loci sequenced is identified. A trained computational model to determine pathogenicity of each variant of the set of variants identified is obtained. The pathogenicity of each variant is based upon an aggregation of the variant's effects upon the at least one biochemical regulatory process. The computational model is trained utilizing a set of known pathogenic variants and a set of null variants. Utilizing the trained computational model, a diagnosis of the individual is determined based upon a cumulative pathogenicity score of the individual. The diagnosis indicates an ability to metabolize the medication. The cumulative pathogenicity score is determined by aggregating pathogenicity of the individual's variants within the set of genomic loci. When the individual is determined to have a diagnosis indicating a reduced ability to metabolize the medication, a lower dose of the medication or an alternative medication is administered.

In another embodiment, the medication is selected from the group consisting of: abacavir, acenocoumarol, allopurinol, am itriptyline, aripiprazole, atazanavir, atomoxetine, azathioprine, capecitabine, carbamazepine, carvedilol, cisplatin, citalopram, clomipramine, clopidogrel, clozapine, codeine, daunorubicin, desflurane, desipramine, doxepin, duloxetine, enflurane, escitalopram, esomeprazole, flecainide, fluoruracil, flupenthixol, fluvoxamine, flibenclamide, glicazide, glimepiride, haloperidol, halothane, imipramine, irinotecan, isoflurane, ivacaftor, lansoprazole, mercaptopurine, methoxyflurane, metoprolol, mirtazpine, moclobemide, nortriptyline, olanzapine, omeprazole, ondansetron, oxcarbazepine, oxycodone, pantoprazole, paroxetine, peginterferon alpha-2a, pegineterferon alpha-2b, phenprocoumon, phenytoin, propafenone, rabeprazole, raburicase, ribavirin, risperidone, sertraline, sevoflurane, simvastin, succinylcholine, tacrolimus, tamoxifen, tegafur, thioguanine, tolbutamide, tramadol, trimipramine, tropisetron, venlafaxine, voriconazole, warfarin, and zuclopenthixol.

In yet another embodiment, the medication is risperidone. Low biochemical activity of the gene CYP2D6 indicates the reduced ability to metabolize risperidone.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a process to determine pathogenicity of variants in relation to a trait in accordance with an embodiment of the invention.

FIG. 2 provides a process to determine transcriptional and/or posttranscriptional regulatory effects of variants in accordance with an embodiment of the invention.

FIG. 3 provides a process to determine pathogenicity of a set of regulatory variants associated with a trait in accordance with various embodiments of the invention.

FIG. 4A provides a process to determine the transcriptional and/or posttranscriptional regulatory effects of an individual's variants in accordance with an embodiment of the invention.

FIG. 4B provides a process to determine the trait pathogenicity of an individual's regulatory variants in accordance with an embodiment of the invention.

FIG. 5 provides a process to diagnose and treat an individual in regards to a particular trait based upon the cumulative pathogenicity of the individual's variants in accordance with an embodiment of the invention.

FIG. 6 provides an illustration of computer systems for various applications in accordance with various embodiments of the invention.

FIG. 7 provides an illustration of a process to determine regulatory effects of ASD variants and determine disease impact scores that represent pathogenicity in accordance with various embodiments of the invention.

FIG. 8 provides a graph detailing the performance of a new model with more features, generated in accordance with various embodiments of the invention.

FIG. 9 provides accuracies of DNA models as evaluated by whole chromosome holdout, generated in accordance with various embodiments of the invention.

FIG. 10 provides a graph comparing de novo mutation type of probands and unaffected siblings, utilized in accordance with a number of embodiments of the invention.

FIG. 11 provides conceptualization of transcriptional and posttranscriptional impacts of proband and unaffected sibling variants, generated in accordance with various embodiments of the invention.

FIG. 12 provides graphs detailing disease impact scores as determined by variants that affect transcriptional and posttranscriptional regulation, generated in accordance with various embodiments of the invention.

FIG. 13 provides observed p-value as compared to expected p-value of biochemical disruptions as determined by variants that affect transcriptional regulation, generated in accordance with several embodiments of the invention.

FIG. 14 provides observed p-value as compared to expected p-value of biochemical disruptions as determined by variants that affect posttranscriptional regulation, generated in accordance with several embodiments of the invention.

FIG. 15 provides graphs detailing disease impact scores as determined by variants that affect transcriptional and posttranscriptional regulation, generated in accordance with various embodiments of the invention.

FIG. 16 provides graphs comparing observed and expected disease impact scores and a graph comparing observed and expected mutation count based on parental age, utilized in accordance with various embodiments of the invention. DNA impact scores

FIG. 17 provides a schematic of alternative splicing exon region regulatory regions, utilized in accordance with various embodiments of the invention.

FIG. 18 provides a graph detailing genomic variant set analysis of mutational burden for transcriptional and posttranscriptional disruptions, generated in accordance with various embodiments of the invention.

FIG. 19 provides graphs detailing disease impact scores as determined by variants that affect transcriptional and posttranscriptional regulation in various SSC cohorts, generated in accordance with various embodiments of the invention.

FIG. 20 provides a graph detailing average disease odds ratio in relation to average disease impact score per individual, generated in accordance with various embodiments of the invention.

FIG. 21 provides a graph detailing mutation burden in various tissues comparing probands and unaffected siblings, generated in accordance with various embodiments of the invention.

FIG. 22 provides a schematic overview of network-based differential enrichment test, utilized in accordance with various embodiments of the invention.

FIG. 23 provides a graph detailing mutation burden in various molecular processes comparing probands and unaffected siblings, generated in accordance with various embodiments of the invention.

FIG. 24 provides a neighborhood map detailing genes with significant network neighborhood excess of high-impact proband mutations form two functionally coherent clusters, generated in accordance with various embodiments of the invention.

FIG. 25 provides a graph detailing experimentally-determined differential expression of various genomic regions with predicted high impact mutations between proband and siblings, generated in accordance with various embodiments of the invention.

FIG. 26 provides experimental data detailing differential splicing of the gene SMEK1 between unaffected siblings and probands, generated in accordance with various embodiments of the invention.

FIG. 27 provides a graph associating IQ with de novo coding mutation effect, utilized in accordance with various embodiments of the invention.

FIG. 28 provides graphs associating IQ with de novo mutations that affect transcriptional and posttranscriptional regulation, generated in accordance with various embodiments of the invention.

FIG. 29 provides a data graph evaluating different sequence context windows for Seqweaver RBP models, utilized in accordance with various embodiments of the invention.

FIG. 30 provides a schematic diagram of Seqweaver in accordance with various embodiments of the invention.

FIG. 31 provides a graph of aggregate accuracy of RBP models, generated in accordance with various embodiments of the invention.

FIG. 32 provides an image of CLIP autoradiogram showing separation of radiolabeled nElavl-RNA complexes, generated in accordance with various embodiments of the invention.

FIG. 33 provides a graph detailing the accuracy of Seqweaver trained on mouse data to call human variants, generated in accordance with various embodiments of the invention.

FIG. 34 provides a graph detailing the ability of Seqweaver to prioritize deleterious SNPs that exhibited evidence of selection, generated in accordance with various embodiments of the invention.

FIG. 35 provides a graph detailing total number of de novo mutations in probands and unaffected siblings, generated in accordance with various embodiments of the invention.

FIGS. 36 and 37 each provides a graph detailing posttranscriptional mutation dysregulation in probands and unaffected siblings, generated in accordance with various embodiments of the invention.

FIG. 38 provides a graph detailing enrichment of noncoding de novo mutations that affect posttranscriptional regulation in constrained genes and FMRP targets, generated in accordance with various embodiments of the invention.

FIG. 39 provides a graph detailing enrichment of large effect noncoding de novo RRD mutation in LGD genes, generated in accordance with various embodiments of the invention.

FIG. 40 provides a graph detailing enrichment of large effect noncoding de novo RRD mutation in schizophrenia coding LGD genes, generated in accordance with various embodiments of the invention.

FIG. 41 provides a graph detailing FMRP targets and constrained genes noncoding de novo RRD mutation burden in alternatively spliced exonic regions, generated in accordance with various embodiments of the invention.

FIG. 42 provides data graphs and schematics of the spliceosome component EFTUD2 and SFB4 ASD burden among FMRP targets, generated in accordance with various embodiments of the invention.

FIG. 43 provides a graph detailing the clustering of noncoding de novo mutations that affect posttranscriptional regulation among functional processes, generated in accordance with various embodiments of the invention.

FIG. 44 provides a graph highlighting autism risk signature in genes harboring proband de novo mutations in various developmental stages, generated in accordance with various embodiments of the invention.

FIG. 45 provides a graph detailing de novo mutations that affect posttranscriptional regulation in male and female probands, generated in accordance with various embodiments of the invention.

FIG. 46 provides a graphs detailing de novo mutations that affect posttranscriptional regulation of probands having various social parameters and I.Q., generated in accordance with various embodiments of the invention.

FIG. 47 provides a graph detailing parent age at proband birth and predicted effect of noncoding de novo RRD mutations, generated in accordance with various embodiments of the invention.

FIG. 48 provides a graphs detailing de novo mutations that affect posttranscriptional regulation of probands having various verbal communication skills, generated in accordance with various embodiments of the invention.

### DETAILED DESCRIPTION

Turning now to the drawings and data, a number of processes for genetic data extrapolation that can be utilized in diagnostics, medicament development, and/or treatments in accordance with various embodiments of the invention are illustrated. Numerous embodiments are directed towards a general framework and methods for scoring the functional impact of variants from genetic data. In several embodiments, methods are utilized to determine biochemical regulatory effects of genetic variants in various regions of a genome, including noncoding regions. In various embodiments, methods further use biochemical regulatory effect scores to infer variant pathogenicity scores. In some embodiments, the trait to be examined is a medical disorder and thus a trait pathogenicity score infers diagnostic and medical information. In some embodiments, methods utilize an individual's genetic information to determine biochemical impact of genetic variants of an individual's genome in order to diagnose the individual. And in some embodiments, an individual can be treated based on her diagnosis.

Great progress has been made in the past decade in understanding genetics of complex traits (e.g., autism spectrum disorder (ASD), bipolar disorder, coronary artery disease, diabetes, stroke, and schizophrenia), establishing that particular variants, including copy number variants (CNVs) and single nucleotide variants (SNVs) that likely disrupt protein-coding genes, as causal in the development of a complex trait. In the particular case of ASD, however, all known ASD-associated genes together explain a small fraction of new cases, and it is estimated that overall de novo protein coding mutations, including CNVs, contribute to no more than 30% of simplex ASD cases (i.e., single affected ASD individual in a family). It's been found that the vast majority of identified de novo variants are not within the coding region, yet instead located within intronic and intergenic regions. Despite their prevalence, very little is known regarding the contribution of intronic and intergenic variants to the genetic architecture of ASD and other complex traits. Mutations in coding sequences of genes are interpretable because the genetic code translates DNA mutations into changes in the protein sequence that yields predictable effects on the protein.

It has been suggested that no significant noncoding proband-specific signal was observed in the complex trait of ASD, and that any approach would require a very large cohort to detect signal. Accordingly, the challenge is to move beyond simple mutation counts, which are susceptible to both statistical power challenges and confounding factors, such as the rise in mutation counts with parental age. This difficulty is shared in other complex traits, including various psychiatric diseases, such as (for example) intellectual disabilities and schizophrenia. In fact, little is known about the contribution of noncoding rare variants or de novo mutations to human diseases beyond the less common cases with Mendelian inheritance patterns.

Herein, a potential role for variants, including noncoding variants, has been found in complex disorders, as detailed in various examples described. In fact, variants are likely to be causal in development of complex human traits. It has been found that variants within genetic regulatory regions lead to deleterious effects. Furthermore, variants can impact transcriptional and/or post-transcriptional biochemical function, resulting in causation of complex human traits. Furthermore, mutations within noncoding regions are hard to interpret because there is no “code” like the amino acid codon code, which provides an ability to predict biological effects when a mutation lies within a coding region.

A number of method embodiments have been developed to overcome the problems associated with the difficulty of identifying impactful variants of complex traits. Several of these embodiments enable comparison of variant burden between affected and unaffected individuals not simply in terms of number of variants, but in terms of their biochemical impact and overall pathogenicity (i.e., disease impact). Specifically, in some embodiments, biochemical data demarcating DNA and RNA binding protein interactions were used to train and deploy a deep convolutional-neural-network-based framework that predicts the functional and pathogenicity of variants, with independent models trained for DNA and RNA. This framework, in accordance with various embodiments, can estimate with single nucleotide resolution, the quantitative impact of each variant on transcriptional and post-transcriptional regulatory features, including histone marks, transcription factors and RNA-binding protein (RBP) profiles.

Furthermore, various embodiments are directed to examining variants using a computational model to determine transcriptional and/or posttranscriptional regulatory effect of variants. Computational models, in accordance with a number embodiments, are also used to determine a trait pathogenicity score based on cumulative transcriptional and/or posttranscriptional regulatory effect of variants. In some embodiments, an individual's genome is entered into the computational models to predict a likelihood of trait manifestation, including manifestation of medical disorders. And in several embodiments, diagnostics and/or treatments are performed based upon a likelihood of complex disease manifestation. In some embodiments, a threshold is used to diagnose and determine treatment options.

A number of embodiments are also directed to utilizing an individual's sequencing data and examining various loci known to be involved with pathogenic transcriptional and/or posttranscriptional regulatory effects associated with a trait. By examining specific loci, many embodiments determine an individual's cumulative variant pathogenicity. In some embodiments, when a trait to be examined is a medical disorder, an individual is diagnosed and treated based upon the individual's cumulative variant pathogenicity.

### Overview of Variant Biochemical Regulation and Pathogenicity

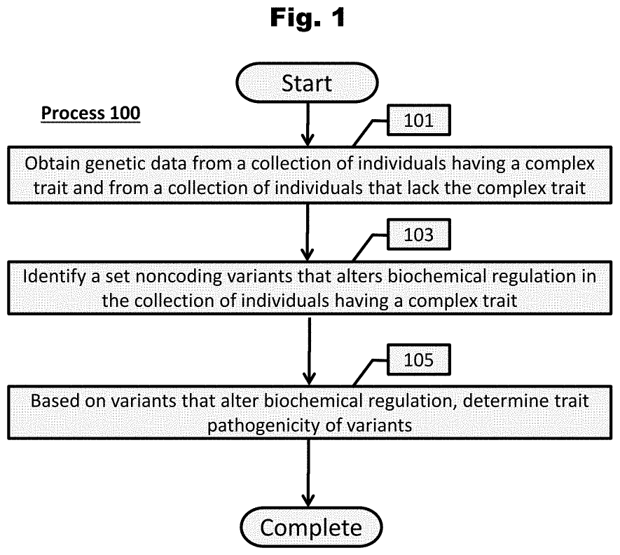

A conceptual illustration of a process to determine pathogenicity of variants related to a particular trait in accordance with an embodiment of the invention is illustrated in FIG. 1. In some embodiments, a process is utilized to identify sets of variants, including noncoding variants, that are indicative of a particular trait, as determined by their alteration of biochemical regulation. Identified variants can be used in various applications downstream in accordance with a number of embodiments of the invention, including (but not limited to) diagnosing an individual based on their genetic data.

Process 100, in accordance with a number of embodiments, begins with obtaining (101) genetic data from a collection of individuals sharing a complex trait and from a collection of unaffected individuals. In some embodiments, the individuals sharing a complex trait are probands in a simplex family. It is to be understood that a simplex family is a family with a single affected child having a complex trait and the parents and any siblings are unaffected. It should be further understood that a proband refers to the affected child, which is likely to have a set of de novo variants that in the aggregate give rise to the trait. Furthermore, it is to be understood that the aggregate of variants within the unaffected family members is unlikely to give rise to the trait.

In accordance with various embodiments, genetic data can be derived from a number of sources. In some instances, these genetic data are obtained de novo by extracting the DNA from a biological source and sequencing it. Alternatively, genetic sequence data can be obtained from publicly or privately available databases. Many databases exist that store datasets of sequences from which a user can extract the data to perform experiments upon, such as the Simons Simplex Collection. In many embodiments, the genetic sequence data include whole or partial genomes that include noncoding DNA to be examined; accordingly, any genetic data set as appropriate to the requirements of a given application could be used.

As shown in FIG. 1, sequence data to be obtained should be divided into a collection of individuals having a complex trait and a collection of unaffected individuals. The particular trait to be examined depends on the task on hand. For example, if process 100 is used to determine pathogenicity of variants of a particular medical disorder, each individual having the complex trait should be diagnosed with the disorder and each unaffected member should have not manifested the disorder.

The number of individuals within a collection can depend on the application and trait to be examined. It should be noted that increasing the number individuals in a collection can improve machine learning and variant aggregation models. Accordingly, in a number of embodiments, collections should include at least several hundred individuals.

Once genetic data are obtained, process 100 can then identify (103) a set of variants that alter biochemical regulation in the collection of individuals sharing a trait. In many embodiments, a variant is a single nucleotide variant (SNV), a copy number variant (CNV), an insertion, or a deletion. Accordingly, a profile of variants that exist all along the genetic data set can be determined for each collection of individuals.

In some embodiments, utilizing unaffected family members of simplex families, de novo variants can be determined for probands and unaffected siblings, which can be used to compare. In several embodiments, de novo noncoding variants are examined for their effect on biochemical regulation (e.g., transcriptional and/or posttranscriptional regulation). Accordingly, the biochemical effects noncoding variants of probands can be differentiated from the biochemical effects of noncoding variants of unaffected family members.

In some embodiments, a computational model is trained utilizing biochemical effect variant profiles such that the model can be used to predict the biochemical effect of variants of affected and unaffected individuals. Biochemical effect variant profile datasets can include (but are not limited to) genome-wide chromatin and RNA-binding profiles. These data sets can yield genomic loci that are important in regulating transcription and/or posttranscriptional processing.

Process 100 determines (105) trait pathogenicity of variants based on variants that alter biochemical regulation. In some embodiments, the pathogenicity of each variant from a collection of individuals is determined. In some embodiments, variant pathogenicity is aggregated to yield a pathogenicity score for a particular trait. In a number of embodiments, a computational model is utilized to determine the pathogenicity of variants, which can be trained using a set of pathogenic regulatory variants and a set of null variants.

In several embodiments, processes to determine trait pathogenicity of variants is utilized in various downstream applications, including (but not limited to) diagnosis of an individual, treatment of individual and/or development of diagnostic assays. These embodiments are described in greater detail in subsequent sections.

### Processes to Yield Transcriptional and Posttranscriptional Regulatory Effects of Variants

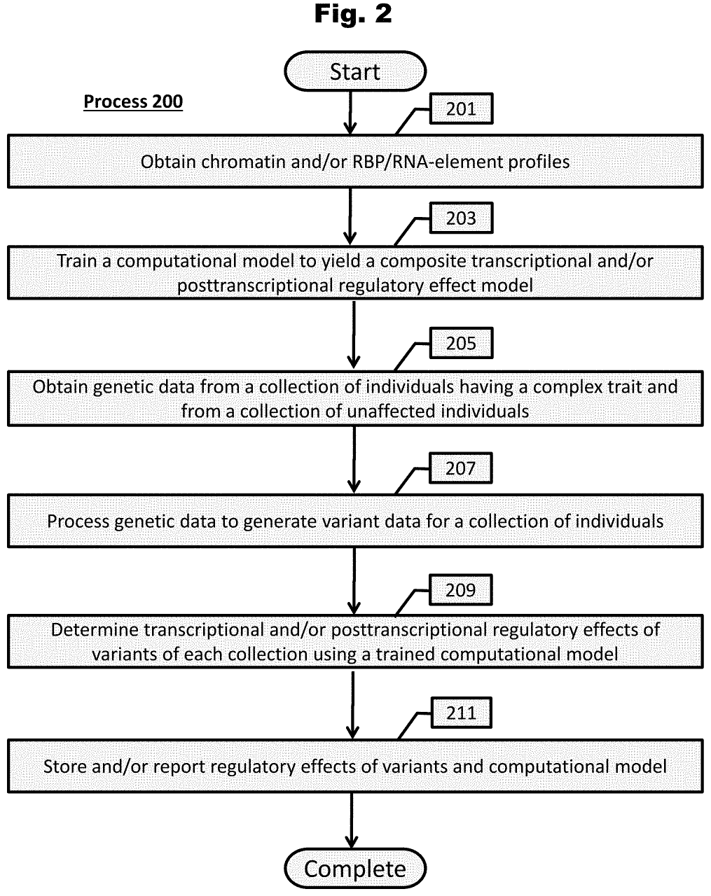

A conceptual illustration of a process to determine transcriptional and/or posttranscriptional regulatory effects of variants utilizing computing systems is provided in FIG. 2. As shown, in a number of embodiments, the process can begin with by obtaining (201) genome-wide chromatin and/or RBP and RNA element profiles. A chromatin profile is a collection of data indicating where various factors and elements that affect transcription interact with DNA along a genomic sequence. In many embodiments, chromatin features are cell-type specific and include (but are not limited to) sites of chromatin accessibility (e.g., DNase I hypersensitivity), chromatin marks (e.g., histone code), transcription factor binding sites, and other epigenetic factors. Likewise, in several embodiments, a RBP and RNA element profile is a collection of data indicating where RNA-binding proteins (RBPs) and other factors (e.g., sequences surrounding splice sites) that modulate RNA activity interact with RNA along transcriptomic sequences.

Methods to generate chromatin and RBP/RNA-element profiles are well known in the art. Generally, chromatin profiles can be determined utilizing various epigenetic assays including (but not limited to) chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and methyl array. RBP/RNA-element profiles can be determined utilizing various RNA-binding assays, including (but not limited to) cross-linking immunoprecipitation sequencing (CLIP-seq) and RNA immunoprecipitation sequencing (RIP-seq). Several databases store chromatin and RBP/RNA-element profiles which can be used, including (but not limited to) Encyclopedia of DNA Elements (ENCODE) (https://www.encodeproject.org/), NIH Roadmap Epigenomics Mapping Consortium (http://www.roadmapepigenomics.org/), and the International Human Epigenome Consortium (IHEC) (https://epigenomesportal.ca/ihec/).

Utilizing chromatin and/or RBP/RNA-element regulatory effects profiles, a computational model is trained (203) to yield a composite transcriptional and/or posttranscriptional regulatory effect model with a number of features. In several embodiments, the computational model is a deep neural network. In some embodiments, the computational model is a convolutional neural network.

Process 200 also obtains (205) genetic data from a collection of individuals having a complex trait and from a collection of unaffected individuals. The particular trait to be examined depends on the task on hand. For example, if process 200 is used to determine regulatory effects of variants of a particular medical disorder, each individual having the trait should be diagnosed with the disorder and each unaffected individual should have not manifested the disorder.

The number of individuals within a collection can depend on the application and trait to be examined. It should be noted that increasing the number individuals in a collection can improve machine learning and variant aggregation models. Accordingly, in a number of embodiments, collections should include at least several hundred individuals.

In many embodiments, genetic data to be obtained can be any sequence data that contain genetic variants, especially variants within noncoding regions. In several embodiments, genetic data are whole or partial genomes inclusive of noncoding regions. In some embodiments, sequencing data is directed to cover various regulatory regions important for the trait to be examined.

In accordance with various embodiments of the invention, genetic data can be derived from a number of sources. In some embodiments, these sources include sequences derived from DNA of a biological source that are subsequently processed and sequenced. In some embodiments, sequences are obtained from a publicly or privately available database. Many databases exist that store datasets of sequences from which a user can extract the data to perform experiments upon.

In many embodiments, biological samples of DNA can be used for sequencing that are each derived from a biopsy of an individual. In particular embodiments, the DNA to be acquired can be derived from biopsies of human patients associated with a phenotype or a disease state and derived from unaffected individuals as well. In some embodiments, DNA can be derived from common research sources, such as in vitro tissue culture cell lines or research mouse models. In many embodiments involving sample extraction, DNA molecules are extracted, processed and sequenced according to methods commonly understood in the field.

In accordance with various embodiments, genetic data are processed (207) to generate variant data for a collection of individuals. In many embodiments, variant profiles are further analyzed and trimmed, often dependent on the application. In some embodiments, variant calls within repeat regions are removed. In some embodiments, indels are removed. In some embodiments, only variants of a particular frequency (e.g., rare variants with MAF 1.0%) are examined and thus all other variants are excluded. In some embodiments, known and/or pre-classified variants from known various databases are removed. For example, when examining variants related to a disorder, it may be ideal to remove known variants that exist in databases of healthy individuals, as it may be reasonable to presume that these variants are not related to a disordered state.

In some embodiments, variant profiles are trimmed to specifically only keep de novo variants (i.e., variants that are not within parental genomes and thus arose in gametes and/or early in development). Many methods are known within the art to trim variant profiles to only de novo variants, which can be performed by a number methods. In some embodiments, the GATK pipeline is used to trim variants (https://software.broadinstitute.org/gatk/). Accordingly, de novo noncoding variant profiles can be created for various collections of individuals. In some embodiments, a de novo noncoding variant profile is generated for a collection of probands. In some embodiments, a de novo noncoding variant profile is generated for a collection of unaffected individuals. In some embodiments, a classifier can be used to score each candidate de novo noncoding variant to obtain a comparable number of high-confidence de novo noncoding variant calls. In some embodiments, the classifier DNMFilter (https://github.com/yongzhuang/DNMFilter) is used to score candidate de novo noncoding variants, utilizing an appropriate threshold of probability (e.g., >0.75; or e.g., >0.5) as determined for each experimental set of variant collections

Process 200 also utilizes variants of a collection of individuals and the trained model of step 203 to determine (209) transcriptional and/or posttranscriptional regulatory effects of the variants. Accordingly, variants that affect transcriptional and/or posttranscriptional regulation are likely causal in complex trait manifestation.

In accordance with several embodiments, variant profiles of collections of individuals, their regulatory effects, and the computational model are stored and/or reported (211). In some embodiments, these profiles and regulatory effects may be used in many further downstream applications, including (but not limited to) identifying regions of regulation that are often affected in a complex trait and determining variant pathogenicity.

While a specific example of a process for determining transcriptional and/or posttranscriptional regulatory effects of variants is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

### Processes to Yield Pathogenicity Scores

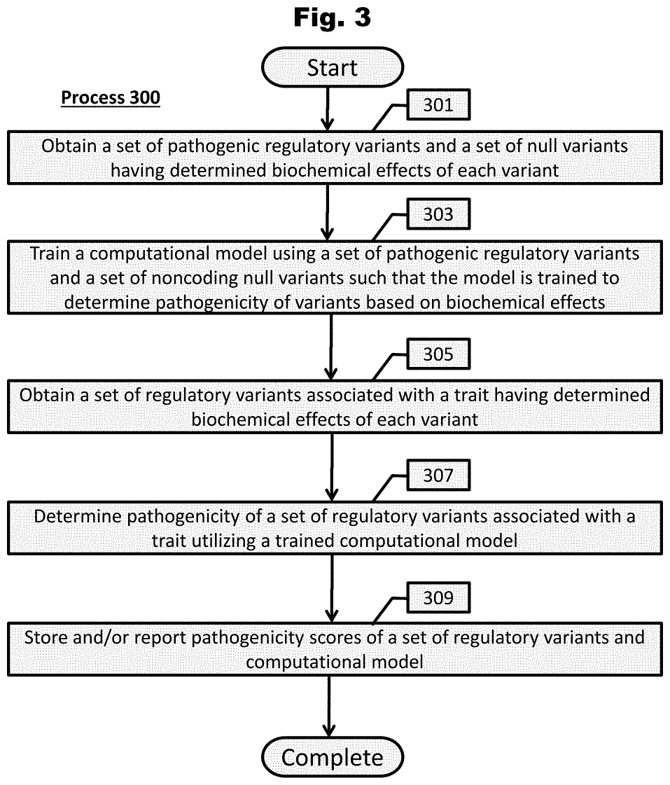

Depicted in FIG. 3 is a conceptual illustration of a process to determine pathogenicity of a set of regulatory variants via a machine-learning framework, which can performed on various computing systems. The process utilizes the regulatory effects of individual variants to determine their individual pathogenicity towards a complex trait, which can be aggregated to determine the pathogenicity of a set of variants.

Process 300 can begin with obtaining (301) a set of pathogenic regulatory variant and a set of null variants (i.e., variants not determined to be a pathogenic regulatory variant). In some embodiments, pathogenic regulatory variants are retrieved from an appropriate database, such as (for example) the Human Gene Mutation Database. Pathogenic regulatory variants should be variants annotated as “regulatory” and known to be involved in pathogenesis of a trait (e.g., medical disorder). In a number of embodiments, null variants are any variants that is not involved with pathogenesis of trait. In some instances, null variants are retrieved from healthy individuals such as (for example) data of the International Genome Sample Resource (IGSR) 1000 Genomes project (http://www.internationalgenome.org/). In some instances, null variants are common variants with no expected pathogenicity are used. In some instances, null variants are generated randomly by in silico methods.

In several embodiments, a set of pathogenic regulatory variant and a set of null variants each have determined biochemical effects. In some embodiments, biochemical effects include transcriptional and/or posttranscriptional effects. In some embodiments, transcriptional and/or posttranscriptional effects are determined as described in FIG. 2. In some embodiments, biochemical effects include translational effects that arise amino acid coding sequence alterations (e.g., missense, nonsense mutations, and in-frame indels). It should be noted however, that any appropriate biochemical effect and any appropriate method to determine biochemical effects may be used within various embodiments.

A set of pathogenic regulatory variants and a set of null variants are used to train (303) a computational model to be able to determine pathogenicity of variants based on the variant's aggregated biochemical effects. In several embodiments, a pathogenicity computational model is trained to delineate which biochemical effects are associated with pathogenic variants as opposed to null variants. In many embodiments, a linear regression model is used. In some instances, a linear regression model is L2 regularized and trained using an appropriate package, such as (for example) the xgboost package (https://github.com/dmlc/xgboost). In some embodiments, predicted probabilities are z-transformed to have a particular mean and standard deviation.

Process 300 also obtains (305) a set of regulatory variants associated with a trait, each variant having a determined biochemical effect. A set of regulatory variants can be any set to be examined. In some instances, a set of regulatory variants are associated with a particular medical disorder. In some instances, a set of regulatory variants are associated with ASD. In some instances, a set of regulatory variants and their biochemical effects are determined in accordance with Process 200 described herein. In some instances, a set of regulatory variants are associated with traits shared by a collection of individuals. In some instances, a set of regulatory variants are associated with unaffected individuals, which can be useful for comparing pathogenicity of variants associated with a trait.

Utilizing the trained computational model of Step 303, the pathogenicity of each variant of a set of regulatory variants is determined (307) based upon each variant's aggregated biochemical effect. In some embodiments, a cumulative pathogenicity score for each trait is determined. In some embodiments, a cumulative pathogenicity score for a set of variants is determined by various statistical methods, which may include an aggregate score. In some embodiments, a pathogenicity score is compared between a set of trait associated variants and a set of null variants.

Pathogenicity scores of a set of regulatory variants and a trained computational model is stored and/or reported (309). In a number of embodiments, pathogenicity scores of a set of regulatory variants are used in a number of downstream applications, including (but not limited to) clinical classification of individuals (e.g., clinical diagnostics), further molecular research into the trait, and identification of functionality and tissue specificity. In many embodiments, a trained classification model is used to classify individuals in regards to a trait.

While a specific example of a process for determining pathogenicity scores of regulatory variants is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

### Processes to Interpret Regulatory Effects and Pathogenicity of an Individual's Variants

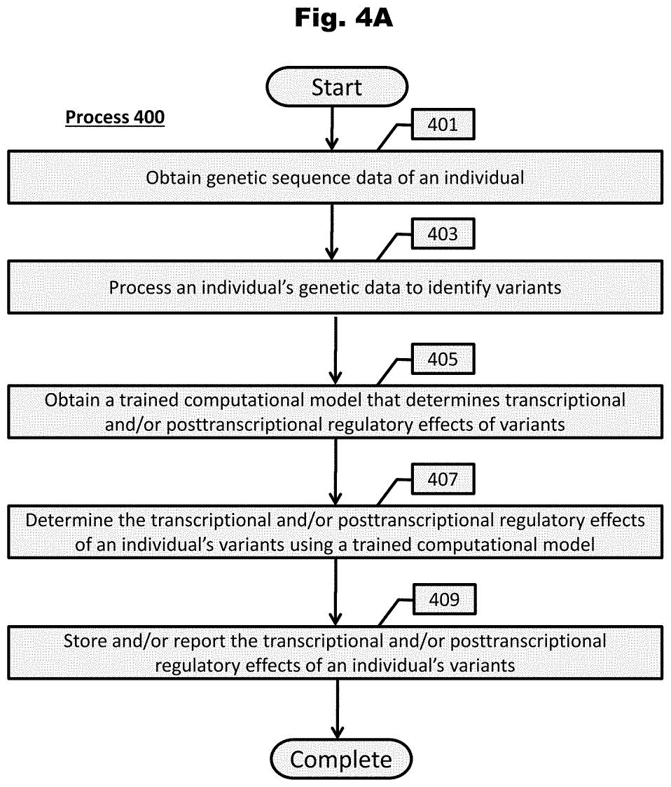

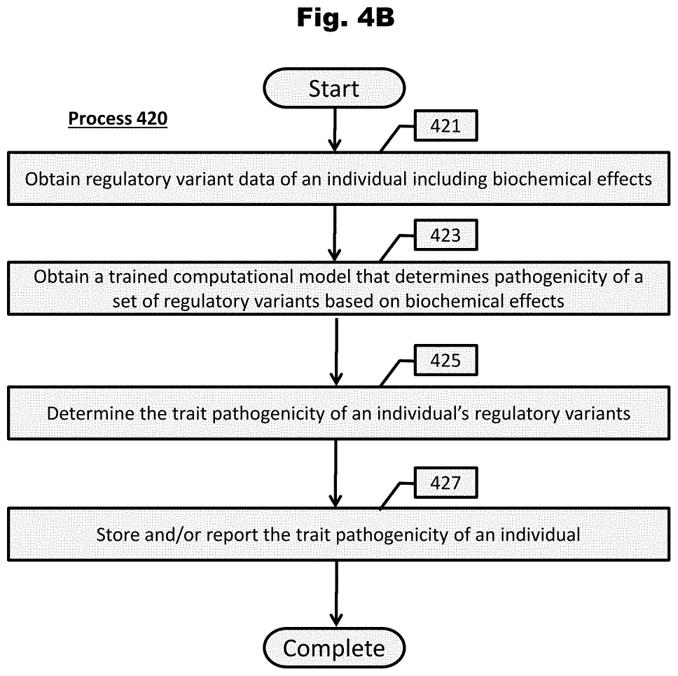

FIG. 4A provides a conceptual illustration of a process to determine the transcriptional and/or posttranscriptional regulatory effects of an individual's variants via computer systems using the individual's genetic sequence data and a trained computational model. Various embodiments utilize this process to classify an individual based upon the individual's variants and their effects on transcriptional and/or posttranscriptional regulation.

As shown in FIG. 4A, Process 400 obtains (401) an individual's genetic sequence data. The data, in accordance with many embodiments, is any DNA sequence data of individual that is inclusive of regulatory regions to be analyzed. In some embodiments, genetic data is an individual's whole genome, a partial genome, or other data that is directed towards the regulatory regions of an individual's sequence and is inclusive of variant data. In some embodiments, genetic data is only sequencing data on a set of regulatory loci that have been found to be important to the trait to be analyzed (e.g., capture sequencing). In some embodiments, sequence data are obtained by a biopsy of an individual, in which genetic material is extracted and sequenced in accordance with various protocols known in the art.

In accordance with various embodiments, an individual's genetic sequence data are processed (403) to identify variants. In many embodiments, an individual's variant profile is further analyzed and trimmed, often dependent on the application. In some embodiments, variant calls within repeat regions are removed. In some embodiments, indels are removed. In some embodiments, only variants of a particular frequency (e.g., rare variants with MAF≤1.0%) are examined and thus all other variants are excluded. In some embodiments, known and/or pre-classified variants from known various databases are removed. For example, when examining variants related to a disorder, it may be ideal to remove known variants that exist in databases of healthy individuals, as it may be reasonable to presume that these variants are not related to a disordered state.

In some embodiments, variant profiles of an individual are trimmed to specifically only keep de novo variants (i.e., variants that are not within parental genomes and thus arose in gametes and/or early in development). Many methods are known within the art to trim variant profiles to only de novo variants, which can be performed by a number methods. In some embodiments, the GATK pipeline is used to trim variants (https://software.broadinstitute.org/gatk/). In some embodiments, a classifier can be used to score each candidate de novo variant to obtain a comparable number of high-confidence de novo variant calls. In some embodiments, the classifier DNMFilter (https://github.com/yongzhuang/DNMFilter) is used to score candidate de novo variants, utilizing an appropriate threshold of probability (e.g., >0.75; or e.g., >0.5) as determined for each experimental set of variant collections.

In some embodiments, a variant profile is generated for an individual with no medical diagnosis. In some embodiments, a variant profile is generated for an individual that has received a preliminary diagnosis.

A trained computational model capable of determining transcriptional and/or posttranscriptional regulatory effects of variants is also obtained (405). In some embodiments, a trained classification model is trained as shown and described in FIG. 2, however, in accordance with more embodiments, any classification model capable of determining transcriptional and/or posttranscriptional regulatory effects of variants based on genetic sequence data may be used. In a number of embodiments, an individual's genetic sequence data are entered into a computational model, wherein subsequently the transcriptional and/or posttranscriptional regulatory effects of the individual's variants are determined (407). In some embodiments, the transcriptional and/or posttranscriptional regulatory effects of variants is determined by the genomic loci of the variants, as determined by the transcriptional and/or posttranscriptional regulatory features.

The transcriptional and/or posttranscriptional regulatory effects of an individual's variants are reported and/or stored (409). In numerous embodiments, the transcriptional and/or posttranscriptional regulatory effects can be used in a number of downstream applications, which may include (but is not limited to) determining pathogenicity of the regulatory variants, which may be used for diagnosis of individuals and determination of medical intervention.

While a specific example of a process for determining the transcriptional and/or posttranscriptional regulatory effects of an individual's variants is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

FIG. 4B provides a conceptual illustration of a process to determine the trait pathogenicity of an individual's regulatory variants via computer systems using a trained computational model. Various embodiments utilize this process to determine a pathogenicity of a particular trait within an individual. For example, in some applications, process 420 can be used to determine if an individual as having a propensity for a particular disease or disorder. And in some applications, an individual can be diagnosed and/or treated utilizing various embodiments of a pathogenicity determining system.

As shown in FIG. 4B, regulatory variant data of an individual of the individual's variants are obtained (421), including each variants biochemical effect. An individual's variant data can be any variant data to be examined. In some embodiments, a set of regulatory variants are associated with a particular medical disorder. In some embodiments, a set of regulatory variants are associated with ASD. In some embodiments, a set of regulatory variants are determined in accordance with Process 400 described herein.

In several embodiments, a set of variants to be examined has biochemical effects that have been determined. In some embodiments, biochemical effects include transcriptional and/or posttranscriptional effects. In some embodiments, transcriptional and/or posttranscriptional effects are determined as described in FIG. 4A. In some embodiments, biochemical effects include translational effects that arise amino acid coding sequence alterations (e.g., missense, nonsense mutations, and in-frame indels). It should be noted however, that any appropriate biochemical effect and any appropriate method to determine biochemical effects may be used within various embodiments.

A trained computational model capable of determining pathogenicity of a set of regulatory variants based on each variant's biochemical effect is also obtained (405). In some embodiments, a trained classification model is trained as shown and described in FIG. 3, however, in accordance with more embodiments, any classification model capable of determining pathogenicity of a set of regulatory variants based on an individual's regulatory variant data may be used. In a number of embodiments, an individual's regulatory variant data are entered into a computational model, wherein subsequently the pathogenicity of the individual's regulatory variants are determined (425). In some embodiments, a pathogenicity score for each regulatory variant is determined. In some embodiments, a comprehensive pathogenicity score for a set of regulatory variants is determined by various statistical methods, which may include an aggregation of each variant's pathogenicity score. In some embodiments, a pathogenicity score is used to determine whether a particular trait is likely to manifest. In some embodiments, a threshold is used to determine whether a pathogenicity score will result in a trait. In some embodiments, a pathogenicity score is used to diagnose an individual for a trait (e.g., medical disorder). Pathogenicity scores can be especially useful to diagnose complex diseases that may arise from variants that affect transcriptional and/or posttranscriptional regulation, such as (for example) autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, and stroke.

Trait pathogenicity scores and diagnoses of an individual are stored and/or reported (427). In a number of embodiments, pathogenicity scores of a set of regulatory variants are used in a number of downstream applications, including (but not limited to) diagnoses and treatments of patients.

While a specific example of a process for classifying individuals is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

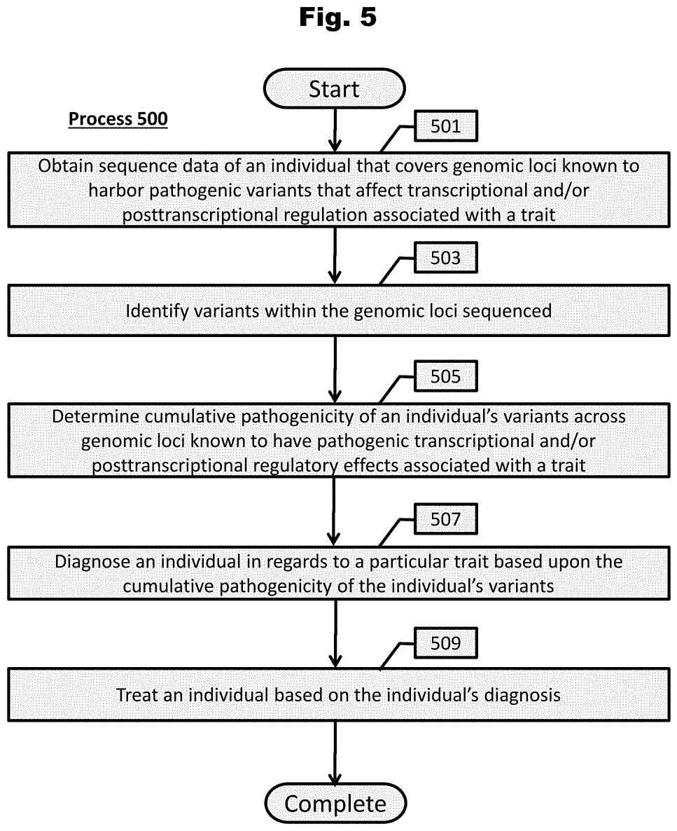

FIG. 5 provides a conceptual illustration of a process to diagnose and treat an individual utilizing pathogenicity scores across genomic loci known to harbor pathogenic variants that affect transcriptional and/or posttranscriptional regulation associated with a trait. In some applications, process 500 can be used to diagnose an individual as having a propensity for a particular disease or disorder. And in some applications, an individual can be diagnosed and/or treated, especially for complex diseases that arise due to alterations in regions that affect transcriptional and/or posttranscriptional regulation.

As shown in FIG. 5, an individual's genetic data are obtained (501). The genetic data, in accordance with many embodiments, is any DNA sequence data of an individual that covers genomic loci known to harbor at least one pathogenic variant that has an effect on a biochemical process (e.g., transcriptional and/or posttranscriptional regulation), and the effect on the biochemical process associated with a trait. In some embodiments, genetic data are an individual's whole genome or a partial genome. In some embodiments, genetic data is only sequencing data covering the genomic loci to be analyzed (e.g., capture sequencing). In some embodiments, sequence data are obtained by a biopsy of an individual, in which genetic material is extracted and sequenced in accordance with various protocols known in the art.

Genomic loci known to harbor pathogenic variants that affect transcriptional and/or posttranscriptional regulation can be identified by any appropriate method. In some instances, genomic loci are identified experimentally. In some instances, genomic loci are identified utilizing a computational model trained to determine transcriptional and/or posttranscriptional regulatory effects and/or pathogenicity of variants, such as (for example) the method portrayed in FIG. 2 or FIG. 3.

Process 500 identifies (503) variants within the genomic loci sequenced. It should be understood the variants identified can be any variant within the loci, and does not have to be the same position of previously identified pathogenic variants. In some embodiments, some of the variants are de novo (i.e., not inherited from parental genome). In some embodiments, at least some of the variants are inherited from a parental genome. In several embodiments, the pathogenicity of some of the variants identified is unknown.

Process 500 also determines (505) cumulative pathogenicity of an individual's variants across genomic loci sequenced. Pathogenicity of variants within genomic loci examined can be scored by an appropriate method. In some embodiments, pathogenicity of each variant is scored utilizing a trained computational model such as (for example) the model described in FIG. 4B. In some embodiments, a cumulative pathogenicity score for regulatory variants across the genomic loci examined is determined by various statistical methods, which may include an aggregation of each variant's pathogenicity score. In some embodiments, a pathogenicity score is used to determine whether a particular trait is likely to manifest. In some embodiments, a threshold is used to determine whether a cumulative pathogenicity score will result in a trait.

An individual is diagnosed (507) in regards to particular trait based upon the cumulative pathogenicity of the individual's variants across genomic loci examined. In some embodiments, then the cumulative pathogenicity is above a certain threshold, a diagnosis for having a particular medical disorder can be made. On the contrary, in some embodiments, when the cumulative pathogenicity is below a certain threshold, an individual is diagnosed as lacking a particular medical disorder. In some instances, a medical disorder is a spectrum and thus diagnoses can be made along the spectrum based on windows of pathogenicity scores. Based on an individual's diagnosis, the individual is treated (509). Treatment will depend on the medical disorder being diagnosed.

While a specific example of a process for diagnosing and treating individuals is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

### Systems of Variant Analysis

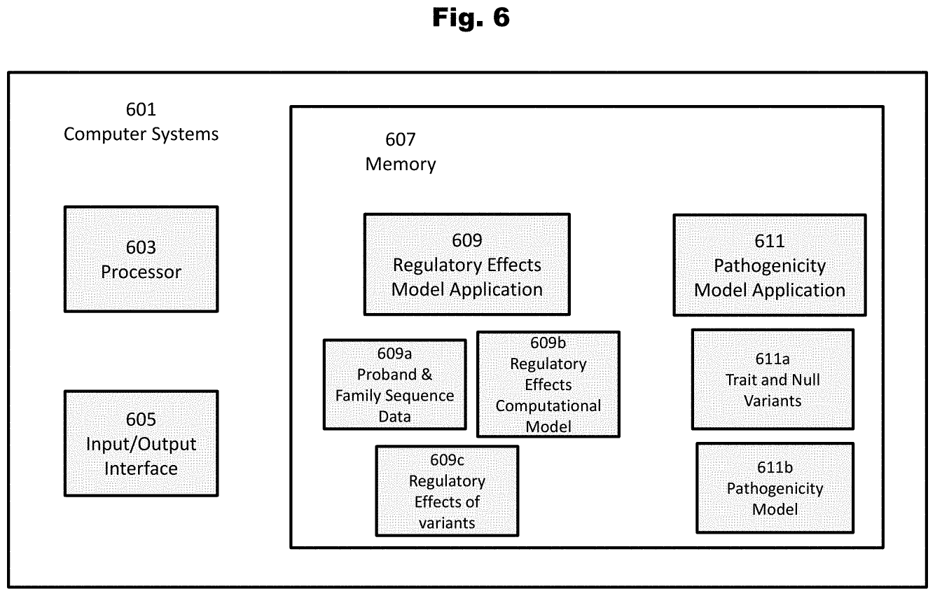

Turning now to FIG. 6, computer systems (601) may be implemented on computing devices in accordance with some embodiments of the invention. The computer systems (601) may include personal computers, a laptop computers, other computing devices, or any combination of devices and computers with sufficient processing power for the processes described herein. The computer systems (601) include a processor (603), which may refer to one or more devices within the computing devices that can be configured to perform computations via machine readable instructions stored within a memory (607) of the computer systems (601). The processor may include one or more microprocessors (CPUs), one or more graphics processing units (GPUs), and/or one or more digital signal processors (DSPs). According to other embodiments of the invention, the computer system may be implemented on multiple computers.

In a number of embodiments of the invention, the memory (607) may contain a regulatory effect model application (609) and a pathogenicity model application (611) that performs all or a portion of various methods according to different embodiments of the invention described throughout the present application. As an example, processor (603) may perform a trait-related variant analyses methods similar to any of the processes described above with reference to FIGS. 2 through 5, which involve the use of various applications such as a regulatory effects model application (609) and a pathogenicity model application (611), during which memory (607) may be used to store various intermediate processing data such as proband and family sequence data (609a), regulatory effects computational model (609b), regulatory effects of variants (609c), trait and null variants (611a), and pathogenicity model (611b).

In some embodiments of the invention, computer systems (601) may include an input/output interface (605) that can be utilized to communicate with a variety of devices, including but not limited to other computing systems, a projector, and/or other display devices. As can be readily appreciated, a variety of software architectures can be utilized to implement a computer system as appropriate to the requirements of specific applications in accordance with various embodiments of the invention.

Although computer systems and processes for variant analyses and performing actions based thereon are described above with respect to FIG. 6, any of a variety of devices and processes for data associated with variant analyses as appropriate to the requirements of a specific application can be utilized in accordance with many embodiments of the invention.

### Biochemical Analysis of Genes

A number of embodiments are directed towards biochemical assays to be performed based on the results of variants identified to affect transcriptional and/or posttranscriptional regulation and/or the results of a variant's pathogenicity. Accordingly, in several embodiments, methods are performed to determine transcriptional and/or posttranscriptional regulatory effects of variants and/or their pathogenicity, and based on those determinations a biochemical assay is performed to assess transcriptional and/or posttranscriptional regulation. In some embodiments, determination of transcriptional and/or posttranscriptional regulatory effects of variants and/or their pathogenicity by performing methods described in FIGS. 2, 3, 4A and 4B. It should be noted, however, that any method capable of determining posttranscriptional regulatory effects of variants and/or their pathogenicity can be utilized within various embodiments.

In many embodiments, biochemical methods are performed as follows:
a) obtain a set of variants (e.g., variants of an individual or collection of individuals)
b) determine transcriptional and/or posttranscriptional regulatory effects of each variant of the set of variants
c) optional: determine the pathogenicity of each variants of a set of variants
d) based on regulatory effects and/or pathogenicity of variants, perform a biochemical assay to assess transcription, RNA processing, translation, or cell function.
   In some embodiments, determination of transcriptional and/or posttranscriptional regulatory effects can be performed in accordance with either FIG. 2 or FIG. 4A. In some embodiments, determination of pathogenicity can be performed in accordance with either FIG. 3 or FIG. 4B. In some embodiments, pathogenicity scores are used to prioritize variants to be assessed. In some embodiments, a single variant is assessed. In some embodiments, a collection of variants are assessed simultaneously to determine their cumulative effect. In some embodiments, a genomic locus is assessed, in which the genomic locus was identified based on at least one determined variant effect and/or pathogenicity within that locus.

A number of biochemical assays can be performed on the basis of the determination of a variant's transcriptional and/or posttranscriptional regulatory effect and/or pathogenicity. Generally, biochemical assays will provide a more in depth assessment of variant and how it affects various biological functions, which include effects on chromatin formation, chromatin binding, nearby gene transcription, binding of RNA binding proteins, RNA stability, RNA processing, translation, cellular function, and disorder pathology. A number of biochemical assays are known in the art to assess variant effect, including (but not limited to) chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis (e.g., luciferase and eGFP), qPCR, RNA hybridization (e.g., ISH), cross-linking immunoprecipitation sequencing (CLIP-seq), RNA immunoprecipitation sequencing (RIP-seq), RNA-seq, western blot, immunodetection, flow cytometry, enzyme-linked immunosorbent assay (ELISA), and mass spectrometry.

Several embodiments are also directed towards manipulating genetic material in order to analyze variants. In some embodiments, a variant is incorporated into a plasmid construct for analysis. In some embodiments, variants are introduced into at least one allele of the DNA of a biological cell. Several methods are well known to introduce variant mutations within an allele, including (but not limited to) CRISPR mutagenesis, Zinc-finger mutagenesis, and TALEN mutagenesis. In some embodiments, a common variant is changed into rare variant. In some embodiments, a rare variant is changed into a common variant, especially when determining the effect of “correcting” a potential pathogenic variant.

Various embodiments are directed towards development of cell lines having a particular set of variants. In some embodiments, a cell line can be manipulated by genetic engineering to harbor a set of variants. In some embodiments, a cell line can be derived from an individual (e.g., from a biopsy) which would harbor the variants identified in that individual. In some embodiments, a cell line from an individual can be genetically manipulated to “correct” a set of pathogenic variants. In some embodiments, a cell line having a set pathogenic variants and a cell line having a set of control or “corrected” variants may be assessed to determine the cumulative effect of the set of variants, especially when modeling a medical disorder that is associated the set of variants.

### Diagnostics and Treatments of Complex Diseases

Various embodiments are directed to development of treatments related to diagnoses of individuals based on their regulatory variant data. As described herein, an individual may be diagnosed as having a particular trait status in relation to a disease. In some embodiments, an individual is diagnosed as having a disorder or having a high propensity for a disorder. Based on the pathogenicity of one's regulatory variant data, an individual can be treated with various medications and therapeutic regimens.

### Diagnostic Methods

A number of embodiments are directed towards diagnosing individuals using pathogenicity scores of regulatory variant data. In some embodiments, a trained pathogenicity model has been trained using genetic data of pathogenic variants. In some embodiments, genomic loci known to harbor variants that alter transcriptional and/or posttranscriptional regulation associated with a medical disorder. And in some embodiments, genomic loci known to harbor pathogenic variants are determined using a computational model utilizing genetic data of individuals known to have the medical disorder.

In a number of embodiments, diagnostics can be performed as follows:
a) obtain genetic data of the individual to be diagnosed
b) determine pathogenicity of variants that affect transcriptional and/or posttranscriptional regulation
c) diagnose the individual based on the pathogenicity of variants.
   Diagnoses, in accordance with various embodiments, can be performed as portrayed and described in any one of FIG. 4A, 4B, or 5.

Many embodiments of diagnostics improve on traditional diagnostic methods, especially in cases of complex disorders. Because the genetic contribution to complex disorders is often obscured by the fact regulatory variants are combined to yield the disorder, traditional genetic tests of examining a single gene, variant, and/or locus have been unavailable. As described herein, however, in some embodiments, a diagnosis is performed for a complex disease utilizing variant pathogenicity data aggregating techniques, such as those described in FIGS. 4A, 4B, and 5. In some embodiments, diagnoses are performed for disorders in which no single variant is diagnostic. In some embodiments, diagnoses are performed for disorders that arise at least in part by variants that affect transcriptional and/or posttranscriptional regulation. Various embodiments are directed to diagnoses of complex (i.e., multifactorial) disorders, including (but not limited to) autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, and stroke.

### Diagnostic Kits

Embodiments are directed towards genomic loci sequencing and/or single nucleotide polymorphism (SNP) array kits to be utilized within various methods as described herein. As described, various methods can diagnose an individual for a complex trait by examining variants in various regulatory genomic loci. Accordingly, a number of embodiments are directed towards genomic loci sequencing and SNP array kits that cover a set of genomic loci to diagnose a particular trait. In some instances, the set of genomic loci are identified by a computational model, such as one described in FIG. 2 and FIG. 3.

A number of targeted gene sequencing protocols are known in the art, including (but not limited to) partial genome sequencing, primer-directed sequencing, and capture sequencing. Generally, targeted sequencing involves selection step either by hybridization and/or amplification of the target sequences prior to sequencing. Therefore, embodiments are directed to sequencing kits that target genomic loci that are known to harbor pathogenic variants to diagnose a particular medical disorder.

Likewise, a number of SNP array protocols are known in the art. In general, chip arrays are set with oligo sequences having a particular SNP. Sample DNA derived from an individual can be processed and then applied to SNP array to determine sites of hybridization, indicating existence of a particular SNP. Thus, embodiments are directed to SNP array kits that target particular SNPs that known to be pathogenic in order to diagnose a particular medical disorder.

The number of genomic loci and/or SNPs to include in a sequencing kit can vary, depending on the genomic loci and/or SNPs to examine for a particular trait and the computational model to be used. In some embodiments, the genomic loci and/or SNPs to be examined are identified by a computational model, such as the computational model described in FIG. 2 and FIG. 3. In various embodiments, the number of genomic loci in a sequencing kit are approximately, 100, 1000, 5000, 10000, 20000, 30000, 40000, 50000, 60000, 70000, 80000, 90000, 100000, 150000, or 200000 loci. In various embodiments, the number of SNPs in an array kit are approximately, 1000, 10000, 50000, 100000, 200000, 300000, 400000, 500000, 600000, 700000, 800000, 900000, 1000000, 1500000, or 2000000 SNPs. In one example, as described in the exemplary embodiments, over 100000 polymorphic positions were examined in the detection of alterations in transcriptional and/or posttranscriptional regulation in the noncoding signal that contributes to ASD. In some embodiments, all identified loci are included in a kit. In some embodiments, only a subset of the loci are included. It should be understood that precise number and positions of loci can vary as the classification model can be updated with new data or recreated with a different data set (especially for different traits, and/or subtypes of traits).

Within the examples described below, a number of genomic loci and variants have been identified that are likely pathogenic in ASD. In particular, Table 3 and Electronic Data Table 3 provide a number of variants with high pathogenicity. Table 4 and Electronic Data Table 4 provide a number of gene loci regions that experience a significant burden of pathogenic variants in ASD probands. Accordingly, these identified variants and/or loci can be utilized to develop capture sequencing and/or SNP array kits. In some embodiments, capture sequencing and/or SNP array kits are developed covering regions that have high variant pathogenicity, as identified in Electronic Data Tables 3 and 4. In some of these embodiments, the variants and/or genomic loci are selected based on their statistical score of relevance and/or pathogenicity score.

### Medications and Supplements

Several embodiments are directed to the use of medications and/or dietary supplements to treat an individual based on their medical disorder diagnosis. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect.

A therapeutically effective amount can be an amount sufficient to prevent reduce, ameliorate or eliminate symptoms of disorders or pathological conditions susceptible to such treatment, such as, for example, autism, bipolar disorder, depression, schizophrenia, or other diseases that are complex. In some embodiments, a therapeutically effective amount is an amount sufficient to reduce the symptoms of a complex disorder.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of neoplastic growth) as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

A number of medications and treatments are known for several complex disorders, especially those that arise (at least in part) due to regulatory variants. Accordingly, embodiments are directed toward treating an individual with a treatment regime and/or medication when diagnosed with a complex disorder as described herein. Various embodiments are directed to treatments of complex (i.e., multifactorial) disorders, including (but not limited to autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, and stroke.

Once diagnosed for having a risk of autism spectrum disorder, medical monitoring (e.g., regular check-ups) can be performed to look for signs of developmental delays. Various treatments include behavioral, communication, and educational therapies, each of which strive to improve a diagnosed individual's social and cognitive skills. Behavioral training, including applied behavior analysis, can be performed, in which ASD subjects are taught behavioral skills across different settings and reinforcing the desirable characteristics, such as appropriate social interactions. In some instances, speech and language pathology can be performed to improve development of language and communication skills, including that ability to articulate words wells, comprehend verbal and none verbal clues in a range of settings, initiate conversation, develop conversational skills (e.g., appropriate time to say “good morning” or responses to questions asked). In some instances, an ASD subject is entered into special education courses. In some instances risperidone can be administered, which treats irritability often associated with ASD individuals.

Once diagnosed for having a risk of Alzheimer's disease, neurological and neuropsychological tests can be performed to check mental status. Imaging (e.g., MRI, CT, and PET) can be performed to check for abnormalities in structure or function. A number of supplements may help brain health and may be prophylactic, including (but not limited to) omega-3 fatty acids, curcumin, ginkgo, and vitamin E. Exercise, diet, and social support can help promote good cognitive health. Medications for Alzheimer's include (but are not limited to) cholinesterase inhibitors and memantine.

Once diagnosed for having a risk of arthritis, laboratory tests on various bodily fluids can be performed to determine the type of arthritis. Imaging (e.g., X-rays, CT, MRI, and ultrasound) can be utilize to detect problems in various joints. Physical therapy may help relieve some complications associated with arthritis. Medications for arthritis include (but are not limited to) analgesics, nonsteroidal anti-inflammatory drugs (NSAIDs), counterirritants, disease-modifying antirheumatics drugs, biologic response modifiers, and corticosteroids. Heat pads, ice packs, acupuncture, glucosamine, yoga, and massage are examples of various home/alternative remedies available.

Once diagnosed for having a risk of asthma, tests can be performed to determine lung function. A chest X-ray of CT scan can be performed to determine any structural abnormalities. Medications for asthma include (but are not limited to) inhaled corticosteroids, leukotriene modifiers, long-acting beta agonists, short-acting beta agonists, theophylline, and ipratropium. In some instances, allergy medications may help asthma and thus allergy shots and/or omalizumab can be administered. Regular exercise and maintaining a healthy wait may help reduce asthma symptoms.

Once diagnosed for having a risk of bipolar disorder, a psychiatric assessment can be performed to determine the feelings and behavior patterns. Psychotherapies and medications are available to treat bipolar disorder. Psychotherapies include (but not limited to) interpersonal and social rhythm therapy (IPSRT), cognitive behavioral therapy (CBT), and psychoeducation. Medications include (but not limited to) mood stabilizers, antipsychotics, antidepressants, and anti-anxiety medications. Some lifestyle changes can help manage some cycles of behavior that may worsen the condition, including (but not limited to) limiting drugs and alcohol, forming healthy relationships with positive influence, and getting regular physical activity.

Once diagnosed for having a risk of cancer, physical exams, laboratory tests and imaging (e.g., CT, MRI, PET) can be performed to determine if cancerous tissue is present. A biopsy can be extracted to confirm a growth is cancerous. Various treatments can be performed, including (but not limited to) adjuvant treatment, palliative treatment, surgery, chemotherapy, radiation therapy, immunotherapy, hormone therapy, and targeted drug therapy. Exercise and a healthy diet can help an individual mitigate cancer onset and progression.

Once diagnosed for having a risk of cleft lip or palate, ultrasound can be performed in utero to determine whether a fetus is developing a cleft lip or palate. Typical treatment is surgery to repair the cleft tissue.

Once diagnosed for having a risk of coronary artery disease, an electrocardiogram and/or echogram can be performed to determine a heart's performance. A stress test can be performed to determine the ability of the heart to respond to physical activity. A heart scan can determine whether calcium deposits. Patients having risk of coronary artery disease would benefit greatly from a few lifestyle changes, including (but not limited to) reduce tobacco use, eat healthy foods, exercise regularly, lose excess weight, and reduce stress. Various medications can also be administered, including (but not limited to) cholesterol-modifying medications, aspirin, beta clockers, calcium channel blockers, ranolazine, nitroglycerin, ACE inhibitors and angiotensin II receptor blockers. Angioplasty and coronary artery bypass can be performed when more aggressive treatment is necessary.

Once diagnosed for having a risk of Crohn's disease, a combination of tests and procedures can be performed to confirm the diagnosis, including (but not limited to) blood tests and various visual procedures such as a colonoscopy, CT scan, MRI, capsule endoscopy and balloon-assisted enteroscopy. Treatments for Crohn's disease includes corticosteroids, oral 5-aminosliclates, azathioprine, mercaptopurine, infliximab, adalimumab, certolizumab pegol, methotrexate, natalizumab and vedolizumab. A special diet may help suppress some inflammation of the bowel.

Once diagnosed for having a risk of dementia, further analysis of mental function can be performed to gauge memory, language skills, ability to focus, ability to reason, and visual perception. These analyses can be performed utilizing cognitive and neuropsychological tests. Brain scan (e.g., CT, MRI, and PET) and laboratory tests can be performed to determine if physiological complications exist. Medications for dementia include cholinesterase inhibitors and memantine.

Once diagnosed for having a risk of diabetes, a number of tests can be performed to determine an individual's glucose levels and regulation, including (but not limited to) glycated hemoglobin A1C test, fasting blood sugar levels, and oral glucose tolerance test. Routine visits may be performed to get a long-term regulatory look at glucose regulation. In addition, a glucose monitor can be utilized to continuously monitor glucose levels. Diabetes can be managed by various options, including (but not limited to) healthy eating, regular exercise, medication, and insulin therapy. Medications for diabetes include (but are not limited to) metformin, sulfonylureas, meglitinides, thiazolidinediones, DPP-4 inhibitors, SGLT inhibitors, and insulin.

Once diagnosed for having a risk of heart disease, various tests can be performed to determine heart function, including (but not limited to) electrocardiogram, Holter monitoring, echocardiogram, stress test, and cardiac catheterization. Lifestyle changes can dramatically improve heart disease, including (but not limited to) limiting tobacco products, controlling blood pressure, keeping cholesterol in check, keeping blood glucose levels in a good range, physical activities, eating healthy, maintaining a healthy weight, managing stress, and coping with depression. A number of medications can be provided, as dependent on the type heart of disease.

Once diagnosed for having a risk of heart failure, various tests can be performed to confirm the diagnosis, including (but not limited to) physical exams, blood tests, chest X-rays, electrocardiogram, stress test, imaging (e.g., CT and MRI), coronary angiogram, and myocardial biopsy. Medications for heart failure include (but are not limited to) ACE inhibitors, angiotensin II receptor blockers, beta blockers, diuretics, aldosterone antagonists, inotropes, and digoxin. Surgical procedures may be necessary, and include (but are not limited to) coronary bypass surgery and heart valve repair/replacement.

Once diagnosed for having a risk of high cholesterol, blood tests can be performed to measure total cholesterol, LDL cholesterol, HDL cholesterol, and triglycerides. Medications to manage cholesterol levels include (but are not limited to) statins, bile-acid-binding resins, cholesterol absorption inhibitors, and fibrates. Supplements can also be taken, including (but not limited to) co-enzyme Q, red yeast rice extract, niacin, soluble fiber, and omega-3-fatty acids. Individuals at risk for high cholesterol should also reduce tobacco products, eat a healthy diet (avoiding saturated fat, trans fat, and salt), and get regular exercise.

Once diagnosed for having a risk of hypertension, blood pressure levels can be monitored periodically (even at home). Elevated blood pressure and hypertension benefit from lifestyle changes including, eating healthy, reducing sodium intake, regular physical activity, maintaining a proper rate, and limiting alcohol intake. Medications for hypertension include (but are not limited to) ACE inhibitors, angiotensin II receptor blockers, calcium channel blockers, alpha blockers, beta blockers, aldosterone antagonists, renin inhibitors, vasodilators, and central-acting agents.

Once diagnosed for having a risk of hypothyroidism, blood tests can be performed to measure the level of TSH and thyroid hormone thyroxine. Medications for hypothyroidism includes (but is not limited to) synthetic thyroid hormone levothyroxine, which may be taken with supplements such as iron, aluminum hydroxide, and calcium to help absorption.

Once diagnosed for having a risk of irritable bowel syndrome (IBS), physical exams can be performed to confirm IBS including determining type of IBS. These exams include (but are not limited to) flexible sigmoidoscopy, colonoscopy, X-ray, and CT scan. A proper diet can be utilized to manage symptoms, including (but not limited to) high fiber fluids, plenty of fluids, and avoiding the following: high-gas foods, gluten, and FODMAPs. Medications for IBS include (but are not limited to) alosetron, eluxadoline, rifaximin, lubiprostone, linaclotide, fiber supplements, laxatives, anti-diarrheal medications, anticholinergic medications, antidepressants, and pain medications.

Once diagnosed for having a risk of obesity, a physiological test to determine body-mass index (BMI) may be performed. Obesity can be managed by various lifestyle remedies including (but not limited to) healthy diet, physical activity, and limiting tobacco products. If obesity is severe, various surgeries can be performed, including (but not limited to) gastric bypass surgery, laparoscopic adjustable gastric banding, biliopancreatic diversion with duodenal switch, and gastric sleeve.

Once diagnosed for having a risk of osteoporosis, bone density can be measured and routinely monitored using X-rays and other devices, as known in the art. Medications for osteoporosis include (but are not limited to) bisphosphonates, estrogen (and estrogen mimics), denosumab, and teriparatide. To reduce the risk of osteoporosis development, individuals can make various lifestyle changes, including (but not limited to) limiting tobacco use, limiting alcohol intake, and taking measures to prevent falls.

Once diagnosed for having a risk of Parkinson's disease, a single-photon emission computerized tomography (SPECT) scan can image dopamine transporter activity in the brain, which can be monitored over time. Medications for Parkinson's includes (but are not limited to) carbidopa-levodopa, dopamine agonists, MAO B inhibitors, COMT inhibitors, anticholinergics and amantadine.

Once diagnosed for having a risk of rhinitis, various tests can be performed to determine if the rhinitis is due to allergies, including (but not limited to) skin tests looking for allergic reaction, blood tests to measure responses to allergies (e.g., IgE levels). Medications for rhinitis include (but are not limited to) saline nasal sprays, corticosteroid nasal sprays, antihistamines, anticholinergic nasal sprays, and decongestants.

Once diagnosed for having a risk of psoriasis, routine physical exams of the skin, scalp and nails can be performed to look for signs of inflammation. A number of topical treatments can be performed for psoriasis, including (but not limited to) topical corticosteroid, vitamin D analogues, anthralin, topical retinoids, calcineurin inhibitors, salicylic acid, coal tar, and moisturizers. A number of phototherapies can also be performed, including (but not limited to) exposure to sunlight, UVB phototherapy, Goeckerman therapy, excimer laser, and psoralen plus ultraviolet A therapy. Medications for psoriasis include (but are not limited to) retinoids, methotrexate, cyclosporine, and biologics that reduce immune-mediated inflammation (e.g., entanercept, infliximab, adalimumab).

Once diagnosed for having a risk of multiple sclerosis (MS), various tests can be performed overtime to monitor symptoms of MS, including (but not limited to) blood tests, lumbar puncture, MRI and evoked potential tests. A number treatments can help treat acute MS symptoms and to mitigate MS progression, including (but not limited to) corticosteroids, plasma exchange, ocrelixumab, beta interferons, glatiramer acetate, dimethyl fumarate, fingolimod, teriflunomide, natalizumab, alemtuzumab, and mitoxantrone. Physical therapy and muscle relaxants also help mitigate (or prevent) MS symptoms.

Once diagnosed for having a risk of schizophrenia, a physical exam and/or psychiatric evaluation may be performed to determine if symptoms of schizophrenia are apparent. Various antipsychotics may be administered, including (but not limited to) aripiprazole, asenapine, brexpiprazole, cariprazine, clozapine, iloperidone, lurasidone, olanzapine, paliperidone, quetiapine, risperidone, and ziprasidone. Individual with risk of schizophrenia may also benefit from various psychosocial interventions, normalizing thought patterns, improving communication skills, and improving the ability to participate in daily activities.

Once diagnosed for having a risk of sleep apnea, an evaluation that monitors an individual's sleep may be performed, including (but not limited to) nocturnal polysomnography, measurements of heart rate, blood oxygen levels, airflow, and breathing patterns. Sleep apnea therapy may include the use of a continuous positive airway pressure (CPAP) device. A number of lifestyle changes have also been shown to mitigate complications associated with sleep apnea, including (but not limited to) losing excess weight, physical activity, mitigating alcohol consumption, and sleeping on side or abdomen.

Once diagnosed for having a risk of spina bifida, prenatal screening tests can be performed and routinely monitored determine if a fetus is developing spina bifida. Blood tests that can be performed include (but are not limited to) maternal serum alpha-fetoprotein test and measurement AFP levels. Routine ultrasound can be performed to screen for spina bifida. Various treatments include (but are not limited to) prenatal surgery to repair the baby's spinal cord and post-birth surgery to put the meninges back in place and close the opening of the vertebrae.

Once diagnosed for having a risk of stroke, routine monitoring can be performed to determine coronary health status, including (but not limited to) blood clotting tests, imaging (e.g., CT and MRI) to look for potential clots, carotid ultrasound, cerebral angiogram, and echocardiogram. Various procedures that can be performed include (but are not limited to) carotid endarterectomy and angioplasty. Patients having risk of stroke would benefit greatly from a few lifestyle changes, including (but not limited to) reduce of tobacco use, eat healthy foods, exercise regularly, lose excess weight, and reduce stress. Various medications can also be administered, including (but not limited to) cholesterol-modifying medications, aspirin, beta clockers, calcium channel blockers, ranolazine, nitroglycerin, ACE inhibitors and angiotensin II receptor blockers.

### Alterations in Dosing Based on Metabolism

A number of embodiments are directed towards altering treatments of individuals based on their biochemical regulation of genes involved with drug metabolism. In some embodiments, a model is trained to identify loci harboring variants that affect regulation of drug metabolizing genes. In some embodiments, genomic loci known to harbor variants that alter transcriptional and/or posttranscriptional regulation are associated with a drug metabolism. In some embodiments, the pathogenicity of the detected variants is determined, which may be used to determine the biochemical activity of a drug metabolizing gene. And in some embodiments, the biochemical activity and/or pathogenicity of variants affected of a drug metabolizing gene are determined using a computational model. Based on results, in some embodiments, dosing can be altered (i.e., high metabolizers are dosed higher and low metabolizers are dosed lower).

Several medications are known to be metabolized differently by individuals based on the expression of a few key genes. Table 5 is a list of medication and genes that are involved with metabolism of that medication. Medications and genes involved in their metabolism can also be found using the PharmGKB database (www.phargkb.org) Accordingly, based on methods described herein that determine alterations biochemical regulation, especially in transcriptional and/or posttranscriptional regulation, an individual can be treated accordingly. For example, the gene CYP2D6 is involved in the metabolism of risperidone. If an individual is found to have regulatory variants that decrease the activity of CYP2D6, then lower doses of oxycodone (or an alternative medication) can be administered. If an individual is found to have regulatory variants that increase the activity of CYP2D6, then higher doses of oxycodone (or an alternative medication) can be administered. In some embodiments, determination of transcriptional and/or posttranscriptional regulatory effects of variants and/or their pathogenicity by performing methods described in FIGS. 2, 3, 4A and 4B. It should be noted, however, that any method capable of determining posttranscriptional regulatory effects of variants and/or their pathogenicity can be utilized within various embodiments.

In many embodiments, dosing alteration methods are performed as follows:
a) obtain a set of variants of an individual
b) determine transcriptional and/or posttranscriptional regulatory effects of each variant of the set of variants on genes that affect metabolism
c) optional: determine the pathogenicity of each variants of a set of variants
d) based on regulatory effects and/or pathogenicity of variants, determine the ability of an individual to metabolize a medication
e) based on metabolism results, administer an appropriate dose of the medication or administer an alternative medication.
   In some embodiments, determination of transcriptional and/or posttranscriptional regulatory effects can be performed in accordance with either FIG. 2 or FIG. 4A. In some embodiments, determination of pathogenicity can be performed in accordance with either FIG. 3 or FIG. 4B.

### Exemplary Embodiments

Bioinformatic and biological data support the methods and systems of determining the contribution of variants on transcriptional and posttranscriptional regulation and further determining a pathogenicity score using the regulatory variants, and applications thereof. In the ensuing sections, exemplary computational methods and exemplary applications related to variant classifications are provided, especially in the context of autism spectrum disorder (ASD). Exemplary methods and applications can also be found in the publication “Whole-genome deep learning analysis reveal causal role of noncoding mutations in autism” of J. Zhou, et al., bioRxiv 319681 (May 11, 2018), the disclosure of which is herein incorporated by reference.

### Whole-Genome Deep Learning Analysis Reveals Causal Role of Noncoding Mutations in Autism

Within the following examples, a deep-learning based approach for quantitatively assessing the impact of noncoding mutations on human disease is provided. The approach addresses the statistical challenge of detecting the contribution of noncoding mutations by predicting their specific effects on transcriptional and post-transcriptional levels. This approach is general and can be applied to study contributions of mutations to any complex disease or phenotype.

In this example, the strategy was applied to ASD using the 1,790 whole genome sequenced families from the Simons Simplex Collection, and for the first time the results demonstrate a significant proband-specific signal in regulatory de novo noncoding sequence. Importantly, this signal was not only independently detected at the transcriptional level, but the proband-specific posttranscriptional burden was also found to be significant. Previously, there has been limited evidence for disease contribution of mutations disrupting posttranscriptional mechanisms outside of the canonical splice sites. Here, it is demonstrated that significant ASD disease association at the de novo mutation level for variants impacting a large collection of RBPs regulating posttranscriptional regulation. Overall, the results suggest that both transcriptional and posttranscriptional mechanisms play a significant role in complex disorders such as ASD.

The analyses also demonstrate the ability to diagnose complex traits from genetic information, including de novo noncoding mutations that affect transcriptional and posttranscriptional regulation.

### Contribution of Transcriptional and Post-Transcriptional Regulatory Mutation to ASD

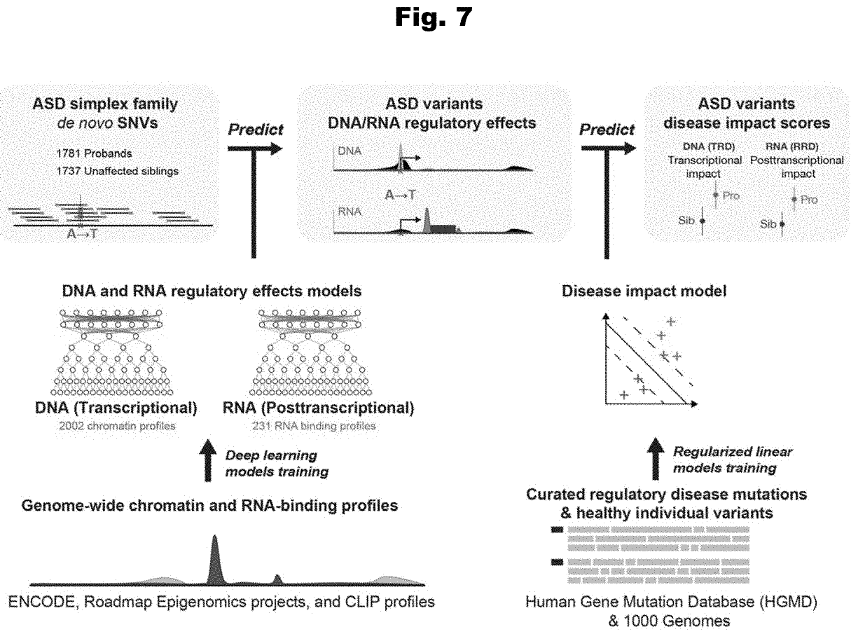

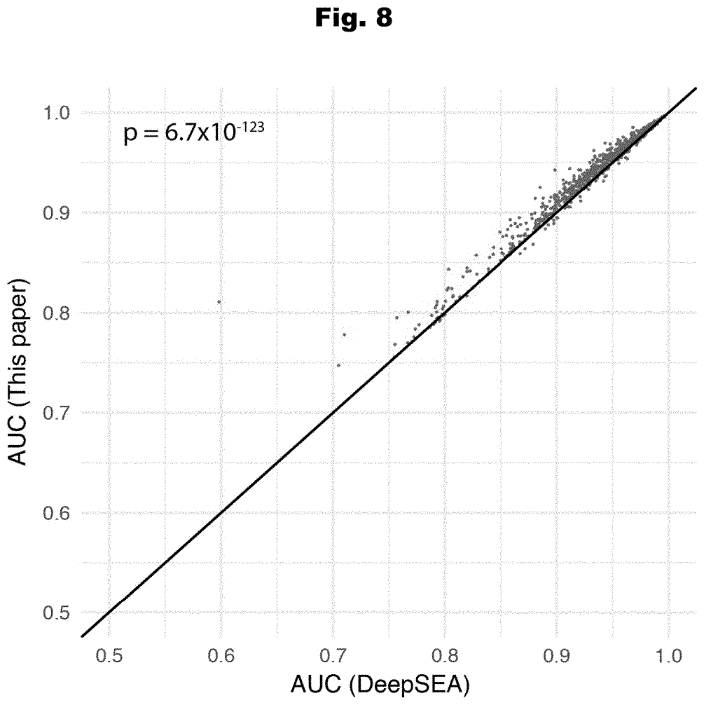

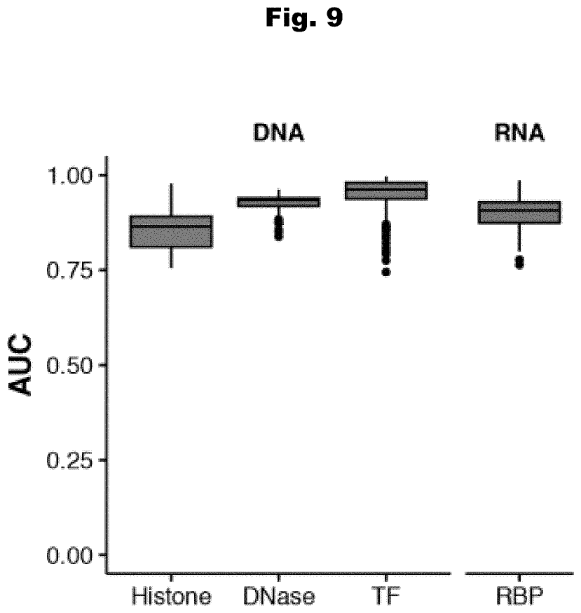

Analysis of the noncoding mutation contribution to ASD is challenging due to the difficulty of assessing which noncoding mutations are functional, and further, which of those contribute to the disease phenotype. For predicting the regulatory impact of noncoding mutations, a deep convolutional network-based framework was constructed to directly model the functional impact of each mutation and provide a biochemical interpretation including the disruption of transcription factor binding and chromatin mark establishment at the DNA level and of RBP binding at the RNA level (FIG. 7). At the DNA level, the framework includes cell-type specific transcriptional regulatory effect models from over 2,000 genome-wide histone marks, transcription factor binding and chromatin accessibility profiles (from ENCODE and Roadmap Epigenomics projects, extending the deep learning-based method of a previously described model with redesigned architecture (J. Zhou & O. G. Troyanksaya Nat. Methods 12, 931-4 (2015); T. N. Turner, et al., Am. J. Hum. Genet. 98, 58-74 (2016); and for more on Roadmap Epigenomics projects see B. E. Bernstein, et al. Nat. Biotechnol. 28, 1045-8 (2010); the disclosures of which are each herein incorporated by reference). These modifications provided significantly improved performance, p=6.7×10⁻¹²³, Wilcoxon rank-sum test, FIG. 8). At the RNA level, the deep learning-based method was trained on the precise biochemical profiles of over 230 RBP-RNA interactions (derived from CLIP data); such data can identify a wide range of post-transcriptional regulatory binding sites, including those involved in RNA splicing, localization and stability (see J. Ule, H. W. Hwang, and R. B. Darnell, Cold Spring Harb. Perspect. Biol. 10, (2018), the disclosure of which is herein incorporated by reference). At both transcriptional and post-transcriptional levels, the models are accurate and robust in whole chromosome holdout evaluations (FIG. 9). The models utilize a large sequence context to provide single nucleotide resolution to their predictions, while also capturing dependencies and interactions between various biochemical factors (e.g. histone marks or RBPs). This approach is data-driven, does not rely on known sequence information, such as transcription factor binding motifs, and it can predict impact of any mutation regardless of whether it has been previously observed, which is essential for the analysis of ASD de novo mutations.

To illustrate the capabilities of the transcriptional and posttranscriptional models and pathogenicity computational model, an analysis of the noncoding mutation contribution to ASD was performed using whole genome sequencing (WGS) data was derived from the Simons Simplex Collection (SSC), available via Simons Foundation Autism Research Initiative (SFARI). The data was processed to generate variant calls via the standard GATK pipeline (https://software.broadinstitute.org/gatk/). To call de novo single nucleotide substitutions, inherited mutations were removed, and candidate de novo mutations were selected from the GATK variant calls where the alleles were not present in parents and the parents were homozygous with the same allele. DNMFilter classifier was then used to score each candidate de novo mutation and a threshold of probability>0.75 was applied for SSC phasel-2 and probability>0.5 cutoff for phase3 to obtain a comparable number of high-confidence DNM calls across phases (for more on DNMFilter, see Gene Ontology Consortium, Nucleic Acid Res. 43, D1049-56 (2015), the disclosure of which is herein incorporated by reference).

The DNMFilter classifier was trained with an expanded training set combining the original training standards with the verified DNMs from the SSC pilot WGS studies for the initial 40 SSC families. For final analysis, de novo mutation calls within the low complexity repeat regions from UCSC browser table RepeatMasker were removed (see H. Mi, et al., Nucleic Acids Res. 45, D183-D189 (2017), the disclosure of which is herein incorporated by reference. Also, de novo mutations appearing in multiple SSC families (i.e., non-singleton de novo mutations) or individuals with outlier numbers of mutations (greater than 3 standard deviation more than average) were excluded from the analysis.

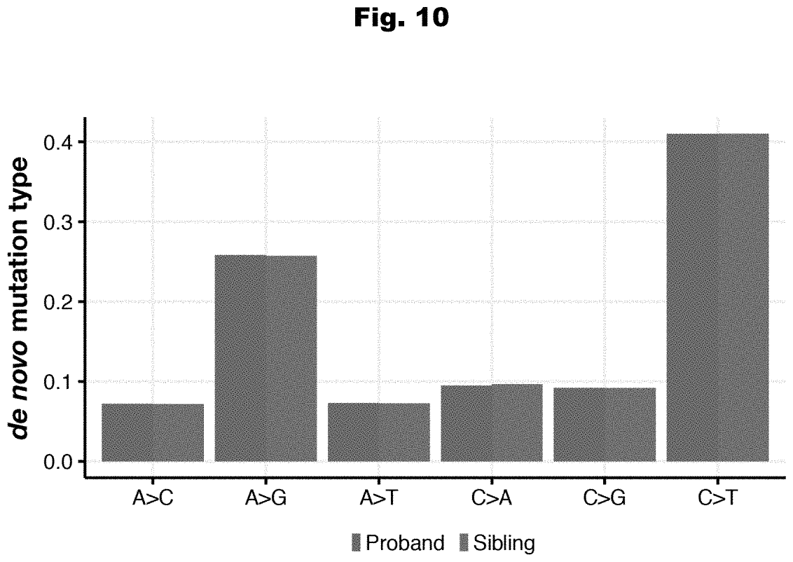

Overall genome-wide, 77.7 mutations per individual were detected with Ti/Tv ratio 2.01 [2.00, 2.03] (78.7 for probands with Ti/Tv=2.02 [1.99, 2.04], 76.7 for siblings with Ti/Tv=2.01 [1.99, 2.03]), with no significant difference in mutation substitution patterns between proband and sibling (FIG. 10). The WGS de novo mutation calls were compared against exome sequencing de novo mutations calls and previously validated SSC de novo mutations. 87.9% of the exome sequencing mutations calls and 90.3% of the validated mutations were rediscovered in the mutations calls in this model.

For training the transcriptional regulatory effects model, training labels, such as histone marks, transcription factors, and DNase I profiles, were processed from uniformly processed ENCODE and Roadmap Epigenomics data releases. The training procedure is similar to previously described (J. Zhou & O. G. Troyanskaya (2015), cited supra) with several modifications. The model architecture was extended to double the number of convolution layers for increased model depth (see below for details). Input features were expanded to include all of the released Roadmap Epigenomics histone marks and DNase I profiles, resulting in 2,002 total features (subset provided in Table 1; full list is provided in electronic format via Electronic Data Table 1).

The model architecture for transcriptional regulatory effects model:

Input (Size: 4 bases×1000 bp)=>

(#1): Convolution(4→320, kernel size=8)

(#2): ReLU

(#3): Convolution(320→320, kernel size=8)

(#4): ReLU

(#5): Dropout(Probability=0.2)

(#6): Max pooling(pooling size=4)

(#7): Convolution(320→480, kernel size=8)

(#8): ReLU

(#9): Convolution(480→480, kernel size=8)

(#10): ReLU

(#11): Dropout(Probability=0.2)

(#12): Max pooling(pooling size=4)

(#13): Convolution(480→960, kernel size=8)

(#14): ReLU

(#15): Convolution(960→960, kernel size=8)

(#16): ReLU

(#17): Dropout(Probability=0.2)

(#18): Linear(42240→2003)

(#19): ReLU

(#20): Linear(2003→2002)

(#21): Sigmoid

=>Output (Size: 2002 transcriptional regulatory features)

ReLU indicates the rectified linear unit activation function. Sigmoid indicates the Sigmoid activation function. Notations such as ‘4→320’ indicate the input and output channel size for each layer. When not indicated, the output channel size is equal to the input channel size.

For training the posttranscriptional regulatory effects model, the Seqweaver network architecture and training procedure with RNA-binding protein (RBP) profiles as training labels we utilized (see below for architecture and parameters). RNA features, composed of 231 CLIP binding profiles for 82 unique RBPs (ENCODE and previously published CLIP datasets), were uniformly processed. A branch-point mapping profile was used as input features (subset provided in Table 2; full list is provided in electronic format via Electronic Data Table 2). CLIP data processing followed a previously detailed pipeline (J. M. Moore, et al., Nat. protoc. 9, 263-293 (2014), the disclosure of which is herein incorporated by reference). All CLIP peaks with p-value<0.1 were used for training with an additional filter requirement of two-fold enrichment over input for ENCODE eCLIP data. In contrast to the DeepSEA, only transcribed genic regions were considered as training labels for the post-transcriptional regulatory effects model. Specifically, all gene regions defined by Ensemble (mouse build 80, human build 75) were split into 50 nt bins in the transcribed strand sequence. For each sequence bin, RBP profiles that overlapped more than half were assigned a positive label for the corresponding RBP model. Negative labels for a given RBP model were assigned to sequence bins where other RBP's non-overlapping peaks were observed. Note that the deep learning models, both transcriptional and posttranscriptional, each do not use any mutation data for training, and thus each can predict mutation impact regardless of whether it has been previously observed.

The model architecture and parameters for posttranscriptional regulatory effects model:
1. Convolution layer—160 kernels. Window size: 8. Step size: 1.
2. Pooling layer—Window size: 4. Step size: 4.
3. Convolution layer—320 kernels. Window size: 8. Step size: 1.
4. Pooling layer—Window size: 4. Step size: 4.
5. Convolution layer—480 kernels. Window size: 8. Step size: 1.
6. Fully connected layer—human model 217 neurons, mouse model 43 neurons
7. Sigmoid output layer

### Parameters:

Dropout Proportion:
Layer 2: 10%
Layer 4: 10%
Layer 5: 30%
All other layers 0%

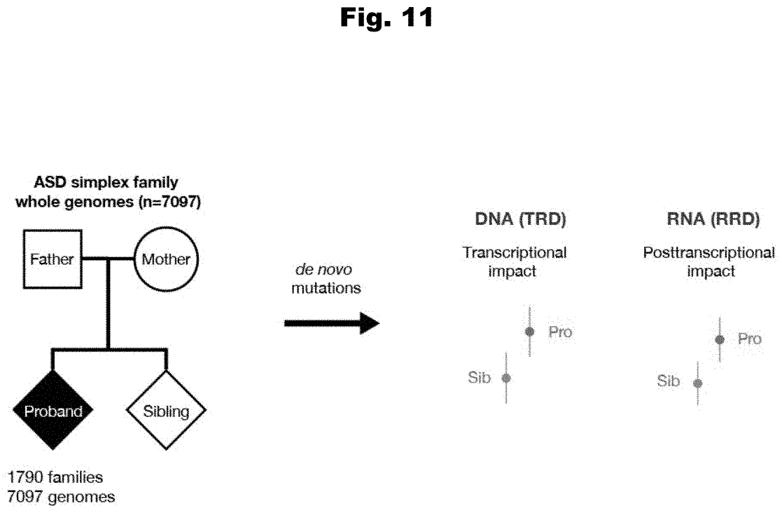

Overall design and results of the trained transcriptional (TRD) and posttranscriptional (RRD) models are provided in FIG. 11. As can be seen, probands on average had more accumulation of variants with higher transcriptional and posttranscriptional impact.

To link the biochemical disruption caused by a variant with phenotypic impact, a regularized linear model was trained using a set of curated human disease regulatory noncoding mutations and rare variants from healthy individuals to generate a predicted disease impact score (DIS) (i.e., pathogenicity) for each autism mutation independently based on its predicted transcriptional and post-transcriptional regulatory effects. As mutation-positive examples, 4,401 regulatory noncoding mutations curated in the Human Gene Mutation Database (HGMD) with mutation type “regulatory” (DM, DM?, DFP, DP and FP) were used for training (for more on HGMD and mutation type see P. D Stenson, et al., Hum. Genet. 132, 1-9 (2014), the disclosure of which is herein incorporated by reference). For negative examples of background mutations, 999,668 rare variants that were only observed once within the healthy individuals from the 1000 Genomes project were used (see 1000 Genomes Project Consortium et al., Nature, 526, 68-74 (2015), the disclosure of which is herein incorporated by reference). It was also showed that using common variants with AF>0.01 and within 100 kb to a mutation-positive hit as negative training labels yields similar results to the use of the 1000 Genomes project data. Absolute predicted probability differences computed by the convolutional network transcriptional regulatory effects model were used as input features for each of the 2,002 transcriptional regulatory features and for the 232 post-transcriptional regulatory features in the disease impact model. Input features were standardized to unit variance and zero mean before being used for training. An L2 regularized logistic regression model was separately trained for transcriptional effect model (lambda=10) and post-transcriptional effect model (lambda=10, using only genic region variant examples) with the xgboost package (https://github.com/dmlc/xgboost). The predicted probabilities are z-transformed to have mean 0 and standard deviation 1 across all proband and sibling mutations.

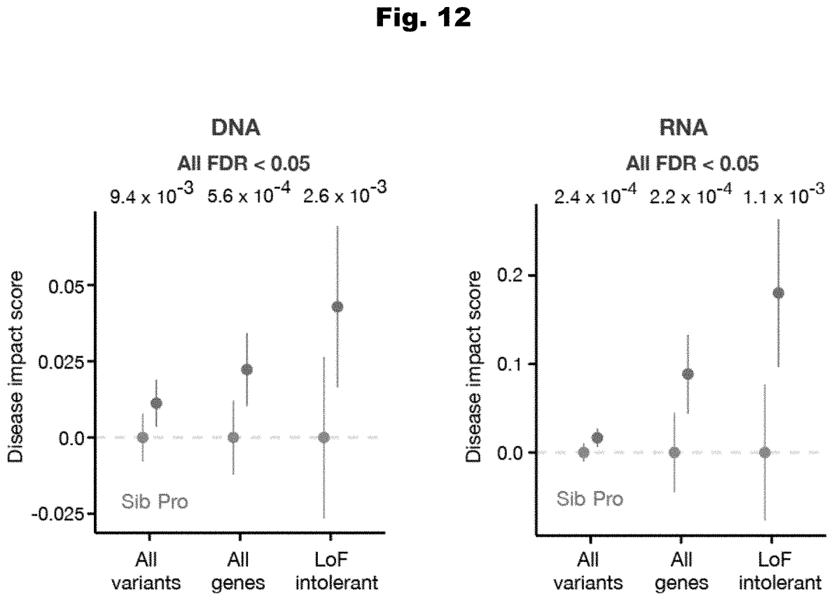

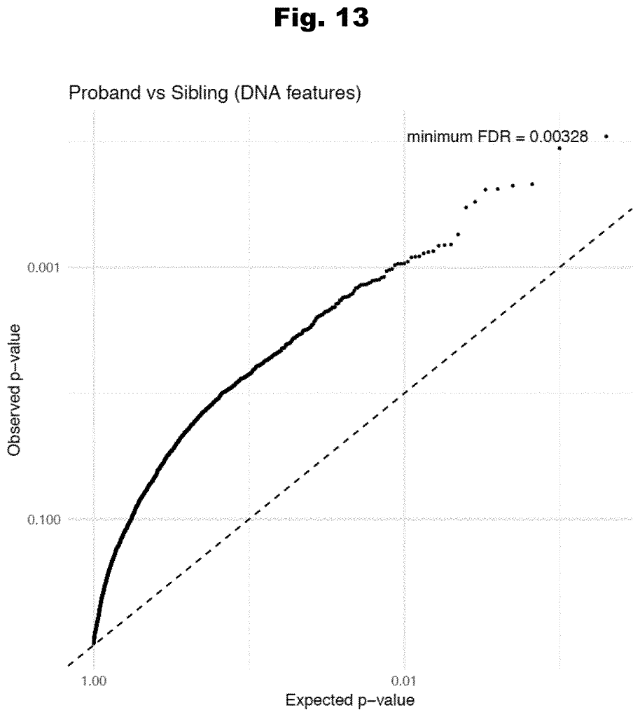

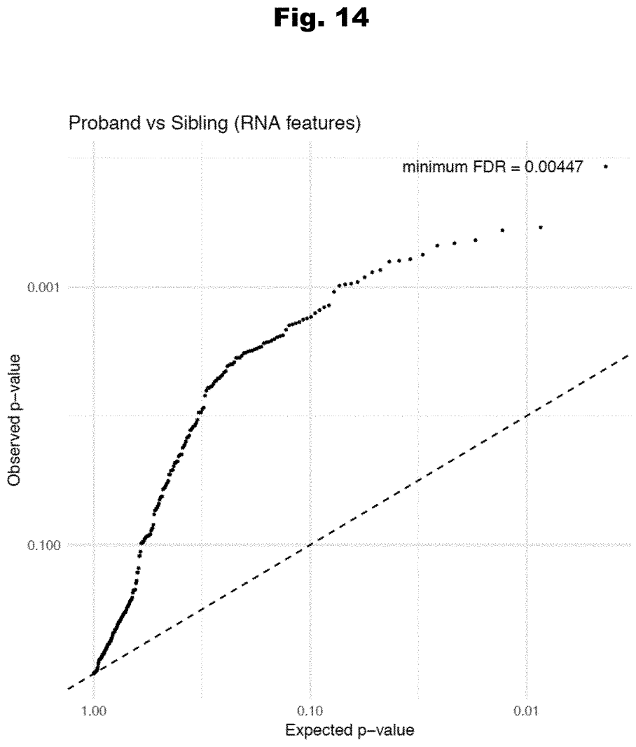

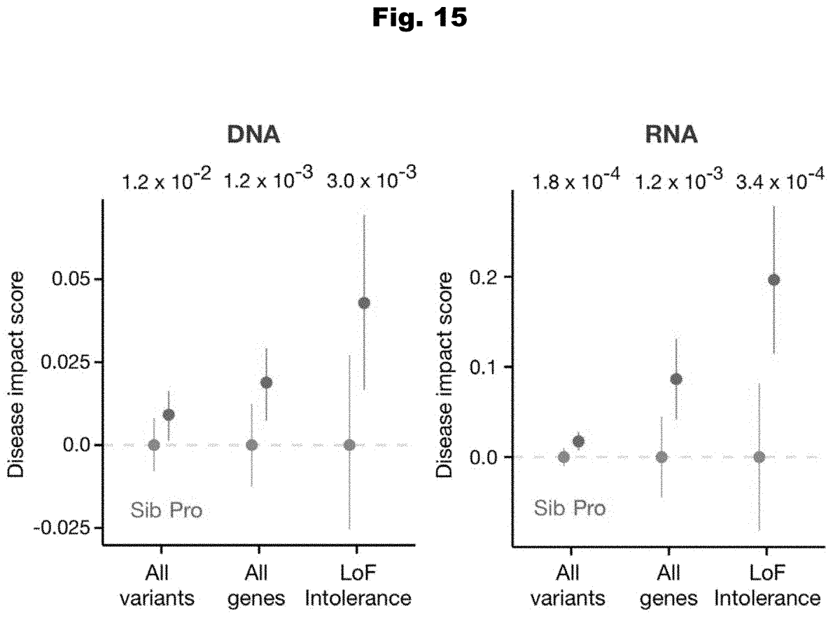

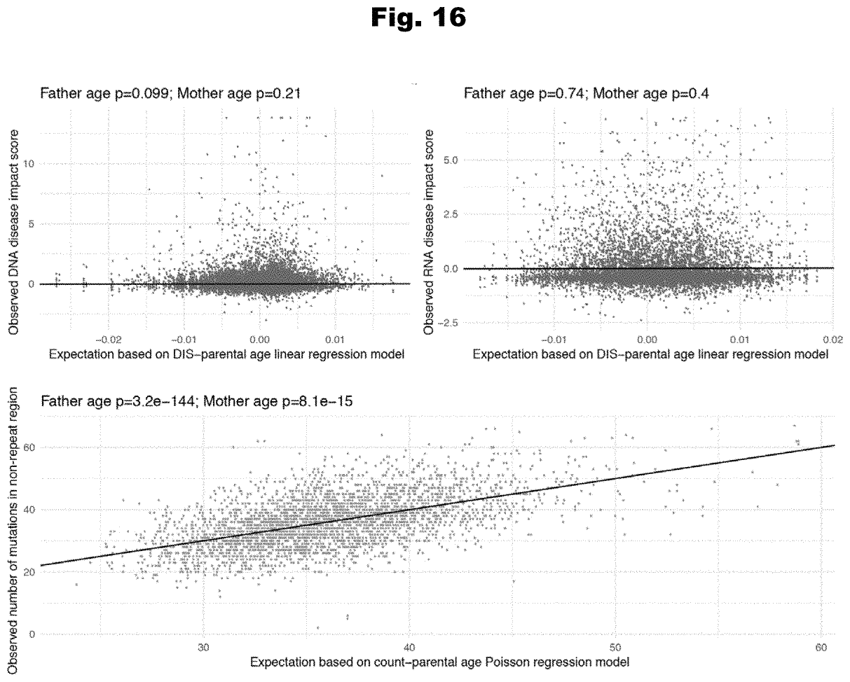

With these approaches, the functional impact of de novo mutations on regulatory factor binding and chromatin properties were systematically assessed using data derived from 7,097 whole genomes from the SSC cohort (total 127,139 non-repeat region SNVs; subset provided in Table 3; full list is provided in electronic format via Electronic Data Table 3). When considering all de novo mutations, a significantly higher functional impact in probands was observed compared to unaffected siblings, independently at the transcriptional (p=9.4×10⁻³, one-side Wilcoxon rank-sum test for all; FDR=0.033, corrected for all mutation sets tested) and post-transcriptional (p=2.4×10⁻⁴, FDR=0.0049) levels (FIG. 12, all variants). This finding is robust and significant directly at the level of biochemical disruptions predicted by DNA and RNA deep learning models as well as with alternative DIS training sets (FIGS. 13-15). Notably, these results do not rely on any selection of variant subsets (e.g., variants near predicted ASD-associated genes), and are significant even after conservative multiple hypothesis correction. Unlike the mutation counts, the predicted mutation effects are not correlated with parental age (FIG. 16).

To gain further insight into the ASD noncoding regulatory landscape, a comprehensive analysis was performed with full multiple hypothesis correction for all combinations of 14 gene-sets and 10 genomic regions tested (e.g., TSS or exon proximal) previously described in D. M. Werling et al. (Nat. Genet. 50, 727-736 (2018), the disclosure of which is herein incorporated by reference).

The 14 gene-sets include GENCODE protein coding genes, Antisense, lincRNAs, Pseudogenes, genes with loss-of-function intolerance (pLI) score>0.9 from ExAC, predicted ASD risk genes (FDR<0.3), FMRP target genes, Genes associated with developmental delay and CHD8 target genes. For genes with expression specific to each 53 GTEx tissue, expression table from GTEx v7 (gene median TPM per tissue) was used to select genes for which expression in a given tissue was five times higher than the median expression across all tissues.

The representative TSS for each gene was determined based on FANTOM CAGE transcription initiation counts relative to GENCODE gene models. Specifically, a CAGE peak is associated to a GENCODE gene if it is within 1000 bp from a GENCODE v24 annotated transcription start site. Peaks within 1000 bp to rRNA, snRNA, snoRNA or tRNA genes were removed to avoid confusion. Next, the most abundant CAGE peak for each gene was selected, and the TSS position reported for the CAGE peak was used as the selected representative TSS for the Gene. For genes with no CAGE peaks assigned, the GENCODE annotated gene start position was used as the representative TSS. FANTOM CAGE peak abundance data were downloaded at http://fantom.gsc.riken.jp/5/datafiles/latest/extra/CAGE_peaks/ and the CAGE read counts were aggregated over all FANTOM 5 tissue or cell types. GENCODE v24 annotation lifted to GRCh37 coordinates were downloaded from http://www.gencodegenes.org/releases/24lift37.html. All chromatin profiles used from ENCODE and Roadmap Epigenomics projects were listed in Electronic Data Table 1. The HGMD mutations are from HGMD professional version 2018.1.

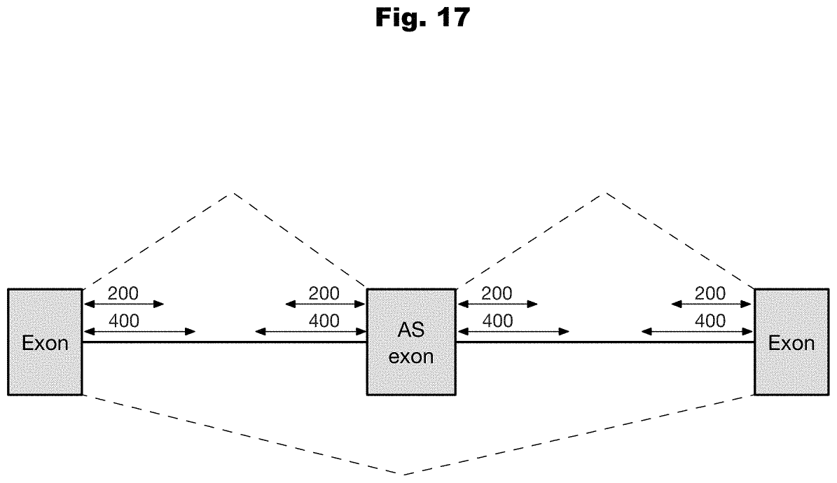

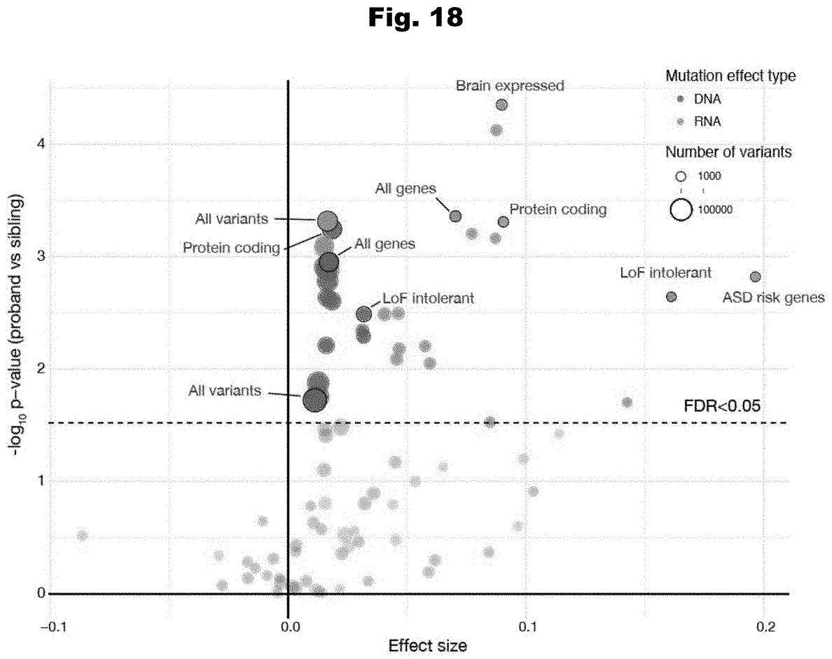

Human exons that are alternatively spliced (AS) were obtained from a recent study that has examined publicly available human RNA-seq data to annotate an extensive catalog of AS events (Q. Yan, et al., Proc. Natl. Acad. Sci. 111, 3445-3450 (2015), the disclosure of which is herein incorporated by reference). Internal exon regions (both 5′SS & 3′SS flanking introns), upstream exon (5′SS flanking introns), and downstream terminal exon (3′SS flanking introns) were used for alternative exon definition types of cassette, mutually exclusive, tandem cassette exons. Terminal exon region was used for intron retention, alternative 3′ or 5′ exon AS exon types. All selected exon-flanking intronic regions were collapsed into a final set of genomic intervals used to subset SNVs that are located within alternative splicing exon region (200 or 400 nts from exon boundary), illustrated in FIG. 17.

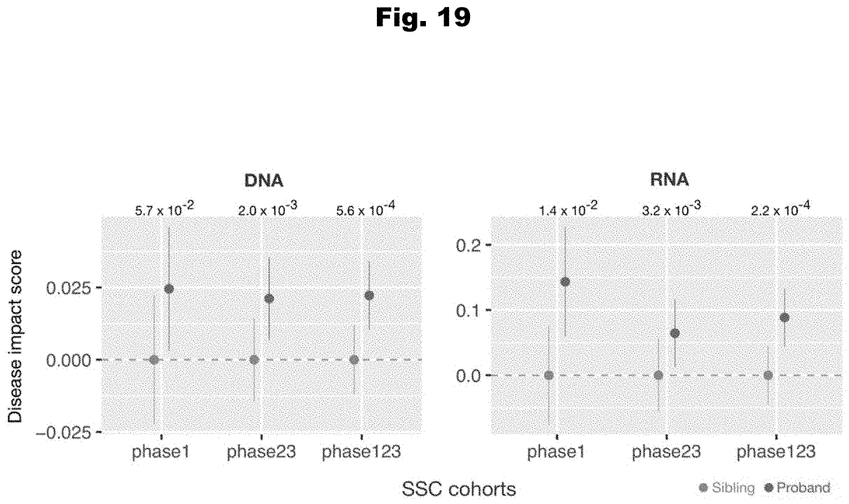

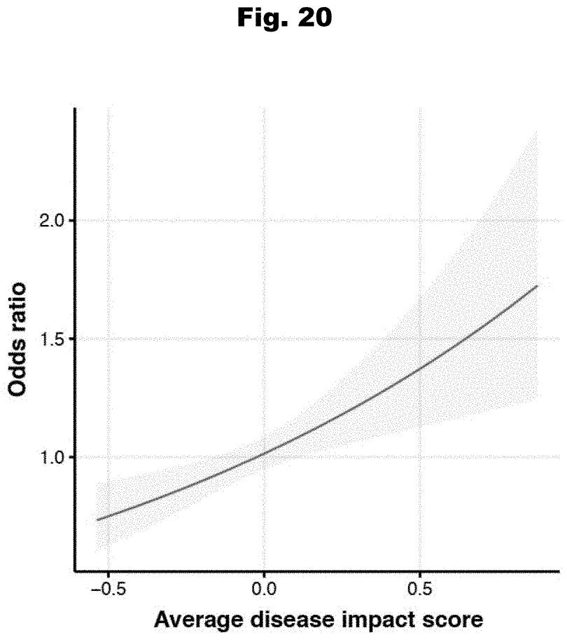

When restricted to genomic regions of higher regulatory potential (i.e. near TSS or alternatively spliced exons), an increased dysregulation effect size was observed (FIGS. 12 & 18, all genes, TRD p=5.6×10⁻⁴, FDR=0.0056; RRD p=2.2×10⁻⁴, FDR=0.0048). Among gene sets, an elevated proband burden of high effect mutations close to loss-of-function (LoF) intolerant genes was observed (pLI>0.9 from ExAC, 3,230 genes, TRD p=2.6×10⁻³, FDR=0.013; RRD p=1.1×10⁻³, FDR=0.0078) (FIGS. 12 & 18, ExAC LoF), suggesting LoF intolerant genes are highly vulnerable to noncoding disruptive mutations in ASD. Importantly, a convergent signal was found at both transcriptional and post-transcriptional levels, thus providing further evidence for the casual role of noncoding effects in ASD. These signals were consistently observed across SSC cohort subsets that were sequenced in different phases (FIG. 19). In addition, at the individual level, the cumulative effects of noncoding mutations lead to a significantly higher ASD risk odds ratios (FIG. 20).

### Tissue Specificity and Functional Landscape of Noncoding ASD-Associated De Novo Mutations

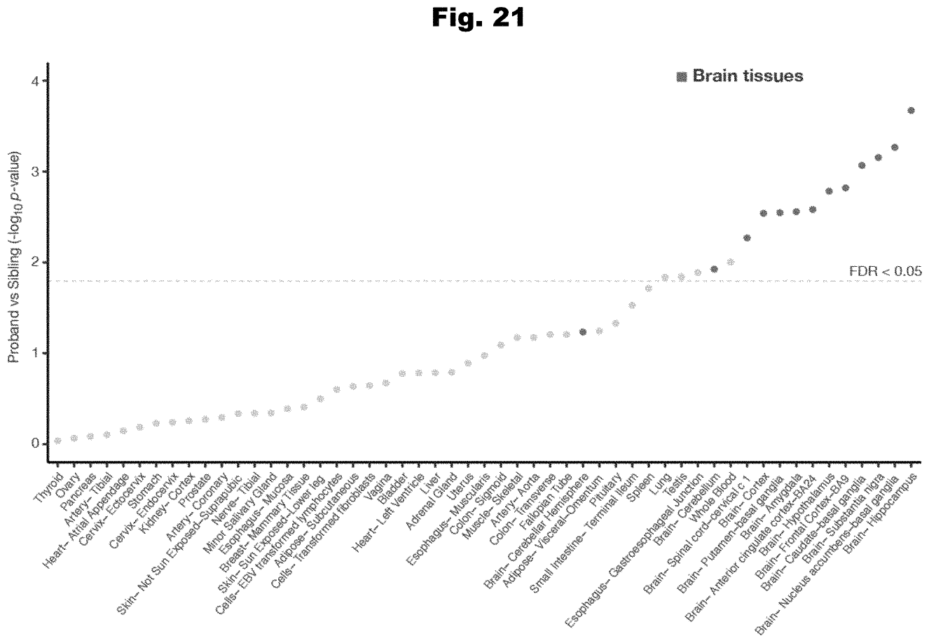

Although one of the hallmarks of autism is altered brain development, a comprehensive tissue association has not been established for de novo noncoding variants. To explore the proband-specific tissue signal, the variant effects for tissue-specific genes derived from all 53 GTEx tissues and cell types was systematically tested (for more GTEx tissues and cell types, see F. Aguet, et al., Nature 550, 204-213 (2017), the disclosure of which is herein incorporated by reference). A consistent significant proband-specific mutation effect associated with brain tissues was observed, with brain regions constituting the top 11 ranked tissues (by difference in proband vs sibling noncoding mutation effect) (FIG. 21, all with FDR<0.05). This provides strong evidence that high impact variants from the noncoding genome of ASD probands likely disrupt brain-specific gene regulation.

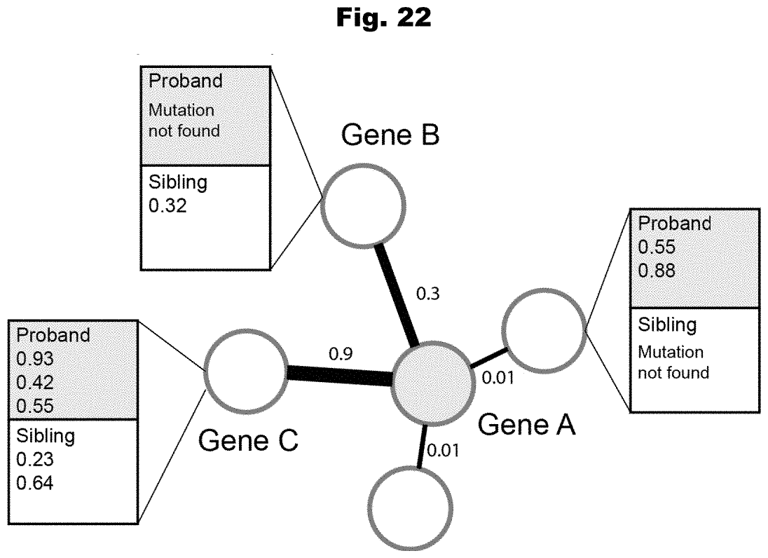

The underlying processes and pathways impacted by de novo noncoding mutations in ASD was investigated. Such analysis is challenging because in addition to the variability in functional impact of mutations, ASD probands appear highly heterogeneous in underlying causal genetic perturbations and single mutations could cause a widespread effect on downstream genes. Thus to detect genes and pathways relevant to the pathogenicity of ASD TRD and RRD mutations, a network-based statistical approach was developed, NDEA (Network-neighborhood Differential Enrichment Analysis) (FIG. 22). A brain-specific functional network that probabilistically integrates a large compendium of public omics data was used (e.g. expression, PPI, motifs) to represent how likely two genes are to act together in a biological process (see C. S. Greene, et al., Nat. Genet. 47, 569-576 (2015), the disclosure of which is herein incorporated by reference). This network was filtered to only include edges with >0.01 probability (above Bayesian prior) to reduce the impact of noisy low-confidence edges.

NDEA was used to test the differential (proband vs sibling) impact of mutations on each gene or gene set. Intuitively, this test generates a p-value that reflects the proband-specific impact of mutations on that gene or gene set, including through its network neighborhood. This also enables statistical assessment of which gene sets (e.g. pathways) are significantly more affected by proband mutations compared to sibling mutations. Technically, NDEA performs a weighed two-sample (proband vs sibling mutations) test, where the weight for each observation is defined based on network connectivity scores (to the gene or gene sets) and two samples are compared based on weighted averages. Each weight is a non-negative constant number that is used to specify the relative contribution of an observation to the test statistic. When all weights are the same, it reduces to regular two-sample t tests; when the weights are different, it adjusted the standard t statistic to use appropriate variance resulting from weighting. Note, unlike some other weighted t-tests, the weights are not random variables and do not represent sample sizes. The assumptions of the NDEA test are analogous to those of the standard two-sample t test, including that samples in each set are i.i.d. and the weighted sample means are normally distributed.

For each gene i, the NDEA t statistic is computed by

$${t}_{i} = ( {μ}_{{P}_{i}} - {μ}_{{S}_{i}} ) / {S}_{i}$$

$${μ}_{{P}_{i}} = \frac{{\sum }_{m ∈ P} {W}_{ij ( m )} {d}_{m}}{{\sum }_{m ∈ P} {W}_{ij ( m )}} , {μ}_{{S}_{i}} = \frac{{\sum }_{m ∈ S} {W}_{ij ( m )} {d}_{m}}{{\sum }_{m ∈ S} {W}_{ij ( m )}}$$

$${S}_{i} = \sqrt{\frac{{V}_{{P}_{i}}}{{N}_{{P}_{i}}} + \frac{{V}_{{S}_{i}}}{{N}_{{S}_{i}}}}$$

$${V}_{{P}_{i}} = \frac{{\sum }_{m ∈ P} {{W}_{ij ( m )} ( {d}_{m} - {μ}_{{P}_{i}} )}^{2}}{{\sum }_{m ∈ P} {W}_{ij ( m )} - \frac{{\sum }_{m ∈ P} {W}_{ij ( m )}^{2}}{{\sum }_{m ∈ P} {W}_{ij ( m )}}} , \\ {V}_{{S}_{i}} = \frac{{\sum }_{m ∈ S} {{W}_{ij ( m )} ( {d}_{m} - {μ}_{{S}_{i}} )}^{2}}{{\sum }_{m ∈ S} {W}_{ij ( m )} - \frac{{\sum }_{m ∈ S} {W}_{ij ( m )}^{2}}{{\sum }_{m ∈ S} {W}_{ij ( m )}}}$$

$${N}_{{P}_{i}} = \frac{{( {\sum }_{m ∈ P} {W}_{ij ( m )} )}^{2}}{{\sum }_{m ∈ P} {W}_{ij ( m )}^{2}} , {N}_{{S}_{i}} = \frac{{( {\sum }_{m ∈ S} {W}_{ij ( m )} )}^{2}}{{\sum }_{m ∈ S} {W}_{ij ( m )}^{2}}$$

in which μ_{P}_{ᵢ} and μ_{S}_{ᵢ} are weighted averages of disease impact scores dₘ of all proband mutations P or all sibling mutations S. Wᵢⱼ₍ₘ₎ is the network edge score (interpreted as functional relationship probability) between gene i and gene j(m) divided by the number of proband (if m is a proband mutation) or sibling (if m is a sibling mutation) mutations gene j(m) is associated to, where j(m) indicate the implicated gene of the mutation m. P and S are the set of all proband mutations and the set of all sibling mutations included in the analysis. V_{P}_{ᵢ} and V_{S}_{ᵢ} are the unbiased estimates of population variance of μ_{P}_{ᵢ} and μ_{S}_{ᵢ}. N_{P}_{ᵢ} and N_{S}_{ᵢ} are the effective sample sizes of proband and sibling mutations after network-based weighting for gene i.

Under null hypothesis of the two groups have no difference, the above t statistic approximately follows a t-distribution with the following degree of freedom:

$$df = \frac{{( \frac{{V}_{{P}_{i}}}{{N}_{{P}_{i}}} + \frac{{V}_{{S}_{i}}}{{N}_{{S}_{i}}} )}^{2}}{\frac{{V}_{{P}_{i}}^{2}}{{N}_{{P}_{i}}^{2} ( {N}_{{P}_{i}} - 1 )} + \frac{{V}_{{S}_{i}}^{2}}{{N}_{{S}_{i}}^{2} ( {N}_{{S}_{i}} - 1 )}}$$

For testing significance difference between proband and sibling mutations, mutations within 100 kb of the representative TSS of all genes and all intronic mutations within 400 bp to exon boundary were included in this analysis. RNA model disease impact scores were used as the mutation score for intronic mutations within 400 bp to exon boundary and DNA model disease impact scores were used for other mutations.

For gene set level NDEA, the gene set was considered as a meta-node that contains all genes that are annotated to the gene set (e.g. GO term). Then, to any given gene the average of network edge scores for all genes in the meta-node is used as the weights. GO term annotations were pooled from human (EBI May 9, 2017), mouse (MGI May 26, 2017) and rat (RGD Apr. 8, 2017). Query GO terms were obtained from the merged set of curated GO consortium slims from Generic, Synapse, ChEMBL, and supplemented by PANTHER GO-slim and terms from NIGO (see Gene Ontology Consortium, Nucleic Acids Res. 43, D1049-56 (2015); H. Mi, et al., Nucleic Acids Res. 45, D183-D189 (2017); and N. Geifman, A Monsonego & E. Rubin BMC Bioinformatics 11, (2010), the disclosures of which are each herein incorporated by reference).

For network-based analysis of correlation between coding and noncoding TRD and RRD mutations, the NDEA t-statistic was first computed for every gene for all protein coding mutations from SSC exome sequencing study, all SSC WGS noncoding mutations within 100 kb to a gene, and all SSC WGS genic noncoding mutations within 400 bp to an exon, respectively. Correlation across all resulting gene-specific t-statistics between all three pairs of mutation types was then computed. For testing statistical significance of the correlation, proband and sibling labels were permuted for all mutations to compute the null distributions of correlations for each pair of mutation type. 1000 permutations were performed.

For network visualization, a two-dimensional embedding with t-SNE was computed by directly taking a distance matrix of all pairs of genes as the input (see L. Van Der Maaten & G. Hinton, J. Mach. Learn. Res. 1 620, 267-84 (2008), the disclosure of which is herein incorporated by reference). The distance matrix was computed as—log(probability) from the edge probability score matrix in the brain-specific functional relationship network. The Barnes-Hut t-SNE algorithm implemented in the Rtsne package was used for the computation. Louvain community clustering were performed on the subnetwork containing all protein-coding genes with top 10% NDEA FDR.

When applied to ASD de novo mutations, the NDEA approach identifies genes whose functional network neighborhood is significantly enriched for genes with stronger predicted disease impact in proband mutations compared to sibling mutations (50 most significant genes provided in Table 4; full list is provided in electronic format via Electronic Data Table 4).

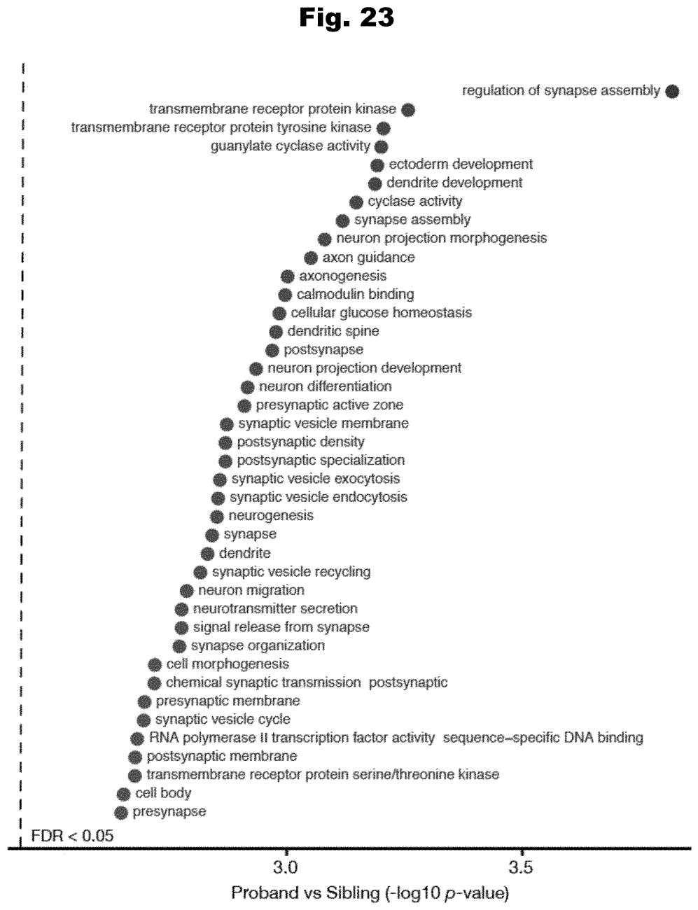

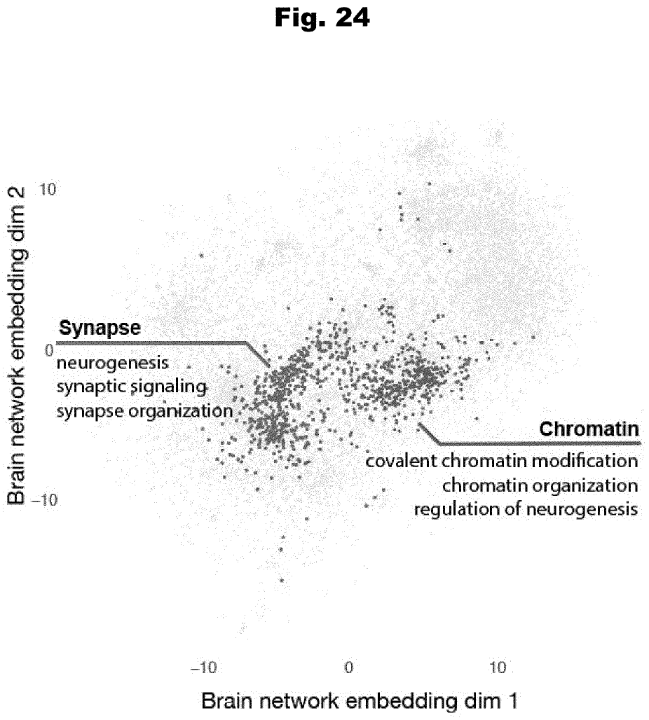

Globally, NDEA enrichment analysis pointed to a proband-specific role for noncoding mutations in affecting neuronal development, including in synaptic transmission and chromatin regulation (FIG. 23). Genes with significant NDEA enrichment were specifically involved in neurogenesis and grouped into two functionally coherent clusters with Louvain community detection algorithm (FIG. 24). The synaptic cluster is enriched in ion channels and receptors involved in neurogenesis (p=5.6×10⁻³⁸), synaptic signaling (p=4.8×10⁻³⁵) and synapse organization (p=1.5×10⁻¹⁸), including previously known ASD-associated genes such as those involved in synapse organization SHANK2, NLGN2, NRXN2, synaptic signaling NTRK2 and NTRK3, ion channels CACNA1A/C/E/G, KCNQ2, and neurotransmission SYNGAP1, GABRB3, GRIA1, GRIN2A²⁷. The synapse cluster is also significantly enriched for plasma membrane proteins (p=3.9×10⁻²⁴). In contrast, the chromatin cluster, representing chromatin regulation related processes, displayed an overrepresentation of nucleoplasm (p=2.1×10⁻⁹) proteins, with diverse functional roles including covalent chromatin modification (p=2.5×10⁻⁹), chromatin organization (5.2×10⁻⁸) and regulation of neurogenesis (p=6.4×10⁻⁵). The chromatin cluster also includes many known ASD-associated genes such as chromatin remodeling protein CHD8, chromatin modifiers KMT2A, KDM6B, and Parkinson's disease causal mutation gene PINK1 which is also associated with ASD. Overall, the results demonstrate pathway-level TRD and RRD mutation burden and identify distinct network level hot spots for high impact de novo mutations.

Next, the genetic landscape of ASD-associated de novo noncoding and coding mutations was examined. Specifically, in addition to the network analysis of noncoding mutations at the transcriptional and post-transitional level, it was also applied to the de novo coding mutations. The gene-specific NDEA statistic of elevated proband-specific noncoding mutation burden was compared to that of the coding mutations, finding a significant positive correlation for both TRD and RRD (p=0.004 for TRD, p=0.042 for RRD; two-sided permutation test). Moreover, by network analysis, TRD and RRD are themselves significantly correlated (p=0.034 two-sided permutation test). This demonstrates that coding and noncoding mutations affect overlapping processes and pathways, indicating a convergent genetic landscape, and highlights the potential of ASD gene discovery by combining coding and noncoding mutations.

### Experimental Study of ASD Noncoding Mutation Effects on Gene Regulation

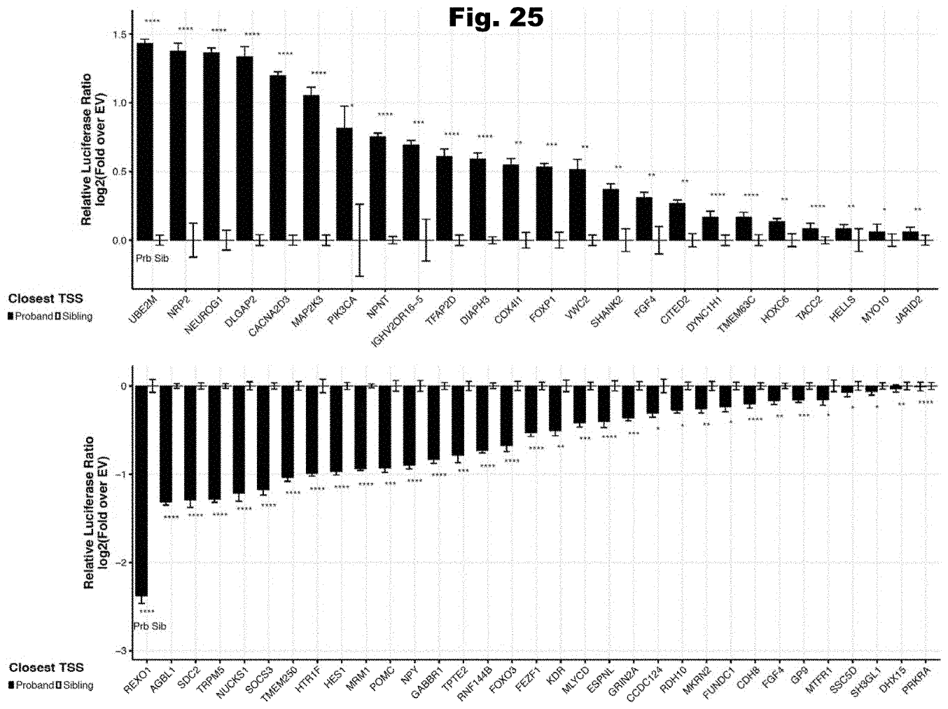

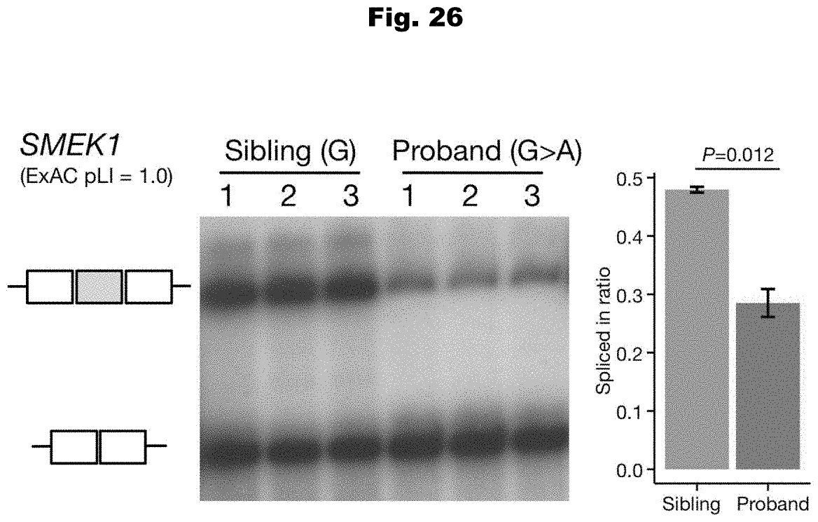

The gene network analysis identified new candidate noncoding disease mutations with potential impact on ASD through regulation of gene expression. In order to add further evidence to a set of high confidence causal mutations, allele-specific effects of predicted high-impact mutations was examined in cell-based assays (See Table 3 for variants tested). For TRD mutations, fifty nine genomic regions showed strong transcriptional activity with 96% proband variants (57 variants) showing robust differential activity (FIG. 25); demonstrating that the prioritized de novo TRD mutations do indeed lie in regions with transcriptional regulatory potential and the predicted effects translate to measurable allele-specific expression effects. To select and clone variant allele genomic regions, variants of high predicted disease impact scores larger than 0 and included mutations near genes with evidence for ASD association, including those with LGD mutations (e.g. CACNA2D3) and a proximal structural variant (e.g. SDC2). Mutations based on proximity to TSSs were not explicitly selected, and the chosen mutations lie from between 7 bp and 324 kbp away from nearest TSS, with most variants lying farther than 5 k from nearest TSS. For each allele (sibling or proband), either 230 nucleotides of genomic sequence amplified from proband lymphoblastoid cell lines was cloned or FragmentGenes synthesized by Genewiz were used. In both cases, 15 nucleotide flanks on 5′ and 3′ ends matched each flank of the plasmid cloning sites. The 5′ sequence was TGGCCGGTACCTGAG (Seq. ID No. 1) and the 3′ sequence was ATCAAGATCTGGCCT (Seq. ID No. 2). Synthesized fragments were cut with KpnI and BgIII and cloned into pGL4.23 (Promega) cut with the same enzymes. PCR-amplified genomic DNA was cloned into pGL4.23 blunt-end cut with EcoRV and Eco53kI using GeneArtCloning method from Thermofisher Scientific. All constructs were verified by Sanger sequencing.

To perform the luciferase reporter assays, human neuroblastoma BE(2)-C cells were plated at 2×10⁴ cells/well in 96-well plates and 24 hours later were transfected with Lipofectamine 3000 (L3000-015, Thermofisher Scientific) together with 75 ng of Promega pGL4.23 firefly luciferase vector containing the 230 nt of human genomic DNA from the loci of interest, and 4 ng of pNL3.1 NanoLuc (shrimp luciferase) plasmid, for normalization of transfection conditions. 42 hours after transfection, luminescence was detected with the Promega NanoGlo Dual Luciferase assay system (N1630) and BioTek Synergy plate reader. Four to six replicates per variant were tested in each experiment. For each sequence tested, the ratio of firefly luminescence (ASD allele) to NanoLuc luminescence (transfection control) was calculated and then normalized to empty vector (pGL4.23 with no insert). Statistics were calculated from fold over empty vector values from each biological replicate. High-confidence differentially-expressing alleles were defined by their ability to show the same effect in each biological replicate (n=3, minimum), drive higher than control empty-vector level gene expression, and the two alleles had significantly different level of luciferase activity by two-sided t-test. The data were normalized the fold over empty vector value of the proband allele to that of the sibling allele as shown in FIG. 25.

Among these genes with the demonstrated strong differential activity mutations, NEUROG1 is an important regulator of initiation of neuronal differentiation and in the NDEA analysis had significant network neighborhood proband excess (p=8.5×10⁻⁴), and DLGAP2 a guanylate kinase localized to the post-synaptic density in neurons. Mutations near HES1 and FEZF1 also carried significant differential effect on activator activities: neurogenin, HES, and FEZF family transcription factors act in concert during development, both receiving and sending inputs to Wnt and Notch signaling in the developing central nervous system and interestingly, the gut, to control stem cell fate decisions; and Wnt and Notch pathways have been previously associated with autism. SDC2 is a synaptic syndecan protein involved in dendritic spine formation and synaptic maturation, and a structural variant near the 3′ end of the gene was reported in an autistic individual. Thus, the method described herein identified alleles of high predicted impact that do indeed show changes in transcriptional regulatory activity in cells. Since many autism genes are under strong evolutionary selection, only effects exerted through (more subtle) gene expression changes may be observable because complete loss of function mutations may be lethal. This implies that further study of the prioritized noncoding regulatory mutations should yield insights into the range of dysregulations associated with autism.

In addition, as a case study for prioritized RRD mutations, the effect of an ASD proband de novo noncoding mutation laying outside of a canonical splice site that was predicted to disrupt splicing of SMEK1 was experimentally validated (ExAC pLI=1.0; FIG. 10). SMEK1 has previously been shown to regulate cortical neurogenesis through the Wnt signaling pathway.

For this mutation, a >40% reduction in the inclusion of the exon for the ASD proband allele compared to the sibling allele was observed in a minigene assay, which is in agreement with the high predicted RRD impact. This demonstrates the highly disruptive biochemical impact a non-splice site de novo mutation can have on RNA splicing.

The minigene assay was performed by first constructing the SMEK1 minigene by amplifying the genomic region with primers:—upstream exon+˜1,400 nt intron (TGTGTGGAGCACCATACCTACCA/CCACACTTGAACAAAACTCTATTGTCAAC) (Seq. ID Nos. 3 and 4) and alternative exon, downstream exon+˜1,400 nt intron (GGTAGGACACAAGTCTCCACAAAGC/GGCAGAGTTCATCAGATTGTAGCG) (Seq. ID Nos. 5 and 6). The produce was then cloned into pSG5 vector. Minigene (2 μg) was transfected into SH-SYSY cells. Cells were harvested 48 h post-transfection for immunoblotting or RT-qPCR following standard protocols. Three independent experiments were performed for statistical comparison.

Case Study: Association of IQ with De Novo Noncoding Mutation in ASD Individuals

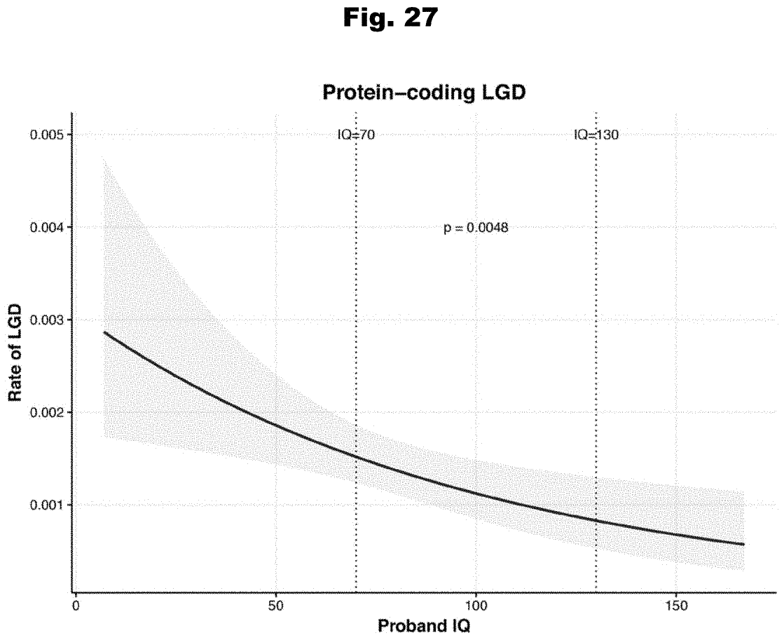

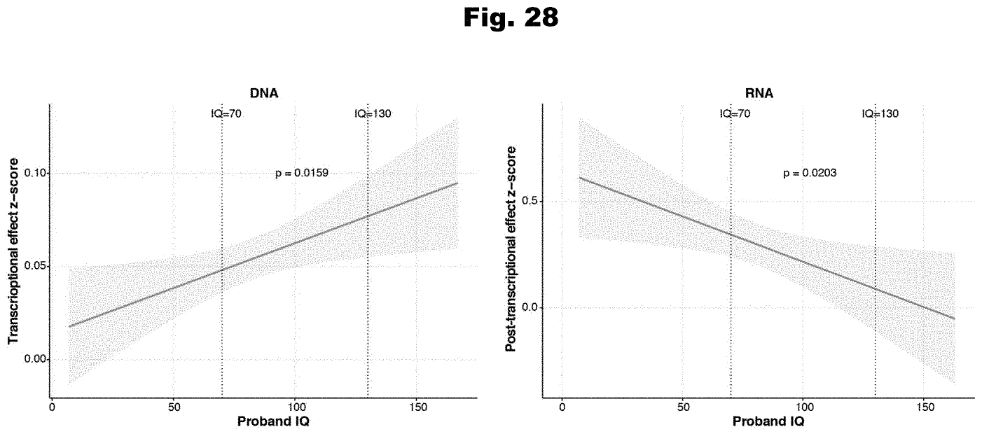

De novo noncoding mutations provide a vast space for exploration of phenotype heterogeneity in ASD. To illustrate the potential of such analyses, a case study focused on IQ was performed. Intellectual disability is estimated to impact 40-60% of autistic children, and ASD individuals can also over-inherit common variants associated with high education attainment. The genetic basis of this variation is not well understood. Despite the genetic complexity observed in association with ASD proband IQ, past efforts to identify mutations that contribute to ASD found that these mutations are also negatively correlated with IQ. Specifically, in analyses of exome sequencing data from different ASD cohorts, a significant association was observed between lower IQ and higher burden of de novo coding likely-gene-disrupting (LGD) (see FIG. 27) and large copy number variation (CNV) mutations. For de novo noncoding mutations analyzed in this study, a significant association between noncoding mutations and IQ in ASD individuals was observed. Intriguingly, it was found that higher IQ ASD individuals have a higher burden of TRDs, whereas lower IQ ASD individuals have a higher burden of RRDs in ExAC LoF intolerant genes (FIG. 28, DNA p=0.016, RNA p=0.020). Thus, it is tempting to speculate that while mutations that are damaging to the protein through disruption of coding (LGD or large CNVs) or RNA processing (RRD) are likely to increase the risk of lower IQ in ASD context, mutations affecting transcriptional regulation (TRDs) can affect ASD without the coupled negative effect on IQ. This analysis was performed by computing the maximum probability differences across features for each mutation, and testing for its association with IQ using linear regression with two-sided Wald test on the slope coefficient. For DNA analysis, all variants that are within 100 kb from the TSS were used. For RNA analysis, the mutations were restricted to genes with ExAC pLI>0.9 and are intronic within 400 nts to an exon in an alternatively splicing regulatory region.

### Further Analysis of Posttranscriptional Variants in ASD

A pathogenic role of RBP dysregulation in ASD and other complex disorders has been proposed based on observations of deleterious mutations present within coding sequences of genes encoding RBPs. However, little is known with regard to the downstream role that variants along an RNA sequence might play in disrupting RBP-RNA interactions, especially for rare and de novo mutations, primarily due to the difficulty in interpreting the functional impact of RNA dysregulation at scale. To approach this problem, a new machine learning framework, Seqweaver, was developed that incorporates a collection of in vivo mapped RBP binding maps and couples this data with a deep learning algorithm to predict noncoding variant effects on RBP-RNA interaction. The resulting methodology enabled investigation into the impact of noncoding de novo mutations at single nucleotide resolution simultaneously on hundreds of RBPs in a case-control ASD cohort of 2,075 whole genomes. Using Seqweaver, a previously undiscovered excess burden of noncoding de novo RRD mutations among ASD probands compared to their unaffected siblings (a control set providing the critical matching backgrounds) was found, impacting a large collection of RBPs and target transcripts involved in numerous brain developmental processes. Further evidence of a causal role in ASD etiology, it was found that high impact noncoding RRD mutations are associated with the severity of specific phenotypes observed within ASD children, supporting the value of noncoding variants in clinical applications.

### Quantitative Prioritization of RBP Altering Noncoding Variants

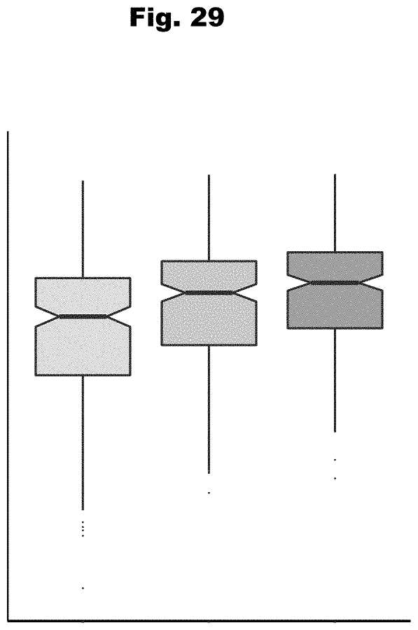

Noncoding nucleotide substitutions comprise the largest fraction of autism de novo variants, however, prioritizing clinically relevant variants in noncoding sequences, including those that disrupt RBP binding, has been challenging, especially at a single nucleotide resolution. Modeling RBP binding sites is difficult due to their short degenerate motifs, so a deep learning-based method Seqweaver was developed, which was trained on precise biochemical profiles of RBP-RNA interactions. This training set was used to generate a quantitative model to estimate the binding of RBPs from RNA sequence features alone. Seqweaver leverages a deep convolution network to then integrate evidence beyond a single motif and include surrounding sequence features located up to 500 nucleotides (nt) away. This allows it to take into account features such as potential sites of multiple trans-acting factor binding sites and locations of splice sites (FIG. 29). These sequence features provide the basis of a network of interweaving dependencies that collectively lead to the ability to accurately predict RBP binding sites. Disruption of any subset of these sequence features can be modeled by Seqweaver to predict the functional effect of variants on RBP target binding, and ultimately their effect on specific phenotypes.

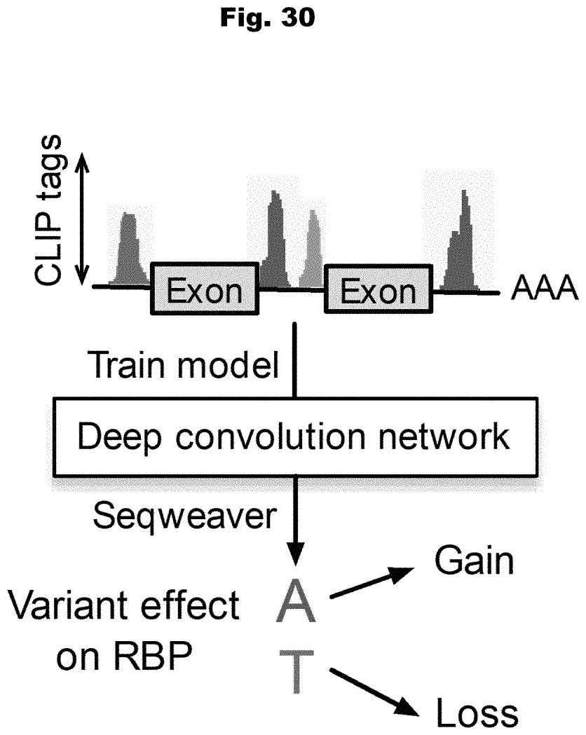

To build a sequence feature models for each RBP, Seqweaver was trained using in vivo RBP binding profiles mapped using cross-linking immunoprecipitation (CLIP) from a large set of previously published and newly available Encyclopedia of DNA Elements (ENCODE) datasets (FIG. 30). In total, a comprehensive compendium of 231 CLIP binding profiles and a branch-point mapping profile was used to build the Seqweaver RBP models (full list of input datasets are available electronically via Electronic Data Table 2), thus allowing simultaneous prediction of the genomic variant effect on each RBP by quantifying the predicted probability difference of RBP binding between the reference and alternative allele.

A systematic evaluation of Seqweaver's ability to predict variant effect on RBP binding was conducted by leveraging allelic imbalance occurring at single nucleotide polymorphisms (SNPs) observed in the human population. When a heterozygous SNP overlaps a RBP binding site, the RBP binding preference of the RNA transcribed by the two alleles can be measured by the allelic imbalance of the observed CLIP sequenced reads. A non-disruptive SNP should generate comparable number of RNA CLIP reads from each SNP allele, while a high impact SNP would cause an imbalance in RNA CLIP reads. To generate these evaluation SNPs, the initial analysis was conservatively restricted to heterozygous 1000 Genomes Project variants for which the genotypes for each allele independently in both CLIP and RNA-seq data could be observed from the same sample cells or individual (total 34,781 allelic imbalanced SNPs).

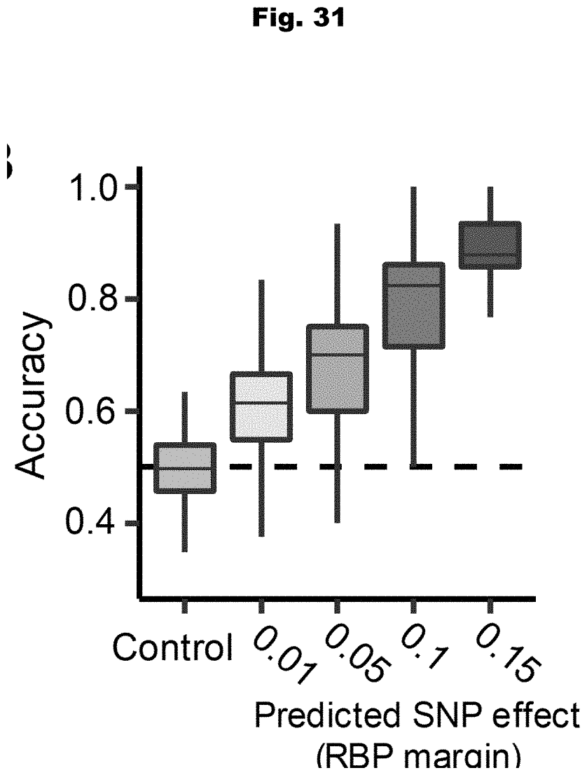

Using these SNPs as an evaluation set, Seqweaver was able to accurately predict the allele with greater RBP affinity, and did so with increasing accuracy as the threshold was increased for the predicted binding difference between the two alleles (FIG. 31). As a control, the accuracy trend could not be detected when only using the observed RNA-seq allele frequency (i.e., RNA-seq reads quantifying allele-specific expression of the RNA transcript) as a predictor for RBP binding.

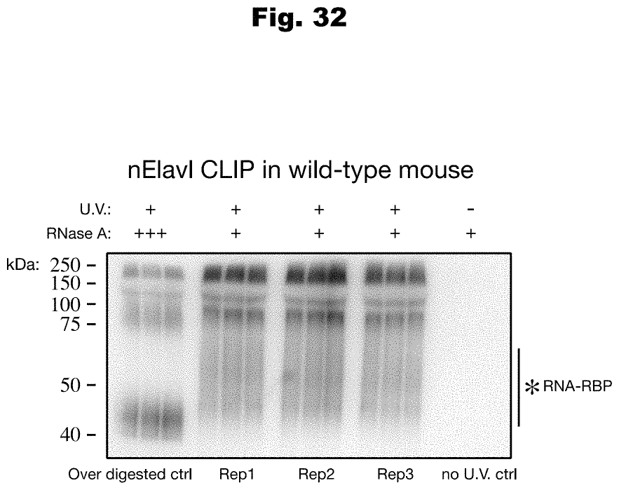

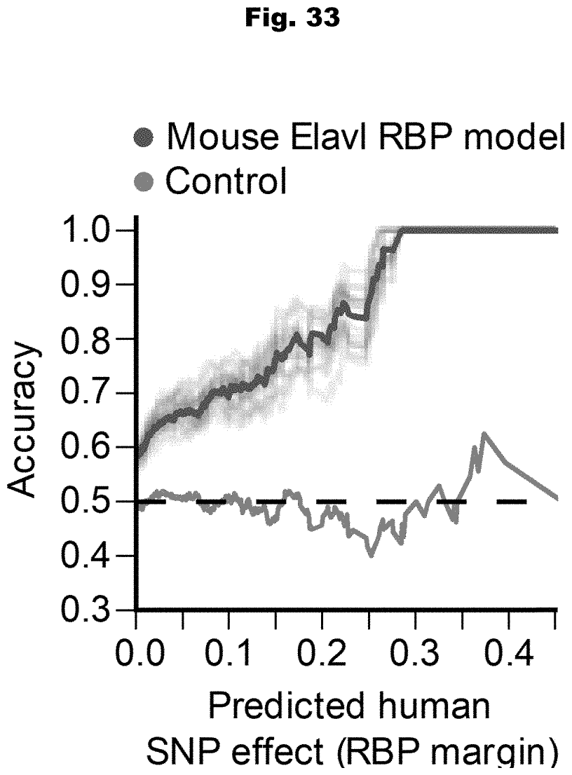

Seqweaver was tested to see if it could accurately predict the variant effect in the human brain, an important task due to the major role neuronal cells are believed to play in determining autism pathogenicity. In a previous work, the in vivo neuronal ELAVL (nELAVL) RBP binding sites in the human prefrontal cortex was mapped by conducting nELAVL-CLIP in 17 postmortem individuals in which the same samples were also subjected to RNA-Seq. Using this data, a total of 1,725 1000 Genomes Project SNPs were identified that overlapped with nELAVL binding profiles in human neuronal cells in vivo. Neuronal RBPs and RNA processing are highly conserved, thus it was hypothesized that Seqweaver trained on mouse nElavl profiles should be able to predict the higher affinity human allele despite being trained on mouse sequence data. The nElavl-CLIP method was performed in adult mouse cortex (3 biological replicates, FIG. 32) and Seqweaver was trained with only the mouse RBP sequence profiles. Consistent with the human RBP profile models, the mouse Seqweaver results accurately predicted the higher affinity human allele (FIG. 33)—demonstrating that Seqweaver can learn the deep sequence dependency required for RBP binding conserved from mouse to human.

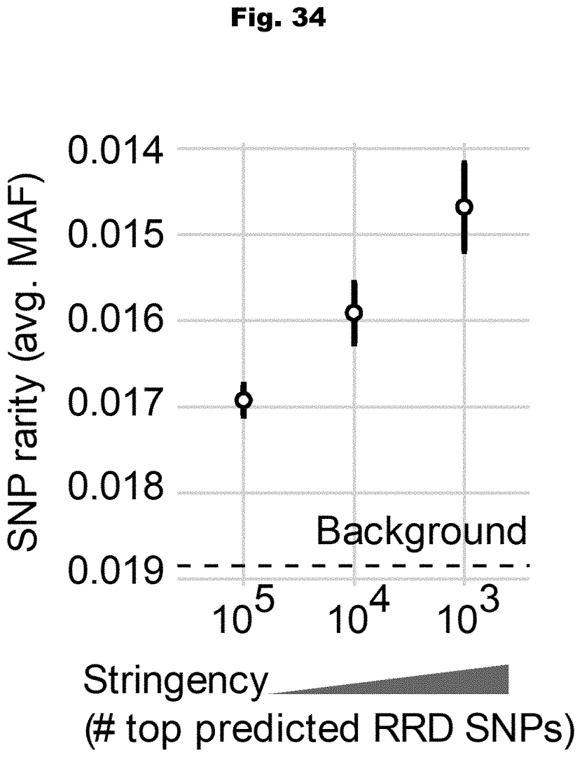

Furthermore, Seqweaver predicted the effect on RBP binding interactions for the human genetic variation captured by the 1000 Genomes Project, comprising all SNPs in noncoding exonic regions or introns flanking exons (up to 500 nt, total of 5,504,053 SNPs). SNPs predicted by Seqweaver to be RRD variants were also more likely to be under purifying selection based on their lower minor allele frequency (MAF, compared to regional background) and therefore more likely to be deleterious (FIG. 34). This result demonstrates an important capability of Seqweaver: prioritizing variants with biochemically interpretable impact that are under negative selection in the human population. This is a crucial task in understanding human disease, particularly developmental disorders such as autism that are associated with disruptive variants that are likely to be under strong selection.

### The Burden of Noncoding De Novo Mutations in Autism

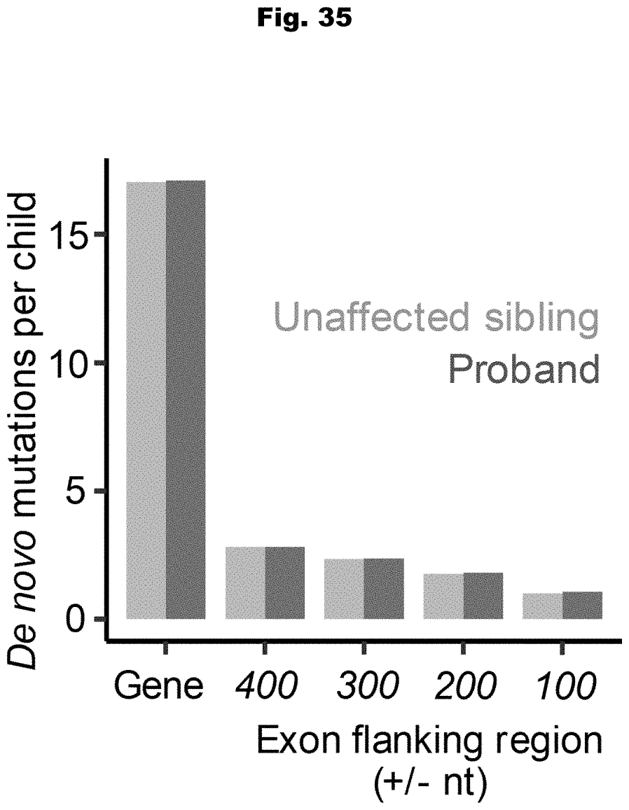

The burden of RBP dysregulation in autism was investigated by applying Seqweaver to de novo variants called from whole genome sequencing (WGS) in a cohort of total 2,075 individuals from the Simons Simplex Collection (SSC). These individuals include 528 ASD probands, 487 unaffected siblings and unaffected parents. Because only one member of these simplex families was diagnosed with autism, the relative contribution of de novo mutations in probands is likely to be high. Previously, whole exome sequencing (WES) on SSC families was used to identify an association between coding de novo likely-gene-disrupting (LGD) mutations and autism pathogenicity. To date, efforts to identify noncoding variant categories linked to ASD pathogenesis have been very limited. Indeed, the number of de novo variants per proband in gene regions and small window surrounding exons showed no significant difference compared to the unaffected siblings when used as control (FIG. 35). Despite the observation that the total number of de novo variants showed minimal differences, it was reasoned that mutations that alter RBP-binding in noncoding sequence could nonetheless be enriched in the proband compared to their unaffected siblings. To test this hypothesis, Seqweaver was used to estimate the maximum variant effect on RBP binding for each noncoding de novo variant within genic noncoding regions observed in the proband and their siblings.

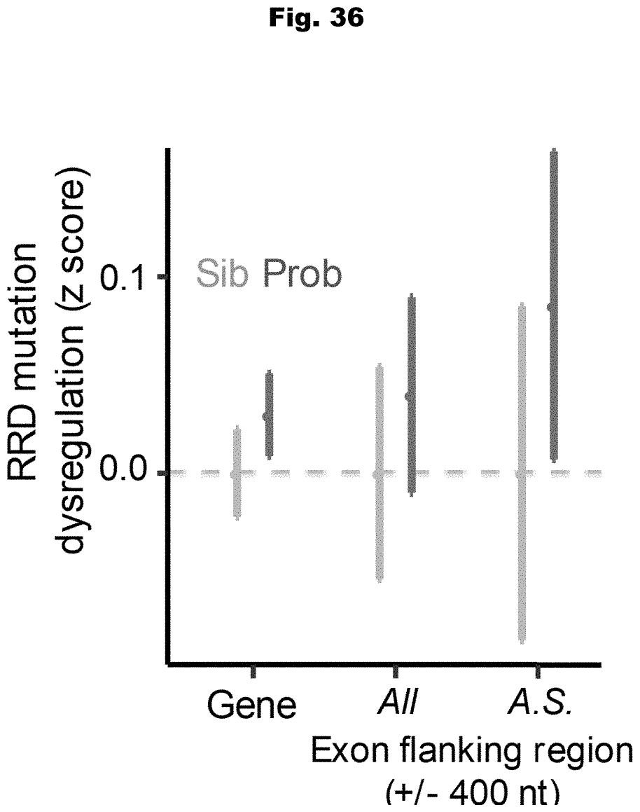

Indeed, the proband burden of large effect RRD mutations in noncoding genic regions was significantly larger than the sibling burden (one-sided Wilcoxon rank-sum test p-value=0.02, FIG. 36). When analysis was restricted to a smaller window flanking exons (400 nt, all following analysis focused on this region), based on prior estimates of regions of high-density RNA regulatory elements, it was observed more severe RRD mutations in the proband compared to control siblings. Alternatively spliced (AS) exon regions are believed to have a higher susceptibility to deleterious mutations, highlighted by their greater intronic conservation surroundings. As predicted, a stronger statistical enrichment of high impact RRD mutations was detected in probands when assessing only exonic regions that were previously discovered to be alternatively spliced (p-value=0.035, FIG. 36). These included RRD mutations within previously identified strong candidate ASD disease genes such as SYNGAP1, SETDS and INTS6.

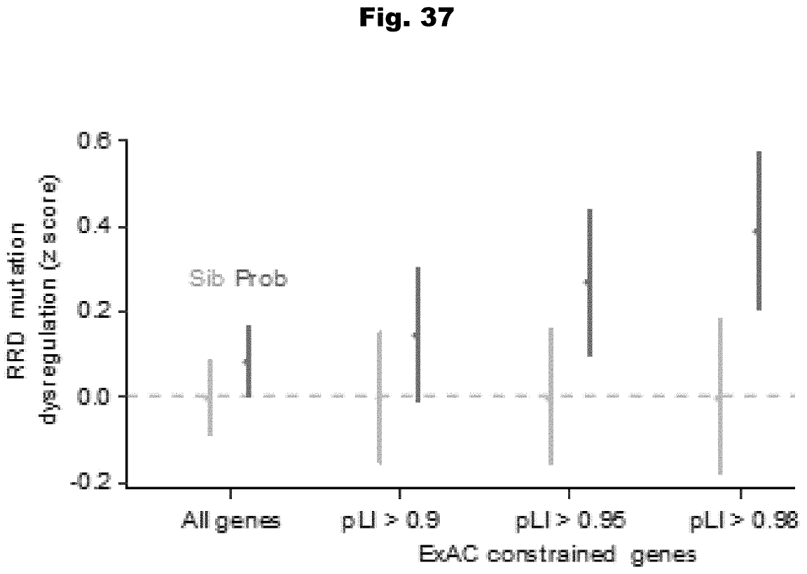

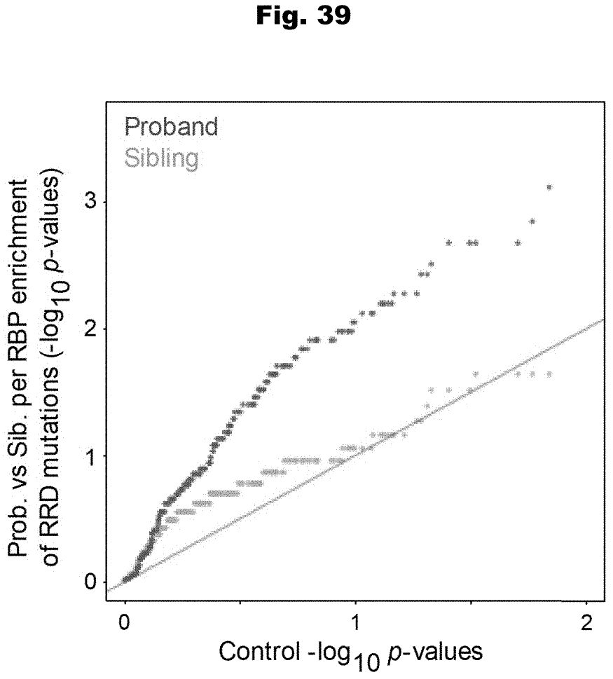

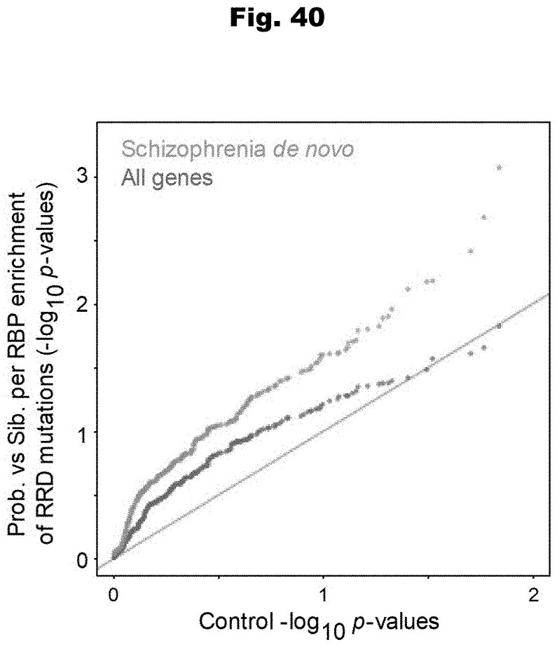

Previous reports in autism, schizophrenia and developmental disorders have presented findings of the clustering of rare disruptive coding variants in a collection of genes that are under high purifying selection. It was tested whether highly constrained genes were also enriched for large effect noncoding de novo RRD mutations. Using constrained genes, as defined by the Exome Aggregation Consortium (ExAC), a greater enrichment signature was observed with increasing constraint stringency (FIGS. 37 & 38, probability of loss-of-function intolerance—pLI; constrained genes pLI>0.9: p-value=0.05; pLI>0.95: p-value=0.013; pLI>0.98: p-value=7.8×10-4; one-sided Wilcoxon rank-sum test), reflecting strong selection against noncoding disruptive variants within these constrained genes, as defined by whole exome sequencing. Furthermore, the group of constrained or recurrent genes harboring de novo coding LGD mutations in the probands (127 genes) showed a higher statistical enrichment of RRD mutations compared to genes with LGD mutations found in the unaffected siblings (175 genes, FIG. 39). This trend of a higher burden of RRD mutations in probands was also observed among published de novo coding variant harboring genes linked to schizophrenia (609 genes, FIG. 40).

### FMRP Targets to Link ASD in Noncoding Genomic Regions

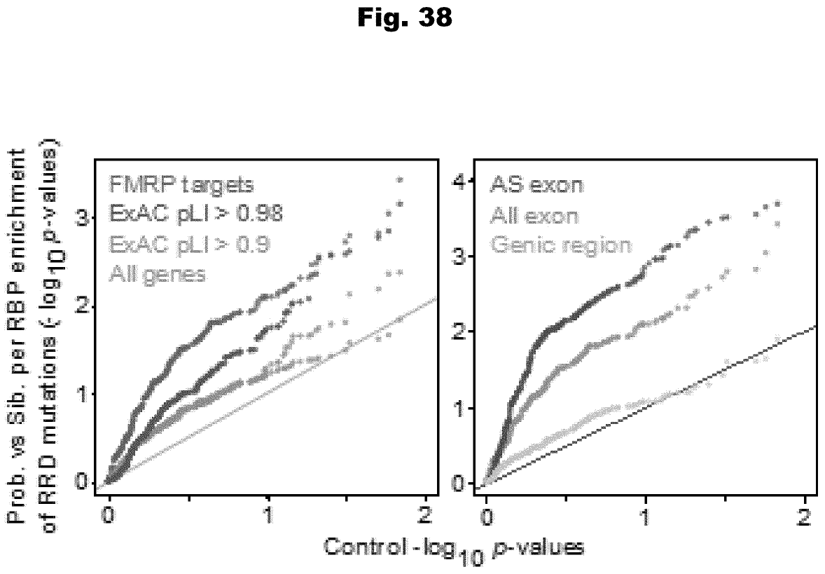

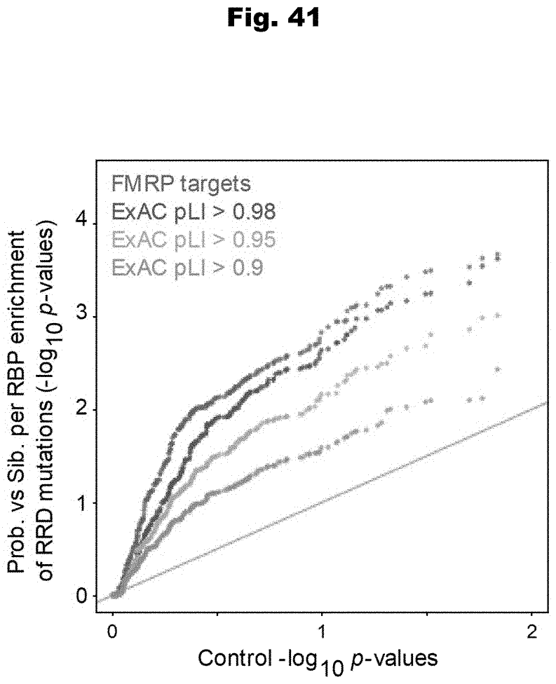

Because fragile X mental retardation protein (FMRP) has been found to be disrupted in ˜2% of ASD patients and is the most common monogenic cause of ASD the targets of FMRP were examined. It was previously demonstrated that FMRP regulates translation of a network of brain mRNAs by stalling ribosome elongation. These FMRP mRNA targets have been subsequently found to be encoded by one of the most highly enriched sets of genetically linked loci in both autism and schizophrenia studies. It was found that the biochemically identified FMRP targets have significant overlap with the highest constrained genes in ExAC (682/1,498 genes overlap with ExAC pLI>0.98 2,130 genes, hypergeometric p-value<1×10⁻¹⁴). In concert with previous ASD studies examining coding regions, it was further found that FMRP targets showed strong proband enrichment for noncoding RRD mutations disrupting numerous RBPs in exon-flanking regions and this enrichment was highest surrounding AS exons (FIG. 38, AS exon region comparison FIG. 41).

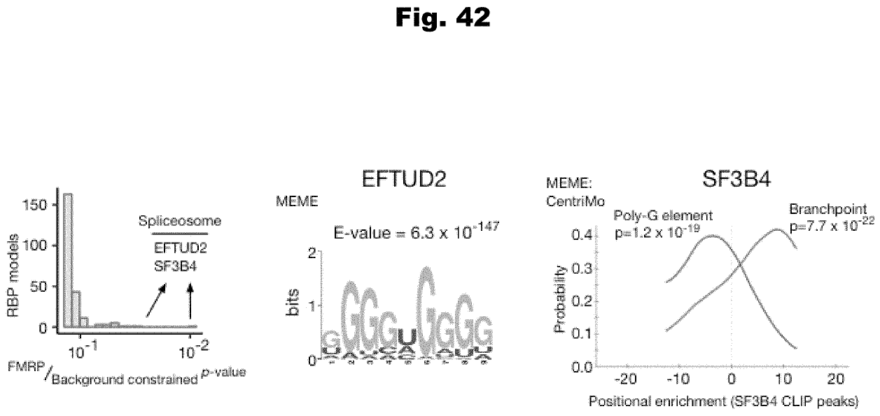

The etiology of fragile X syndrome (FXS) demonstrates the importance of precise stoichiometry and dosage control for the collection of FMRP targets in the brain. Consequently, it was reasoned that FMRP targets might be subjected to an additional layer of regulation during RNA processing (i.e., upstream of translation) and therefore constitute hotspots for ASD RBP dysregulation. It was tested whether any RBPs' enrichment of high impact proband RRD mutations compared to siblings were more likely to occur in FMRP targets compared to the background constrained genes. Interestingly, two spliceosome associated RBPs, EFTUD2 and SF3B4, were found to have the largest differential burden among FMRP targets (differential burden enrichment for both factors p-value<0.05, permutation test; FMRP targets proband RRD enrichment EFTUD2 p-value=2.2×10⁻⁴, SF3B4 p-value=7.6×10⁻⁴, one-sided Wilcoxon rank-sum test, FIG. 42). Haploinsufficiency of either EFTUD2 or SF3B4 have previously been found to cause severe disorders including craniofacial malformation, microcephaly and developmental delay, features shared in part with FXS. Furthermore, analysis of CLIP profiles of the two spliceosome components suggest a concentrated regulation of FMRP targets by these factors compared to the background constrained genes surrounding intronic poly-G elements (FIG. 42), which have been previously reported to act as splicing enhancer elements.

### Functional Clustering of Noncoding De Novo RRD Mutations in ASD

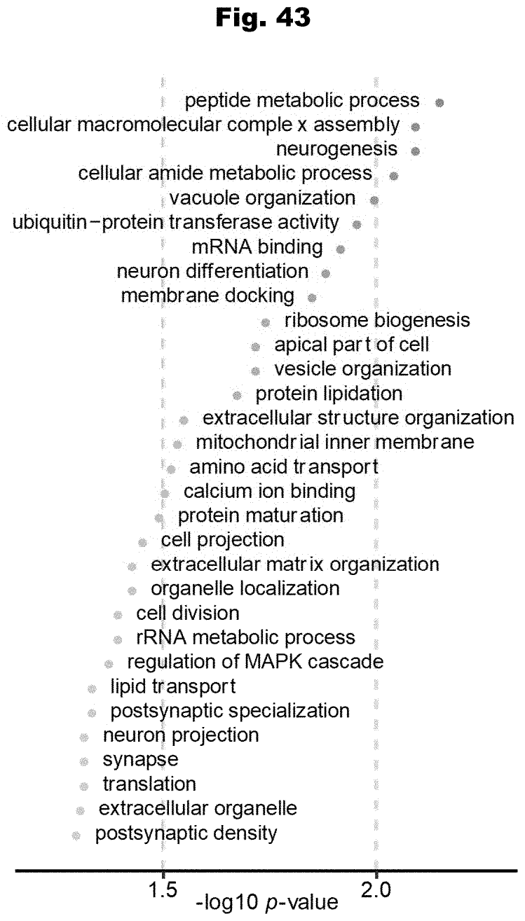

An enrichment analysis was conducted to identify cellular functions and pathways that show an excess burden of high impact RRD mutations (FIG. 43, GO terms p-value<0.05, FDR<0.1). Consistent with the model of neuronal dysregulation, a significant enrichment among neuronal processes was found, including neurogenesis, neuronal projection, synaptic, and postsynaptic density associated genes. The MAPK pathway and its downstream regulatory processes (e.g., cell cycle) were also identified. In addition, an enrichment among a collection of core cellular processes was found, including RNA processing (mRNA binding proteins p-value=0.012), translation pathways (e.g., translational regulation p-value=0.048) and downstream pathways controlling posttranslational modification (ubiquitination p-value=0.011 and protein maturation p-value=0.032). This result supports and extends observations suggesting an intricate interconnection between core pathways and ASD etiology, as made for constrained genes in the ExAC study, and as previously observed in the functional role of ASD risk genes TOP1 (topoisomerases, transcriptional activator), FMRP (translational repressor) and CUL3 (ubiquitin ligase complex, posttranslational regulator).

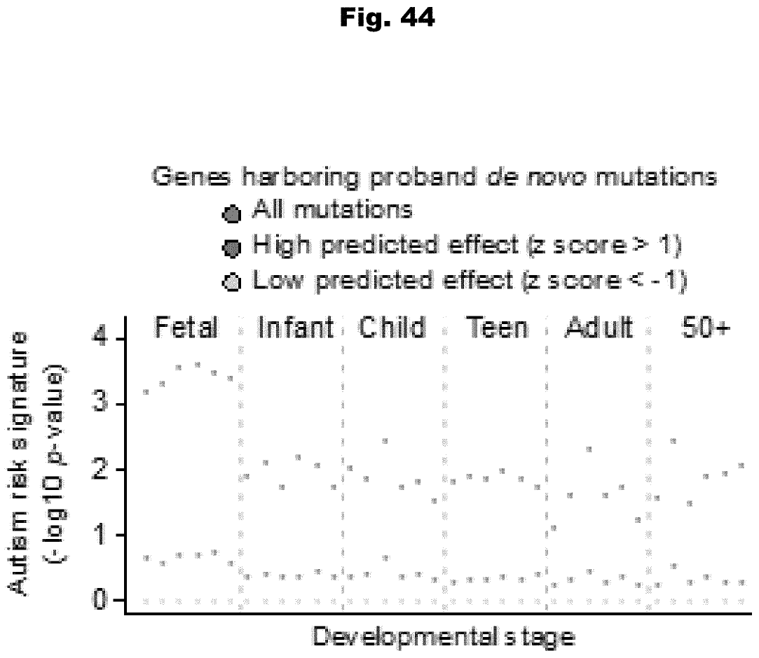

One of the hallmarks of autism is altered brain development, and a major focus of research has been to understand embryonic or early postnatal development in autism. The noncoding RRD mutations discovered were used together with gene expression RNA-seq data of the developing human brain to conduct an unbiased investigation into the temporal window of autism pathogenicity. For each RNA-seq dataset from an unaffected human brain specimen (prefrontal cortex), an autism risk signature was calculated by testing the up-regulation of expression for genes harboring a proband RRD mutation compared to the control set of mutated genes from siblings. Our analysis (FIG. 44), showed a general trend of up-regulation of RRD mutation harboring genes—with the fetal stage demonstrating the highest autism risk signature (one-sided Wilcoxon rank-sum test p-value<0.001). This pattern was only observed for de novo mutations predicted to have a large RBP dysregulation effect in ASD. In addition, we found that the collection of proband de novo mutations was consistently enriched among genomic regions with significantly higher embryonic stage expression during development compared to sibling mutations (Fisher's exact test p-value=0.01543, odds ratio=1.8).

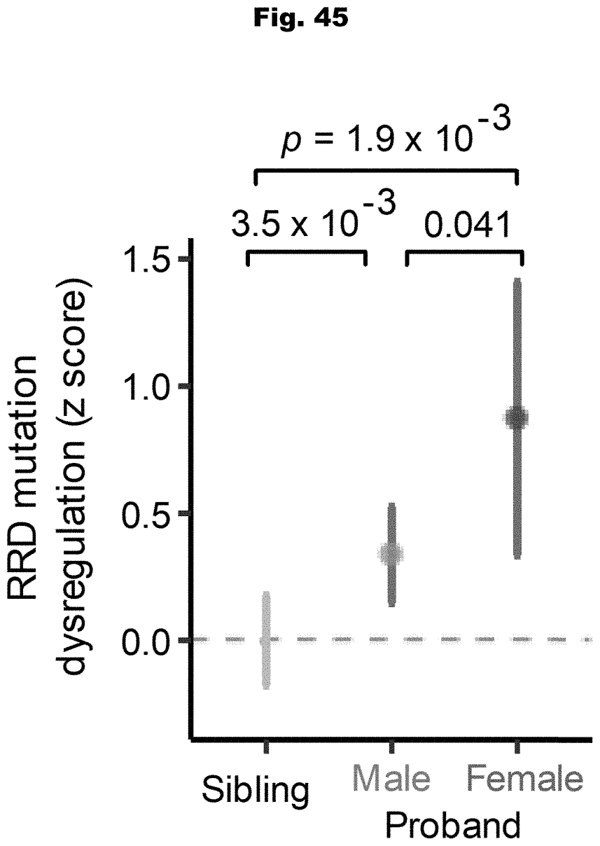

The clustering of noncoding RRD mutations in connection to gender disparity observed in ASD was also examined. The occurrence of autism is ˜5 times higher among males than females. Previous genetic studies have suggested that females may possess protection against ASD risk variants. When comparing the predicted effects of RRD mutations among constrained genes, the female probands exhibited a significantly higher enrichment of large effect RRD mutations compared to both male probands (p-value=0.041, FIG. 45) and unaffected siblings (p-value=1.9×10⁻³). Hence, females may have a higher threshold of tolerance for dosage and stoichiometry perturbations among these highly constrained genes, potentially due in part to sexual dimorphism.

Noncoding Mutations are Associated with Clinical Phenotype in ASD

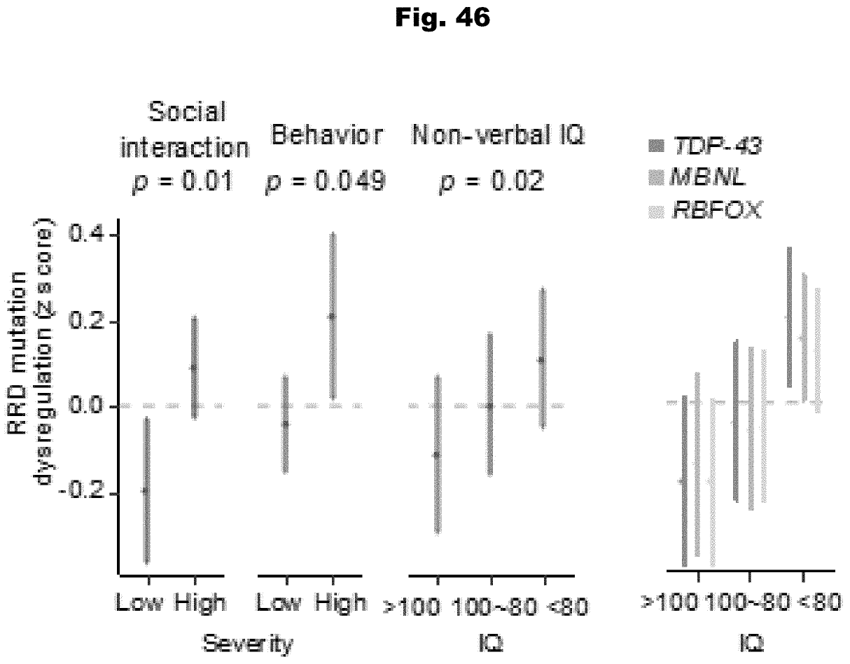

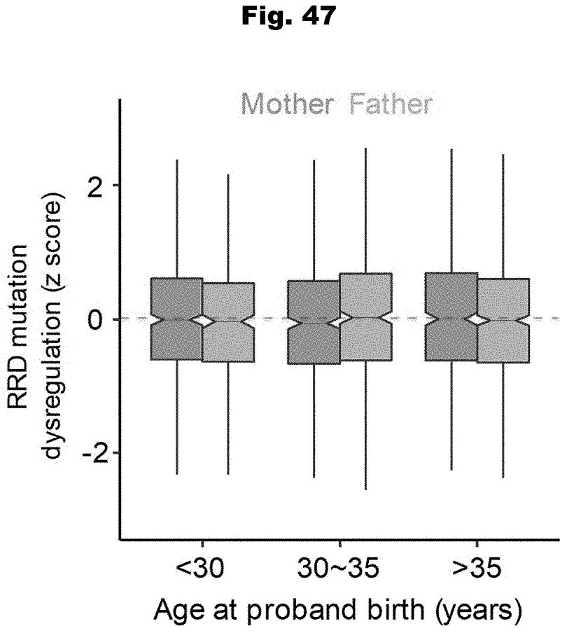

Large collections of studies examining ASD cohorts have identified substantial heterogeneity in their clinical phenotypes. Thus, RBP dysregulation association with clinical diversity among the probands was investigated. Altered social interaction and repetitive or stereotyped behavior are the key clinical indications for diagnosing autism spectrum disorder. Among constrained genes, it was found that probands with high impact noncoding RRD mutations displayed a greater alteration in both social interaction (ADI-R social total, p-value=0.01, Pearson product-moment correlation coefficient test for all) and behavior (ADI-R behavior total, p-value=0.049) (FIG. 46), consistent with the trend of an increased burden in comparison to unaffected siblings. Conversely, as a control, we observed no association between the parent ages at proband birth and the predicted effect of a de novo mutation (FIG. 47, the total count of de novo mutations is correlated with parent age).

Intellectual disability is estimated to impact 40-60% of autism children. Accordingly, non-verbal IQ has previously been associated with the ascertainment of de novo coding LGD mutations. Similar to LGD mutations, a significant correlation between non-verbal IQ and the predicted effect of noncoding RRD mutations was observed (p-value=0.02). Among individual RBP models, probands harboring RRD mutations for RBP TDP-43, MBNL and RBFOX showed the greatest association with non-verbal IQ (FIG. 46). TDP-43 has previously been linked to amyotrophic lateral sclerosis (ALS) and frontotemporal dementia, and has been shown to regulate long pre-mRNA abundance levels and splicing in the brain. The highly constrained TDP-43 (ExAC pLI=0.98) also appears to have a crucial developmental role reflected by the embryonic lethal phenotype of TDP-43 knockout mice, coupled with our observed association with early intellectual disability.

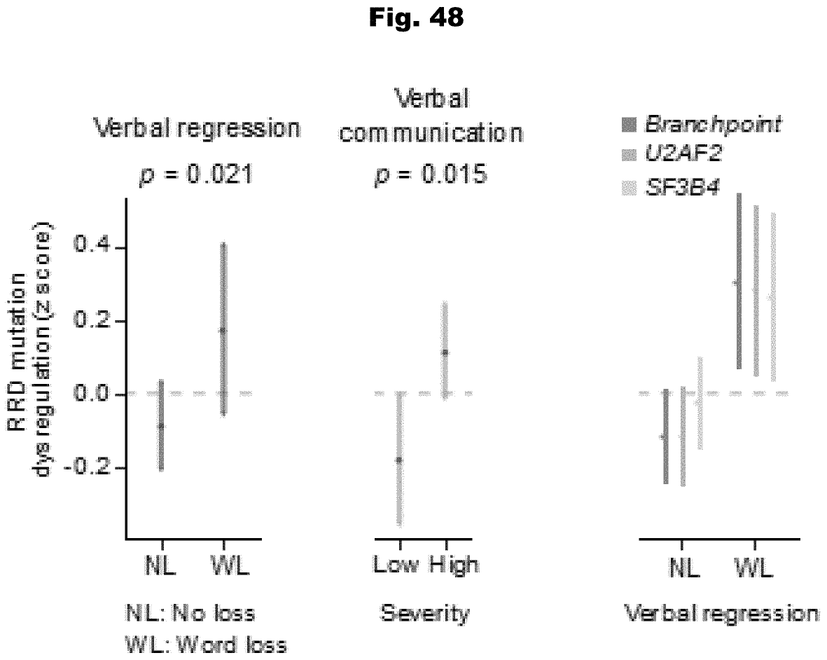

A heterogeneous aspect of phenotypic outcome in autistic children is verbal communication. Specifically, verbal regression is characterized by the loss of word and communication skills after the first few years. Unlike IQ, the existence of a genetic link and the subsequent molecular basis of this phenotype has been uncertain. The de novo mutations within constrained genes into two groups based on the probands verbal regression phenotype (word loss or no loss of verbal communication) were segregated). After de novo mutations were stratified by proband phenotype, a statistically significant association between verbal regression and the predicted effect of noncoding RRD mutations was observed (p-value=0.021, FIG. 48). Notably, RBP models with connections to the RNA branch-point showed the greatest association with the verbal regression phenotype (branch-point, U2AF2 and SF3B4, FIG. 48). Further evidence of a genetic link connecting various verbal communication phenotypes, revealed that large effect RRD mutations were also significantly associated with probands that had past incidences of abnormal verbal communication behavior (ADI-R verbal communication total, p-value=0.015). The significant correlation between the predicted effect of noncoding RRD mutations and various ASD verbal phenotypes indicates a possible genetic contribution to these clinical conditions and warrants further investigation into the etiology of verbal regression.

### Seqweaver Method Design

A machine learning approach of deep convolutional neuronal networks (ConvNet) was utilized to build a quantitative model of the RNA sequence features required for each RBP binding. ConvNets allow researchers to design network architectures that can leverage information of high order motifs at different spatial scales but with optimal parameter sharing to avoid overfitting. The ConvNet architecture consists of an initial input layer followed by a series of convolution and pooling layers. The input layer contains a 4×1,000 matrix that encodes the input RNA sequence of U, A, G, C across the 1,000 nt window anchored around the RBP binding site. The subsequent convolution layer looks at 8 nts at a time shifting by 1 nt and computes the convolution operation of 160 kernels. At this first convolution level, the kernels are equivalent to searching for a collection of local sequence motifs in a one-dimensional RNA sequence. Analogues to neurons, a rectifier activation function (ReLU) was then applied such that sets the convolution layer output to a scale of minimum of 0 (i.e. ReLU(x)=max(0,x)). Thus formally, input S results in convolution layer output location n for kernel k as the following:

$$Convolution {( S )}_{n , k} = ReL U ( {\sum }_{i}^{I} {\sum }_{d}^{D} {w}_{i , j}^{k} {S}_{n + i , d} )$$

where I is the window size and J is the input depth (e.g., for the fist convolution layer I corresponds to the local sequence motif length and J represents the four RNA bases).

Next, a pooling layer that allows the reduction of the dimensional size of the network and parameters was added. Specifically, every window of 4 for a kernel output are collapsed into the maximum value observed in that span. Subsequently, the resulting output is used as input for a sequence of convolution (2^{nd}), ReLU, pooling and convolution layer (3^{rd}) in which higher order sequence motifs can be derived based on the first layer local motifs (2^{nd} cony. layer 320 kernels, 3^{rd} cony. layer 480 kernels with identical ReLU and pooling layer).

Finally, a fully connected layer (size human 217, mouse 43) that can now take the resulting output from the three convolution steps to integrate across the entire 1,000 nt context was added to derive a final set of high order sequence motifs. These high order sequence motifs are shared across all RBP models that allow optimal parameter reduction, but also are based on the biological intuition that many RNA sequence features are shared in the cell (e.g., splice sites and branchpoints). The fully connected layer outputs (i.e., high order sequence features) are then subjected to RBP-specific weighted logistic functions (sigmoid, [0,1] scale) allowing for the simultaneous prediction of each RBP binding propensity to the input RNA sequence.

Training the ConvNet for all parameters were conducted using primarily a CLIP-derived training set to minimize the objective function of the following loss function:

$$Objective ( w , h ) = {NLL}_{w , h} + {λ}_{1} {‖ w ‖}_{2}$$

$${NLL}_{w , h} = - {\sum }_{i} {\sum }_{j} {L}_{j}^{i} log ( {f}_{j} ( {S}^{i} ) ) + ( 1 - {L}_{j}^{1} ) log ( {f}_{j} ( {S}^{i} ) )$$

Here, i indicate the training examples and j indicates the RBP features. Lⱼⁱ is the training label (0 or 1) for example i and RBP feature j. fⱼ(Sⁱ) represents the ConvNet predicted probability of RNA sequence Sⁱ of being a binding site for RBP j. For regularization, L2 regularization (λ₁) was used for all weighted matrix values, and random dropout of outputs following each convolution-pooling series was applied. The loss function was optimized using a stochastic gradient decent. Full list of parameters used in model is provided below:
1. Convolution layer—160 kernels. Window size: 8. Step size: 1.
2. Pooling layer—Window size: 4. Step size: 4.
3. Convolution layer—320 kernels. Window size: 8. Step size: 1.
4. Pooling layer—Window size: 4. Step size: 4.
5. Convolution layer—480 kernels. Window size: 8. Step size: 1.
6. Fully connected layer—human Seqweaver 217 neurons, mouse Seqweaver 43 neurons
7. Sigmoid output layer

### Parameters:

### Dropout Proportion:

Layer 2: 10%

Layer 4: 10%

Layer 5: 30%

All other layers: 0%
L2 regularization (λ₁): 8e⁻⁷
Max kernel norm: 0.9.

### Training Data for Seqweaver

231 CLIP binding profiles for 82 unique RBPs and a branchpoint mapping profile were used as input features. In addition, 28 annotated splice site (3′ and 5′) features were including as experimental features, but were not included for subsequent ASD variant impact analysis. ENCODE processed CLIP data was downloaded for uniform peak calling together with non-ENCODE data. All gene regions defined by Ensembl (mouse build 80, human build 75) were split into 50 nts bins. All bins that overlap repeat regions were removed (RepeatMasker). For each bin, RBP features that overlapped more than half were assigned a corresponding positive label. Negative labels were assigned to bins with at least one RBP peak (excluding the RBP of training). CLIP peaks from chromosome 4, 9, 13 and 16 were used for evaluation of input sequence context window. Seqweaver code and input data is available at seqweaver.princeton.edu.

### Generating Evaluation Set of 1000 Genome Project SNPs

Genome Analysis Toolkit was used and following GATK best practice guidelines for RNA-Seq based genotyping the biological samples (17 postmortem human prefrontal cortex specimens, HeLa, 293T, ENCODE tier 1 cell lines—HepG2 and K562). All raw sequencing files were aligned to the genome using STAR aligner (2.4) followed by HaplotypeCaller (RNA-seq mode) to call variants. To reduce false positive calls, only heterozygous 1000 Genome Project SNPs were used for subsequent analysis. As an additional filter for both accurate variant calling and quantifying allele-specific reads, the WASP methodology that utilizes a post-processing remapping strategy of all reads with the alternative allele to reduce any biases was applied. Any SNP following WASP post-processing (i.e., remapping test of alt. allele reads) that did not have a MAF of >0.01 (ratio of RNA-seq reads derived from minor allele) or read coverage more than 10 were removed from the pool of SNPs for each sample.

Next, the sample specific SNPs were overlaid to the alignment files from CLIP experiments of the same corresponding sample type (total 102 RBP-sample type combinations) using GATK ASEReadCounter tool. Analogues to RNA-Seq, the WASP method was applied to each CLIP derived reads to produce the final CLIP observed genotype and allele-specific read count for each sample. Conservatively, only SNPs that had the same observed genotype from both RNA-Seq and CLIP were used, despite the loss of the most impactful SNPs that lead to complete loss of RBP binding. Additionally, only 1000 Genome Project SNPs were used, excluding any indels that are more challenging to genotype but also might be the result of UV cross-linking process during a CLIP experiment (compared to indels, substitutions do not show locational enrichment within RBP CLIP reads). Finally, only SNPs with >0.5 or <−0.5 log2 odds ratio of CLIP vs RNA-seq allelic ratio were labeled as either reference-biased or alternative-biased SNP (defined based on odds ratio, total 34,781 observed allelic imbalance unique SNPs, Additional Data table S2). All SNPs discovered from each human brain specimens (paired RNA-seq+nELAVL-CLIP) were pooled into one final evaluation set, which resulted in roughly equal ratio of allele biased variants (1.1 ratio of ref. vs alt. biased SNPs—total 1,725 SNPs).

### Mouse Brain Elayl-CLIP

Three biological replicates of adult C57BL/6J mice were used to conduct cortex Elavl-CLIP. Elavl was immunoprecipitated from UV cross-linked cortex samples using an anti-Hu serum that recognizes all three neuronal Elavl isoforms.

Genotyping SSC Families from Whole Genome Sequencing

The Simons Foundation Autism Research Initiative (SFARI) WGS data phase 1 release was used in our study that includes raw data and WGS genotyping according to previous SSC report. Candidate SNVs were further filtered by DNMFilter to identify de novo mutations in proband and siblings with threshold of probability>0.75. The de novo mutations were further isolated by removing any overlap with the 1000 Genomes Project SNVs. In addition, all SVNs located within low complexity regions (RepeatMasker) were removed. Using GENCODE gene annotations (build 25), the final number of de novo SNVs located in gene regions for proband was 9,040 and 8,304 for unaffected siblings.

### RRD Mutation Dysregulation Metric

To make the variant effects across RBP models more comparable within the ASD context, a RBP model specific modified e-value and a p-value was first assigned to each de novo variant. The modified e-value is calculated by merging all proband and sibling de novo variants from the category of interest (e.g., AS exons in FMRP targets) into one pool and assigned the following,

Pr(X_{pos,i}≥x_{pos,i}|∀Vₚₒₛ)ᵢ or Pr(X_{neg,i}≤x_{neg,i}|∀V_{neg})ᵢ

where i is the RBP model, x is the variant margin (i.e., predicted RBPᵢ binding probability difference between reference allele and alternative allele) and V is all de novo variants in the query category. The −log10 margin was modeled as a normal distribution separately for positive and negative margin variants (i.e., predicted gain or loss of binding) but without distinction of proband and sibling origin. The modified e-value provides a measurement of the rarity of a variant's predicted effect with equal treatment to proband and sibling variants, thus ideal when assessing the differential burden between the two groups. P-values were assigned using the same procedure but with a distinction that we model a null distribution by only using sibling variants −log10 margin. A combined score of maximum variant effect on RBP binding was calculated by assigning the minimum e-value across all RBP models to the variant. Finally, z scores were derived after converting the minimum e-values of all variants within the query category into a standard normal distribution (inverse of the normal CDF function using 1—e-value statistics), then computing the z score for each variant.

### Annotation and Gene Sets

Human exons that are alternatively spliced were obtained from a recent study that has examined publically available human RNA-seq data to annotate an extensive catalog of AS events. Internal exon region was used for alternative exon definition types of cassette, mutually exclusive, tandem cassette exons. Terminal exon region was used for intron retention, alternative 3′ or 5′ exon AS exon types. All exon-flanking regions, allowing intervals to span across exons, were collapsed into a final set of genomic intervals used to subset SNVs. SNVs were allowed to overlap noncoding exon regions, if the flanking regions overlapped a UTR segment of the gene.

The most updated list of autism coding de novo LGD genes were obtained from Krishnan et al. {Krishnan:2016da}, and release 1.0 of the ExAC functional gene constrained scores were used to obtain pLI (probability of loss-of-function intolerance). An extend list of FMRP targets were used derived from 3 additional biological replicates and including the original 7 replicates FMRP-CLIP {Darnell:2011cy} (1,498 genes, manuscript in preparation, gene list and additional replicate data available upon request prior to publication). Transcripts with FDR<0.05 and coverage of at least 6 biological replicates were defined as FMRP targets and mouse genes were mapped to human genes that satisfy the ENSEMBL defined 1-to-1 or 1-to-many orthologues (i.e., expansion in human lineage) for subsequent analyses.

### Analysis for RBP EFTUD2 and SF3B4

The differential enrichment of large effect RRD mutations for EFTUD2 and SF3B4 within FMRP targets compared to the background constrained genes (non-targets) was computed by using the difference in t-statistics (predicted effect of proband vs sibling) of the two gene sets as a test statistic. A null distribution was computed by permuting the FMRP target membership label for the collection of de novo mutations within constrained genes for 1,000 iterations. The top 1,000 CLIP peaks for EFTUD2 and SF3B4 (ENCODE CLIP HepG2) were used to conduct motif analysis using the MEME suites {Bailey:2009eu} (MEME and CentriMo) to find significantly enriched sequence elements. Nucleotide level enrichment of motifs was conducted by first searching each instance of the motif using MEME tool FIMO up and downstream 200 nts of AS exons within the gene set. The final enrichment score E was computed as following,

$${E}_{i} = \frac{{\sum }_{j}^{{m}_{i}} {S}_{i , j}}{N}$$

where i is the nt to compute enrichment, mᵢ is the total number of exons with FIMO motif hits overlapping nt location i and S_{i,j} is the FIMO score at nt i in exon j. N is the total number of AS exons examined.

### Functions and Pathways Enrichment

Each GO term test statistic was computed as the following. First proband and sibling de novo mutations that are located within the GO term annotated genes were isolated (400 nt flanking exon regions). Next, each RBP model was tested for increased RBP dysregulation, one-sided Wilcoxon rank-sum test of the predicted effects of proband vs. sibling, for the GO term gene set specific de novo mutations. The summation of the −log₁₀(p-value) of all RBP models was used as the GO term test statistic for the ASD burden of RRD mutations. GO term test statistic was converted to an enrichment p-value by generating a null distribution with 1,000 iterations of permuting the proband/sibling labels for the de novo mutations and repeating the same procedure of obtaining the null test statistic (from random proband/sib labels). Finally, GO terms with p-value<0.05 and FDR<0.1 were reported as enriched for proband RRD mutations. Local FDR was computed using the q-value package. GO term annotations were pooled from human (EBI May 9, 2017), mouse (MGI May 26, 2017) and rat (RGD Apr. 8, 2017) and terms with annotation size of less than 150 or greater than 3,000 genes were removed. Query GO terms were obtained from the merged set of curated GO consortium slims from Generic, Protein Information Resource (PIR), Synapse, Chembl, and supplemented by PANTHER GO-slim and terms from NIGO.

### Developmental Stage Autism Risk Signature

Unaffected human brain (i.e., non-ASD, prefrontal cortex) developmental stage RNA-seq data was used to examine the autism risk signature. For each RNA-Seq biological replicate, gene level abundance was estimated by aligning reads with STAR aligner and estimating the TPM values with RSEM. Genes harboring a proband de novo mutation in 400 nt exon-flanking regions were segregated based on the predicted effect (all, z score>1 or z score<−1) and differential expression statistic was calculated comparing to the expression level of sibling-mutated genes (one-sided Wilcoxon rank-sum test). The level of up-regulation of expression for the proband RRD mutation-harboring genes compared to control (sibling mutated genes) was used as a measure of autism risk signature for the developmental time point.

### ASD Proband Phenotype Analysis

All proband phenotype information was obtained from the Simons foundation core descriptive variables (version 15, provides summary statistics for each proband clinical phenotypes). The scores were derived from the Autism Diagnostic Interview-Revised (ADI-R) algorithm as described in the SSC phenotype descriptions. Social interaction severity measurement was obtained from the “adi_r_soc_a_total” metric that is the total score for the Reciprocal Social Interaction Domain on the ADI-R algorithm. Behavior severity measurement, the “adi_r_rrb_c_total” metric, is the total score for the Restricted, Repetitive, and Stereotyped Patterns of Behavior Domain. The “regression” phenotype distinction was made, according to the SSC core description, from loss items on the ADI-R loss insert or questions. Verbal communication severity was obtained from the “adi_r_b_comm_verbal_total” metric, which provides the total score for the Verbal Communication Domain on ADI-R. The severity of phenotypes was tested for a positive association with de novo variant predicted effects within constrained genes (ExAC pLI>0.95, consistent significant results p-value<0.05 for each category was also observed for ExAC pLI>0.98). The R implementation of Pearson product-moment correlation coefficient test was used for all.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1
Chromatin Profiles

| Cell | Feature | Treatment | Type |
|---|---|---|---|
| 8988T | DNase | | DNase |
| AoSMC | DNase | | DNase |
| Chorion | DNase | | DNase |
| CLL | DNase | | DNase |
| Fibrobl | DNase | | DNase |
| FibroP | DNase | | DNase |
| Gliobla | DNase | | DNase |
| GM12891 | DNase | | DNase |
| GM12892 | DNase | | DNase |
| GM18507 | DNase | | DNase |
| GM19238 | DNase | | DNase |
| GM19239 | DNase | | DNase |
| GM19240 | DNase | | DNase |
| H9ES | DNase | | DNase |
| HeLa-S3 | DNase | IFNa4h | DNase |
| Hepatocytes | DNase | | DNase |
| HPDE6-E6E7 | DNase | | DNase |
| HSMM_emb | DNase | | DNase |
| HTR8svn | DNase | | DNase |
| Huh-7.5 | DNase | | DNase |
| Huh-7 | DNase | | DNase |
| iPS | DNase | | DNase |
| Ishikawa | DNase | Estradiol_100nM_1hr | DNase |
| Ishikawa | DNase | 4OHTAM_20nM_72hr | DNase |
| LNCaP | DNase | androgen | DNase |
| MCF-7 | DNase | Hypoxia_LacAcid | DNase |
| Medullo | DNase | | DNase |
| Melano | DNase | | DNase |
| Myometr | DNase | | DNase |
| Osteobl | DNase | | DNase |
| PanlsletD | DNase | | DNase |
| Panlslets | DNase | | DNase |
| pHTE | DNase | | DNase |
| ProgFib | DNase | | DNase |
| RWPE1 | DNase | | DNase |
| Stellate | DNase | | DNase |
| T-47D | DNase | | DNase |
| Adult_CD4_Th0 | DNase | | DNase |
| Urothelia | DNase | | DNase |
| Urothelia | DNase | UT189 | DNase |
| AG04449 | DNase | | DNase |
| AG04450 | DNase | | DNase |
| AG09309 | DNase | | DNase |
| AG09319 | DNase | | DNase |
| AG10803 | DNase | | DNase |
| AoAF | DNase | | DNase |
| BE2_C | DNase | | DNase |
| BJ | DNase | | DNase |
| Caco-2 | DNase | | DNase |
| CD20+ | DNase | | DNase |
| CD34+_Mobilized | DNase | | DNase |
| CMK | DNase | | DNase |
| A549 | DNase | | DNase |
| GM12878 | DNase | | DNase |
| H1-hESC | DNase | | DNase |
| HeLa-S3 | DNase | | DNase |
| HepG2 | DNase | | DNase |
| HMEC | DNase | | DNase |
| HSMMtube | DNase | | DNase |
| HSMM | DNase | | DNase |
| HUVEC | DNase | | DNase |
| K562 | DNase | | DNase |
| LNCaP | DNase | | DNase |
| MCF-7 | DNase | | DNase |
| NHEK | DNase | | DNase |
| Th1 | DNase | | DNase |
| GM06990 | DNase | | DNase |
| GM12864 | DNase | | DNase |
| GM12865 | DNase | | DNase |
| H7-hESC | DNase | | DNase |
| HAc | DNase | | DNase |
| HAEpiC | DNase | | DNase |
| HA-h | DNase | | DNase |
| HA-sp | DNase | | DNase |
| HBMEC | DNase | | DNase |
| HCFaa | DNase | | DNase |
| HGF | DNase | | DNase |
| HCM | DNase | | DNase |
| HConF | DNase | | DNase |
| HCPEpiC | DNase | | DNase |
| HCT-116 | DNase | | DNase |
| HEEpiC | DNase | | DNase |
| HFF-Myc | DNase | | DNase |
| HFF | DNase | | DNase |
| HGF | DNase | | DNase |
| HIPEpiC | DNase | | DNase |
| HL-60 | DNase | | DNase |
| HMF | DNase | | DNase |
| HMVEC-dAd | DNase | | DNase |
| HMVEC-dBl-Ad | DNase | | DNase |
| HMVEC-dBl-Neo | DNase | | DNase |
| HMVEC-dLy-Ad | DNase | | DNase |
| HMVEC-dLy-Neo | DNase | | DNase |
| HMVEC-dNeo | DNase | | DNase |
| HMVEC-LBl | DNase | | DNase |
| HMVEC-LLy | DNase | | DNase |
| HNPCEpiC | DNase | | DNase |
| HPAEC | DNase | | DNase |
| HPAF | DNase | | DNase |
| HPdLF | DNase | | DNase |
| HPF | DNase | | DNase |
| HRCEpiC | DNase | | DNase |
| HRE | DNase | | DNase |
| HRGEC | DNase | | DNase |
| HRPEpiC | DNase | | DNase |
| HVMF | DNase | | DNase |
| Jurkat | DNase | | DNase |
| Monocytes-CD14+_RO01746 | DNase | | DNase |
| NB4 | DNase | | DNase |
| NH-A | DNase | | DNase |
| NHDF-Ad | DNase | | DNase |
| NHDF-neo | DNase | | DNase |
| NHLF | DNase | | DNase |
| NT2-D1 | DNase | | DNase |
| PANC-1 | DNase | | DNase |
| PrEC | DNase | | DNase |
| RPTEC | DNase | | DNase |
| SAEC | DNase | | DNase |
| SKMC | DNase | | DNase |
| SK-N-MC | DNase | | DNase |
| SK-N-SH_RA | DNase | | DNase |
| Th2 | DNase | | DNase |
| WERI-Rb-1 | DNase | | DNase |
| WI-38 | DNase | 4OHTAM_20nM_72hr | DNase |
| WI-38 | DNase | | DNase |
| Dnd41 | CTCF | | TF |
| Dnd41 | EZH2 | | TF |
| GM12878 | CTCF | | TF |
| GM12878 | EZH2 | | TF |
| H1-hESC | CHD1 | | TF |
| H1-hESC | CTCF | | TF |
| H1-hESC | EZH2 | | TF |
| H1-hESC | JARID1A | | TF |
| H1-hESC | RBBP5 | | TF |
| HeLa-S3 | CTCF | | TF |
| HeLa-S3 | EZH2 | | TF |
| HeLa-S3 | Pol2(b) | | TF |
| HepG2 | CTCF | | TF |
| HepG2 | EZH2 | | TF |
| HMEC | CTCF | | TF |
| HMEC | EZH2 | | TF |
| HSMM | CTCF | | TF |
| HSMM | EZH2 | | TF |
| HSMMtube | CTCF | | TF |
| HSMMtube | EZH2 | | TF |
| HUVEC | CTCF | | TF |
| HUVEC | EZH2 | | TF |
| HUVEC | Pol2(b) | | TF |
| K562 | CHD1 | | TF |
| K562 | CTCF | | TF |
| K562 | EZH2 | | TF |
| K562 | HDAC1 | | TF |
| K562 | HDAC2 | | TF |
| K562 | HDAC6 | | TF |
| K562 | p300 | | TF |
| K562 | PHF8 | | TF |
| K562 | PLU1 | | TF |
| K562 | Pol2(b) | | TF |
| K562 | RBBP5 | | TF |
| K562 | SAP30 | | TF |
| NH-A | CTCF | | TF |
| NH-A | EZH2 | | TF |
| NHDF-Ad | CTCF | | TF |
| NHDF-Ad | EZH2 | | TF |
| NHEK | CTCF | | TF |
| NHEK | EZH2 | | TF |
| NHEK | Pol2(b) | | TF |
| NHLF | CTCF | | TF |
| NHLF | EZH2 | | TF |
| Osteobl | CTCF | | TF |
| A549 | ATF3 | EtOH_0.02pct | TF |
| A549 | BCL3 | EtOH_0.02pct | TF |
| A549 | CREB1 | DEX_100nM | TF |
| A549 | CTCF | DEX_100nM | TF |
| A549 | CTCF | EtOH_0.02pct | TF |
| A549 | ELF1 | EtOH_0.02pct | TF |
| A549 | ETS1 | EtOH_0.02pct | TF |
| A549 | FOSL2 | EtOH_0.02pct | TF |
| A549 | FOXA1 | DEX_100nM | TF |
| A549 | GABP | EtOH_0.02pct | TF |
| A549 | GR | DEX_500pM | TF |
| A549 | GR | DEX_50nM | TF |
| A549 | GR | DEX_5nM | TF |
| A549 | GR | DEX_100nM | TF |
| A549 | NRSF | EtOH_0.02pct | TF |
| A549 | p300 | EtOH_0.02pct | TF |
| A549 | Pol2 | DEX_100nM | TF |
| A549 | Pol2 | EtOH_0.02pct | TF |
| A549 | Sin3Ak-20 | EtOH_0.02pct | TF |
| A549 | SIX5 | EtOH_0.02pct | TF |
| A549 | TAF1 | EtOH_0.02pct | TF |
| A549 | TCF12 | EtOH_0.02pct | TF |
| A549 | USF-1 | DEX_100nM | TF |
| A549 | USF-1 | EtOH_0.02pct | TF |
| A549 | USF-1 | EtOH_0.02pct | TF |
| A549 | YY1 | EtOH_0.02pct | TF |
| A549 | ZBTB33 | EtOH_0.02pct | TF |
| ECC-1 | CTCF | DMSO_0.02pct | TF |
| ECC-1 | ERalpha | BPA_100nM | TF |
| ECC-1 | ERalpha | Estradiol_10nM | TF |
| ECC-1 | ERalpha | Genistein_100nM | TF |
| ECC-1 | FOXA1 | DMSO_0.02pct | TF |
| ECC-1 | GR | DEX_100nM | TF |
| ECC-1 | Pol2 | DMSO_0.02pct | TF |
| GM12878 | ATF2 | | TF |
| GM12878 | ATF3 | | TF |
| GM12878 | BATF | | TF |
| GM12878 | BCL11A | | TF |
| GM12878 | BCL3 | | TF |
| GM12878 | BCLAF1 | | TF |
| GM12878 | CEBPB | | TF |
| GM12878 | EBF1 | | TF |
| GM12878 | Egr-1 | | TF |
| GM12878 | ELF1 | | TF |
| GM12878 | ETS1 | | TF |
| GM12878 | FOXM1 | | TF |
| GM12878 | GABP | | TF |
| GM12878 | IRF4 | | TF |
| GM12878 | MEF2A | | TF |
| GM12878 | MEF2C | | TF |
| GM12878 | MTA3 | | TF |
| GM12878 | NFATC1 | | TF |
| GM12878 | NFIC | | TF |
| GM12878 | NRSF | | TF |
| GM12878 | p300 | | TF |
| GM12878 | PAX5-C20 | | TF |
| GM12878 | PAX5-N19 | | TF |
| GM12878 | Pbx3 | | TF |
| GM12878 | PML | | TF |
| GM12878 | Pol2-4H8 | | TF |
| GM12878 | Pol2 | | TF |
| GM12878 | POU2F2 | | TF |
| GM12878 | PU.1 | | TF |
| GM12878 | Rad21 | | TF |
| GM12878 | RUNX3 | | TF |
| GM12878 | RXRA | | TF |
| GM12878 | SIX5 | | TF |
| GM12878 | SP1 | | TF |
| GM12878 | SRF | | TF |
| GM12878 | STAT5A | | TF |
| GM12878 | TAF1 | | TF |
| GM12878 | TCF12 | | TF |
| GM12878 | TCF3 | | TF |
| GM12878 | USF-1 | | TF |
| GM12878 | YY1 | | TF |
| GM12878 | ZBTB33 | | TF |
| GM12878 | ZEB1 | | TF |
| GM12891 | PAX5-C20 | | TF |
| GM12891 | Pol2-4H8 | | TF |
| GM12891 | Pol2 | | TF |
| GM12891 | POU2F2 | | TF |
| GM12891 | PU.1 | | TF |
| GM12891 | TAF1 | | TF |
| GM12891 | YY1 | | TF |
| GM12892 | PAX5-C20 | | TF |
| GM12892 | Pol2-4H8 | | TF |
| GM12892 | Pol2 | | TF |
| GM12892 | TAF1 | | TF |
| GM12892 | YY1 | | TF |
| H1-hESC | ATF2 | | TF |
| H1-hESC | ATF3 | | TF |
| H1-hESC | BCL11A | | TF |
| H1-hESC | CTCF | | TF |
| H1-hESC | Egr-1 | | TF |
| H1-hESC | FOSL1 | | TF |
| H1-hESC | GABP | | TF |
| H1-hESC | HDAC2 | | TF |
| H1-hESC | JunD | | TF |
| H1-hESC | NANOG | | TF |
| H1-hESC | NRSF | | TF |
| H1-hESC | p300 | | TF |
| H1-hESC | Pol2-4H8 | | TF |
| H1-hESC | Pol2 | | TF |
| H1-hESC | POU5F1 | | TF |
| H1-hESC | Rad21 | | TF |
| H1-hESC | RXRA | | TF |
| H1-hESC | Sin3Ak-20 | | TF |
| H1-hESC | SIX5 | | TF |
| H1-hESC | SP1 | | TF |
| H1-hESC | SP2 | | TF |
| H1-hESC | SP4 | | TF |
| H1-hESC | SRF | | TF |
| H1-hESC | TAF1 | | TF |
| H1-hESC | TAF7 | | TF |
| H1-hESC | TCF12 | | TF |
| H1-hESC | TEAD4 | | TF |
| H1-hESC | USF-1 | | TF |
| H1-hESC | YY1 | | TF |
| HCT-116 | Pol2-4H8 | | TF |
| HCT-116 | YY1 | | TF |
| HCT-116 | ZBTB33 | | TF |
| HeLa-S3 | GABP | | TF |
| HeLa-S3 | NRSF | | TF |
| HeLa-S3 | Pol2 | | TF |
| HeLa-S3 | TAF1 | | TF |
| HepG2 | ATF3 | | TF |
| HepG2 | BHLHE40 | | TF |
| HepG2 | CEBPB | | TF |
| HepG2 | CEBPD | | TF |
| HepG2 | CTCF | | TF |
| HepG2 | ELF1 | | TF |
| HepG2 | FOSL2 | | TF |
| HepG2 | FOXA1 | | TF |
| HepG2 | FOXA1 | | TF |
| HepG2 | FOXA2 | | TF |
| HepG2 | GABP | | TF |
| HepG2 | HDAC2 | | TF |
| HepG2 | HNF4A | | TF |
| HepG2 | HNF4G | | TF |
| HepG2 | JunD | | TF |
| HepG2 | MBD4 | | TF |
| HepG2 | MYBL2 | | TF |
| HepG2 | NFIC | | TF |
| HepG2 | NRSF | | TF |
| HepG2 | NRSF | | TF |
| HepG2 | p300 | | TF |
| HepG2 | Pol2-4H8 | | TF |
| HepG2 | Pol2 | | TF |
| HepG2 | Rad21 | | TF |
| HepG2 | RXRA | | TF |
| HepG2 | Sin3Ak-20 | | TF |
| HepG2 | SP1 | | TF |
| HepG2 | SP2 | | TF |
| HepG2 | SRF | | TF |
| HepG2 | TAF1 | | TF |
| HepG2 | TCF12 | | TF |
| HepG2 | TEAD4 | | TF |
| HepG2 | USF-1 | | TF |
| HepG2 | YY1 | | TF |
| HepG2 | ZBTB33 | | TF |
| HepG2 | ZBTB7A | | TF |
| HUVEC | Pol2-4H8 | | TF |
| HUVEC | Pol2 | | TF |
| K562 | ATF3 | | TF |
| K562 | BCL3 | | TF |
| K562 | BCLAF1 | | TF |
| K562 | CBX3 | | TF |
| K562 | CEBPB | | TF |
| K562 | CTCF | | TF |
| K562 | CTCFL | | TF |
| K562 | E2F6 | | TF |
| K562 | Egr-1 | | TF |
| K562 | ELF1 | | TF |
| K562 | ETS1 | | TF |
| K562 | FOSL1 | | TF |
| K562 | GABP | | TF |
| K562 | GATA2 | | TF |
| K562 | HDAC2 | | TF |
| K562 | Max | | TF |
| K562 | MEF2A | | TF |
| K562 | NR2F2 | | TF |
| K562 | NRSF | | TF |
| K562 | PML | | TF |
| K562 | Pol2-4H8 | | TF |
| K562 | Pol2 | | TF |
| K562 | PU.1 | | TF |
| K562 | Rad21 | | TF |
| K562 | Sin3Ak-20 | | TF |
| K562 | SIX5 | | TF |
| K562 | SP1 | | TF |
| K562 | SP2 | | TF |
| K562 | SRF | | TF |
| K562 | STAT5A | | TF |
| K562 | TAF1 | | TF |
| K562 | TAF7 | | TF |
| K562 | TEAD4 | | TF |
| K562 | THAP1 | | TF |
| K562 | TRIM28 | | TF |
| K562 | USF-1 | | TF |
| K562 | YY1 | | TF |
| K562 | YY1 | | TF |
| K562 | ZBTB33 | | TF |
| K562 | ZBTB7A | | TF |
| PANC-1 | NRSF | | TF |
| PANC-1 | Pol2-4H8 | | TF |
| PANC-1 | Sin3Ak-20 | | TF |
| PFSK-1 | FOXP2 | | TF |
| PFSK-1 | NRSF | | TF |
| PFSK-1 | Sin3Ak-20 | | TF |
| PFSK-1 | TAF1 | | TF |
| SK-N-MC | FOXP2 | | TF |
| SK-N-MC | Pol2-4H8 | | TF |
| SK-N-SH | NRSF | | TF |
| SK-N-SH | NRSF | | TF |
| SK-N-SH | Pol2-4H8 | | TF |
| SK-N-SH_RA | CTCF | | TF |
| SK-N-SH_RA | p300 | | TF |
| SK-N-SH_RA | Rad21 | | TF |
| SK-N-SH_RA | USF1 | | TF |
| SK-N-SH_RA | YY1 | | TF |
| SK-N-SH | Sin3Ak-20 | | TF |
| SK-N-SH | TAF1 | | TF |
| T-47D | CTCF | DMSO_0.02pct | TF |
| T-47D | ERalpha | BPA_100nM | TF |
| T-47D | ERalpha | Genistein_100nM | TF |
| T-47D | ERalpha | Estradiol_10nM | TF |
| T-47D | FOXA1 | DMSO_0.02pct | TF |
| T-47D | GATA3 | DMSO_0.02pct | TF |
| T-47D | p300 | DMSO_0.02pct | TF |
| U87 | NRSF | | TF |
| U87 | Pol2-4H8 | | TF |
| A549 | BHLHE40 | | TF |
| A549 | CEBPB | | TF |
| A549 | Max | | TF |
| A549 | Pol2(phosphoS2) | | TF |
| A549 | Rad21 | | TF |
| GM08714 | ZNF274 | | TF |
| GM10847 | NFKB | TNFa | TF |
| GM10847 | Pol2 | | TF |
| GM12878 | BHLHE40 | | TF |
| GM12878 | BRCA1 | | TF |
| GM12878 | c-Fos | | TF |
| GM12878 | CHD1 | | TF |
| GM12878 | CHD2 | | TF |
| GM12878 | COREST | | TF |
| GM12878 | CTCF | | TF |
| GM12878 | E2F4 | | TF |
| GM12878 | EBF1 | | TF |
| GM12878 | ELK1 | | TF |
| GM12878 | IKZF1 | | TF |
| GM12878 | JunD | | TF |
| GM12878 | Max | | TF |
| GM12878 | MAZ | | TF |
| GM12878 | Mxi1 | | TF |
| GM12878 | NF-E2 | | TF |
| GM12878 | NFKB | TNFa | TF |
| GM12878 | NF-YA | | TF |
| GM12878 | NF-YB | | TF |
| GM12878 | Nrf1 | | TF |
| GM12878 | p300 | | TF |
| GM12878 | p300 | | TF |
| GM12878 | Pol2 | | TF |
| GM12878 | Pol2(phosphoS2) | | TF |
| GM12878 | Pol2 | | TF |
| GM12878 | Pol3 | | TF |
| GM12878 | Rad21 | | TF |
| GM12878 | RFX5 | | TF |
| GM12878 | SIN3A | | TF |
| GM12878 | SMC3 | | TF |
| GM12878 | STAT1 | | TF |
| GM12878 | STAT3 | | TF |
| GM12878 | TBLR1 | | TF |
| GM12878 | TBP | | TF |
| GM12878 | TR4 | | TF |
| GM12878 | USF2 | | TF |
| GM12878 | WHIP | | TF |
| GM12878 | YY1 | | TF |
| GM12878 | Znf143 | | TF |
| GM12878 | ZNF274 | | TF |
| GM12878 | ZZZ3 | | TF |
| GM12891 | NFKB | TNFa | TF |
| GM12891 | Pol2 | | TF |
| GM12892 | NFKB | TNFa | TF |
| GM12892 | Pol2 | | TF |
| GM15510 | NFKB | TNFa | TF |
| GM15510 | Pol2 | | TF |
| GM18505 | NFKB | TNFa | TF |
| GM18505 | Pol2 | | TF |
| GM18526 | NFKB | TNFa | TF |
| GM18526 | Pol2 | | TF |
| GM18951 | NFKB | TNFa | TF |
| GM18951 | Pol2 | | TF |
| GM19099 | NFKB | TNFa | TF |
| GM19099 | Pol2 | | TF |
| GM19193 | NFKB | TNFa | TF |
| GM19193 | Pol2 | | TF |
| H1-hESC | Bach1 | | TF |
| H1-hESC | BRCA1 | | TF |
| H1-hESC | CEBPB | | TF |
| H1-hESC | CHD1 | | TF |
| H1-hESC | CHD2 | | TF |
| H1-hESC | c-Jun | | TF |
| H1-hESC | c-Myc | | TF |
| H1-hESC | CtBP2 | | TF |
| H1-hESC | GTF2F1 | | TF |
| H1-hESC | JunD | | TF |
| H1-hESC | MafK | | TF |
| H1-hESC | Max | | TF |
| H1-hESC | Mxi1 | | TF |
| H1-hESC | Nrf1 | | TF |
| H1-hESC | Rad21 | | TF |
| H1-hESC | RFX5 | | TF |
| H1-hESC | SIN3A | | TF |
| H1-hESC | SUZ12 | | TF |
| H1-hESC | TBP | | TF |
| H1-hESC | USF2 | | TF |
| H1-hESC | Znf143 | | TF |
| HCT-116 | Pol2 | | TF |
| HCT-116 | TCF7L2 | | TF |
| HEK293 | ELK4 | | TF |
| HEK293 | KAP1 | | TF |
| HEK293 | Pol2 | | TF |
| HEK293 | TCF7L2 | | TF |
| HEK293-T-REx | ZNF263 | | TF |
| HeLa-S3 | AP-2alpha | | TF |
| HeLa-S3 | AP-2gamma | | TF |
| HeLa-S3 | BAF155 | | TF |
| HeLa-S3 | BAF170 | | TF |
| HeLa-S3 | BDP1 | | TF |
| HeLa-S3 | BRCA1 | | TF |
| HeLa-S3 | BRF1 | | TF |
| HeLa-S3 | BRF2 | | TF |
| HeLa-S3 | Brg1 | | TF |
| HeLa-S3 | CEBPB | | TF |
| HeLa-S3 | c-Fos | | TF |
| HeLa-S3 | CHD2 | | TF |
| HeLa-S3 | c-Jun | | TF |
| HeLa-S3 | c-Myc | | TF |
| HeLa-S3 | COREST | | TF |
| HeLa-S3 | E2F1 | | TF |
| HeLa-S3 | E2F4 | | TF |
| HeLa-S3 | E2F6 | | TF |
| HeLa-S3 | ELK1 | | TF |
| HeLa-S3 | ELK4 | | TF |
| HeLa-S3 | GTF2F1 | | TF |
| HeLa-S3 | HA-E2F1 | | TF |
| HeLa-S3 | Ini1 | | TF |
| HeLa-S3 | IRF3 | | TF |
| HeLa-S3 | JunD | | TF |
| HeLa-S3 | MafK | | TF |
| HeLa-S3 | Max | | TF |
| HeLa-S3 | MAZ | | TF |
| HeLa-S3 | Mxi1 | | TF |
| HeLa-S3 | NF-YA | | TF |
| HeLa-S3 | NF-YB | | TF |
| HeLa-S3 | Nrf1 | | TF |
| HeLa-S3 | p300 | | TF |
| HeLa-S3 | Pol2(phosphoS2) | | TF |
| HeLa-S3 | Pol2 | | TF |
| HeLa-S3 | PRDM1 | | TF |
| HeLa-S3 | Rad21 | | TF |
| HeLa-S3 | RFX5 | | TF |
| HeLa-S3 | RPC155 | | TF |
| HeLa-S3 | SMC3 | | TF |
| HeLa-S3 | SPT20 | | TF |
| HeLa-S3 | STAT1 | IFNg30 | TF |
| HeLa-S3 | STAT3 | | TF |
| HeLa-S3 | TBP | | TF |
| HeLa-S3 | TCF7L2 | | TF |
| HeLa-S3 | TCF7L2 | | TF |
| HeLa-S3 | TFIIIC-110 | | TF |
| HeLa-S3 | TR4 | | TF |
| HeLa-S3 | USF2 | | TF |
| HeLa-S3 | ZKSCAN1 | | TF |
| HeLa-S3 | Znf143 | | TF |
| HeLa-S3 | ZNF274 | | TF |
| HeLa-S3 | ZZZ3 | | TF |
| HepG2 | ARID3A | | TF |
| HepG2 | BHLHE40 | | TF |
| HepG2 | BRCA1 | | TF |
| HepG2 | CEBPB | forskolin | TF |
| HepG2 | CEBPB | | TF |
| HepG2 | CHD2 | | TF |
| HepG2 | c-Jun | | TF |
| HepG2 | COREST | | TF |
| HepG2 | ERRA | forskolin | TF |
| HepG2 | GRp20 | forskolin | TF |
| HepG2 | HNF4A | forskolin | TF |
| HepG2 | HSF1 | forskolin | TF |
| HepG2 | IRF3 | | TF |
| HepG2 | JunD | | TF |
| HepG2 | MafF | | TF |
| HepG2 | MafK | | TF |
| HepG2 | MafK | | TF |
| HepG2 | Max | | TF |
| HepG2 | MAZ | | TF |
| HepG2 | Mxi1 | | TF |
| HepG2 | Nrf1 | | TF |
| HepG2 | p300 | | TF |
| HepG2 | PGC1A | forskolin | TF |
| HepG2 | Pol2 | forskolin | TF |
| HepG2 | Pol2 | | TF |
| HepG2 | Pol2(phosphoS2) | | TF |
| HepG2 | Rad21 | | TF |
| HepG2 | RFX5 | | TF |
| HepG2 | SMC3 | | TF |
| HepG2 | SREBP1 | insulin | TF |
| HepG2 | TBP | | TF |
| HepG2 | TCF7L2 | | TF |
| HepG2 | TR4 | | TF |
| HepG2 | USF2 | | TF |
| HepG2 | ZNF274 | | TF |
| HUVEC | c-Fos | | TF |
| HUVEC | c-Jun | | TF |
| HUVEC | GATA-2 | | TF |
| HUVEC | Max | | TF |
| HUVEC | Pol2 | | TF |
| IMR90 | CEBPB | | TF |
| IMR90 | CTCF | | TF |
| IMR90 | MafK | | TF |
| IMR90 | Pol2 | | TF |
| IMR90 | Rad21 | | TF |
| K562 | ARID3A | | TF |
| K562 | ATF1 | | TF |
| K562 | ATF3 | | TF |
| K562 | Bach1 | | TF |
| K562 | BDP1 | | TF |
| K562 | BHLHE40 | | TF |
| K562 | BRF1 | | TF |
| K562 | BRF2 | | TF |
| K562 | Brg1 | | TF |
| K562 | CCNT2 | | TF |
| K562 | CEBPB | | TF |
| K562 | c-Fos | | TF |
| K562 | CHD2 | | TF |
| K562 | c-Jun | IFNa30 | TF |
| K562 | c-Jun | IFNa6h | TF |
| K562 | c-Jun | IFNg30 | TF |
| K562 | c-Jun | IFNg6h | TF |
| K562 | c-Jun | | TF |
| K562 | c-Myc | IFNa30 | TF |
| K562 | c-Myc | IFNa6h | TF |
| K562 | c-Myc | IFNg30 | TF |
| K562 | c-Myc | IFNg6h | TF |
| K562 | c-Myc | | TF |
| K562 | c-Myc | | TF |
| K562 | COREST | | TF |
| K562 | COREST | | TF |
| K562 | CTCF | | TF |
| K562 | E2F4 | | TF |
| K562 | E2F6 | | TF |
| K562 | ELK1 | | TF |
| K562 | GATA-1 | | TF |
| K562 | GATA-2 | | TF |
| K562 | GTF2B | | TF |
| K562 | GTF2F1 | | TF |
| K562 | HMGN3 | | TF |
| K562 | Ini1 | | TF |
| K562 | IRF1 | IFNa30 | TF |
| K562 | IRF1 | IFNa6h | TF |
| K562 | IRF1 | IFNg30 | TF |
| K562 | IRF1 | IFNg6h | TF |
| K562 | JunD | | TF |
| K562 | KAP1 | | TF |
| K562 | MafF | | TF |
| K562 | MafK | | TF |
| K562 | Max | | TF |
| K562 | MAZ | | TF |
| K562 | Mxi1 | | TF |
| K562 | NELFe | | TF |
| K562 | NF-E2 | | TF |
| K562 | NF-YA | | TF |
| K562 | NF-YB | | TF |
| K562 | Nrf1 | | TF |
| K562 | p300 | | TF |
| K562 | Pol2 | IFNa30 | TF |
| K562 | Pol2 | IFNa6h | TF |
| K562 | Pol2 | IFNg30 | TF |
| K562 | Pol2 | IFNg6h | TF |
| K562 | Pol2 | | TF |
| K562 | Pol2(phosphoS2) | | TF |
| K562 | Pol2(phosphoS2) | | TF |
| K562 | Pol2 | | TF |
| K562 | Pol3 | | TF |
| K562 | Rad21 | | TF |
| K562 | RFX5 | | TF |
| K562 | RPC155 | | TF |
| K562 | SETDB1 | MNaseD | TF |
| K562 | SETDB1 | | TF |
| K562 | SIRT6 | | TF |
| K562 | SMC3 | | TF |
| K562 | STAT1 | IFNa30 | TF |
| K562 | STAT1 | IFNa6h | TF |
| K562 | STAT1 | IFNg30 | TF |
| K562 | STAT1 | IFNg6h | TF |
| K562 | STAT2 | IFNa30 | TF |
| K562 | STAT2 | IFNa6h | TF |
| K562 | TAL1 | | TF |
| K562 | TBLR1 | | TF |
| K562 | TBLR1 | | TF |
| K562 | TBP | | TF |
| K562 | TFIIIC-110 | | TF |
| K562 | TR4 | | TF |
| K562 | UBF | | TF |
| K562 | UBTF | | TF |
| K562 | USF2 | | TF |
| K562 | YY1 | | TF |
| K562 | Znf143 | | TF |
| K562 | ZNF263 | | TF |
| K562 | ZNF274 | | TF |
| K562 | ZNF274 | | TF |
| MCF10A-Er-Src | c-Fos | EtOH_0.01pct | TF |
| MCF10A-Er-Src | c-Fos | 4OHTAM_1uM_12hr | TF |
| MCF10A-Er-Src | c-Fos | 4OHTAM_1uM_4hr | TF |
| MCF10A-Er-Src | c-Fos | 4OHTAM_1uM_36hr | TF |
| MCF10A-Er-Src | c-Myc | EtOH_0.01pct | TF |
| MCF10A-Er-Src | c-Myc | 4OHTAM_1uM_4hr | TF |
| MCF10A-Er-Src | E2F4 | 4OHTAM_1uM_36hr | TF |
| MCF10A-Er-Src | Pol2 | EtOH_0.01pct | TF |
| MCF10A-Er-Src | Pol2 | 4OHTAM_1uM_36hr | TF |
| MCF10A-Er-Src | STAT3 | EtOH_0.01pct_4hr | TF |
| MCF10A-Er-Src | STAT3 | EtOH_0.01pct_12hr | TF |
| MCF10A-Er-Src | STAT3 | EtOH_0.01pct | TF |
| MCF10A-Er-Src | STAT3 | 4OHTAM_1uM_12hr | TF |
| MCF10A-Er-Src | STAT3 | 4OHTAM_1uM_36hr | TF |
| MCF-7 | GATA3 | | TF |
| MCF-7 | GATA3 | | TF |
| MCF-7 | HA-E2F1 | | TF |
| MCF-7 | TCF7L2 | | TF |
| MCF-7 | ZNF217 | | TF |
| NB4 | c-Myc | | TF |
| NB4 | Max | | TF |
| NB4 | Pol2 | | TF |
| NT2-D1 | SUZ12 | | TF |
| NT2-D1 | YY1 | | TF |
| NT2-D1 | ZNF274 | | TF |
| PANC-1 | TCF7L2 | | TF |
| PBDEFetal | GATA-1 | | TF |
| PBDE | GATA-1 | | TF |
| PBDE | Pol2 | | TF |
| Raji | Pol2 | | TF |
| SH-SY5Y | GATA-2 | | TF |
| SH-SY5Y | GATA3 | | TF |
| U2OS | KAP1 | | TF |
| U2OS | SETDB1 | | TF |
| K562 | eGFP-FOS | | TF |
| K562 | eGFP-GATA2 | | TF |
| K562 | eGFP-HDAC8 | | TF |
| K562 | eGFP-JunB | | TF |
| K562 | eGFP-JunD | | TF |
| A549 | CTCF | | TF |
| A549 | Pol2 | | TF |
| Fibrobl | CTCF | | TF |
| Gliobla | CTCF | | TF |
| Gliobla | Pol2 | | TF |
| GM12878 | c-Myc | | TF |
| GM12878 | CTCF | | TF |
| GM12878 | Pol2 | | TF |
| GM12891 | CTCF | | TF |
| GM12892 | CTCF | | TF |
| GM19238 | CTCF | | TF |
| GM19239 | CTCF | | TF |
| GM19240 | CTCF | | TF |
| H1-hESC | c-Myc | | TF |
| H1-hESC | CTCF | | TF |
| H1-hESC | Pol2 | | TF |
| HeLa-S3 | c-Myc | | TF |
| HeLa-S3 | CTCF | | TF |
| HeLa-S3 | Pol2 | | TF |
| HepG2 | c-Myc | | TF |
| HepG2 | CTCF | | TF |
| HepG2 | Pol2 | | TF |
| HUVEC | c-Myc | | TF |
| HUVEC | CTCF | | TF |
| HUVEC | Pol2 | | TF |
| K562 | c-Myc | | TF |
| K562 | CTCF | | TF |
| K562 | Pol2 | | TF |
| MCF-7 | c-Myc | estrogen | TF |
| MCF-7 | c-Myc | serum_stimulated_media | TF |
| MCF-7 | c-Myc | serum_starved_media | TF |
| MCF-7 | c-Myc | vehicle | TF |
| MCF-7 | CTCF | estrogen | TF |
| MCF-7 | CTCF | serum_stimulated_media | TF |
| MCF-7 | CTCF | serum_starved_media | TF |
| MCF-7 | CTCF | | TF |
| MCF-7 | CTCF | vehicle | TF |
| MCF-7 | Pol2 | serum_stimulated_media | TF |
| MCF-7 | Pol2 | serum_starved_media | TF |
| MCF-7 | Pol2 | | TF |
| NHEK | CTCF | | TF |
| ProgFib | CTCF | | TF |
| ProgFib | Pol2 | | TF |
| A549 | CTCF | | TF |
| AG04449 | CTCF | | TF |
| AG04450 | CTCF | | TF |
| AG09309 | CTCF | | TF |
| AG09319 | CTCF | | TF |
| AG10803 | CTCF | | TF |
| AoAF | CTCF | | TF |
| BE2_C | CTCF | | TF |
| BJ | CTCF | | TF |
| Caco-2 | CTCF | | TF |
| GM06990 | CTCF | | TF |
| GM12801 | CTCF | | TF |
| GM12864 | CTCF | | TF |
| GM12865 | CTCF | | TF |
| GM12872 | CTCF | | TF |
| GM12873 | CTCF | | TF |
| GM12874 | CTCF | | TF |
| GM12875 | CTCF | | TF |
| GM12878 | CTCF | | TF |
| HAc | CTCF | | TF |
| HA-sp | CTCF | | TF |
| HBMEC | CTCF | | TF |
| HCFaa | CTCF | | TF |
| HCM | CTCF | | TF |
| HCPEpiC | CTCF | | TF |
| HCT-116 | CTCF | | TF |
| HEEpiC | CTCF | | TF |
| HEK293 | CTCF | | TF |
| HeLa-S3 | CTCF | | TF |
| HepG2 | CTCF | | TF |
| HFF | CTCF | | TF |
| HFF-Myc | CTCF | | TF |
| HL-60 | CTCF | | TF |
| HMEC | CTCF | | TF |
| HMF | CTCF | | TF |
| HPAF | CTCF | | TF |
| HPF | CTCF | | TF |
| HRE | CTCF | | TF |
| HRPEpiC | CTCF | | TF |
| HUVEC | CTCF | | TF |
| HVMF | CTCF | | TF |
| K562 | CTCF | | TF |
| MCF-7 | CTCF | | TF |
| NB4 | CTCF | | TF |
| NHDF-neo | CTCF | | TF |
| NHEK | CTCF | | TF |
| NHLF | CTCF | | TF |
| RPTEC | CTCF | | TF |
| SAEC | CTCF | | TF |
| SK-N-SH_RA | CTCF | | TF |
| WERI-Rb-1 | CTCF | | TF |
| WI-38 | CTCF | | TF |
| ES-I3_Cell_Line | H3K27me3 | | Histone |
| ES-I3_Cell_Line | H3K36me3 | | Histone |
| ES-I3_Cell_Line | H3K4me1 | | Histone |
| ES-I3_Cell_Line | H3K4me3 | | Histone |
| ES-I3_Cell_Line | H3K9ac | | Histone |
| ES-I3_Cell_Line | H3K9me3 | | Histone |
| ES-WA7_Cell_Line | H3K27me3 | | Histone |
| ES-WA7_Cell_Line | H3K36me3 | | Histone |
| ES-WA7_Cell_Line | H3K4me1 | | Histone |
| ES-WA7_Cell_Line | H3K4me3 | | Histone |
| ES-WA7_Cell_Line | H3K9ac | | Histone |
| ES-WA7_Cell_Line | H3K9me3 | | Histone |
| H1-hESC | DNase.all.peaks | | DNase |
| H1-hESC | DNase.fdr0.01.hot | | DNase |
| H1-hESC | DNase.fdr0.01.peaks | | DNase |
| H1-hESC | DNase.hot | | DNase |
| H1-hESC | DNase | | DNase |
| H1-hESC | H2AK5ac | | Histone |
| H1-hESC | H2A.Z | | Histone |
| H1-hESC | H2BK120ac | | Histone |
| H1-hESC | H2BK12ac | | Histone |
| H1-hESC | H2BK15ac | | Histone |
| H1-hESC | H2BK20ac | | Histone |
| H1-hESC | H2BK5ac | | Histone |
| H1-hESC | H3K14ac | | Histone |
| H1-hESC | H3K18ac | | Histone |
| H1-hESC | H3K23ac | | Histone |
| H1-hESC | H3K23me2 | | Histone |
| H1-hESC | H3K27ac | | Histone |
| H1-hESC | H3K27me3 | | Histone |
| H1-hESC | H3K36me3 | | Histone |
| H1-hESC | H3K4ac | | Histone |
| H1-hESC | H3K4me1 | | Histone |
| H1-hESC | H3K4me2 | | Histone |
| H1-hESC | H3K4me3 | | Histone |
| H1-hESC | H3K56ac | | Histone |
| H1-hESC | H3K79me1 | | Histone |
| H1-hESC | H3K79me2 | | Histone |
| H1-hESC | H3K9ac | | Histone |
| H1-hESC | H3K9me3 | | Histone |
| H1-hESC | H4K20me1 | | Histone |
| H1-hESC | H4K5ac | | Histone |
| H1-hESC | H4K8ac | | Histone |
| H1-hESC | H4K91ac | | Histone |
| H1_BMP4_Derived_Mesendoderm_Cultured_Cells | DNase.all.peaks | | DNase |
| H1_BMP4_Derived_Mesendoderm_Cultured_Cells | DNase.fdr0.01.hot | | DNase |
| H1_BMP4_Derived_Mesendoderm_Cultured_Cells | DNase.fdr0.01.peaks | | DNase |
| H1_BMP4_Derived_Mesendoderm_Cultured_Cells | DNase.hot | | DNase |
| H1_BMP4_Derived_Mesendoderm_Cultured_Cells | DNase | | DNase |
| H1_BMP4_Derived_Mesendoderm_Cultured_Cells | H2AK5ac | | Histone |
| H1_BMP4_Derived_Mesendoderm_Cultured_Cells | H2BK120ac | | Histone |
| H1_BMP4_Derived_Mesendoderm_Cultured_Cells | H2BK15ac | | Histone |
| H1_BMP4_Derived_Mesendoderm_Cultured_Cells | H2BK5ac | | Histone |
| H1_BMP4_Derived_Mesendoderm_Cultured_Cells | H3K18ac | | Histone |
| H1_BMP4_Derived_Mesendoderm_Cultured_Cells | H3K23ac | | Histone |
| H1_BMP4_Derived_Mesendoderm_Cultured_Cells | H3K27ac | | Histone |
| H1_BMP4_Derived_Mesendoderm_Cultured_Cells | H3K27me3 | | Histone |
| H1_BMP4_Derived_Mesendoderm_Cultured_Cells | H3K36me3 | | Histone |
| H1_BMP4_Derived_Mesendoderm_Cultured_Cells | H3K4ac | | Histone |
| H1_BMP4_Derived_Mesendoderm_Cultured_Cells | H3K4me1 | | Histone |
| H1_BMP4_Derived_Mesendoderm_Cultured_Cells | H3K4me2 | | Histone |
| H1_BMP4_Derived_Mesendoderm_Cultured_Cells | H3K4me3 | | Histone |
| H1_BMP4_Derived_Mesendoderm_Cultured_Cells | H3K79me1 | | Histone |
| H1_BMP4_Derived_Mesendoderm_Cultured_Cells | H3K79me2 | | Histone |
| H1_BMP4_Derived_Mesendoderm_Cultured_Cells | H3K9ac | | Histone |
| H1_BMP4_Derived_Mesendoderm_Cultured_Cells | H3K9me3 | | Histone |
| H1_BMP4_Derived_Mesendoderm_Cultured_Cells | H4K8ac | | Histone |
| H1_BMP4_Derived_Trophoblast_Cultured_Cells | DNase.all.peaks | | DNase |
| H1_BMP4_Derived_Trophoblast_Cultured_Cells | DNase.fdr0.01.hot | | DNase |
| H1_BMP4_Derived_Trophoblast_Cultured_Cells | DNase.fdr0.01.peaks | | DNase |
| H1_BMP4_Derived_Trophoblast_Cultured_Cells | DNase.hot | | DNase |
| H1_BMP4_Derived_Trophoblast_Cultured_Cells | DNase | | DNase |
| H1_BMP4_Derived_Trophoblast_Cultured_Cells | H2AK5ac | | Histone |
| H1_BMP4_Derived_Trophoblast_Cultured_Cells | H2A.Z | | Histone |
| H1_BMP4_Derived_Trophoblast_Cultured_Cells | H2BK120ac | | Histone |
| H1_BMP4_Derived_Trophoblast_Cultured_Cells | H2BK12ac | | Histone |
| H1_BMP4_Derived_Trophoblast_Cultured_Cells | H2BK5ac | | Histone |
| H1_BMP4_Derived_Trophoblast_Cultured_Cells | H3K14ac | | Histone |
| H1_BMP4_Derived_Trophoblast_Cultured_Cells | H3K18ac | | Histone |
| H1_BMP4_Derived_Trophoblast_Cultured_Cells | H3K23ac | | Histone |
| H1_BMP4_Derived_Trophoblast_Cultured_Cells | H3K27ac | | Histone |
| H1_BMP4_Derived_Trophoblast_Cultured_Cells | H3K27me3 | | Histone |
| H1_BMP4_Derived_Trophoblast_Cultured_Cells | H3K36me3 | | Histone |
| H1_BMP4_Derived_Trophoblast_Cultured_Cells | H3K4ac | | Histone |
| H1_BMP4_Derived_Trophoblast_Cultured_Cells | H3K4me1 | | Histone |
| H1_BMP4_Derived_Trophoblast_Cultured_Cells | H3K4me2 | | Histone |
| H1_BMP4_Derived_Trophoblast_Cultured_Cells | H3K4me3 | | Histone |
| H1_BMP4_Derived_Trophoblast_Cultured_Cells | H3K79me1 | | Histone |
| H1_BMP4_Derived_Trophoblast_Cultured_Cells | H3K79me2 | | Histone |
| H1_BMP4_Derived_Trophoblast_Cultured_Cells | H3K9ac | | Histone |
| H1_BMP4_Derived_Trophoblast_Cultured_Cells | H3K9me3 | | Histone |
| H1_BMP4_Derived_Trophoblast_Cultured_Cells | H4K12ac | | Histone |
| H1_BMP4_Derived_Trophoblast_Cultured_Cells | H4K8ac | | Histone |
| H1_BMP4_Derived_Trophoblast_Cultured_Cells | H4K91ac | | Histone |
| H1_Derived_Mesenchymal_Stem_Cells | DNase.all.peaks | | DNase |
| H1_Derived_Mesenchymal_Stem_Cells | DNase.fdr0.01.hot | | DNase |
| H1_Derived_Mesenchymal_Stem_Cells | DNase.fdr0.01.peaks | | DNase |
| H1_Derived_Mesenchymal_Stem_Cells | DNase.hot | | DNase |
| H1_Derived_Mesenchymal_Stem_Cells | DNase | | DNase |
| H1_Derived_Mesenchymal_Stem_Cells | H2AK5ac | | Histone |
| H1_Derived_Mesenchymal_Stem_Cells | H2A.Z | | Histone |
| H1_Derived_Mesenchymal_Stem_Cells | H2BK120ac | | Histone |
| H1_Derived_Mesenchymal_Stem_Cells | H2BK12ac | | Histone |
| H1_Derived_Mesenchymal_Stem_Cells | H2BK5ac | | Histone |
| H1_Derived_Mesenchymal_Stem_Cells | H3K14ac | | Histone |
| H1_Derived_Mesenchymal_Stem_Cells | H3K18ac | | Histone |
| H1_Derived_Mesenchymal_Stem_Cells | H3K23ac | | Histone |
| H1_Derived_Mesenchymal_Stem_Cells | H3K27ac | | Histone |
| H1_Derived_Mesenchymal_Stem_Cells | H3K27me3 | | Histone |
| H1_Derived_Mesenchymal_Stem_Cells | H3K36me3 | | Histone |
| H1_Derived_Mesenchymal_Stem_Cells | H3K4ac | | Histone |
| H1_Derived_Mesenchymal_Stem_Cells | H3K4me1 | | Histone |
| H1_Derived_Mesenchymal_Stem_Cells | H3K4me2 | | Histone |
| H1_Derived_Mesenchymal_Stem_Cells | H3K4me3 | | Histone |
| H1_Derived_Mesenchymal_Stem_Cells | H3K79me1 | | Histone |
| H1_Derived_Mesenchymal_Stem_Cells | H3K9ac | | Histone |
| H1_Derived_Mesenchymal_Stem_Cells | H3K9me3 | | Histone |
| H1_Derived_Mesenchymal_Stem_Cells | H4K8ac | | Histone |
| H1_Derived_Mesenchymal_Stem_Cells | H4K91ac | | Histone |
| H1_Derived_Neuronal_Progenitor_Cultured_Cells | DNase.all.peaks | | DNase |
| H1_Derived_Neuronal_Progenitor_Cultured_Cells | DNase.fdr0.01.hot | | DNase |
| H1_Derived_Neuronal_Progenitor_Cultured_Cells | DNase.fdr0.01.peaks | | DNase |
| H1_Derived_Neuronal_Progenitor_Cultured_Cells | DNase.hot | | DNase |
| H1_Derived_Neuronal_Progenitor_Cultured_Cells | DNase | | DNase |
| H1_Derived_Neuronal_Progenitor_Cultured_Cells | H2AK5ac | | Histone |
| H1_Derived_Neuronal_Progenitor_Cultured_Cells | H2BK120ac | | Histone |
| H1_Derived_Neuronal_Progenitor_Cultured_Cells | H2BK12ac | | Histone |
| H1_Derived_Neuronal_Progenitor_Cultured_Cells | H2BK15ac | | Histone |
| H1_Derived_Neuronal_Progenitor_Cultured_Cells | H2BK5ac | | Histone |
| H1_Derived_Neuronal_Progenitor_Cultured_Cells | H3K14ac | | Histone |
| H1_Derived_Neuronal_Progenitor_Cultured_Cells | H3K18ac | | Histone |
| H1_Derived_Neuronal_Progenitor_Cultured_Cells | H3K23ac | | Histone |
| H1_Derived_Neuronal_Progenitor_Cultured_Cells | H3K27ac | | Histone |
| H1_Derived_Neuronal_Progenitor_Cultured_Cells | H3K27me3 | | Histone |
| H1_Derived_Neuronal_Progenitor_Cultured_Cells | H3K36me3 | | Histone |
| H1_Derived_Neuronal_Progenitor_Cultured_Cells | H3K4ac | | Histone |
| H1_Derived_Neuronal_Progenitor_Cultured_Cells | H3K4me1 | | Histone |
| H1_Derived_Neuronal_Progenitor_Cultured_Cells | H3K4me2 | | Histone |
| H1_Derived_Neuronal_Progenitor_Cultured_Cells | H3K4me3 | | Histone |
| H1_Derived_Neuronal_Progenitor_Cultured_Cells | H3K79me1 | | Histone |
| H1_Derived_Neuronal_Progenitor_Cultured_Cells | H3K9ac | | Histone |
| H1_Derived_Neuronal_Progenitor_Cultured_Cells | H3K9me3 | | Histone |
| H1_Derived_Neuronal_Progenitor_Cultured_Cells | H4K8ac | | Histone |
| H1_Derived_Neuronal_Progenitor_Cultured_Cells | H4K91ac | | Histone |
| H9_Cell_Line | DNase.all.peaks | | DNase |
| H9_Cell_Line | DNase.fdr0.01.hot | | DNase |
| H9_Cell_Line | DNase.fdr0.01.peaks | | DNase |
| H9_Cell_Line | DNase.hot | | DNase |
| H9_Cell_Line | DNase | | DNase |
| H9_Cell_Line | H2AK5ac | | Histone |
| H9_Cell_Line | H2A.Z | | Histone |
| H9_Cell_Line | H2BK120ac | | Histone |
| H9_Cell_Line | H2BK12ac | | Histone |
| H9_Cell_Line | H2BK15ac | | Histone |
| H9_Cell_Line | H2BK20ac | | Histone |
| H9_Cell_Line | H2BK5ac | | Histone |
| H9_Cell_Line | H3K14ac | | Histone |
| H9_Cell_Line | H3K18ac | | Histone |
| H9_Cell_Line | H3K23ac | | Histone |
| H9_Cell_Line | H3K23me2 | | Histone |
| H9_Cell_Line | H3K27ac | | Histone |
| H9_Cell_Line | H3K27me3 | | Histone |
| H9_Cell_Line | H3K36me3 | | Histone |
| H9_Cell_Line | H3K4ac | | Histone |
| H9_Cell_Line | H3K4me1 | | Histone |
| H9_Cell_Line | H3K4me2 | | Histone |
| H9_Cell_Line | H3K4me3 | | Histone |
| H9_Cell_Line | H3K56ac | | Histone |
| H9_Cell_Line | H3K79me1 | | Histone |
| H9_Cell_Line | H3K79me2 | | Histone |
| H9_Cell_Line | H3K9ac | | Histone |
| H9_Cell_Line | H3K9me3 | | Histone |
| H9_Cell_Line | H3T11ph | | Histone |
| H9_Cell_Line | H4K20me1 | | Histone |
| H9_Cell_Line | H4K5ac | | Histone |
| H9_Cell_Line | H4K8ac | | Histone |
| H9_Cell_Line | H4K91ac | | Histone |
| H9_Derived_Neuronal_Progenitor_Cultured_Cells | H2A.Z | | Histone |
| H9_Derived_Neuronal_Progenitor_Cultured_Cells | H3K27me3 | | Histone |
| H9_Derived_Neuronal_Progenitor_Cultured_Cells | H3K36me3 | | Histone |
| H9_Derived_Neuronal_Progenitor_Cultured_Cells | H3K4me1 | | Histone |
| H9_Derived_Neuronal_Progenitor_Cultured_Cells | H3K4me3 | | Histone |
| H9_Derived_Neuronal_Progenitor_Cultured_Cells | H3K9me3 | | Histone |
| H9_Derived_Neuron_Cultured_Cells | H2A.Z | | Histone |
| H9_Derived_Neuron_Cultured_Cells | H3K27me3 | | Histone |
| H9_Derived_Neuron_Cultured_Cells | H3K36me3 | | Histone |
| H9_Derived_Neuron_Cultured_Cells | H3K4me1 | | Histone |
| H9_Derived_Neuron_Cultured_Cells | H3K4me3 | | Histone |
| H9_Derived_Neuron_Cultured_Cells | H3K9me3 | | Histone |
| hESC_Derived_CD184+_Endoderm_Cultured_Cells | H3K27ac | | Histone |
| hESC_Derived_CD184+_Endoderm_Cultured_Cells | H3K27me3 | | Histone |
| hESC_Derived_CD184+_Endoderm_Cultured_Cells | H3K36me3 | | Histone |
| hESC_Derived_CD184+_Endoderm_Cultured_Cells | H3K4me1 | | Histone |
| hESC_Derived_CD184+_Endoderm_Cultured_Cells | H3K4me3 | | Histone |
| hESC_Derived_CD184+_Endoderm_Cultured_Cells | H3K9ac | | Histone |
| hESC_Derived_CD184+_Endoderm_Cultured_Cells | H3K9me3 | | Histone |
| hESC_Derived_CD56+_Ectoderm_Cultured_Cells | H3K27ac | | Histone |
| hESC_Derived_CD56+_Ectoderm_Cultured_Cells | H3K27me3 | | Histone |
| hESC_Derived_CD56+_Ectoderm_Cultured_Cells | H3K36me3 | | Histone |
| hESC_Derived_CD56+_Ectoderm_Cultured_Cells | H3K4me1 | | Histone |
| hESC_Derived_CD56+_Ectoderm_Cultured_Cells | H3K4me3 | | Histone |
| hESC_Derived_CD56+_Ectoderm_Cultured_Cells | H3K9me3 | | Histone |
| hESC_Derived_CD56+_Mesoderm_Cultured_Cells | H3K27ac | | Histone |
| hESC_Derived_CD56+_Mesoderm_Cultured_Cells | H3K27me3 | | Histone |
| hESC_Derived_CD56+_Mesoderm_Cultured_Cells | H3K36me3 | | Histone |
| hESC_Derived_CD56+_Mesoderm_Cultured_Cells | H3K4me1 | | Histone |
| hESC_Derived_CD56+_Mesoderm_Cultured_Cells | H3K4me3 | | Histone |
| hESC_Derived_CD56+_Mesoderm_Cultured_Cells | H3K9me3 | | Histone |
| HUES48_Cell_Line | H3K27ac | | Histone |
| HUES48_Cell_Line | H3K27me3 | | Histone |
| HUES48_Cell_Line | H3K36me3 | | Histone |
| HUES48_Cell_Line | H3K4me1 | | Histone |
| HUES48_Cell_Line | H3K4me3 | | Histone |
| HUES48_Cell_Line | H3K9ac | | Histone |
| HUES48_Cell_Line | H3K9me3 | | Histone |
| HUES6_Cell_Line | H3K27ac | | Histone |
| HUES6_Cell_Line | H3K27me3 | | Histone |
| HUES6_Cell_Line | H3K36me3 | | Histone |
| HUES6_Cell_Line | H3K4me1 | | Histone |
| HUES6_Cell_Line | H3K4me3 | | Histone |
| HUES6_Cell_Line | H3K9ac | | Histone |
| HUES6_Cell_Line | H3K9me3 | | Histone |
| HUES641Cell_Line | H3K27ac | | Histone |
| HUES64_Cell_Line | H3K27me3 | | Histone |
| HUES64_Cell_Line | H3K36me3 | | Histone |
| HUES64_Cell_Line | H3K4me1 | | Histone |
| HUES64_Cell_Line | H3K4me3 | | Histone |
| HUES64_Cell_Line | H3K9ac | | Histone |
| HUES64_Cell_Line | H3K9me3 | | Histone |
| IMR90_Cell_Line | DNase.all.peaks | | DNase |
| IMR90_Cell_Line | DNase.fdr0.01.hot | | DNase |
| IMR90_Cell_Line | DNase.fdr0.01.peaks | | DNase |
| IMR90_Cell_Line | DNase.hot | | DNase |
| IMR90_Cell_Line | DNase | | DNase |
| IMR90_Cell_Line | H2AK5ac | | Histone |
| IMR90_Cell_Line | H2AK9ac | | Histone |
| IMR90_Cell_Line | H2A.Z | | Histone |
| IMR90_Cell_Line | H2BK120ac | | Histone |
| IMR90_Cell_Line | H2BK12ac | | Histone |
| IMR90_Cell_Line | H2BK15ac | | Histone |
| IMR90_Cell_Line | H2BK20ac | | Histone |
| IMR90_Cell_Line | H2BK5ac | | Histone |
| IMR90_Cell_Line | H3K14ac | | Histone |
| IMR90_Cell_Line | H3K18ac | | Histone |
| IMR90_Cell_Line | H3K23ac | | Histone |
| IMR90_Cell_Line | H3K27ac | | Histone |
| IMR90_Cell_Line | H3K27me3 | | Histone |
| IMR90_Cell_Line | H3K36me3 | | Histone |
| IMR90_Cell_Line | H3K4ac | | Histone |
| IMR90_Cell_Line | H3K4me1 | | Histone |
| IMR90_Cell_Line | H3K4me2 | | Histone |
| IMR90_Cell_Line | H3K4me3 | | Histone |
| IMR90_Cell_Line | H3K56ac | | Histone |
| IMR90_Cell_Line | H3K79me1 | | Histone |
| IMR90_Cell_Line | H3K79me2 | | Histone |
| IMR90_Cell_Line | H3K9ac | | Histone |
| IMR90_Cell_Line | H3K9me1 | | Histone |
| IMR90_Cell_Line | H3K9me3 | | Histone |
| IMR90_Cell_Line | H4K20me1 | | Histone |
| IMR90_Cell_Line | H4K5ac | | Histone |
| IMR90_Cell_Line | H4K8ac | | Histone |
| IMR90_Cell_Line | H4K91ac | | Histone |
| iPS-15b_Cell_Line | H3K27me3 | | Histone |
| iPS-15b_Cell_Line | H3K36me3 | | Histone |
| iPS-15b_Cell_Line | H3K4me1 | | Histone |
| iPS-15b_Cell_Line | H3K4me3 | | Histone |
| iPS-15b_Cell_Line | H3K9ac | | Histone |
| iPS-15b_Cell_Line | H3K9me3 | | Histone |
| iPS-18_Cell_Line | H3K27ac | | Histone |
| iPS-18_Cell_Line | H3K27me3 | | Histone |
| iPS-18_Cell_Line | H3K36me3 | | Histone |
| iPS-18_Cell_Line | H3K4me1 | | Histone |
| iPS-18_Cell_Line | H3K4me3 | | Histone |
| iPS-18_Cell_Line | H3K9ac | | Histone |
| iPS-18_Cell_Line | H3K9me3 | | Histone |
| iPS-20b_Cell_Line | H3K27ac | | Histone |
| iPS-20b_Cell_Line | H3K27me3 | | Histone |
| iPS-20b_Cell_Line | H3K36me3 | | Histone |
| iPS-20b_Cell_Line | H3K4me1 | | Histone |
| iPS-20b_Cell_Line | H3K4me3 | | Histone |
| iPS-20b_Cell_Line | H3K9ac | | Histone |
| iPS-20b_Cell_Line | H3K9me3 | | Histone |
| iPS_DF_6.9_Cell_Line | DNase.all.peaks | | DNase |
| iPS_DF_6.9_Cell_Line | DNase.fdr0.01.hot | | DNase |
| iPS_DF_6.9_Cell_Line | DNase.fdr0.01.peaks | | DNase |
| iPS_DF_6.9_Cell_Line | DNase.hot | | DNase |
| iPS_DF_6.9_Cell_Line | DNase | | DNase |
| iPS_DF_6.9_Cell_Line | H3K27ac | | Histone |
| iPS_DF_6.9_Cell_Line | H3K27me3 | | Histone |
| iPS_DF_6.9_Cell_Line | H3K36me3 | | Histone |
| iPS_DF_6.9_Cell_Line | H3K4me1 | | Histone |
| iPS_DF_6.9_Cell_Line | H3K4me3 | | Histone |
| iPS_DF_6.9_Cell_Line | H3K9me3 | | Histone |
| iPS_DF_19.11_Cell_Line | DNase.all.peaks | | DNase |
| iPS_DF_19.11_Cell_Line | DNase.fdr0.01.hot | | DNase |
| iPS_DF_19.11_Cell_Line | DNase.fdr0.01.peaks | | DNase |
| iPS_DF_19.11_Cell_Line | DNase.hot | | DNase |
| iPS_DF_19.11_Cell_Line | DNase | | DNase |
| iPS_DF_19.11_Cell_Line | H3K27ac | | Histone |
| iPS_DF_19.11_Cell_Line | H3K27me3 | | Histone |
| iPS_DF_19.11_Cell_Line | H3K36me3 | | Histone |
| iPS_DF_19.11_Cell_Line | H3K4me1 | | Histone |
| iPS_DF_19.11_Cell_Line | H3K4me3 | | Histone |
| iPS_DF_19.11_Cell_Line | H3K9me3 | | Histone |
| Mesenchymal_Stem_Cell_Derived_ | H3K27me3 | | Histone |
| Adipocyte_Cultured_Cells | | | |
| Mesenchymal_Stem_Cell_Derived_ | H3K36me3 | | Histone |
| Adipocyte_Cultured_Cells | | | |
| Mesenchymal_Stem_Cell_Derived_ | H3K4me1 | | Histone |
| Adipocyte_Cultured_Cells | | | |
| Mesenchymal_Stem_Cell_Derived_ | H3K4me3 | | Histone |
| Adipocyte_Cultured_Cells | | | |
| Mesenchymal_Stem_Cell_Derived_ | H3K9ac | | Histone |
| Adipocyte_Cultured_Cells | | | |
| Mesenchymal_Stem_Cell_Derived_ | H3K9me3 | | Histone |
| Adipocyte_Cultured_Cells | | | |
| 4star | H3K27me3 | | Histone |
| 4star | H3K36me3 | | Histone |
| 4star | H3K4me1 | | Histone |
| 4star | H3K4me3 | | Histone |
| 4star | H3K9me3 | | Histone |
| Adipose_Derived_Mesenchymal_ | H3K27me3 | | Histone |
| Stem_Cell_Cultured_Cells | | | |
| Adipose_Derived_Mesenchymal_ | H3K36me3 | | Histone |
| Stem_Cell_Cultured_Cells | | | |
| Adipose_Derived_Mesenchymal_ | H3K4me1 | | Histone |
| Stem_Cell_Cultured_Cells | | | |
| Adipose_Derived_Mesenchymal_ | H3K4me3 | | Histone |
| Stem_Cell_Cultured_Cells | | | |
| Adipose_Derived_Mesenchymal_ | H3K9ac | | Histone |
| Stem_Cell_Cultured_Cells | | | |
| Adipose_Derived_Mesenchymal_ | H3K9me3 | | Histone |
| Stem_Cell_Cultured_Cells | | | |
| Bone_Marrow_Derived_Mesenchymal_ | H3K27ac | | Histone |
| Stem_Cell_Cultured_Cells | | | |
| Bone_Marrow_Derived_Mesenchymal_ | H3K27me3 | | Histone |
| Stem_Cell_Cultured_Cells | | | |
| Bone_Marrow_Derived_Mesenchymal_ | H3K36me3 | | Histone |
| Stem_Cell_Cultured_Cells | | | |
| Bone_Marrow_Derived_Mesenchymal_ | H3K4me1 | | Histone |
| Stem_Cell_Cultured_Cells | | | |
| Bone_Marrow_Derived_Mesenchymal_ | H3K4me3 | | Histone |
| Stem_Cell_Cultured_Cells | | | |
| Bone_Marrow_Derived_Mesenchymal_ | H3K9ac | | Histone |
| Stem_Cell_Cultured_Cells | | | |
| Bone_Marrow_Derived_Mesenchymal_ | H3K9me3 | | Histone |
| Stem_Cell_Cultured_Cells | | | |
| Breast_Myoepithelial_Cells | H3K27me3 | | Histone |
| Breast_Myoepithelial_Cells | H3K36me3 | | Histone |
| Breast_Myoepithelial_Cells | H3K4me1 | | Histone |
| Breast_Myoepithelial_Cells | H3K4me3 | | Histone |
| Breast_Myoepithelial_Cells | H3K9ac | | Histone |
| Breast_Myoepithelial_Cells | H3K9me3 | | Histone |
| Breast_vHMEC | DNase.all.peaks | | DNase |
| Breast_vHMEC | DNase.fdr0.01.hot | | DNase |
| Breast_vHMEC | DNase.fdr0.01.peaks | | DNase |
| Breast_vHMEC | DNase.hot | | DNase |
| Breast_vHMEC | DNase | | DNase |
| Breast_vHMEC | H3K27me3 | | Histone |
| Breast_vHMEC | H3K36me3 | | Histone |
| Breast_vHMEC | H3K4me1 | | Histone |
| Breast_vHMEC | H3K4me3 | | Histone |
| Breast_vHMEC | H3K9me3 | | Histone |
| CD14_Primary_Cells | DNase.all.peaks | | DNase |
| CD14_Primary_Cells | DNase.fdr0.01.hot | | DNase |
| CD14_Primary_Cells | DNase.fdr0.01.peaks | | DNase |
| CD14_Primary_Cells | DNase.hot | | DNase |
| CD14_Primary_Cells | DNase | | DNase |
| CD14_Primary_Cells | H3K27ac | | Histone |
| CD14_Primary_Cells | H3K27me3 | | Histone |
| CD14_Primary_Cells | H3K36me3 | | Histone |
| CD14_Primary_Cells | H3K4me1 | | Histone |
| CD14_Primary_Cells | H3K4me3 | | Histone |
| CD14_Primary_Cells | H3K9me3 | | Histone |
| CD15_Primary_Cells | H3K27me3 | | Histone |
| CD15_Primary_Cells | H3K36me3 | | Histone |
| CD15_Primary_Cells | H3K4me1 | | Histone |
| CD15_Primary_Cells | H3K4me3 | | Histone |
| CD15_Primary_Cells | H3K9me3 | | Histone |
| CD19_Primary_Cells_Cord_BI | H3K27me3 | | Histone |
| CD19_Primary_Cells_Cord_BI | H3K36me3 | | Histone |
| CD19_Primary_Cells_Cord_BI | H3K4me1 | | Histone |
| CD19_Primary_Cells_Cord_BI | H3K4me3 | | Histone |
| CD19_Primary_Cells_Cord_BI | H3K9me3 | | Histone |
| CD19_Primary_Cells_Peripheral_ | DNase.all.peaks | | DNase |
| CD19_Primary_Cells_Peripheral_ | DNase.fdr0.01.hot | | DNase |
| CD19_Primary_Cells_Peripheral_ | DNase.fdr0.01.peaks | | DNase |
| CD19_Primary_Cells_Peripheral_ | DNase.hot | | DNase |
| CD19_Primary_Cells_Peripheral_ | DNase | | DNase |
| CD19_Primary_Cells_Peripheral_ | H3K27ac | | Histone |
| CD19_Primary_Cells_Peripheral_ | H3K27me3 | | Histone |
| CD19_Primary_Cells_Peripheral_ | H3K36me3 | | Histone |
| CD19_Primary_Cells_Peripheral_ | H3K4me1 | | Histone |
| CD19_Primary_Cells_Peripheral_ | H3K4me3 | | Histone |
| CD19_Primary_Cells_Peripheral_ | H3K9me3 | | Histone |
| CD3_Primary_Cells_Cord_BI | DNase.all.peaks | | DNase |
| CD3_Primary_Cells_Cord_BI | DNase.fdr0.01.hot | | DNase |
| CD3_Primary_Cells_Cord_BI | DNase.fdr0.01.peaks | | DNase |
| CD3_Primary_Cells_Cord_BI | DNase.hot | | DNase |
| CD3_Primary_Cells_Cord_BI | DNase | | DNase |
| CD3_Primary_Cells_Cord_BI | H3K27me3 | | Histone |
| CD3_Primary_Cells_Cord_BI | H3K36me3 | | Histone |
| CD3_Primary_Cells_Cord_BI | H3K4me1 | | Histone |
| CD3_Primary_Cells_Cord_BI | H3K4me3 | | Histone |
| CD3_Primary_Cells_Cord_BI | H3K9me3 | | Histone |
| CD3_Primary_Cells_Peripheral_ | DNase.all.peaks | | DNase |
| CD3_Primary_Cells_Peripheral_ | DNase.fdr0.01.hot | | DNase |
| CD3_Primary_Cells_Peripheral_ | DNase.fdr0.01.peaks | | DNase |
| CD3_Primary_Cells_Peripheral_ | DNase.hot | | DNase |
| CD3_Primary_Cells_Peripheral_ | DNase | | DNase |
| CD3_Primary_Cells_Peripheral_ | H3K27ac | | Histone |
| CD3_Primary_Cells_Peripheral_ | H3K27me3 | | Histone |
| CD3_Primary_Cells_Peripheral_ | H3K36me3 | | Histone |
| CD3_Primary_Cells_Peripheral_ | H3K4me1 | | Histone |
| CD3_Primary_Cells_Peripheral_ | H3K4me3 | | Histone |
| CD3_Primary_Cells_Peripheral_ | H3K9me3 | | Histone |
| CD34_Primary_Cells | H3K27me3 | | Histone |
| CD34_Primary_Cells | H3K36me3 | | Histone |
| CD34_Primary_Cells | H3K4me1 | | Histone |
| CD34_Primary_Cells | H3K4me3 | | Histone |
| CD34_Primary_Cells | H3K9me3 | | Histone |
| CD34_Cultured_Cells | H3K27me3 | | Histone |
| CD34_Cultured_Cells | H3K36me3 | | Histone |
| CD34_Cultured_Cells | H3K4me1 | | Histone |
| CD34_Cultured_Cells | H3K4me3 | | Histone |
| CD34_Cultured_Cells | H3K9me3 | | Histone |
| CD4_Memory_Primary_Cells | H3K27ac | | Histone |
| CD4_Memory_Primary_Cells | H3K27me3 | | Histone |
| CD4_Memory_Primary_Cells | H3K36me3 | | Histone |
| CD4_Memory_Primary_Cells | H3K4me1 | | Histone |
| CD4_Memory_Primary_Cells | H3K4me3 | | Histone |
| CD4_Memory_Primary_Cells | H3K9me3 | | Histone |
| CD4_Naive_Primary_Cells | H3K27ac | | Histone |
| CD4_Naive_Primary_Cells | H3K27me3 | | Histone |
| CD4_Naive_Primary_Cells | H3K36me3 | | Histone |
| CD4_Naive_Primary_Cells | H3K4me1 | | Histone |
| CD4_Naive_Primary_Cells | H3K4me3 | | Histone |
| CD4_Naive_Primary_Cells | H3K9ac | | Histone |
| CD4_Naive_Primary_Cells | H3K9me3 | | Histone |
| CD4+_CD25_CD45RA+_Naive_Primary_Cells | H3K27ac | | Histone |
| CD4+_CD25_CD45RA+_Naive_Primary_Cells | H3K27me3 | | Histone |
| CD4+_CD25_CD45RA+_Naive_Primary_Cells | H3K36me3 | | Histone |
| CD4+_CD25_CD45RA+_Naive_Primary_Cells | H3K4me1 | | Histone |
| CD4+_CD25_CD45RA+_Naive_Primary_Cells | H3K4me3 | | Histone |
| CD4+_CD25_CD45RA+_Naive_Primary_Cells | H3K9me3 | | Histone |
| CD4+_CD25_CD45RO+_Memory_Primary_Cells | H3K27ac | | Histone |
| CD4+_CD25_CD45RO+_Memory_Primary_Cells | H3K27me3 | | Histone |
| CD4+_CD25_CD45RO+_Memory_Primary_Cells | H3K36me3 | | Histone |
| CD4+_CD25_CD45RO+_Memory_Primary_Cells | H3K4me1 | | Histone |
| CD4+_CD25_CD45RO+_Memory_Primary_Cells | H3K4me3 | | Histone |
| CD4+_CD25_CD45RO+_Memory_Primary_Cells | H3K9me3 | | Histone |
| CD4+_CD25_IL17_PMA-lonomycin_stimulated_ | H3K27ac | | Histone |
| MACS_purified_Th_Primary_Cells | | | |
| CD4+_CD25_IL17_PMA-lonomycin_stimulated_ | H3K27me3 | | Histone |
| MACS_purified_Th_Primary_Cells | | | |
| CD4+_CD25_IL17_PMA-lonomycin_stimulated_ | H3K36me3 | | Histone |
| MACS_purified_Th_Primary_Cells | | | |
| CD4+_CD25_IL17_PMA-lonomycin_ | H3K4me1 | | Histone |
| stimulated_MACS_purified_Th_Primary_Cells | | | |
| CD4+_CD25_IL17_PMA-lonomycin_ | H3K4me3 | | Histone |
| stimulated_MACS_purified_Th_Primary_Cells | | | |
| CD4+_CD25_IL17_PMA-lonomycin_ | H3K9me3 | | Histone |
| stimulated_MACS_purified_Th_Primary_Cells | | | |
| CD4+_CD25_IL17+_PMA-lonomycin_ | H3K27ac | | Histone |
| stimulated_Th17_Primary_Cells | | | |
| CD4+_CD25_IL17+_PMA-lonomycin_ | H3K27me3 | | Histone |
| stimulated_Th17_Primary_Cells | | | |
| CD4+_CD25_IL17+_PMA-lonomycin_ | H3K36me3 | | Histone |
| stimulated_Th17_Primary_Cells | | | |
| CD4+_CD25_IL17+_PMA-lonomycin_ | H3K4me1 | | Histone |
| stimulated_Th17_Primary_Cells | | | |
| CD4+_CD25_IL17+_PMA-lonomycin_ | H3K4me3 | | Histone |
| stimulated_Th17_Primary_Cells | | | |
| CD4+_CD25_IL17+_PMA-lonomycin_ | H3K9me3 | | Histone |
| stimulated_Th17_Primary_Cells | | | |
| CD4+_CD25_Th_Primary_Cells | H3K27ac | | Histone |
| CD4+_CD25_Th_Primary_Cells | H3K27me3 | | Histone |
| CD4+_CD25_Th_Primary_Cells | H3K36me3 | | Histone |
| CD4+_CD25_Th_Primary_Cells | H3K4me1 | | Histone |
| CD4+_CD25_Th_Primary_Cells | H3K4me3 | | Histone |
| CD4+_CD25_Th_Primary_Cells | H3K9me3 | | Histone |
| CD4+_CD25+_CD127_Treg_Primary_Cells | H3K27ac | | Histone |
| CD4+_CD25+_CD127_Treg_Primary_Cells | H3K27me3 | | Histone |
| CD4+_CD25+_CD127_Treg_Primary_Cells | H3K36me3 | | Histone |
| CD4+_CD25+_CD127_Treg_Primary_Cells | H3K4me1 | | Histone |
| CD4+_CD25+_CD127_Treg_Primary_Cells | H3K4me3 | | Histone |
| CD4+_CD25+_CD127_Treg_Primary_Cells | H3K9me3 | | Histone |
| CD4+_CD25int_CD127+_Tmem_Primary_Cells | H3K27ac | | Histone |
| CD4+_CD25int_CD127+_Tmem_Primary_Cells | H3K27me3 | | Histone |
| CD4+_CD25int_CD127+_Tmem_Primary_Cells | H3K36me3 | | Histone |
| CD4+_CD25int_CD127+_Tmem_Primary_Cells | H3K4me1 | | Histone |
| CD4+_CD25int_CD127+_Tmem_Primary_Cells | H3K4me3 | | Histone |
| CD4+_CD25int_CD127+_Tmem_Primary_Cells | H3K9me3 | | Histone |
| CD56_Primary_Cells | DNase.all.peaks | | DNase |
| CD56_Primary_Cells | DNase.fdr0.01.hot | | DNase |
| CD56_Primary_Cells | DNase.fdr0.01.peaks | | DNase |
| CD56_Primary_Cells | DNase.hot | | DNase |
| CD56_Primary_Cells | DNase | | DNase |
| CD56_Primary_Cells | H3K27ac | | Histone |
| CD56_Primary_Cells | H3K27me3 | | Histone |
| CD56_Primary_Cells | H3K36me3 | | Histone |
| CD56_Primary_Cells | H3K4me1 | | Histone |
| CD56_Primary_Cells | H3K4me3 | | Histone |
| CD8_Naive_Primary_Cells | H3K27ac | | Histone |
| CD8_Naive_Primary_Cells | H3K27me3 | | Histone |
| CD8_Naive_Primary_Cells | H3K36me3 | | Histone |
| CD8_Naive_Primary_Cells | H3K4me1 | | Histone |
| CD8_Naive_Primary_Cells | H3K4me3 | | Histone |
| CD8_Naive_Primary_Cells | H3K9ac | | Histone |
| CD8_Naive_Primary_Cells | H3K9me3 | | Histone |
| CD8_Memory_Primary_Cells | H3K27ac | | Histone |
| CD8_Memory_Primary_Cells | H3K27me3 | | Histone |
| CD8_Memory_Primary_Cells | H3K36me3 | | Histone |
| CD8_Memory_Primary_Cells | H3K4me1 | | Histone |
| CD8_Memory_Primary_Cells | H3K4me3 | | Histone |
| CD8_Memory_Primary_Cells | H3K9me3 | | Histone |
| Chondrocytes_from_Bone_Marrow_Derived_ | H3K27ac | | Histone |
| Mesenchymal_Stem_Cell_Cultured_Cells | | | |
| Chondrocytes_from_Bone_Marrow_Derived_ | H3K27me3 | | Histone |
| Mesenchymal_Stem_Cell_Cultured_Cells | | | |
| Chondrocytes_from_Bone_Marrow_Derived_ | H3K36me3 | | Histone |
| Mesenchymal_Stem_Cell_Cultured_Cells | | | |
| Chondrocytes_from_Bone_Marrow_Derived_ | H3K4me1 | | Histone |
| Mesenchymal_Stem_Cell_Cultured_Cells | | | |
| Chondrocytes_from_Bone_Marrow_Derived_ | H3K4me3 | | Histone |
| Mesenchymal_Stem_Cell_Cultured_Cells | | | |
| Chondrocytes_from_Bone_Marrow_Derived_ | H3K9ac | | Histone |
| Mesenchymal_Stem_Cell_Cultured_Cells | | | |
| Chondrocytes_from_Bone_Marrow_Derived_ | H3K9me3 | | Histone |
| Mesenchymal_Stem_Cell_Cultured_Cells | | | |
| Mobilized_CD34_Primary_Cells_Female | DNase.all.peaks | | DNase |
| Mobilized_CD34_Primary_Cells_Female | DNase.fdr0.01.hot | | DNase |
| Mobilized_CD34_Primary_Cells_Female | DNase.fdr0.01.peaks | | DNase |
| Mobilized_CD34_Primary_Cells_Female | DNase.hot | | DNase |
| Mobilized_CD34_Primary_Cells_Female | DNase | | DNase |
| Mobilized_CD34_Primary_Cells_Female | H3K27ac | | Histone |
| Mobilized_CD34_Primary_Cells_Female | H3K27me3 | | Histone |
| Mobilized_CD34_Primary_Cells_Female | H3K36me3 | | Histone |
| Mobilized_CD34_Primary_Cells_Female | H3K4me1 | | Histone |
| Mobilized_CD34_Primary_Cells_Female | H3K4me3 | | Histone |
| Mobilized_CD34_Primary_Cells_Female | H3K9me3 | | Histone |
| Mobilized_CD34_Primary_Cells_Male | DNase.all.peaks | | DNase |
| Mobilized_CD34_Primary_Cells_Male | DNase.fdr0.01.hot | | DNase |
| Mobilized_CD34_Primary_Cells_Male | DNase.fdr0.01.peaks | | DNase |
| Mobilized_CD34_Primary_Cells_Male | DNase.hot | | DNase |
| Mobilized_CD34_Primary_Cells_Male | DNase | | DNase |
| Mobilized_CD34_Primary_Cells_Male | H3K27me3 | | Histone |
| Mobilized_CD34_Primary_Cells_Male | H3K36me3 | | Histone |
| Mobilized_CD34_Primary_Cells_Male | H3K4me1 | | Histone |
| Mobilized_CD34_Primary_Cells_Male | H3K4me3 | | Histone |
| Mobilized_CD34_Primary_Cells_Male | H3K9me3 | | Histone |
| Muscle_Satellite_Cultured_Cells | H3K27me3 | | Histone |
| Muscle_Satellite_Cultured_Cells | H3K36me3 | | Histone |
| Muscle_Satellite_Cultured_Cells | H3K4me1 | | Histone |
| Muscle_Satellite_Cultured_Cells | H3K4me2 | | Histone |
| Muscle_Satellite_Cultured_Cells | H3K4me3 | | Histone |
| Muscle_Satellite_Cultured_Cells | H3K9ac | | Histone |
| Muscle_Satellite_Cultured_Cells | H3K9me3 | | Histone |
| Neurosphere_Cultured_Cells_Cortex_Derived | H3K27me3 | | Histone |
| Neurosphere_Cultured_Cells_Cortex_Derived | H3K36me3 | | Histone |
| Neurosphere_Cultured_Cells_Cortex_Derived | H3K4me1 | | Histone |
| Neurosphere_Cultured_Cells_Cortex_Derived | H3K4me3 | | Histone |
| Neurosphere_Cultured_Cells_Cortex_Derived | H3K9me3 | | Histone |
| Neurosphere_Cultured_Cells_Ganglionic_ | H3K27me3 | | Histone |
| Eminence_Derived | | | |
| Neurosphere_Cultured_Cells_Ganglionic_ | H3K36me3 | | Histone |
| Eminence_Derived | | | |
| Neurosphere_Cultured_Cells_Ganglionic_ | H3K4me1 | | Histone |
| Eminence_Derived | | | |
| Neurosphere_Cultured_Cells_Ganglionic_ | H3K4me3 | | Histone |
| Eminence_Derived | | | |
| Neurosphere_Cultured_Cells_Ganglionic_ | H3K9me3 | | Histone |
| Eminence_Derived | | | |
| Penis_Foreskin_Fibroblast_Primary_Cells_skin01 | DNase.all.peaks | | DNase |
| Penis_Foreskin_Fibroblast_Primary_Cells_skin01 | DNase.fdr0.01.hot | | DNase |
| Penis_Foreskin_Fibroblast_Primary_Cells_skin01 | DNase.fdr0.01.peaks | | DNase |
| Penis_Foreskin_Fibroblast_Primary_Cells_skin01 | DNase.hot | | DNase |
| Penis_Foreskin_Fibroblast_Primary_Cells_skin01 | DNase | | DNase |
| Penis_Foreskin_Fibroblast_Primary_Cells_skin01 | H3K27ac | | Histone |
| Penis_Foreskin_Fibroblast_Primary_Cells_skin01 | H3K27me3 | | Histone |
| Penis_Foreskin_Fibroblast_Primary_Cells_skin01 | H3K36me3 | | Histone |
| Penis_Foreskin_Fibroblast_Primary_Cells_skin01 | H3K4me1 | | Histone |
| Penis_Foreskin_Fibroblast_Primary_Cells_skin01 | H3K4me3 | | Histone |
| Penis_Foreskin_Fibroblast_Primary_Cells_skin01 | H3K9me3 | | Histone |
| Penis_Foreskin_Fibroblast_Primary_Cells_skin02 | DNase.all.peaks | | DNase |
| Penis_Foreskin_Fibroblast_Primary_Cells_skin02 | DNase.fdr0.01.hot | | DNase |
| Penis_Foreskin_Fibroblast_Primary_Cells_skin02 | DNase.fdr0.01.peaks | | DNase |
| Penis_Foreskin_Fibroblast_Primary_Cells_skin02 | DNase.hot | | DNase |
| Penis_Foreskin_Fibroblast_Primary_Cells_skin02 | DNase | | DNase |
| Penis_Foreskin_Fibroblast_Primary_Cells_skin02 | H3K27ac | | Histone |
| Penis_Foreskin_Fibroblast_Primary_Cells_skin02 | H3K27me3 | | Histone |
| Penis_Foreskin_Fibroblast_Primary_Cells_skin02 | H3K36me3 | | Histone |
| Penis_Foreskin_Fibroblast_Primary_Cells_skin02 | H3K4me1 | | Histone |
| Penis_Foreskin_Fibroblast_Primary_Cells_skin02 | H3K4me3 | | Histone |
| Penis_Foreskin_Fibroblast_Primary_Cells_skin02 | H3K9me3 | | Histone |
| Penis_Foreskin_Keratinocyte_Primary_Cells_skin02 | DNase.all.peaks | | DNase |
| Penis_Foreskin_Keratinocyte_Primary_Cells_skin02 | DNase.fdr0.01.hot | | DNase |
| Penis_Foreskin_Keratinocyte_Primary_Cells_skin02 | DNase.fdr0.01.peaks | | DNase |
| Penis_Foreskin_Keratinocyte_Primary_Cells_skin02 | DNase.hot | | DNase |
| Penis_Foreskin_Keratinocyte_Primary_Cells_skin02 | DNase | | DNase |
| Penis_Foreskin_Keratinocyte_Primary_Cells_skin02 | H3K27me3 | | Histone |
| Penis_Foreskin_Keratinocyte_Primary_Cells_skin02 | H3K36me3 | | Histone |
| Penis_Foreskin_Keratinocyte_Primary_Cells_skin02 | H3K4me1 | | Histone |
| Penis_Foreskin_Keratinocyte_Primary_Cells_skin02 | H3K4me3 | | Histone |
| Penis_Foreskin_Keratinocyte_Primary_Cells_skin02 | H3K9me3 | | Histone |
| Penis_Foreskin_Keratinocyte_Primary_Cells_skin03 | H3K27ac | | Histone |
| Penis_Foreskin_Keratinocyte_Primary_Cells_skin03 | H3K27me3 | | Histone |
| Penis_Foreskin_Keratinocyte_Primary_Cells_skin03 | H3K36me3 | | Histone |
| Penis_Foreskin_Keratinocyte_Primary_Cells_skin03 | H3K4me1 | | Histone |
| Penis_Foreskin_Keratinocyte_Primary_Cells_skin03 | H3K4me3 | | Histone |
| Penis_Foreskin_Keratinocyte_Primary_Cells_skin03 | H3K9me3 | | Histone |
| Penis_Foreskin_Melanocyte_Primary_Cells_skin01 | DNase.all.peaks | | DNase |
| Penis_Foreskin_Melanocyte_Primary_Cells_skin01 | DNase.fdr0.01.hot | | DNase |
| Penis_Foreskin_Melanocyte_Primary_Cells_skin01 | DNase.fdr0.01.peaks | | DNase |
| Penis_Foreskin_Melanocyte_Primary_Cells_skin01 | DNase.hot | | DNase |
| Penis_Foreskin_Melanocyte_Primary_Cells_skin01 | DNase | | DNase |
| Penis_Foreskin_Melanocyte_Primary_Cells_skin01 | H3K27ac | | Histone |
| Penis_Foreskin_Melanocyte_Primary_Cells_skin01 | H3K27me3 | | Histone |
| Penis_Foreskin_Melanocyte_Primary_Cells_skin01 | H3K36me3 | | Histone |
| Penis_Foreskin_Melanocyte_Primary_Cells_skin01 | H3K4me1 | | Histone |
| Penis_Foreskin_Melanocyte_Primary_Cells_skin01 | H3K4me3 | | Histone |
| Penis_Foreskin_Melanocyte_Primary_Cells_skin01 | H3K9me3 | | Histone |
| Penis_Foreskin_Melanocyte_Primary_Cells_skin03 | H3K27ac | | Histone |
| Penis_Foreskin_Melanocyte_Primary_Cells_skin03 | H3K27me3 | | Histone |
| Penis_Foreskin_Melanocyte_Primary_Cells_skin03 | H3K36me3 | | Histone |
| Penis_Foreskin_Melanocyte_Primary_Cells_skin03 | H3K4me1 | | Histone |
| Penis_Foreskin_Melanocyte_Primary_Cells_skin03 | H3K4me3 | | Histone |
| Penis_Foreskin_Melanocyte_Primary_Cells_skin03 | H3K9me3 | | Histone |
| Peripheral_Blood_Mononuclear_Primary_Cells | H3K27ac | | Histone |
| Peripheral_Blood_Mononuclear_Primary_Cells | H3K27me3 | | Histone |
| Peripheral_Blood_Mononuclear_Primary_Cells | H3K36me3 | | Histone |
| Peripheral_Blood_Mononuclear_Primary_Cells | H3K4me1 | | Histone |
| Peripheral_Blood_Mononuclear_Primary_Cells | H3K4me3 | | Histone |
| Peripheral_Blood_Mononuclear_Primary_Cells | H3K9ac | | Histone |
| Peripheral_Blood_Mononuclear_Primary_Cells | H3K9me3 | | Histone |
| Adipose_Nuclei | H3K27ac | | Histone |
| Adipose_Nuclei | H3K27me3 | | Histone |
| Adipose_Nuclei | H3K36me3 | | Histone |
| Adipose_Nuclei | H3K4me1 | | Histone |
| Adipose_Nuclei | H3K4me3 | | Histone |
| Adipose_Nuclei | H3K9ac | | Histone |
| Adipose_Nuclei | H3K9me3 | | Histone |
| Aorta | H3K27ac | | Histone |
| Aorta | H3K27me3 | | Histone |
| Aorta | H3K36me3 | | Histone |
| Aorta | H3K4me1 | | Histone |
| Aorta | H3K4me3 | | Histone |
| Aorta | H3K9me3 | | Histone |
| Adult_Liver | H3K27ac | | Histone |
| Adult_Liver | H3K27me3 | | Histone |
| Adult_Liver | H3K36me3 | | Histone |
| Adult_Liver | H3K4me1 | | Histone |
| Adult_Liver | H3K4me3 | | Histone |
| Adult_Liver | H3K9ac | | Histone |
| Adult_Liver | H3K9me3 | | Histone |
| Brain_Angular_Gyrus | H3K27ac | | Histone |
| Brain_Angular_Gyrus | H3K27me3 | | Histone |
| Brain_Angular_Gyrus | H3K36me3 | | Histone |
| Brain_Angular_Gyrus | H3K4me1 | | Histone |
| Brain_Angular_Gyrus | H3K4me3 | | Histone |
| Brain_Angular_Gyrus | H3K9ac | | Histone |
| Brain_Angular_Gyrus | H3K9me3 | | Histone |
| Brain_Anterior_Caudate | H3K27ac | | Histone |
| Brain_Anterior_Caudate | H3K27me3 | | Histone |
| Brain_Anterior_Caudate | H3K36me3 | | Histone |
| Brain_Anterior_Caudate | H3K4me1 | | Histone |
| Brain_Anterior_Caudate | H3K4me3 | | Histone |
| Brain_Anterior_Caudate | H3K9ac | | Histone |
| Brain_Anterior_Caudate | H3K9me3 | | Histone |
| Brain_Cingulate_Gyrus | H3K27ac | | Histone |
| Brain_Cingulate_Gyrus | H3K27me3 | | Histone |
| Brain_Cingulate_Gyrus | H3K36me3 | | Histone |
| Brain_Cingulate_Gyrus | H3K4me1 | | Histone |
| Brain_Cingulate_Gyrus | H3K4me3 | | Histone |
| Brain_Cingulate_Gyrus | H3K9ac | | Histone |
| Brain_Cingulate_Gyrus | H3K9me3 | | Histone |
| Brain_Germinal_Matrix | H3K27me3 | | Histone |
| Brain_Germinal_Matrix | H3K36me3 | | Histone |
| Brain_Germinal_Matrix | H3K4me1 | | Histone |
| Brain_Germinal_Matrix | H3K4me3 | | Histone |
| Brain_Germinal_Matrix | H3K9me3 | | Histone |
| Brain_Hippocampus_Middle | H3K27ac | | Histone |
| Brain_Hippocampus_Middle | H3K27me3 | | Histone |
| Brain_Hippocampus_Middle | H3K36me3 | | Histone |
| Brain_Hippocampus_Middle | H3K4me1 | | Histone |
| Brain_Hippocampus_Middle | H3K4me3 | | Histone |
| Brain_Hippocampus_Middle | H3K9me3 | | Histone |
| Brain_Inferior_Temporal_Lobe | H3K27ac | | Histone |
| Brain_Inferior_Temporal_Lobe | H3K27me3 | | Histone |
| Brain_Inferior_Temporal_Lobe | H3K36me3 | | Histone |
| Brain_Inferior_Temporal_Lobe | H3K4me1 | | Histone |
| Brain_Inferior_Temporal_Lobe | H3K4me3 | | Histone |
| Brain_Inferior_Temporal_Lobe | H3K9ac | | Histone |
| Brain_Inferior_Temporal_Lobe | H3K9me3 | | Histone |
| Brain_Mid_Frontal_Lobe | H3K27ac | | Histone |
| Brain_Mid_Frontal_Lobe | H3K27me3 | | Histone |
| Brain_Mid_Frontal_Lobe | H3K36me3 | | Histone |
| Brain_Mid_Frontal_Lobe | H3K4me1 | | Histone |
| Brain_Mid_Frontal_Lobe | H3K4me3 | | Histone |
| Brain_Mid_Frontal_Lobe | H3K9ac | | Histone |
| Brain_Mid_Frontal_Lobe | H3K9me3 | | Histone |
| Brain_Substantia_Nigra | H3K27ac | | Histone |
| Brain_Substantia_Nigra | H3K27me3 | | Histone |
| Brain_Substantia_Nigra | H3K36me3 | | Histone |
| Brain_Substantia_Nigra | H3K4me1 | | Histone |
| Brain_Substantia_Nigra | H3K4me3 | | Histone |
| Brain_Substantia_Nigra | H3K9ac | | Histone |
| Brain_Substantia_Nigra | H3K9me3 | | Histone |
| Colonic_Mucosa | H3K27ac | | Histone |
| Colonic_Mucosa | H3K27me3 | | Histone |
| Colonic_Mucosa | H3K36me3 | | Histone |
| Colonic_Mucosa | H3K4me1 | | Histone |
| Colonic_Mucosa | H3K4me3 | | Histone |
| Colonic_Mucosa | H3K9ac | | Histone |
| Colonic_Mucosa | H3K9me3 | | Histone |
| Colon_Smooth_Muscle | H3K27ac | | Histone |
| Colon_Smooth_Muscle | H3K27me3 | | Histone |
| Colon_Smooth_Muscle | H3K36me3 | | Histone |
| Colon_Smooth_Muscle | H3K4me1 | | Histone |
| Colon_Smooth_Muscle | H3K4me3 | | Histone |
| Colon_Smooth_Muscle | H3K9ac | | Histone |
| Colon_Smooth_Muscle | H3K9me3 | | Histone |
| Duodenum_Mucosa | H3K27me3 | | Histone |
| Duodenum_Mucosa | H3K36me3 | | Histone |
| Duodenum_Mucosa | H3K4me1 | | Histone |
| Duodenum_Mucosa | H3K4me3 | | Histone |
| Duodenum_Mucosa | H3K9ac | | Histone |
| Duodenum_Mucosa | H3K9me3 | | Histone |
| Duodenum_Smooth_Muscle | H3K27ac | | Histone |
| Duodenum_Smooth_Muscle | H3K27me3 | | Histone |
| Duodenum_Smooth_Muscle | H3K36me3 | | Histone |
| Duodenum_Smooth_Muscle | H3K4me1 | | Histone |
| Duodenum_Smooth_Muscle | H3K4me3 | | Histone |
| Duodenum_Smooth_Muscle | H3K9me3 | | Histone |
| Esophagus | H3K27ac | | Histone |
| Esophagus | H3K27me3 | | Histone |
| Esophagus | H3K36me3 | | Histone |
| Esophagus | H3K4me1 | | Histone |
| Esophagus | H3K4me3 | | Histone |
| Esophagus | H3K9me3 | | Histone |
| Fetal_Adrenal_Gland | DNase.all.peaks | | DNase |
| Fetal_Adrenal_Gland | DNase.fdr0.01.hot | | DNase |
| Fetal_Adrenal_Gland | DNase.fdr0.01.peaks | | DNase |
| Fetal_Adrenal_Gland | DNase.hot | | DNase |
| Fetal_Adrenal_Gland | DNase | | DNase |
| Fetal_Adrenal_Gland | H3K27ac | | Histone |
| Fetal_Adrenal_Gland | H3K27me3 | | Histone |
| Fetal_Adrenal_Gland | H3K36me3 | | Histone |
| Fetal_Adrenal_Gland | H3K4me1 | | Histone |
| Fetal_Adrenal_Gland | H3K4me3 | | Histone |
| Fetal_Adrenal_Gland | H3K9me3 | | Histone |
| Fetal_Brain_Male | DNase.all.peaks | | DNase |
| Fetal_Brain_Male | DNase.fdr0.01.hot | | DNase |
| Fetal_Brain_Male | DNase.fdr0.01.peaks | | DNase |
| Fetal_Brain_Male | DNase.hot | | DNase |
| Fetal_Brain_Male | DNase | | DNase |
| Fetal_Brain_Male | H3K27me3 | | Histone |
| Fetal_Brain_Male | H3K36me3 | | Histone |
| Fetal_Brain_Male | H3K4me1 | | Histone |
| Fetal_Brain_Male | H3K4me3 | | Histone |
| Fetal_Brain_Male | H3K9me3 | | Histone |
| Fetal_Brain_Female | DNase.all.peaks | | DNase |
| Fetal_Brain_Female | DNase.fdr0.01.hot | | DNase |
| Fetal_Brain_Female | DNase.fdr0.01.peaks | | DNase |
| Fetal_Brain_Female | DNase.hot | | DNase |
| Fetal_Brain_Female | DNase | | DNase |
| Fetal_Brain_Female | H3K27me3 | | Histone |
| Fetal_Brain_Female | H3K36me3 | | Histone |
| Fetal_Brain_Female | H3K4me1 | | Histone |
| Fetal_Brain_Female | H3K4me3 | | Histone |
| Fetal_Brain_Female | H3K9me3 | | Histone |
| Fetal_Heart | DNase.all.peaks | | DNase |
| Fetal_Heart | DNase.fdr0.01.hot | | DNase |
| Fetal_Heart | DNase.fdr0.01.peaks | | DNase |
| Fetal_Heart | DNase.hot | | DNase |
| Fetal_Heart | DNase | | DNase |
| Fetal_Heart | H3K27me3 | | Histone |
| Fetal_Heart | H3K36me3 | | Histone |
| Fetal_Heart | H3K4me1 | | Histone |
| Fetal_Heart | H3K4me3 | | Histone |
| Fetal_Heart | H3K9ac | | Histone |
| Fetal_Heart | H3K9me3 | | Histone |
| Fetal_Intestine_Large | DNase.all.peaks | | DNase |
| Fetal_Intestine_Large | DNase.fdr0.01.hot | | DNase |
| Fetal_Intestine_Large | DNase.fdr0.01.peaks | | DNase |
| Fetal_Intestine_Large | DNase.hot | | DNase |
| Fetal_Intestine_Large | DNase | | DNase |
| Fetal_Intestine_Large | H3K27ac | | Histone |
| Fetal_Intestine_Large | H3K27me3 | | Histone |
| Fetal_Intestine_Large | H3K36me3 | | Histone |
| Fetal_Intestine_Large | H3K4me1 | | Histone |
| Fetal_Intestine_Large | H3K4me3 | | Histone |
| Fetal_Intestine_Large | H3K9me3 | | Histone |
| Fetal_Intestine_Small | DNase.all.peaks | | DNase |
| Fetal_Intestine_Small | DNase.fdr0.01.hot | | DNase |
| Fetal_Intestine_Small | DNase.fdr0.01.peaks | | DNase |
| Fetal_Intestine_Small | DNase.hot | | DNase |
| Fetal_Intestine_Small | DNase | | DNase |
| Fetal_Intestine_Small | H3K27ac | | Histone |
| Fetal_Intestine_Small | H3K27me3 | | Histone |
| Fetal_Intestine_Small | H3K36me3 | | Histone |
| Fetal_Intestine_Small | H3K4me1 | | Histone |
| Fetal_Intestine_Small | H3K4me3 | | Histone |
| Fetal_Intestine_Small | H3K9me3 | | Histone |
| Fetal_Kidney | DNase.all.peaks | | DNase |
| Fetal_Kidney | DNase.fdr0.01.hot | | DNase |
| Fetal_Kidney | DNase.fdr0.01.peaks | | DNase |
| Fetal_Kidney | DNase.hot | | DNase |
| Fetal_Kidney | DNase | | DNase |
| Fetal_Kidney | H3K27me3 | | Histone |
| Fetal_Kidney | H3K36me3 | | Histone |
| Fetal_Kidney | H3K4me1 | | Histone |
| Fetal_Kidney | H3K4me3 | | Histone |
| Fetal_Kidney | H3K9ac | | Histone |
| Fetal_Kidney | H3K9me3 | | Histone |
| Pancreatic_Islets | H3K27ac | | Histone |
| Pancreatic_Islets | H3K27me3 | | Histone |
| Pancreatic_Islets | H3K36me3 | | Histone |
| Pancreatic_Islets | H3K4me1 | | Histone |
| Pancreatic_Islets | H3K4me3 | | Histone |
| Pancreatic_Islets | H3K9ac | | Histone |
| Pancreatic_Islets | H3K9me3 | | Histone |
| Fetal_Lung | DNase.all.peaks | | DNase |
| Fetal_Lung | DNase.fdr0.01.hot | | DNase |
| Fetal_Lung | DNase.fdr0.01.peaks | | DNase |
| Fetal_Lung | DNase.hot | | DNase |
| Fetal_Lung | DNase | | DNase |
| Fetal_Lung | H3K27me3 | | Histone |
| Fetal_Lung | H3K36me3 | | Histone |
| Fetal_Lung | H3K4me1 | | Histone |
| Fetal_Lung | H3K4me3 | | Histone |
| Fetal_Lung | H3K9ac | | Histone |
| Fetal_Lung | H3K9me3 | | Histone |
| Fetal_Muscle_Trunk | DNase.all.peaks | | DNase |
| Fetal_Muscle_Trunk | DNase.fdr0.01.hot | | DNase |
| Fetal_Muscle_Trunk | DNase.fdr0.01.peaks | | DNase |
| Fetal_Muscle_Trunk | DNase.hot | | DNase |
| Fetal_Muscle_Trunk | DNase | | DNase |
| Fetal_Muscle_Trunk | H3K27ac | | Histone |
| Fetal_Muscle_Trunk | H3K27me3 | | Histone |
| Fetal_Muscle_Trunk | H3K36me3 | | Histone |
| Fetal_Muscle_Trunk | H3K4me1 | | Histone |
| Fetal_Muscle_Trunk | H3K4me3 | | Histone |
| Fetal_Muscle_Trunk | H3K9me3 | | Histone |
| Fetal_Muscle_Leg | DNase.all.peaks | | DNase |
| Fetal_Muscle_Leg | DNase.fdr0.01.hot | | DNase |
| Fetal_Muscle_Leg | DNase.fdr0.01.peaks | | DNase |
| Fetal_Muscle_Leg | DNase.hot | | DNase |
| Fetal_Muscle_Leg | DNase | | DNase |
| Fetal_Muscle_Leg | H3K27ac | | Histone |
| Fetal_Muscle_Leg | H3K27me3 | | Histone |
| Fetal_Muscle_Leg | H3K36me3 | | Histone |
| Fetal_Muscle_Leg | H3K4me1 | | Histone |
| Fetal_Muscle_Leg | H3K4me3 | | Histone |
| Fetal_Muscle_Leg | H3K9me3 | | Histone |
| Fetal_Placenta | DNase.all.peaks | | DNase |
| Fetal_Placenta | DNase.fdr0.01.hot | | DNase |
| Fetal_Placenta | DNase.fdr0.01.peaks | | DNase |
| Fetal_Placenta | DNase.hot | | DNase |
| Fetal_Placenta | DNase | | DNase |
| Fetal_Placenta | H3K27ac | | Histone |
| Fetal_Placenta | H3K27me3 | | Histone |
| Fetal_Placenta | H3K36me3 | | Histone |
| Fetal_Placenta | H3K4me1 | | Histone |
| Fetal_Placenta | H3K4me3 | | Histone |
| Fetal_Placenta | H3K9me3 | | Histone |
| Fetal_Stomach | DNase.all.peaks | | DNase |
| Fetal_Stomach | DNase.fdr0.01.hot | | DNase |
| Fetal_Stomach | DNase.fdr0.01.peaks | | DNase |
| Fetal_Stomach | DNase.hot | | DNase |
| Fetal_Stomach | DNase | | DNase |
| Fetal_Stomach | H3K27ac | | Histone |
| Fetal_Stomach | H3K27me3 | | Histone |
| Fetal_Stomach | H3K36me3 | | Histone |
| Fetal_Stomach | H3K4me1 | | Histone |
| Fetal_Stomach | H3K4me3 | | Histone |
| Fetal_Stomach | H3K9me3 | | Histone |
| Fetal_Thymus | DNase.all.peaks | | DNase |
| Fetal_Thymus | DNase.fdr0.01.hot | | DNase |
| Fetal_Thymus | DNase.fdr0.01.peaks | | DNase |
| Fetal_Thymus | DNase.hot | | DNase |
| Fetal_Thymus | DNase | | DNase |
| Fetal_Thymus | H3K27ac | | Histone |
| Fetal_Thymus | H3K27me3 | | Histone |
| Fetal_Thymus | H3K36me3 | | Histone |
| Fetal_Thymus | H3K4me1 | | Histone |
| Fetal_Thymus | H3K4me3 | | Histone |
| Fetal_Thymus | H3K9me3 | | Histone |
| Gastric | DNase.all.peaks | | DNase |
| Gastric | DNase.fdr0.01.hot | | DNase |
| Gastric | DNase.fdr0.01.peaks | | DNase |
| Gastric | DNase.hot | | DNase |
| Gastric | DNase | | DNase |
| Gastric | H3K27ac | | Histone |
| Gastric | H3K27me3 | | Histone |
| Gastric | H3K36me3 | | Histone |
| Gastric | H3K4me1 | | Histone |
| Gastric | H3K4me3 | | Histone |
| Gastric | H3K9me3 | | Histone |
| Left_Ventricle | H3K27ac | | Histone |
| Left_Ventricle | H3K27me3 | | Histone |
| Left_Ventricle | H3K36me3 | | Histone |
| Left_Ventricle | H3K4me1 | | Histone |
| Left_Ventricle | H3K4me3 | | Histone |
| Left_Ventricle | H3K9me3 | | Histone |
| Lung | H3K27ac | | Histone |
| Lung | H3K27me3 | | Histone |
| Lung | H3K36me3 | | Histone |
| Lung | H3K4me1 | | Histone |
| Lung | H3K4me3 | | Histone |
| Lung | H3K9me3 | | Histone |
| Ovary | DNase.all.peaks | | DNase |
| Ovary | DNase.fdr0.01.hot | | DNase |
| Ovary | DNase.fdr0.01.peaks | | DNase |
| Ovary | DNase.hot | | DNase |
| Ovary | DNase | | DNase |
| Ovary | H3K27ac | | Histone |
| Ovary | H3K27me3 | | Histone |
| Ovary | H3K36me3 | | Histone |
| Ovary | H3K4me1 | | Histone |
| Ovary | H3K4me3 | | Histone |
| Ovary | H3K9me3 | | Histone |
| Pancreas | DNase.all.peaks | | DNase |
| Pancreas | DNase.fdr0.01.hot | | DNase |
| Pancreas | DNase.fdr0.01.peaks | | DNase |
| Pancreas | DNase.hot | | DNase |
| Pancreas | DNase | | DNase |
| Pancreas | H3K27ac | | Histone |
| Pancreas | H3K27me3 | | Histone |
| Pancreas | H3K36me3 | | Histone |
| Pancreas | H3K4me1 | | Histone |
| Pancreas | H3K4me3 | | Histone |
| Pancreas | H3K9me3 | | Histone |
| Placenta_Amnion | H3K27ac | | Histone |
| Placenta_Amnion | H3K27me3 | | Histone |
| Placenta_Amnion | H3K36me3 | | Histone |
| Placenta_Amnion | H3K4me1 | | Histone |
| Placenta_Amnion | H3K4me3 | | Histone |
| Placenta_Amnion | H3K9me3 | | Histone |
| Psoas_Muscle | DNase.all.peaks | | DNase |
| Psoas_Muscle | DNase.fdr0.01.hot | | DNase |
| Psoas_Muscle | DNase.fdr0.01.peaks | | DNase |
| Psoas_Muscle | DNase.hot | | DNase |
| Psoas_Muscle | DNase | | DNase |
| Psoas_Muscle | H3K27ac | | Histone |
| Psoas_Muscle | H3K27me3 | | Histone |
| Psoas_Muscle | H3K36me3 | | Histone |
| Psoas_Muscle | H3K4me1 | | Histone |
| Psoas_Muscle | H3K4me3 | | Histone |
| Psoas_Muscle | H3K9me3 | | Histone |
| Rectal_Mucosa.Donor_29 | H3K27ac | | Histone |
| Rectal_Mucosa.Donor_29 | H3K27me3 | | Histone |
| Rectal_Mucosa.Donor_29 | H3K36me3 | | Histone |
| Rectal_Mucosa.Donor_29 | H3K4me1 | | Histone |
| Rectal_Mucosa.Donor_29 | H3K4me3 | | Histone |
| Rectal_Mucosa.Donor_29 | H3K9ac | | Histone |
| Rectal_Mucosa.Donor_29 | H3K9me3 | | Histone |
| Rectal_Mucosa.Donor_31 | H3K27ac | | Histone |
| Rectal_Mucosa.Donor_31 | H3K27me3 | | Histone |
| Rectal_Mucosa.Donor_31 | H3K36me3 | | Histone |
| Rectal_Mucosa.Donor_31 | H3K4me1 | | Histone |
| Rectal_Mucosa.Donor_31 | H3K4me3 | | Histone |
| Rectal_Mucosa.Donor_31 | H3K9ac | | Histone |
| Rectal_Mucosa.Donor_31 | H3K9me3 | | Histone |
| Rectal_Smooth_Muscle | H3K27ac | | Histone |
| Rectal_Smooth_Muscle | H3K27me3 | | Histone |
| Rectal_Smooth_Muscle | H3K36me3 | | Histone |
| Rectal_Smooth_Muscle | H3K4me1 | | Histone |
| Rectal_Smooth_Muscle | H3K4me3 | | Histone |
| Rectal_Smooth_Muscle | H3K9ac | | Histone |
| Rectal_Smooth_Muscle | H3K9me3 | | Histone |
| Right_Atrium | H3K27ac | | Histone |
| Right_Atrium | H3K27me3 | | Histone |
| Right_Atrium | H3K36me3 | | Histone |
| Right_Atrium | H3K4me1 | | Histone |
| Right_Atrium | H3K4me3 | | Histone |
| Right_Atrium | H3K9me3 | | Histone |
| Right_Ventricle | H3K27ac | | Histone |
| Right_Ventricle | H3K27me3 | | Histone |
| Right_Ventricle | H3K36me3 | | Histone |
| Right_Ventricle | H3K4me1 | | Histone |
| Right_Ventricle | H3K4me3 | | Histone |
| Right_Ventricle | H3K9me3 | | Histone |
| Sigmoid_Colon | H3K27ac | | Histone |
| Sigmoid_Colon | H3K27me3 | | Histone |
| Sigmoid_Colon | H3K36me3 | | Histone |
| Sigmoid_Colon | H3K4me1 | | Histone |
| Sigmoid_Colon | H3K4me3 | | Histone |
| Sigmoid_Colon | H3K9me3 | | Histone |
| Skeletal_Muscle_Male | H3K27me3 | | Histone |
| Skeletal_Muscle_Male | H3K36me3 | | Histone |
| Skeletal_Muscle_Male | H3K4me1 | | Histone |
| Skeletal_Muscle_Male | H3K4me3 | | Histone |
| Skeletal_Muscle_Male | H3K9ac | | Histone |
| Skeletal_Muscle_Male | H3K9me3 | | Histone |
| Skeletal_Muscle_Female | H3K27ac | | Histone |
| Skeletal_Muscle_Female | H3K27me3 | | Histone |
| Skeletal_Muscle_Female | H3K36me3 | | Histone |
| Skeletal_Muscle_Female | H3K4me1 | | Histone |
| Skeletal_Muscle_Female | H3K4me3 | | Histone |
| Skeletal_Muscle_Female | H3K9ac | | Histone |
| Skeletal_Muscle_Female | H3K9me3 | | Histone |
| Small_Intestine | DNase.all.peaks | | DNase |
| Small_Intestine | DNase.fdr0.01.hot | | DNase |
| Small_Intestine | DNase.fdr0.01.peaks | | DNase |
| Small_Intestine | DNase.hot | | DNase |
| Small_Intestine | DNase | | DNase |
| Small_Intestine | H3K27ac | | Histone |
| Small_Intestine | H3K27me3 | | Histone |
| Small_Intestine | H3K36me3 | | Histone |
| Small_Intestine | H3K4me1 | | Histone |
| Small_Intestine | H3K4me3 | | Histone |
| Small_Intestine | H3K9me3 | | Histone |
| Stomach_Mucosa | H3K27me3 | | Histone |
| Stomach_Mucosa | H3K36me3 | | Histone |
| Stomach_Mucosa | H3K4me1 | | Histone |
| Stomach_Mucosa | H3K4me3 | | Histone |
| Stomach_Mucosa | H3K9ac | | Histone |
| Stomach_Mucosa | H3K9me3 | | Histone |
| Stomach_Smooth_Muscle | H3K27ac | | Histone |
| Stomach_Smooth_Muscle | H3K27me3 | | Histone |
| Stomach_Smooth_Muscle | H3K36me3 | | Histone |
| Stomach_Smooth_Muscle | H3K4me1 | | Histone |
| Stomach_Smooth_Muscle | H3K4me3 | | Histone |
| Stomach_Smooth_Muscle | H3K9ac | | Histone |
| Stomach_Smooth_Muscle | H3K9me3 | | Histone |
| Thymus | H3K27ac | | Histone |
| Thymus | H3K27me3 | | Histone |
| Thymus | H3K36me3 | | Histone |
| Thymus | H3K4me1 | | Histone |
| Thymus | H3K4me3 | | Histone |
| Thymus | H3K9me3 | | Histone |
| Spleen | H3K27ac | | Histone |
| Spleen | H3K27me3 | | Histone |
| Spleen | H3K36me3 | | Histone |
| Spleen | H3K4me1 | | Histone |
| Spleen | H3K4me3 | | Histone |
| Spleen | H3K9me3 | | Histone |
| A549_EtOH_0.02pct_Lung_Carcinoma | DNase | | DNase |
| A549_EtOH_0.02pct_Lung_Carcinoma | H2A.Z | | Histone |
| A549_EtOH_0.02pct_Lung_Carcinoma | H3K27ac | | Histone |
| A549_EtOH_0.02pct_Lung_Carcinoma | H3K27me3 | | Histone |
| A549_EtOH_0.02pct_Lung_Carcinoma | H3K36me3 | | Histone |
| A549_EtOH_0.02pct_Lung_Carcinoma | H3K4me1 | | Histone |
| A549_EtOH_0.02pct_Lung_Carcinoma | H3K4me2 | | Histone |
| A549_EtOH_0.02pct_Lung_Carcinoma | H3K4me3 | | Histone |
| A549_EtOH_0.02pct_Lung_Carcinoma | H3K79me2 | | Histone |
| A549_EtOH_0.02pct_Lung_Carcinoma | H3K9ac | | Histone |
| A549_EtOH_0.02pct_Lung_Carcinoma | H3K9me3 | | Histone |
| A549_EtOH_0.02pct_Lung_Carcinoma | H4K20me1 | | Histone |
| Dnd41_TCell_Leukemia | H2A.Z | | Histone |
| Dnd41_TCell_Leukemia | H3K27ac | | Histone |
| Dnd41_TCell_Leukemia | H3K27me3 | | Histone |
| Dnd41_TCell_Leukemia | H3K36me3 | | Histone |
| Dnd41_TCell_Leukemia | H3K4me1 | | Histone |
| Dnd41_TCell_Leukemia | H3K4me2 | | Histone |
| Dnd41_TCell_Leukemia | H3K4me3 | | Histone |
| Dnd41_TCell_Leukemia | H3K79me2 | | Histone |
| Dnd41_TCell_Leukemia | H3K9ac | | Histone |
| Dnd41_TCell_Leukemia | H3K9me3 | | Histone |
| Dnd41_TCell_Leukemia | H4K20me1 | | Histone |
| GM12878_Lymphoblastoid | DNase | | DNase |
| GM12878_Lymphoblastoid | H2A.Z | | Histone |
| GM12878_Lymphoblastoid | H3K27ac | | Histone |
| GM12878_Lymphoblastoid | H3K27me3 | | Histone |
| GM12878_Lymphoblastoid | H3K36me3 | | Histone |
| GM12878_Lymphoblastoid | H3K4me1 | | Histone |
| GM12878_Lymphoblastoid | H3K4me2 | | Histone |
| GM12878_Lymphoblastoid | H3K4me3 | | Histone |
| GM12878_Lymphoblastoid | H3K79me2 | | Histone |
| GM12878_Lymphoblastoid | H3K9ac | | Histone |
| GM12878_Lymphoblastoid | H3K9me3 | | Histone |
| GM12878_Lymphoblastoid | H4K20me1 | | Histone |
| HeLa | DNase | | DNase |
| HeLa | H2A.Z | | Histone |
| HeLa | H3K27ac | | Histone |
| HeLa | H3K27me3 | | Histone |
| HeLa | H3K36me3 | | Histone |
| HeLa | H3K4me1 | | Histone |
| HeLa | H3K4me2 | | Histone |
| HeLa | H3K4me3 | | Histone |
| HeLa | H3K79me2 | | Histone |
| HeLa | H3K9ac | | Histone |
| HeLa | H3K9me3 | | Histone |
| HeLa | H4K20me1 | | Histone |
| HepG2_Hepatocellular_Carcinoma | DNase | | DNase |
| HepG2_Hepatocellular_Carcinoma | H2A.Z | | Histone |
| HepG2_Hepatocellular_Carcinoma | H3K27ac | | Histone |
| HepG2_Hepatocellular_Carcinoma | H3K27me3 | | Histone |
| HepG2_Hepatocellular_Carcinoma | H3K36me3 | | Histone |
| HepG2_Hepatocellular_Carcinoma | H3K4me1 | | Histone |
| HepG2_Hepatocellular_Carcinoma | H3K4me2 | | Histone |
| HepG2_Hepatocellular_Carcinoma | H3K4me3 | | Histone |
| HepG2_Hepatocellular_Carcinoma | H3K79me2 | | Histone |
| HepG2_Hepatocellular_Carcinoma | H3K9ac | | Histone |
| HepG2_Hepatocellular_Carcinoma | H3K9me3 | | Histone |
| HepG2_Hepatocellular_Carcinoma | H4K20me1 | | Histone |
| HMEC_Mammary_Epithelial | DNase | | DNase |
| HMEC_Mammary_Epithelial | H2A.Z | | Histone |
| HMEC_Mammary_Epithelial | H3K27ac | | Histone |
| HMEC_Mammary_Epithelial | H3K27me3 | | Histone |
| HMEC_Mammary_Epithelial | H3K36me3 | | Histone |
| HMEC_Mammary_Epithelial | H3K4me1 | | Histone |
| HMEC_Mammary_Epithelial | H3K4me2 | | Histone |
| HMEC_Mammary_Epithelial | H3K4me3 | | Histone |
| HMEC_Mammary_Epithelial | H3K79me2 | | Histone |
| HMEC_Mammary_Epithelial | H3K9ac | | Histone |
| HMEC_Mammary_Epithelial | H3K9me3 | | Histone |
| HMEC_Mammary_Epithelial | H4K20me1 | | Histone |
| HSMM_Skeletal_Muscle_Myoblasts | DNase | | DNase |
| HSMM_Skeletal_Muscle_Myoblasts | H2A.Z | | Histone |
| HSMM_Skeletal_Muscle_Myoblasts | H3K27ac | | Histone |
| HSMM_Skeletal_Muscle_Myoblasts | H3K27me3 | | Histone |
| HSMM_Skeletal_Muscle_Myoblasts | H3K36me3 | | Histone |
| HSMM_Skeletal_Muscle_Myoblasts | H3K4me1 | | Histone |
| HSMM_Skeletal_Muscle_Myoblasts | H3K4me2 | | Histone |
| HSMM_Skeletal_Muscle_Myoblasts | H3K4me3 | | Histone |
| HSMM_Skeletal_Muscle_Myoblasts | H3K79me2 | | Histone |
| HSMM_Skeletal_Muscle_Myoblasts | H3K9ac | | Histone |
| HSMM_Skeletal_Muscle_Myoblasts | H3K9me3 | | Histone |
| HSMM_Skeletal_Muscle_Myoblasts | H4K20me1 | | Histone |
| HSMMtube_Skeletal_Muscle_ | DNase | | DNase |
| Myotubes_Derived_from_HSMM | | | |
| HSMMtube_Skeletal_Muscle_ | H2A.Z | | Histone |
| Myotubes_Derived_from_HSMM | | | |
| HSMMtube_Skeletal_Muscle_ | H3K27ac | | Histone |
| Myotubes_Derived_from_HSMM | | | |
| HSMMtube_Skeletal_Muscle_ | H3K27me3 | | Histone |
| Myotubes_Derived_from_HSMM | | | |
| HSMMtube_Skeletal_Muscle_ | H3K36me3 | | Histone |
| Myotubes_Derived_from_HSMM | | | |
| HSMMtube_Skeletal_Muscle_ | H3K4me1 | | Histone |
| Myotubes_Derived_from_HSMM | | | |
| HSMMtube_Skeletal_Muscle_ | H3K4me2 | | Histone |
| Myotubes_Derived_from_HSMM | | | |
| HSMMtube_Skeletal_Muscle_ | H3K4me3 | | Histone |
| Myotubes_Derived_from_HSMM | | | |
| HSMMtube_Skeletal_Muscle_ | H3K79me2 | | Histone |
| Myotubes_Derived_from_HSMM | | | |
| HSMMtube_Skeletal_Muscle_ | H3K9ac | | Histone |
| Myotubes_Derived_from_HSMM | | | |
| HSMMtube_Skeletal_Muscle_ | H3K9me3 | | Histone |
| Myotubes_Derived_from_HSMM | | | |
| HSMMtube_Skeletal_Muscle_ | H4K20me1 | | Histone |
| Myotubes_Derived_from_HSMM | | | |
| HUVEC_Umbilical_Vein_Endothelial_Cells | DNase | | DNase |
| HUVEC_Umbilical_Vein_Endothelial_Cells | H2A.Z | | Histone |
| HUVEC_Umbilical_Vein_Endothelial_Cells | H3K27ac | | Histone |
| HUVEC_Umbilical_Vein_Endothelial_Cells | H3K27me3 | | Histone |
| HUVEC_Umbilical_Vein_Endothelial_Cells | H3K36me3 | | Histone |
| HUVEC_Umbilical_Vein_Endothelial_Cells | H3K4me1 | | Histone |
| HUVEC_Umbilical_Vein_Endothelial_Cells | H3K4me2 | | Histone |
| HUVEC_Umbilical_Vein_Endothelial_Cells | H3K4me3 | | Histone |
| HUVEC_Umbilical_Vein_Endothelial_Cells | H3K79me2 | | Histone |
| HUVEC_Umbilical_Vein_Endothelial_Cells | H3K9ac | | Histone |
| HUVEC_Umbilical_Vein_Endothelial_Cells | H3K9me1 | | Histone |
| HUVEC_Umbilical_Vein_Endothelial_Cells | H3K9me3 | | Histone |
| HUVEC_Umbilical_Vein_Endothelial_Cells | H4K20me1 | | Histone |
| K562 | DNase | | DNase |
| K562 | H2A.Z | | Histone |
| K562 | H3K27ac | | Histone |
| K562 | H3K27me3 | | Histone |
| K562 | H3K36me3 | | Histone |
| K562 | H3K4me1 | | Histone |
| K562 | H3K4me2 | | Histone |
| K562 | H3K4me3 | | Histone |
| K562 | H3K79me2 | | Histone |
| K562 | H3K9ac | | Histone |
| K562 | H3K9me1 | | Histone |
| K562 | H3K9me3 | | Histone |
| K562 | H4K20me1 | | Histone |
| Monocytes-CD14+_RO01746 | DNase | | DNase |
| Monocytes-CD14+_RO01746 | H2A.Z | | Histone |
| Monocytes-CD14+_RO01746 | H3K27ac | | Histone |
| Monocytes-CD14+_RO01746 | H3K27me3 | | Histone |
| Monocytes-CD14+_RO01746 | H3K36me3 | | Histone |
| Monocytes-CD14+_RO01746 | H3K4me1 | | Histone |
| Monocytes-CD14+_RO01746 | H3K4me2 | | Histone |
| Monocytes-CD14+_RO01746 | H3K4me3 | | Histone |
| Monocytes-CD14+_RO01746 | H3K79me2 | | Histone |
| Monocytes-CD14+_RO01746 | H3K9ac | | Histone |
| Monocytes-CD14+_RO01746 | H3K9me3 | | Histone |
| Monocytes-CD14+_RO01746 | H4K20me1 | | Histone |
| NH_A_Astrocytes | DNase | | DNase |
| NH_A_Astrocytes | H2A.Z | | Histone |
| NH_A_Astrocytes | H3K27ac | | Histone |
| NH_A_Astrocytes | H3K27me3 | | Histone |
| NH_A_Astrocytes | H3K36me3 | | Histone |
| NH_A_Astrocytes | H3K4me1 | | Histone |
| NH_A_Astrocytes | H3K4me2 | | Histone |
| NH_A_Astrocytes | H3K4me3 | | Histone |
| NH_A_Astrocytes | H3K79me2 | | Histone |
| NH_A_Astrocytes | H3K9ac | | Histone |
| NH_A_Astrocytes | H3K9me3 | | Histone |
| NH_A_Astrocytes | H4K20me1 | | Histone |
| NHDF_Ad_Adult_Dermal_Fibroblasts | DNase | | DNase |
| NHDF_Ad_Adult_Dermal_Fibroblasts | H2A.Z | | Histone |
| NHDF_Ad_Adult_Dermal_Fibroblasts | H3K27ac | | Histone |
| NHDF_Ad_Adult_Dermal_Fibroblasts | H3K27me3 | | Histone |
| NHDF_Ad_Adult_Dermal_Fibroblasts | H3K36me3 | | Histone |
| NHDF_Ad_Adult_Dermal_Fibroblasts | H3K4me1 | | Histone |
| NHDF_Ad_Adult_Dermal_Fibroblasts | H3K4me2 | | Histone |
| NHDF_Ad_Adult_Dermal_Fibroblasts | H3K4me3 | | Histone |
| NHDF_Ad_Adult_Dermal_Fibroblasts | H3K79me2 | | Histone |
| NHDF_Ad_Adult_Dermal_Fibroblasts | H3K9ac | | Histone |
| NHDF_Ad_Adult_Dermal_Fibroblasts | H3K9me3 | | Histone |
| NHDF_Ad_Adult_Dermal_Fibroblasts | H4K20me1 | | Histone |
| NHEK_Epidermal_Keratinocytes | DNase | | DNase |
| NHEK_Epidermal_Keratinocytes | H2A.Z | | Histone |
| NHEK_Epidermal_Keratinocytes | H3K27ac | | Histone |
| NHEK_Epidermal_Keratinocytes | H3K27me3 | | Histone |
| NHEK_Epidermal_Keratinocytes | H3K36me3 | | Histone |
| NHEK_Epidermal_Keratinocytes | H3K4me1 | | Histone |
| NHEK_Epidermal_Keratinocytes | H3K4me2 | | Histone |
| NHEK_Epidermal_Keratinocytes | H3K4me3 | | Histone |
| NHEK_Epidermal_Keratinocytes | H3K79me2 | | Histone |
| NHEK_Epidermal_Keratinocytes | H3K9ac | | Histone |
| NHEK_Epidermal_Keratinocytes | H3K9me1 | | Histone |
| NHEK_Epidermal_Keratinocytes | H3K9me3 | | Histone |
| NHEK_Epidermal_Keratinocytes | H4K20me1 | | Histone |
| NHLF_Lung_Fibroblasts | DNase | | DNase |
| NHLF_Lung_Fibroblasts | H2A.Z | | Histone |
| NHLF_Lung_Fibroblasts | H3K27ac | | Histone |
| NHLF_Lung_Fibroblasts | H3K27me3 | | Histone |
| NHLF_Lung_Fibroblasts | H3K36me3 | | Histone |
| NHLF_Lung_Fibroblasts | H3K4me1 | | Histone |
| NHLF_Lung_Fibroblasts | H3K4me2 | | Histone |
| NHLF_Lung_Fibroblasts | H3K4me3 | | Histone |
| NHLF_Lung_Fibroblasts | H3K79me2 | | Histone |
| NHLF_Lung_Fibroblasts | H3K9ac | | Histone |
| NHLF_Lung_Fibroblasts | H3K9me3 | | Histone |
| NHLF_Lung_Fibroblasts | H4K20me1 | | Histone |
| Osteoblasts | H2A.Z | | Histone |
| Osteoblasts | H3K27ac | | Histone |
| Osteoblasts | H3K27me3 | | Histone |
| Osteoblasts | H3K36me3 | | Histone |
| Osteoblasts | H3K4me1 | | Histone |
| Osteoblasts | H3K4me2 | | Histone |
| Osteoblasts | H3K4me3 | | Histone |
| Osteoblasts | H3K79me2 | | Histone |
| Osteoblasts | H3K9me3 | | Histone |
| Osteoblasts | H4K20me1 | | Histone |

TABLE 2
RBP/RNA element Profiles

| RBP_model | Species | RBP |
|---|---|---|
| AGO_adult_brain.BA4.human | Homo sapiens | AGO |
| AGO_adult_brain.Cingulate.gyrus.human | Homo sapiens | AGO |
| ELAVL_Adult_brain.all_human_samples.human | Homo sapiens | ELAVL |
| ELAVL_Adult_brain.BA9_Alzheimer.human | Homo sapiens | ELAVL |
| ELAVL_Adult_brain.BA9.human | Homo sapiens | ELAVL |
| HNRNPC_cell.line_HeLa.iCLIP.human | Homo sapiens | HNRNPC |
| LIN28A_cell.line_H9.ESC.human | Homo sapiens | LIN28A |
| MS12_cell.line_NB4.human | Homo sapiens | MS12 |
| NOVA1_cell.line_PrimaryGBM.human | Homo sapiens | NOVA1 |
| NSR100_cell.line_293T.human | Homo sapiens | NSR100 |
| PTBP1_cell.line_HeLa.iCLIP.human | Homo sapiens | PTBP1 |
| RBFOX2_cell.line_293T.human | Homo sapiens | RBFOX2 |
| TIA1_cell.line_HeLa.iCLIP.human | Homo sapiens | TIA1 |
| TIAL1_cell.line_HeLa.iCLIP.human | Homo sapiens | TIAL1 |
| U2AF2_cell.line_HeLa.iCLIP_Hnrnpc_ctrl.human | Homo sapiens | U2AF2 |
| U2AF2_cell.line_HeLa.iCLIP_Hnrnpc_KD.human | Homo sapiens | U2AF2 |
| U2AF2_cell.line_HeLa.iCLIP.human | Homo sapiens | U2AF2 |
| PABP_cell.line_HeLa.human | Homo sapiens | PABP |
| PABP_cell.line_LN229.human | Homo sapiens | PABP |
| AKAP8L_K562_eCLIP.rep1.ENCODE.human | Homo sapiens | AKAP8L |
| AKAP8L_K562_eCLIP.rep2.ENCODE.human | Homo sapiens | AKAP8L |
| AUH_HepG2_eCLIP.rep1.ENCODE.human | Homo sapiens | AUH |
| AUH_HepG2_eCLIP.rep2.ENCODE.human | Homo sapiens | AUH |
| BCCIP_HepG2_eCLIP.rep1.ENCODE.human | Homo sapiens | BCCIP |
| BCCIP_HepG2_eCLIP.rep2.ENCODE.human | Homo sapiens | BCCIP |
| BUD13_HepG2_eCLIP.rep1.ENCODE.human | Homo sapiens | BUD13 |
| BUD13_HepG2_eCLIP.rep2.ENCODE.human | Homo sapiens | BUD13 |
| BUD13_K562_eCLIP.rep1.ENCODE.human | Homo sapiens | BUD13 |
| BUD13_K562_eCLIP.rep2.ENCODE.human | Homo sapiens | BUD13 |
| CPSF6_K562_eCLIP.rep1.ENCODE.human | Homo sapiens | CPSF6 |
| CPSF6_K562_eCLIP.rep2.ENCODE.human | Homo sapiens | CPSF6 |
| CSTF2T_HepG2_eCLIP.rep1.ENCODE.human | Homo sapiens | CSTF2T |
| CSTF2T_HepG2_eCLIP.rep2.ENCODE.human | Homo sapiens | CSTF2T |
| CSTF2T_K562_eCLIP.rep1.ENCODE.human | Homo sapiens | CSTF2T |
| CSTF2T_K562_eCLIP.rep2.ENCODE.human | Homo sapiens | CSTF2T |
| DDX42_K562_eCLIP.rep1.ENCODE.human | Homo sapiens | DDX42 |
| DDX42_K562_eCLIP.rep2.ENCODE.human | Homo sapiens | DDX42 |
| DDX6_HepG2_eCLIP.rep1.ENCODE.human | Homo sapiens | DDX6 |
| DDX6_HepG2_eCLIP.rep2.ENCODE.human | Homo sapiens | DDX6 |
| DDX6_K562_eCLIP.rep1.ENCODE.human | Homo sapiens | DDX6 |
| DDX6_K562_eCLIP.rep2.ENCODE.human | Homo sapiens | DDX6 |
| DKC1_HepG2_eCLIP.rep2.ENCODE.human | Homo sapiens | DKC1 |
| EFTUD2_HepG2_eCLIP.rep1.ENCODE.human | Homo sapiens | EFTUD2 |
| EFTUD2_HepG2_eCLIP.rep2.ENCODE.human | Homo sapiens | EFTUD2 |
| EFTUD2_K562_eCLIP.rep1.ENCODE.human | Homo sapiens | EFTUD2 |
| EFTUD2_K562_eCLIP.rep2.ENCODE.human | Homo sapiens | EFTUD2 |
| EIF3D_HepG2_eCLIP.rep1.ENCODE.human | Homo sapiens | EIF3D |
| EIF3D_HepG2_eCLIP.rep2.ENCODE.human | Homo sapiens | EIF3D |
| EIF4G2_K562_eCLIP.rep1.ENCODE.human | Homo sapiens | EIF4G2 |
| EIF4G2_K562_eCLIP.rep2.ENCODE.human | Homo sapiens | EIF4G2 |
| EWSR1_K562_eCLIP.rep1.ENCODE.human | Homo sapiens | EWSR1 |
| EWSR1_K562_eCLIP.rep2.ENCODE.human | Homo sapiens | EWSR1 |
| FAM120A_HepG2_eCLIP.rep1.ENCODE.human | Homo sapiens | FAM120A |
| FAM120A_HepG2_eCLIP.rep2.ENCODE.human | Homo sapiens | FAM120A |
| FAM120A_K562_eCLIP.rep1.ENCODE.human | Homo sapiens | FAM120A |
| FAM120A_K562_eCLIP.rep2.ENCODE.human | Homo sapiens | FAM120A |
| FASTKD2_K562_eCLIP.rep2.ENCODE.human | Homo sapiens | FASTKD2 |
| GRSF1_HepG2_eCLIP.rep1.ENCODE.human | Homo sapiens | GRSF1 |
| GRSF1_HepG2_eCLIP.rep2.ENCODE.human | Homo sapiens | GRSF1 |
| GTF2F1_HepG2_eCLIP.rep1.ENCODE.human | Homo sapiens | GTF2F1 |
| GTF2F1_HepG2_eCLIP.rep2.ENCODE.human | Homo sapiens | GTF2F1 |
| GTF2F1_K562_eCLIP.rep1.ENCODE.human | Homo sapiens | GTF2F1 |
| GTF2F1_K562_eCLIP.rep2.ENCODE.human | Homo sapiens | GTF2F1 |
| HNRNPA1_HepG2_eCLIP.rep1.ENCODE.human | Homo sapiens | HNRNPA1 |
| HNRNPA1_HepG2_eCLIP.rep2.ENCODE.human | Homo sapiens | HNRNPA1 |
| HNRNPA1_K562_eCLIP.rep1.ENCODE.human | Homo sapiens | HNRNPA1 |
| HNRNPA1_K562_eCLIP.rep2.ENCODE.human | Homo sapiens | HNRNPA1 |
| HNRNPC_HepG2_eCLIP.rep1.ENCODE.human | Homo sapiens | HNRNPC |
| HNRNPC_HepG2_eCLIP.rep2.ENCODE.human | Homo sapiens | HNRNPC |
| HNRNPK_HepG2_eCLIP.rep1.ENCODE.human | Homo sapiens | HNRNPK |
| HNRNPK_HepG2_eCLIP.rep2.ENCODE.human | Homo sapiens | HNRNPK |
| HNRNPK_K562_eCLIP.rep1.ENCODE.human | Homo sapiens | HNRNPK |
| HNRNPK_K562_eCLIP.rep2.ENCODE.human | Homo sapiens | HNRNPK |
| HNRNPM_HepG2_eCLIP.rep1.ENCODE.human | Homo sapiens | HNRNPM |
| HNRNPM_HepG2_eCLIP.rep2.ENCODE.human | Homo sapiens | HNRNPM |
| HNRNPM_K562_eCLIP.rep1.ENCODE.human | Homo sapiens | HNRNPM |
| HNRNPM_K562_eCLIP.rep2.ENCODE.human | Homo sapiens | HNRNPM |
| HNRNPU_adrenal.gland_eCLIP.rep1.ENCODE.human | Homo sapiens | HNRNPU |
| HNRNPU_adrenal.gland_eCLIP.rep2.ENCODE.human | Homo sapiens | HNRNPU |
| HNRNPU_HepG2_eCLIP.rep1.ENCODE.human | Homo sapiens | HNRNPU |
| HNRNPU_HepG2_eCLIP.rep2.ENCODE.human | Homo sapiens | HNRNPU |
| HNRNPU_K562_eCLIP.rep1.ENCODE.human | Homo sapiens | HNRNPU |
| HNRNPU_K562_eCLIP.rep2.ENCODE.human | Homo sapiens | HNRNPU |
| HNRNPUL1_HepG2_eCLIP.rep1.ENCODE.human | Homo sapiens | HNRNPUL1 |
| HNRNPUL1_HepG2_eCLIP.rep2.ENCODE.human | Homo sapiens | HNRNPUL1 |
| HNRNPUL1_K562_eCLIP.rep1.ENCODE.human | Homo sapiens | HNRNPUL1 |
| HNRNPUL1_K562_eCLIP.rep2.ENCODE.human | Homo sapiens | HNRNPUL1 |
| IGF2BP3_HepG2_eCLIP.rep1.ENCODE.human | Homo sapiens | IGF2BP3 |
| IGF2BP3_HepG2_eCLIP.rep2.ENCODE.human | Homo sapiens | IGF2BP3 |
| ILF3_K562_eCLIP.rep1.ENCODE.human | Homo sapiens | ILF3 |
| ILF3_K562_eCLIP.rep2.ENCODE.human | Homo sapiens | ILF3 |
| KHDRBS1_K562_eCLIP.rep1.ENCODE.human | Homo sapiens | KHDRBS1 |
| KHDRBS1_K562_eCLIP.rep2.ENCODE.human | Homo sapiens | KHDRBS1 |
| KHSRP_K562_eCLIP.rep1.ENCODE.human | Homo sapiens | KHSRP |
| KHSRP_K562_eCLIP.rep2.ENCODE.human | Homo sapiens | KHSRP |
| LARP4_HepG2_eCLIP.rep1.ENCODE.human | Homo sapiens | LARP4 |
| LARP4_HepG2_eCLIP.rep2.ENCODE.human | Homo sapiens | LARP4 |
| LARP4_K562_eCLIP.rep1.ENCODE.human | Homo sapiens | LARP4 |
| LARP4_K562_eCLIP.rep2.ENCODE.human | Homo sapiens | LARP4 |
| LSM11_HepG2_eCLIP.rep1.ENCODE.human | Homo sapiens | LSM11 |
| LSM11_HepG2_eCLIP.rep2.ENCODE.human | Homo sapiens | LSM11 |
| LSM11_K562_eCLIP.rep1.ENCODE.human | Homo sapiens | LSM11 |
| LSM11_K562_eCLIP.rep2.ENCODE.human | Homo sapiens | LSM11 |
| MTPAP_K562_eCLIP.rep1.ENCODE.human | Homo sapiens | MTPAP |
| MTPAP_K562_eCLIP.rep2.ENCODE.human | Homo sapiens | MTPAP |
| NCBP2_HepG2_eCLIP.rep1.ENCODE.human | Homo sapiens | NCBP2 |
| NCBP2_HepG2_eCLIP.rep2.ENCODE.human | Homo sapiens | NCBP2 |
| NCBP2_K562_eCLIP.rep1.ENCODE.human | Homo sapiens | NCBP2 |
| NCBP2_K562_eCLIP.rep2.ENCODE.human | Homo sapiens | NCBP2 |
| NKRF_HepG2_eCLIP.rep1.ENCODE.human | Homo sapiens | NKRF |
| NKRF_HepG2_eCLIP.rep2.ENCODE.human | Homo sapiens | NKRF |
| NONO_K562_eCLIP.rep1.ENCODE.human | Homo sapiens | NONO |
| NONO_K562_eCLIP.rep2.ENCODE.human | Homo sapiens | NONO |
| PCBP2_HepG2_eCLIP.rep1.ENCODE.human | Homo sapiens | PCBP2 |
| PCBP2_HepG2_eCLIP.rep2.ENCODE.human | Homo sapiens | PCBP2 |
| PPIL4_K562_eCLIP.rep1.ENCODE.human | Homo sapiens | PPIL4 |
| PPIL4_K562_eCLIP.rep2.ENCODE.human | Homo sapiens | PPIL4 |
| PRPF8_HepG2_eCLIP.rep1.ENCODE.human | Homo sapiens | PRPF8 |
| PRPF8_HepG2_eCLIP.rep2.ENCODE.human | Homo sapiens | PRPF8 |
| PRPF8_K562_eCLIP.rep1.ENCODE.human | Homo sapiens | PRPF8 |
| PRPF8_K562_eCLIP.rep2.ENCODE.human | Homo sapiens | PRPF8 |
| PTBP1_K562_eCLIP.rep1.ENCODE.human | Homo sapiens | PTBP1 |
| PTBP1_K562_eCLIP.rep2.ENCODE.human | Homo sapiens | PTBP1 |
| PUM2_K562_eCLIP.rep1.ENCODE.human | Homo sapiens | PUM2 |
| PUM2_K562_eCLIP.rep2.ENCODE.human | Homo sapiens | PUM2 |
| QKI_HepG2_eCLIP.rep1.ENCODE.human | Homo sapiens | QKI |
| QKI_HepG2_eCLIP.rep2.ENCODE.human | Homo sapiens | QKI |
| QKI_K562_eCLIP.rep1.ENCODE.human | Homo sapiens | QKI |
| QKI_K562_eCLIP.rep2.ENCODE.human | Homo sapiens | QKI |
| RBFOX2_HepG2_eCLIP.rep1.ENCODE.human | Homo sapiens | RBFOX2 |
| RBFOX2_HepG2_eCLIP.rep2.ENCODE.human | Homo sapiens | RBFOX2 |
| RBM15_HepG2_eCLIP.rep1.ENCODE.human | Homo sapiens | RBM15 |
| RBM15_HepG2_eCLIP.rep2.ENCODE.human | Homo sapiens | RBM15 |
| RBM15_K562_eCLIP.rep1.ENCODE.human | Homo sapiens | RBM15 |
| RBM15_K562_eCLIP.rep2.ENCODE.human | Homo sapiens | RBM15 |
| RBM22_HepG2_eCLIP.rep1.ENCODE.human | Homo sapiens | RBM22 |
| RBM22_HepG2_eCLIP.rep2.ENCODE.human | Homo sapiens | RBM22 |
| RBM22_K562_eCLIP.rep1.ENCODE.human | Homo sapiens | RBM22 |
| RBM22_K562_eCLIP.rep2.ENCODE.human | Homo sapiens | RBM22 |
| RBM27_K562_eCLIP.rep1.ENCODE.human | Homo sapiens | RBM27 |
| RBM27_K562_eCLIP.rep2.ENCODE.human | Homo sapiens | RBM27 |
| RPS5_K562_eCLIP.rep1.ENCODE.human | Homo sapiens | RPS5 |
| RPS5_K562_eCLIP.rep2.ENCODE.human | Homo sapiens | RPS5 |
| SAFB2_K562_eCLIP.rep1.ENCODE.human | Homo sapiens | SAFB2 |
| SAFB2_K562_eCLIP.rep2.ENCODE.human | Homo sapiens | SAFB2 |
| SF3A3_HepG2_eCLIP.rep1.ENCODE.human | Homo sapiens | SF3A3 |
| SF3A3_HepG2_eCLIP.rep2.ENCODE.human | Homo sapiens | SF3A3 |
| SF3B4_HepG2_eCLIP.rep1.ENCODE.human | Homo sapiens | SF3B4 |
| SF3B4_HepG2_eCLIP.rep2.ENCODE.human | Homo sapiens | SF3B4 |
| SF3B4_K562_eCLIP.rep1.ENCODE.human | Homo sapiens | SF3B4 |
| SF3B4_K562_eCLIP.rep2.ENCODE.human | Homo sapiens | SF3B4 |
| SFPQ_HepG2_eCLIP.rep1.ENCODE.human | Homo sapiens | SFPQ |
| SFPQ_HepG2_eCLIP.rep2.ENCODE.human | Homo sapiens | SFPQ |
| SLTM_HepG2_eCLIP.rep1.ENCODE.human | Homo sapiens | SLTM |
| SLTM_HepG2_eCLIP.rep2.ENCODE.human | Homo sapiens | SLTM |
| SLTM_K562_eCLIP.rep1.ENCODE.human | Homo sapiens | SLTM |
| SLTM_K562_eCLIP.rep2.ENCODE.human | Homo sapiens | SLTM |
| SMNDC1_HepG2_eCLIP.rep2.ENCODE.human | Homo sapiens | SMNDC1 |
| SMNDC1_K562_eCLIP.rep1.ENCODE.human | Homo sapiens | SMNDC1 |
| SMNDC1_K562_eCLIP.rep2.ENCODE.human | Homo sapiens | SMNDC1 |
| SRSF1_HepG2_eCLIP.rep1.ENCODE.human | Homo sapiens | SRSF1 |
| SRSF1_HepG2_eCLIP.rep2.ENCODE.human | Homo sapiens | SRSF1 |
| SRSF1_K562_eCLIP.rep1.ENCODE.human | Homo sapiens | SRSF1 |
| SRSF1_K562_eCLIP.rep2.ENCODE.human | Homo sapiens | SRSF1 |
| SRSF7_HepG2_eCLIP.rep1.ENCODE.human | Homo sapiens | SRSF7 |
| SRSF7_HepG2_eCLIP.rep2.ENCODE.human | Homo sapiens | SRSF7 |
| SRSF7_K562_eCLIP.rep1.ENCODE.human | Homo sapiens | SRSF7 |
| SRSF7_K562_eCLIP.rep2.ENCODE.human | Homo sapiens | SRSF7 |
| SRSF9_HepG2_eCLIP.rep1.ENCODE.human | Homo sapiens | SRSF9 |
| SRSF9_HepG2_eCLIP.rep2.ENCODE.human | Homo sapiens | SRSF9 |
| TAF15_HepG2_eCLIP.rep1.ENCODE.human | Homo sapiens | TAF15 |
| TAF15_HepG2_eCLIP.rep2.ENCODE.human | Homo sapiens | TAF15 |
| TAF15_K562_eCLIP.rep1.ENCODE.human | Homo sapiens | TAF15 |
| TAF15_K562_eCLIP.rep2.ENCODE.human | Homo sapiens | TAF15 |
| TARDBP_K562_eCLIP.rep1.ENCODE.human | Homo sapiens | TARDBP |
| TARDBP_K562_eCLIP.rep2.ENCODE.human | Homo sapiens | TARDBP |
| TBRG4_K562_eCLIP.rep1.ENCODE.human | Homo sapiens | TBRG4 |
| TBRG4_K562_eCLIP.rep2.ENCODE.human | Homo sapiens | TBRG4 |
| TIA1_HepG2_eCLIP.rep1.ENCODE.human | Homo sapiens | TIA1 |
| TIA1_HepG2_eCLIP.rep2.ENCODE.human | Homo sapiens | TIA1 |
| TNRC6A_K562_eCLIP.rep1.ENCODE.human | Homo sapiens | TNRC6A |
| TNRC6A_K562_eCLIP.rep2.ENCODE.human | Homo sapiens | TNRC6A |
| TRA2A_HepG2_eCLIP.rep2.ENCODE.human | Homo sapiens | TRA2A |
| TRA2A_K562_eCLIP.rep1.ENCODE.human | Homo sapiens | TRA2A |
| TRA2A_K562_eCLIP.rep2.ENCODE.human | Homo sapiens | TRA2A |
| U2AF1_HepG2_eCLIP.rep1.ENCODE.human | Homo sapiens | U2AF1 |
| U2AF1_HepG2_eCLIP.rep2.ENCODE.human | Homo sapiens | U2AF1 |
| U2AF1_K562_eCLIP.rep1.ENCODE.human | Homo sapiens | U2AF1 |
| U2AF1_K562_eCLIP.rep2.ENCODE.human | Homo sapiens | U2AF1 |
| U2AF2_HepG2_eCLIP.rep1.ENCODE.human | Homo sapiens | U2AF2 |
| U2AF2_HepG2_eCLIP.rep2.ENCODE.human | Homo sapiens | U2AF2 |
| U2AF2_K562_eCLIP.rep1.ENCODE.human | Homo sapiens | U2AF2 |
| U2AF2_K562_eCLIP.rep2.ENCODE.human | Homo sapiens | U2AF2 |
| UPF1_K562_eCLIP.rep1.ENCODE.human | Homo sapiens | UPF1 |
| UPF1_K562_eCLIP.rep2.ENCODE.human | Homo sapiens | UPF1 |
| XRCC6_K562_eCLIP.rep1.ENCODE.human | Homo sapiens | XRCC6 |
| XRCC6_K562_eCLIP.rep2.ENCODE.human | Homo sapiens | XRCC6 |
| XRN2_HepG2_eCLIP.rep1.ENCODE.human | Homo sapiens | XRN2 |
| XRN2_HepG2_eCLIP.rep2.ENCODE.human | Homo sapiens | XRN2 |
| XRN2_K562_eCLIP.rep1.ENCODE.human | Homo sapiens | XRN2 |
| XRN2_K562_eCLIP.rep2.ENCODE.human | Homo sapiens | XRN2 |
| ZRANB2_K562_eCLIP.rep2.ENCODE.human | Homo sapiens | ZRANB2 |
| BRANCHPOINT_cell.line_HeLa.K562.human | Homo sapiens | BRANCHPOINT |
| AGO_adult_CD4.T.cells_KO.miR155.mouse | Mus musculus | AGO |
| AGO_adult_CD4.T.cells_WT.miR155.mouse | Mus musculus | AGO |
| AGO_adult_liver_KO.miR122.mouse | Mus musculus | AGO |
| AGO_adult_liver_WT.miR122.mouse | Mus musculus | AGO |
| AGO_adult_spinal.cord_SOD.mouse | Mus musculus | AGO |
| AGO_adult_spinal.cord.mouse | Mus musculus | AGO |
| AGO_P13_cortex.mouse | Mus musculus | AGO |
| CELF1_mix_heart.muscle.c2c12.mouse | Mus musculus | CELF1 |
| ELAVL_Adult_whole.brain.mouse | Mus musculus | ELAVL |
| ELAVL_cell.line_N2A.mouse | Mus musculus | ELAVL |
| MBNL_cell.line_MEF.mouse | Mus musculus | MBNL |
| MBNL1_Adult_quadriceps.muscle.mouse | Mus musculus | MBNL1 |
| MBNL1_cell.line_C2C12.mouse | Mus musculus | MBNL1 |
| MBNL1_M4_whole.brain.mouse | Mus musculus | MBNL1 |
| MBNL2_M3_hippocampus.mouse | Mus musculus | MBNL2 |
| NOVA1_E18.5_cortex.mouse | Mus musculus | NOVA1 |
| NOVA1.NOVA2_mix_brain.mouse | Mus musculus | NOVA1 |
| NOVA1.NOVA2_P16_whole.brain.mouse | Mus musculus | NOVA1 |
| NOVA2_E18.5_cortex.mouse | Mus musculus | NOVA2 |
| PTBP2_E18.5_whole.brain.mouse | Mus musculus | PTBP2 |
| RBFOX1_P15_whole.brain.mouse | Mus musculus | RBFOX1 |
| RBFOX2_P15_whole.brain.mouse | Mus musculus | RBFOX2 |
| RBFOX3_P15_whole.brain.mouse | Mus musculus | RBFOX3 |
| SRSF3_cell.line_P19.embryonic.carcinoma.cells_tag.GFP.mouse | Mus musculus | SRSF3 |
| SRSF4_cell.line_P19.embryonic.carcinoma.cells_tag.GFP.mouse | Mus musculus | SRSF4 |
| TDP43_adult_spinal.cord.mouse | Mus musculus | TDP43 |
| TDP43_P8_whole.brain.mouse | Mus musculus | TDP43 |
| PABP_adult_Cortex.mouse | Mus musculus | PABP |
| PABP_embryo_Cortex.mouse | Mus musculus | PABP |

TABLE 3
Exemplary variants identified — Variant effect on
regulation tested in luciferase assay.

| | | | | Nearest |
|---|---|---|---|---|
| Chr | Pos | Allele | Individual | representative TSS |
| 2 | 25354705 | T | Prb | POMC |
| 2 | 25354705 | G | Sib | POMC |
| 3 | 54158012 | C | Sib | CACNA2D3 |
| 3 | 54158012 | C | Prb | CACNA2D3 |
| 3 | 193788984 | T | Prb | HES1 |
| 3 | 193788984 | C | Sib | HES1 |
| 4 | 106817492 | C | Prb | NPNT |
| 4 | 106817492 | A | Sib | NPNT |
| 4 | 119736624 | T | Prb | SEC24D |
| 4 | 119736624 | C | Sib | SEC24D |
| 5 | 16901228 | T | Sib | MYO10 |
| 5 | 16901228 | T | Prb | MYO10 |
| 5 | 134871851 | G | Prb | NEUROG1 |
| 5 | 134871851 | A | Sib | NEUROG1 |
| 6 | 14921510 | T | Prb | JARID2 |
| 6 | 14921510 | C | Sib | JARID2 |
| 6 | 18585601 | A | Prb | RNF144B |
| 6 | 18585601 | C | Sib | RNF144B |
| 6 | 29600230 | A | Prb | GABBR1 |
| 6 | 29600230 | C | Sib | GABBR1 |
| 6 | 50675449 | G | Prb | TFAP2D |
| 6 | 50675449 | A | Sib | TFAP2D |
| 6 | 108879283 | A | Prb | FOXO3 |
| 6 | 108879283 | G | Sib | FOXO3 |
| 7 | 121950800 | C | Prb | FEZF1 |
| 7 | 121950800 | G | Sib | FEZF1 |
| 8 | 1211566 | C | Prb | DLGAP2 |
| 8 | 1211566 | G | Sib | DLGAP2 |
| 8 | 74206149 | G | Prb | RDH10 |
| 8 | 74206149 | C | Sib | RDH10 |
| 8 | 97507570 | A | Prb | SDC2 |
| 8 | 97507570 | G | Sib | SDC2 |
| 9 | 139025318 | T | Prb | C9orf69 |
| 9 | 139025318 | C | Sib | C9orf69 |
| 10 | 123822933 | T | Prb | TACC2 |
| 10 | 123822933 | A | Sib | TACC2 |
| 11 | 2435681 | T | Prb | TRPM5 |
| 11 | 2435681 | C | Sib | TRPM5 |
| 13 | 60565771 | C | Prb | DIAPH3 |
| 13 | 60565771 | T | Sib | DIAPH3 |
| 14 | 77648134 | T | Prb | TMEM63C |
| 14 | 77648134 | C | Sib | TMEM63C |
| 14 | 102446851 | T | Prb | DYNC1H1 |
| 14 | 102446851 | C | Sib | DYNC1H1 |
| 15 | 29500079 | T | Prb | NDNL2 |
| 15 | 29500079 | C | Sib | NDNL2 |
| 15 | 86547181 | T | Prb | AGBL1 |
| 15 | 86547181 | C | Sib | AGBL1 |
| 16 | 10133442 | A | Prb | GRIN2A |
| 16 | 10133442 | C | Sib | GRIN2A |
| 16 | 85833314 | T | Prb | COX411 |
| 16 | 85833314 | G | Sib | COX411 |
| 17 | 21220566 | T | Prb | MAP2K3 |
| 17 | 21220566 | C | Sib | MAP2K3 |
| 17 | 76352731 | A | Prb | SOCS3 |
| 17 | 76352731 | G | Sib | SOCS3 |
| 19 | 4380358 | T | Prb | SH3GL1 |
| 19 | 4380358 | C | Sib | SH3GL1 |
| 19 | 18059044 | C | Prb | CCDC124 |
| 19 | 18059044 | G | Sib | CCDC124 |
| 19 | 55999138 | A | Prb | SSC5D |
| 19 | 55999138 | G | Sib | SSC5D |
| 19 | 59070227 | A | Prb | UBE2M |
| 19 | 59070227 | C | Sib | UBE2M |
| 20 | 59651190 | T | Prb | CDH4 |
| 20 | 59651190 | C | Sib | CDH4 |

TABLE 4
Network-neighborhood Differential Enrichment Analysis (NDEA) significance levels of proband excess for all genes.

| Gene symbol (HGNC) | | Entrez gene id | ENSEMBL gene id | NDEA p-value | NDEA q-value | Cluster |
|---|---|---|---|---|---|---|
| HIC2 | | 23119 | ENSG00000169635 | 1.68E−06 | 0.027256142 | Chromatin cluster |
| NCOR2 | | 9612 | ENSG00000196498 | 5.97E−06 | 0.027256142 | Chromatin cluster |
| NFASC | | 23114 | ENSG00000163531 | 6.04E−06 | 0.027256142 | Synapse cluster |
| LACTBL1 | | 646262 | ENSG00000215906 | 2.02E−05 | 0.048079035 | |
| LINGO1 | | 84894 | ENSG00000169783 | 2.29E−05 | 0.048079035 | |
| CENPB | | 1059 | ENSG00000125817 | 2.44E−05 | 0.048079035 | Chromatin cluster |
| PPP2R5B | | 5526 | ENSG00000068971 | 2.99E−05 | 0.048079035 | Chromatin cluster |
| WSCD1 | | 23302 | ENSG00000179314 | 3.04E−05 | 0.048079035 | Synapse cluster |
| GFRA2 | | 2675 | ENSG00000168546 | 3.20E−05 | 0.048079035 | Synapse cluster |
| PDE4A | | 5141 | ENSG00000065989 | 8.12E−05 | 0.071827716 | Synapse cluster |
| MIDN | | 90007 | ENSG00000167470 | 8.31E−05 | 0.071827716 | Chromatin cluster |
| PLPPR2 | | 64748 | ENSG00000105520 | 9.23E−05 | 0.071827716 | Chromatin cluster |
| STRN4 | | 29888 | ENSG00000090372 | 0.000109335 | 0.071827716 | Chromatin cluster |
| NTN5 | | 126147 | ENSG00000142233 | 0.000114448 | 0.071827716 | Synapse cluster |
| AGRN | | 375790 | ENSG00000188157 | 0.00011957 | 0.071827716 | Chromatin cluster |
| GNAI2 | | 2771 | ENSG00000114353 | 0.000121823 | 0.071827716 | Chromatin cluster |
| SOX12 | | 6666 | ENSG00000177732 | 0.000125415 | 0.071827716 | Chromatin cluster |
| TMEM8C | | 389827 | ENSG00000187616 | 0.000126093 | 0.071827716 | |
| SLC35D1 | | 23169 | ENSG00000116704 | 0.000135262 | 0.071827716 | |
| XKR9 | | 389668 | ENSG00000221947 | 0.000136275 | 0.071827716 | |
| FAM19A3 | | 284467 | ENSG00000184599 | 0.000137717 | 0.071827716 | |
| KDM6B | | 23135 | ENSG00000132510 | 0.000148562 | 0.071827716 | Chromatin cluster |
| ST18 | | 9705 | ENSG00000147488 | 0.000154287 | 0.071827716 | Synapse cluster |
| TTYH2 | | 94015 | ENSG00000141540 | 0.000154579 | 0.071827716 | Synapse cluster |
| ZNRF3 | | 84133 | ENSG00000183579 | 0.000160942 | 0.071827716 | |
| MYT1L | | 23040 | ENSG00000186487 | 0.000165346 | 0.071827716 | Synapse cluster |
| COL6A1 | | 1291 | ENSG00000142156 | 0.000170365 | 0.071827716 | Synapse cluster |
| GRM7 | | 2917 | ENSG00000196277 | 0.000178347 | 0.071827716 | Synapse cluster |
| MEF2D | | 4209 | ENSG00000116604 | 0.000182156 | 0.071827716 | Chromatin cluster |
| CBLN4 | | 140689 | ENSG00000054803 | 0.000184994 | 0.071827716 | Synapse cluster |
| CTU1 | | 90353 | ENSG00000142544 | 0.00020869 | 0.071827716 | Chromatin cluster |
| CMIP | | 80790 | ENSG00000153815 | 0.000210867 | 0.071827716 | Chromatin cluster |
| XKR4 | | 114786 | ENSG00000206579 | 0.000213469 | 0.071827716 | Synapse cluster |
| PKDCC | | 91461 | ENSG00000162878 | 0.00021449 | 0.071827716 | |
| KRTAP5-3 | | 387266 | ENSG00000196224 | 0.000223679 | 0.071827716 | |
| LING03 | | 645191 | ENSG00000220008 | 0.000231454 | 0.071827716 | Synapse cluster |
| SMARCD1 | | 6602 | ENSG00000066117 | 0.000232964 | 0.071827716 | Chromatin cluster |
| KRTAP20-4 | | 100151643 | ENSG00000206105 | 0.000234571 | 0.071827716 | |
| FBRSL1 | | 57666 | ENSG00000112787 | 0.000254272 | 0.071827716 | Chromatin cluster |
| TCF23 | | 150921 | ENSG00000163792 | 0.000254964 | 0.071827716 | |
| SH1SA6 | | 388336 | ENSG00000188803 | 0.00025531 | 0.071827716 | Synapse cluster |
| MAP3K14 | | 9020 | ENSG00000006062 | 0.000257876 | 0.071827716 | |
| SULT6B1 | | 391365 | ENSG00000138068 | 0.00025815 | 0.071827716 | |
| ULK1 | | 8408 | ENSG00000177169 | 0.000263172 | 0.071827716 | Chromatin cluster |
| SATL1 | | 340562 | ENSG00000184788 | 0.000267917 | 0.071827716 | |
| PRSS48 | | 345062 | ENSG00000189099 | 0.000269202 | 0.071827716 | |
| NCAN | | 1463 | ENSG00000130287 | 0.000272103 | 0.071827716 | Synapse cluster |
| OR51G2 | | 81282 | ENSG00000176893 | 0.000278323 | 0.071827716 | |
| PXN | | 5829 | ENSG00000089159 | 0.000280381 | 0.071827716 | Chromatin cluster |
| DMWD | | 1762 | ENSG00000185800 | 0.000281372 | 0.071827716 | Chromatin cluster |
| GSG1L2 | | 644070 | ENSG00000214978 | 0.000284681 | 0.071827716 | |
| RIMS2 | | 9699 | ENSG00000176406 | 0.000285447 | 0.071827716 | Synapse cluster |
| ZFPM2 | | 23414 | ENSG00000169946 | 0.000296891 | 0.071827716 | |
| BSX | | 390259 | ENSG00000188909 | 0.000303019 | 0.071827716 | |
| EPHB4 | | 2050 | ENSG00000196411 | 0.000305433 | 0.071827716 | Chromatin cluster |
| ADAMTS9 | | 56999 | ENSG00000163638 | 0.000360908 | 0.07615054 | Synapse cluster |
| VAMP2 | | 6844 | ENSG00000220205 | 0.000376107 | 0.07615054 | Chromatin cluster |
| CCNI2 | | 645121 | ENSG00000205089 | 0.000383198 | 0.07615054 | |
| BTBD19 | | 149478 | ENSG00000222009 | 0.000402414 | 0.07615054 | |
| FGFR2 | | 2263 | ENSG00000066468 | 0.000404667 | 0.07615054 | Synapse cluster |
| EGFR | | 1956 | ENSG00000146648 | 0.000404727 | 0.07615054 | Synapse cluster |
| MEX3D | | 399664 | ENSG00000181588 | 0.000407321 | 0.07615054 | Chromatin cluster |
| PRKACA | | 5566 | ENSG00000072062 | 0.00041524 | 0.07615054 | Chromatin cluster |
| GNA11 | | 2767 | ENSG00000088256 | 0.000426973 | 0.07615054 | Chromatin cluster |
| DUSP8 | | 1850 | ENSG00000184545 | 0.000431255 | 0.07615054 | Chromatin cluster |
| SLC9A3R2 | | 9351 | ENSG00000065054 | 0.000451517 | 0.07615054 | Chromatin cluster |
| GFOD2 | | 81577 | ENSG00000141098 | 0.000455915 | 0.07615054 | |
| NKX3-2 | | 579 | ENSG00000109705 | 0.000463323 | 0.07615054 | Synapse cluster |
| KIAA2022 | | 340533 | ENSG00000050030 | 0.00046844 | 0.07615054 | Synapse cluster |
| SNTA1 | | 6640 | ENSG00000101400 | 0.000469691 | 0.07615054 | Chromatin cluster |
| RPUSD1 | | 113000 | ENSG00000007376 | 0.000470932 | 0.07615054 | Chromatin cluster |
| BLACE | | 338436 | ENSG00000204960 | 0.000489806 | 0.07615054 | |
| INA | | 9118 | ENSG00000148798 | 0.000491334 | 0.07615054 | Synapse cluster |
| ASAP3 | | 55616 | ENSG00000088280 | 0.000496763 | 0.07615054 | Chromatin cluster |
| GAS7 | | 8522 | ENSG00000007237 | 0.000497346 | 0.07615054 | Synapse cluster |
| FAM53C | | 51307 | ENSG00000120709 | 0.000499991 | 0.07615054 | |
| TSPAN9 | | 10867 | ENSG00000011105 | 0.000502778 | 0.07615054 | Chromatin cluster |
| PHF12 | | 57649 | ENSG00000109118 | 0.000506547 | 0.07615054 | |
| INPPL1 | | 3636 | ENSG00000165458 | 0.000511581 | 0.07615054 | Chromatin cluster |
| SESN2 | | 83667 | ENSG00000130766 | 0.000519766 | 0.07615054 | |
| NEUROG1 | | 4762 | ENSG00000181965 | 0.000538999 | 0.07615054 | Synapse cluster |
| MAPK8IP1 | | 9479 | ENSG00000121653 | 0.000552924 | 0.07615054 | Synapse cluster |
| SEMA4C | | 54910 | ENSG00000168758 | 0.000561038 | 0.07615054 | Chromatin cluster |
| NPSR1 | | 387129 | ENSG00000187258 | 0.000566945 | 0.07615054 | |
| VMAC | | 400673 | ENSG00000187650 | 0.0005701 | 0.07615054 | |
| FOXS1 | | 2307 | ENSG00000179772 | 0.000585895 | 0.07615054 | Synapse cluster |
| RUFY4 | | 285180 | ENSG00000188282 | 0.000605425 | 0.07615054 | |
| LRFN2 | | 57497 | ENSG00000156564 | 0.000606246 | 0.07615054 | Synapse cluster |
| MT1A | | 4489 | ENSG00000205362 | 0.000609014 | 0.07615054 | |
| MTA1 | | 9112 | ENSG00000182979 | 0.000619575 | 0.07615054 | Chromatin cluster |
| MAPK8IP3 | | 23162 | ENSG00000138834 | 0.000628625 | 0.07615054 | Synapse cluster |
| BACH1 | | 571 | ENSG00000156273 | 0.000636195 | 0.07615054 | |
| CGB7 | | 94027 | ENSG00000196337 | 0.00064518 | 0.07615054 | Synapse cluster |
| AKT1 | | 207 | ENSG00000142208 | 0.000652779 | 0.07615054 | Chromatin cluster |
| PHRF1 | | 57661 | ENSG00000070047 | 0.000653305 | 0.07615054 | Chromatin cluster |
| ARHGEF17 | | 9828 | ENSG00000110237 | 0.000654626 | 0.07615054 | Chromatin cluster |
| KRTAP5-5 | | 439915 | ENSG00000185940 | 0.000658069 | 0.07615054 | |
| SPEN | | 23013 | ENSG00000065526 | 0.000665595 | 0.07615054 | |
| DEFA3 | | 1668 | ENSG00000239839 | 0.000672644 | 0.07615054 | |
| ARID1A | | 8289 | ENSG00000117713 | 0.000704982 | 0.07615054 | Chromatin cluster |
| PLXNA2 | | 5362 | ENSG00000076356 | 0.000710833 | 0.07615054 | Synapse cluster |
| LCE3A | | 353142 | ENSG00000185962 | 0.000710944 | 0.07615054 | |
| VWA5B1 | | 127731 | ENSG00000158816 | 0.000714868 | 0.07615054 | Synapse cluster |
| SLC4A4 | | 8671 | ENSG00000080493 | 0.000721307 | 0.07615054 | Synapse cluster |
| EPHA8 | | 2046 | ENSG00000070886 | 0.000723036 | 0.07615054 | |
| EEFSEC | | 60678 | ENSG00000132394 | 0.00072356 | 0.07615054 | Chromatin cluster |
| CDK13 | | 8621 | ENSG00000065883 | 0.00072827 | 0.07615054 | Synapse cluster |
| C19orf25 | | 148223 | ENSG00000119559 | 0.000733885 | 0.07615054 | Chromatin cluster |
| PDE8B | | 8622 | ENSG00000113231 | 0.000752726 | 0.07615054 | Synapse cluster |
| TSPY4 | | 728395 | ENSG00000233803 | 0.000756037 | 0.07615054 | |
| PCDH9 | | 5101 | ENSG00000184226 | 0.000759079 | 0.07615054 | Synapse cluster |
| NECTIN2 | | 5819 | ENSG00000130202 | 0.000761178 | 0.07615054 | Chromatin cluster |
| C3orf70 | | 285382 | ENSG00000187068 | 0.000767716 | 0.07615054 | |
| SEMA6D | | 80031 | ENSG00000137872 | 0.000773919 | 0.07615054 | Synapse cluster |
| KLRG2 | | 346689 | ENSG00000188883 | 0.000779975 | 0.07615054 | |
| USP42 | | 84132 | ENSG00000106346 | 0.000782394 | 0.07615054 | |
| C10orf105 | | 414152 | ENSG00000214688 | 0.000788255 | 0.07615054 | |
| SPRYD4 | | 283377 | ENSG00000176422 | 0.000790354 | 0.07615054 | |
| SATB2 | | 23314 | ENSG00000119042 | 0.000792325 | 0.07615054 | |
| HSPA12A | | 259217 | ENSG00000165868 | 0.000792521 | 0.07615054 | Synapse cluster |
| MFSD2B | | 388931 | ENSG00000205639 | 0.000794581 | 0.07615054 | |
| MYCN | | 4613 | ENSG00000134323 | 0.000801841 | 0.07615054 | Synapse cluster |
| ARHGDIA | | 396 | ENSG00000141522 | 0.000809124 | 0.076225711 | Chromatin cluster |
| C19orf35 | | 374872 | ENSG00000188305 | 0.000815233 | 0.076225711 | |
| ZNF793 | | 390927 | ENSG00000188227 | 0.000815306 | 0.076225711 | |
| FGFRL1 | | 53834 | ENSG00000127418 | 0.000829159 | 0.077121282 | Chromatin cluster |
| AXIN2 | | 8313 | ENSG00000168646 | 0.000838889 | 0.077286183 | |
| ETV3L | | 440695 | ENSG00000253831 | 0.000855433 | 0.077286183 | |
| CRMP1 | | 1400 | ENSG00000072832 | 0.000856943 | 0.077286183 | Synapse cluster |
| TMEM229A | | 730130 | ENSG00000234224 | 0.000860914 | 0.077286183 | |
| PIANP | | 196500 | ENSG00000139200 | 0.000875731 | 0.077289133 | Synapse cluster |
| RAB11FIP4 | | 84440 | ENSG00000131242 | 0.000906564 | 0.078550757 | Synapse cluster |
| GAGE12C | | 729422 | ENSG00000237671 | 0.000920081 | 0.078550757 | |
| DLX6 | | 1750 | ENSG00000006377 | 0.000920954 | 0.078550757 | |
| NR1D1 | | 9572 | ENSG00000126368 | 0.000925401 | 0.078550757 | Chromatin cluster |
| ACVR1C | | 130399 | ENSG00000123612 | 0.000932172 | 0.078550757 | |
| C1QL1 | | 10882 | ENSG00000131094 | 0.000935962 | 0.078550757 | Synapse cluster |
| MED14 | | 9282 | ENSG00000180182 | 0.000938787 | 0.078550757 | Synapse cluster |
| SYN3 | | 8224 | ENSG00000185666 | 0.00094187 | 0.078550757 | Synapse cluster |
| TMEM246 | | 84302 | ENSG00000165152 | 0.000949005 | 0.078550757 | |
| CSPG4 | | 1464 | ENSG00000173546 | 0.000958312 | 0.0788806 | Synapse cluster |
| FOXB2 | | 442425 | ENSG00000204612 | 0.000961733 | 0.0788806 | |
| LTK | | 4058 | ENSG00000062524 | 0.000969577 | 0.07916409 | Synapse cluster |
| DCDC2C | | 728597 | ENSG00000214866 | 0.000994735 | 0.079187006 | |
| EPHA4 | | 2043 | ENSG00000116106 | 0.001000677 | 0.079187006 | Synapse cluster |
| SHC2 | | 25759 | ENSG00000129946 | 0.001004721 | 0.079187006 | Synapse cluster |
| DNAJB5 | | 25822 | ENSG00000137094 | 0.001017283 | 0.079187006 | Synapse cluster |
| KLHL22 | | 84861 | ENSG00000099910 | 0.001026163 | 0.079187006 | Chromatin cluster |
| AHDC1 | | 27245 | ENSG00000126705 | 0.00102743 | 0.079187006 | Chromatin cluster |
| MEIS3 | | 56917 | ENSG00000105419 | 0.001037108 | 0.079187006 | Synapse cluster |
| NECAB2 | | 54550 | ENSG00000103154 | 0.001040072 | 0.079187006 | Synapse cluster |
| GET4 | | 51608 | ENSG00000239857 | 0.00105264 | 0.079187006 | Chromatin cluster |
| VSTM5 | | 387804 | ENSG00000214376 | 0.001057438 | 0.079187006 | |
| NKX2-3 | | 159296 | ENSG00000119919 | 0.001062862 | 0.079187006 | |
| FGFR1 | | 2260 | ENSG00000077782 | 0.001086486 | 0.079187006 | Synapse cluster |
| GABRB3 | | 2562 | ENSG00000166206 | 0.001086499 | 0.079187006 | Synapse cluster |
| GRIA1 | | 2890 | ENSG00000155511 | 0.001086609 | 0.079187006 | Synapse cluster |
| STK11 | | 6794 | ENSG00000118046 | 0.001094038 | 0.079187006 | Chromatin cluster |
| KIRREL3 | | 84623 | ENSG00000149571 | 0.001097124 | 0.079187006 | |
| JMJD7 | | 100137047 | ENSG00000243789 | 0.001111887 | 0.079344966 | |
| SYDE1 | | 85360 | ENSG00000105137 | 0.001133775 | 0.079753866 | Synapse cluster |
| DCX | | 1641 | ENSG00000077279 | 0.001140337 | 0.079753866 | Synapse cluster |
| PCDHA10 | | 56139 | ENSG00000250120 | 0.001151022 | 0.079917304 | Synapse cluster |
| ST3GAL3 | | 6487 | ENSG00000126091 | 0.001151532 | 0.079917304 | Synapse cluster |
| ELAVL3 | | 1995 | ENSG00000196361 | 0.001159145 | 0.08013744 | Synapse cluster |
| IDS | | 3423 | ENSG00000010404 | 0.00116681 | 0.080359472 | Chromatin cluster |
| MAPT | | 4137 | ENSG00000186868 | 0.001206745 | 0.082384886 | Synapse cluster |
| GRAPL | | 400581 | ENSG00000189152 | 0.00122946 | 0.082470838 | Synapse cluster |
| APOA5 | | 116519 | ENSG00000110243 | 0.001245009 | 0.082897532 | |
| RAB11B | | 9230 | ENSG00000185236 | 0.001262665 | 0.083267961 | Chromatin cluster |
| SPRED3 | | 399473 | ENSG00000188766 | 0.001264417 | 0.083267961 | |
| BCL6 | | 604 | ENSG00000113916 | 0.001271785 | 0.083448649 | |
| TTC34 | | 100287898 | ENSG00000215912 | 0.001277805 | 0.083539878 | |
| PRR36 | | 80164 | ENSG00000183248 | 0.001291411 | 0.083653161 | Synapse cluster |
| ABHD17C | | 58489 | ENSG00000136379 | 0.001294444 | 0.083653161 | |
| NCALD | | 83988 | ENSG00000104490 | 0.00129645 | 0.083653161 | Synapse cluster |
| PRKD2 | | 25865 | ENSG00000105287 | 0.001304519 | 0.083653161 | Chromatin cluster |
| CYP26C1 | | 340665 | ENSG00000187553 | 0.001312819 | 0.083653161 | Synapse cluster |
| EEPD1 | | 80820 | ENSG00000122547 | 0.001342347 | 0.083653161 | |
| SEZ6L2 | | 26470 | ENSG00000174938 | 0.001343644 | 0.083653161 | Chromatin cluster |
| SMTN | | 6525 | ENSG00000183963 | 0.001346135 | 0.083653161 | Chromatin cluster |
| TSPY3 | | 728137 | ENSG00000228927 | 0.001354049 | 0.083653161 | |
| PALM | | 5064 | ENSG00000099864 | 0.0013563 | 0.083653161 | Chromatin cluster |
| LRP6 | | 4040 | ENSG00000070018 | 0.001362986 | 0.083653161 | Synapse cluster |
| WNT10A | | 80326 | ENSG00000135925 | 0.001387876 | 0.084037412 | Synapse cluster |
| SSBP3 | | 23648 | ENSG00000157216 | 0.001392966 | 0.084063523 | Chromatin cluster |
| GAD1 | | 2571 | ENSG00000128683 | 0.001411052 | 0.084434178 | Synapse cluster |
| C5orf38 | | 153571 | ENSG00000186493 | 0.001417825 | 0.084434178 | Synapse cluster |
| MAPRE3 | | 22924 | ENSG00000084764 | 0.001446478 | 0.08516217 | Chromatin cluster |
| ElF4E1B | | 253314 | ENSG00000175766 | 0.001452583 | 0.08516217 | |
| CUX2 | | 23316 | ENSG00000111249 | 0.001456845 | 0.08516217 | |
| AMPH | | 273 | ENSG00000078053 | 0.001468631 | 0.08547054 | Synapse cluster |
| ZNF462 | | 58499 | ENSG00000148143 | 0.001473121 | 0.08547054 | |
| RXRB | | 6257 | ENSG00000204231 | 0.001516123 | 0.086819092 | Chromatin cluster |
| TOB2 | | 10766 | ENSG00000183864 | 0.0015215 | 0.086819092 | Chromatin cluster |
| TAOK2 | | 9344 | ENSG00000149930 | 0.001521972 | 0.086819092 | Chromatin cluster |
| MOB2 | | 81532 | ENSG00000182208 | 0.001526682 | 0.086819092 | Chromatin cluster |
| ADCY5 | | 111 | ENSG00000173175 | 0.001535044 | 0.086829733 | |
| AKAP8 | | 10270 | ENSG00000105127 | 0.001541567 | 0.086926187 | |
| DZANK1 | | 55184 | ENSG00000089091 | 0.001572586 | 0.087037977 | |
| CSNK1E | | 1454 | ENSG00000213923 | 0.001619682 | 0.087037977 | Chromatin cluster |
| ANKRD18B | | 441459 | ENSG00000230453 | 0.001619974 | 0.087037977 | Synapse cluster |
| P1K3R3 | | 8503 | ENSG00000117461 | 0.001621267 | 0.087037977 | Synapse cluster |
| BTBD2 | | 55643 | ENSG00000133243 | 0.001622535 | 0.087037977 | Chromatin cluster |
| RCE1 | | 9986 | ENSG00000173653 | 0.001637336 | 0.087037977 | Chromatin cluster |
| NNAT | | 4826 | ENSG00000053438 | 0.001656861 | 0.087037977 | Synapse cluster |
| NTRK3 | | 4916 | ENSG00000140538 | 0.001657327 | 0.087037977 | Synapse cluster |
| SHKBP1 | | 92799 | ENSG00000160410 | 0.001657521 | 0.087037977 | Chromatin cluster |
| FUT9 | | 10690 | ENSG00000172461 | 0.001658055 | 0.087037977 | Synapse cluster |
| SLC35F3 | | 148641 | ENSG00000183780 | 0.001662708 | 0.087037977 | Synapse cluster |
| LCN9 | | 392399 | ENSG00000148386 | 0.001688699 | 0.087037977 | |
| CERCAM | | 51148 | ENSG00000167123 | 0.001703946 | 0.087037977 | Chromatin cluster |
| GTF3C1 | | 2975 | ENSG00000077235 | 0.001710437 | 0.087037977 | Chromatin cluster |
| MAZ | | 4150 | ENSG00000103495 | 0.001740375 | 0.087037977 | Chromatin cluster |
| KCTD8 | | 386617 | ENSG00000183783 | 0.001740738 | 0.087037977 | |
| PIEZO1 | | 9780 | ENSG00000103335 | 0.001751976 | 0.087037977 | Chromatin cluster |
| SNN | | 8303 | ENSG00000184602 | 0.00176044 | 0.087037977 | Chromatin cluster |
| EIF1AY | | 9086 | ENSG00000198692 | 0.001765264 | 0.087037977 | |
| DENND3 | | 22898 | ENSG00000105339 | 0.001772178 | 0.087037977 | Chromatin cluster |
| CPLX1 | | 10815 | ENSG00000168993 | 0.001795998 | 0.087037977 | |
| SALL3 | | 27164 | ENSG00000256463 | 0.001796502 | 0.087037977 | Synapse cluster |
| CLPSL2 | | 389383 | ENSG00000196748 | 0.001798938 | 0.087037977 | |
| EPHA7 | | 2045 | ENSG00000135333 | 0.001803851 | 0.087037977 | |
| RASSF8 | | 11228 | ENSG00000123094 | 0.001809328 | 0.087037977 | |
| PPP1R3G | | 648791 | ENSG00000219607 | 0.001822197 | 0.087037977 | |
| NFIB | | 4781 | ENSG00000147862 | 0.001839257 | 0.087037977 | Synapse cluster |
| SLIT2 | | 9353 | ENSG00000145147 | 0.001846819 | 0.087037977 | Synapse cluster |
| BRD4 | | 23476 | ENSG00000141867 | 0.001851937 | 0.087037977 | Chromatin cluster |
| ACVR2A | | 92 | ENSG00000121989 | 0.001860803 | 0.087037977 | |
| TAS1R3 | | 83756 | ENSG00000169962 | 0.001865763 | 0.087037977 | |
| TNK2 | | 10188 | ENSG00000061938 | 0.001879081 | 0.087037977 | Chromatin cluster |
| ADGRA2 | | 25960 | ENSG00000020181 | 0.001888314 | 0.087037977 | Synapse cluster |
| CTIF | | 9811 | ENSG00000134030 | 0.001904065 | 0.087037977 | Chromatin cluster |
| SAP25 | | 100316904 | ENSG00000205307 | 0.001905686 | 0.087037977 | |
| CLIP3 | | 25999 | ENSG00000105270 | 0.001909292 | 0.087037977 | Synapse cluster |
| SHANK2 | | 22941 | ENSG00000162105 | 0.00191244 | 0.087037977 | Synapse cluster |
| TSC2 | | 7249 | ENSG00000103197 | 0.001915595 | 0.087037977 | Chromatin cluster |
| BDNF | | 627 | ENSG00000176697 | 0.001921558 | 0.087037977 | |
| RBFOX2 | | 23543 | ENSG00000100320 | 0.001932306 | 0.087037977 | Chromatin cluster |
| RPRM | | 56475 | ENSG00000177519 | 0.001937362 | 0.087037977 | |
| MXD4 | | 10608 | ENSG00000123933 | 0.001940985 | 0.087037977 | Chromatin cluster |
| SBK2 | | 646643 | ENSG00000187550 | 0.001944506 | 0.087037977 | |
| CGB8 | | 94115 | ENSG00000213030 | 0.001945039 | 0.087037977 | Synapse cluster |
| DDTL | | 100037417 | ENSG00000099974 | 0.00196145 | 0.087037977 | |
| SYNGAP1 | | 8831 | ENSG00000197283 | 0.001975807 | 0.087037977 | Synapse cluster |
| CABIN1 | | 23523 | ENSG00000099991 | 0.00197847 | 0.087037977 | Chromatin cluster |
| NFIX | | 4784 | ENSG00000008441 | 0.001983801 | 0.087037977 | Synapse cluster |
| ALB | | 213 | ENSG00000163631 | 0.002013414 | 0.087037977 | Synapse cluster |
| CDK9 | | 1025 | ENSG00000136807 | 0.002013664 | 0.087037977 | Chromatin cluster |
| TUBGCP6 | | 85378 | ENSG00000128159 | 0.002018629 | 0.087037977 | Chromatin cluster |
| RARB | | 5915 | ENSG00000077092 | 0.00201871 | 0.087037977 | Synapse cluster |
| TMPPE | | 643853 | ENSG00000188167 | 0.002019045 | 0.087037977 | |
| PTK7 | | 5754 | ENSG00000112655 | 0.002021913 | 0.087037977 | Chromatin cluster |
| CACNA1E | | 777 | ENSG00000198216 | 0.002023152 | 0.087037977 | Synapse cluster |
| ALS2 | | 57679 | ENSG00000003393 | 0.002028059 | 0.087037977 | |
| FMN2 | | 56776 | ENSG00000155816 | 0.002029541 | 0.087037977 | |
| OTOP3 | | 347741 | ENSG00000182938 | 0.002036704 | 0.087037977 | Synapse cluster |
| SHISA7 | | 729956 | ENSG00000187902 | 0.00204371 | 0.087037977 | |
| ARHGEF2 | | 9181 | ENSG00000116584 | 0.00204564 | 0.087037977 | Chromatin cluster |
| PTPRD | | 5789 | ENSG00000153707 | 0.002048449 | 0.087037977 | Synapse cluster |
| RNF40 | | 9810 | ENSG00000103549 | 0.00205116 | 0.087037977 | Chromatin cluster |
| RNF223 | | 401934 | ENSG00000237330 | 0.002051918 | 0.087037977 | |
| NPAS4 | | 266743 | ENSG00000174576 | 0.002053883 | 0.087037977 | Synapse cluster |
| ESCO1 | | 114799 | ENSG00000141446 | 0.002075339 | 0.087037977 | |
| CCDC97 | | 90324 | ENSG00000142039 | 0.002094572 | 0.087037977 | |
| FAM69B | | 138311 | ENSG00000165716 | 0.002107244 | 0.087037977 | Synapse cluster |
| DGKD | | 8527 | ENSG00000077044 | 0.002131451 | 0.087037977 | Chromatin cluster |
| NUDT8 | | 254552 | ENSG00000167799 | 0.002142597 | 0.087037977 | Chromatin cluster |
| SCYL1 | | 57410 | ENSG00000142186 | 0.00214619 | 0.087037977 | Chromatin cluster |
| STKLD1 | | 169436 | ENSG00000198870 | 0.002147744 | 0.087037977 | Synapse cluster |
| AKAP2 | | 11217 | ENSG00000241978 | 0.002175123 | 0.087037977 | |
| MVB12B | | 89853 | ENSG00000196814 | 0.002177927 | 0.087037977 | Synapse cluster |
| PCDH17 | | 27253 | ENSG00000118946 | 0.002185344 | 0.087037977 | Synapse cluster |
| ZBTB10 | | 65986 | ENSG00000205189 | 0.002185926 | 0.087037977 | |
| ADGRL3 | | 23284 | ENSG00000150471 | 0.002190864 | 0.087037977 | Synapse cluster |
| C2orf91 | | 400950 | ENSG00000205086 | 0.002191915 | 0.087037977 | |
| ZNF821 | | 55565 | ENSG00000102984 | 0.002198345 | 0.087037977 | Synapse cluster |
| LGALS16 | | 148003 | ENSG00000249861 | 0.002201155 | 0.087037977 | |
| PRR20C | | 729240 | ENSG00000229665 | 0.002236772 | 0.087037977 | |
| FAM25A | | 643161 | ENSG00000188100 | 0.00228083 | 0.087037977 | |
| FAM163A | | 148753 | ENSG00000143340 | 0.002283101 | 0.087037977 | Synapse cluster |
| MYPOP | | 339344 | ENSG00000176182 | 0.002283468 | 0.087037977 | Chromatin cluster |
| NFKB2 | | 4791 | ENSG00000077150 | 0.002312902 | 0.087037977 | Chromatin cluster |
| BRINP1 | | 1620 | ENSG00000078725 | 0.002326538 | 0.087037977 | Synapse cluster |
| MRPL55 | | 128308 | ENSG00000162910 | 0.002343031 | 0.087037977 | Chromatin cluster |
| CACNB3 | | 784 | ENSG00000167535 | 0.002365829 | 0.087037977 | Chromatin cluster |
| FAM86B2 | | 653333 | ENSG00000145002 | 0.002368348 | 0.087037977 | |
| POTEB2 | | 100287399 | ENSG00000230031 | 0.002384563 | 0.087037977 | |
| C16orf90 | | 646174 | ENSG00000215131 | 0.002385791 | 0.087037977 | |
| MECOM | | 2122 | ENSG00000085276 | 0.002388141 | 0.087037977 | Synapse cluster |
| KLK5 | | 25818 | ENSG00000167754 | 0.00239785 | 0.087037977 | |
| GDF50S | | 554250 | ENSG00000204183 | 0.002399391 | 0.087037977 | |
| MCIDAS | | 345643 | ENSG00000234602 | 0.002399428 | 0.087037977 | |
| FEV | | 54738 | ENSG00000163497 | 0.002421535 | 0.087037977 | Synapse cluster |
| PRRC2A | | 7916 | ENSG00000204469 | 0.002439492 | 0.087037977 | Chromatin cluster |
| SYN2 | | 6854 | ENSG00000157152 | 0.002447301 | 0.087037977 | Synapse cluster |
| IRF2BP2 | | 359948 | ENSG00000168264 | 0.002454753 | 0.087037977 | |
| AEBP2 | | 121536 | ENSG00000139154 | 0.00247921 | 0.087037977 | |
| ESRRA | | 2101 | ENSG00000173153 | 0.002479674 | 0.087037977 | Chromatin cluster |
| ESPN | | 83715 | ENSG00000187017 | 0.002490004 | 0.087037977 | Synapse cluster |
| EPB41L1 | | 2036 | ENSG00000088367 | 0.002494385 | 0.087037977 | Synapse cluster |
| DNM1 | | 1759 | ENSG00000106976 | 0.002500281 | 0.087037977 | Synapse cluster |
| VSIG10L | | 147645 | ENSG00000186806 | 0.00250121 | 0.087037977 | |
| CACNA1G | | 8913 | ENSG00000006283 | 0.002522138 | 0.087037977 | Synapse cluster |
| GMNC | | 647309 | ENSG00000205835 | 0.002525932 | 0.087037977 | |
| PACRG | | 135138 | ENSG00000112530 | 0.002538412 | 0.087037977 | Synapse cluster |
| ZBTB7A | | 51341 | ENSG00000178951 | 0.002592704 | 0.087037977 | Chromatin cluster |
| VPS18 | | 57617 | ENSG00000104142 | 0.00260381 | 0.087037977 | Chromatin cluster |
| FGFR3 | | 2261 | ENSG00000068078 | 0.002616687 | 0.087037977 | Synapse cluster |
| PRKD1 | | 5587 | ENSG00000184304 | 0.002632 | 0.087037977 | Synapse cluster |
| PLXNA1 | | 5361 | ENSG00000114554 | 0.002659137 | 0.087037977 | Chromatin cluster |
| PDGFB | | 5155 | ENSG00000100311 | 0.002669847 | 0.087037977 | Synapse cluster |
| KMT2C | | 58508 | ENSG00000055609 | 0.002681928 | 0.087037977 | |
| SRRM2 | | 23524 | ENSG00000167978 | 0.002682175 | 0.087037977 | Chromatin cluster |
| CSNK1G2 | | 1455 | ENSG00000133275 | 0.002683022 | 0.087037977 | Chromatin cluster |
| MAPKAPK2 | | 9261 | ENSG00000162889 | 0.00268412 | 0.087037977 | Chromatin cluster |
| LMNA | | 4000 | ENSG00000160789 | 0.002709695 | 0.087037977 | Chromatin cluster |
| C1QTNF8 | | 390664 | ENSG00000184471 | 0.002713635 | 0.087037977 | |
| TLE2 | | 7089 | ENSG00000065717 | 0.002725988 | 0.087037977 | Chromatin cluster |
| EMX1 | | 2016 | ENSG00000135638 | 0.002740415 | 0.087037977 | Synapse cluster |
| MXRA8 | | 54587 | ENSG00000162576 | 0.002741903 | 0.087037977 | Synapse cluster |
| GPR156 | | 165829 | ENSG00000175697 | 0.002742733 | 0.087037977 | Synapse cluster |
| LZTS3 | | 9762 | ENSG00000088899 | 0.002748385 | 0.087037977 | |
| KRTAP10-1 | | 386677 | ENSG00000215455 | 0.00275362 | 0.087037977 | |
| ZNF444 | | 55311 | ENSG00000167685 | 0.002754956 | 0.087037977 | Chromatin cluster |
| PPP1R14B | | 26472 | ENSG00000173457 | 0.002789219 | 0.087037977 | Chromatin cluster |
| CCDC85C | | 317762 | ENSG00000205476 | 0.002796918 | 0.087037977 | |
| ZNF774 | | 342132 | ENSG00000196391 | 0.002801236 | 0.087037977 | |
| ZNF536 | | 9745 | ENSG00000198597 | 0.002810283 | 0.087037977 | Synapse cluster |
| RBMY1B | | 378948 | ENSG00000242875 | 0.002817314 | 0.087037977 | |
| CIZ1 | | 25792 | ENSG00000148337 | 0.002820956 | 0.087037977 | Chromatin cluster |
| NPY1R | | 4886 | ENSG00000164128 | 0.00283383 | 0.087037977 | Synapse cluster |
| DLC1 | | 10395 | ENSG00000164741 | 0.002839205 | 0.087037977 | Synapse cluster |
| LRRC41 | | 10489 | ENSG00000132128 | 0.002840994 | 0.087037977 | Chromatin cluster |
| MGAT5B | | 146664 | ENSG00000167889 | 0.002860901 | 0.087037977 | Synapse cluster |
| NRXN2 | | 9379 | ENSG00000110076 | 0.002863746 | 0.087037977 | Synapse cluster |
| CEACAM16 | | 388551 | ENSG00000213892 | 0.002870714 | 0.087037977 | |
| LYPD2 | | 137797 | ENSG00000197353 | 0.002873318 | 0.087037977 | |
| CLIP2 | | 7461 | ENSG00000106665 | 0.002876812 | 0.087037977 | Chromatin cluster |
| COL7A1 | | 1294 | ENSG00000114270 | 0.002900321 | 0.087037977 | Chromatin cluster |
| TTBK1 | | 84630 | ENSG00000146216 | 0.002906154 | 0.087037977 | |
| ZC3H7B | | 23264 | ENSG00000100403 | 0.002912972 | 0.087037977 | Synapse cluster |
| PCDH10 | | 57575 | ENSG00000138650 | 0.002913883 | 0.087037977 | Synapse cluster |
| ANKRD62 | | 342850 | ENSG00000181626 | 0.002926445 | 0.087037977 | |
| KAZN | | 23254 | ENSG00000189337 | 0.002930762 | 0.087037977 | Synapse cluster |
| PTPRN2 | | 5799 | ENSG00000155093 | 0.002932423 | 0.087037977 | Synapse cluster |
| NOTCH4 | | 4855 | ENSG00000204301 | 0.002947343 | 0.087037977 | Synapse cluster |
| CPSF4L | | 642843 | ENSG00000187959 | 0.00295558 | 0.087037977 | |
| PLEKHD1 | | 400224 | ENSG00000175985 | 0.002968878 | 0.087037977 | |
| ZSWIM8 | | 23053 | ENSG00000214655 | 0.003000517 | 0.087037977 | Chromatin cluster |
| ARID3C | | 138715 | ENSG00000205143 | 0.00301302 | 0.087037977 | Synapse cluster |
| GAGE12G | | 645073 | ENSG00000215269 | 0.003019731 | 0.087037977 | |
| NEK5 | | 341676 | ENSG00000197168 | 0.003023316 | 0.087037977 | |
| AJUBA | | 84962 | ENSG00000129474 | 0.003027251 | 0.087037977 | |
| CDK11B | | 984 | ENSG00000248333 | 0.00303119 | 0.087037977 | Chromatin cluster |
| SFSWAP | | 6433 | ENSG00000061936 | 0.00305438 | 0.087037977 | Chromatin cluster |
| ZNF724 | | 440519 | ENSG00000196081 | 0.003081719 | 0.087037977 | |
| FAM193A | | 8603 | ENSG00000125386 | 0.003083006 | 0.087037977 | Chromatin cluster |
| C2CD2L | | 9854 | ENSG00000172375 | 0.003097117 | 0.087037977 | Chromatin cluster |
| TSPYL2 | | 64061 | ENSG00000184205 | 0.003107812 | 0.087037977 | Chromatin cluster |
| HOXB6 | | 3216 | ENSG00000108511 | 0.003114228 | 0.087037977 | Synapse cluster |
| GAGE12J | | 729396 | ENSG00000224659 | 0.003138883 | 0.087037977 | |
| PDGFRA | | 5156 | ENSG00000134853 | 0.003144657 | 0.087037977 | Synapse cluster |
| MAPK11 | | 5600 | ENSG00000185386 | 0.003151267 | 0.087037977 | Synapse cluster |
| GALNT18 | | 374378 | ENSG00000110328 | 0.003155954 | 0.087037977 | Synapse cluster |
| DAGLA | | 747 | ENSG00000134780 | 0.003199189 | 0.087037977 | |
| MRGPRG | | 386746 | ENSG00000182170 | 0.003207089 | 0.087037977 | |
| AREL1 | | 9870 | ENSG00000119682 | 0.003213242 | 0.087037977 | |
| PTP4A3 | | 11156 | ENSG00000184489 | 0.003238222 | 0.087037977 | Chromatin cluster |
| FAM155A | | 728215 | ENSG00000204442 | 0.003239566 | 0.087037977 | Synapse cluster |
| PPP1R15B | | 84919 | ENSG00000158615 | 0.003247622 | 0.087037977 | |
| FGF9 | | 2254 | ENSG00000102678 | 0.003257067 | 0.087037977 | Synapse cluster |
| MAPKBP1 | | 23005 | ENSG00000137802 | 0.003270338 | 0.087037977 | |
| TAF6L | | 10629 | ENSG00000162227 | 0.003277285 | 0.087037977 | Synapse cluster |
| ZNF823 | | 55552 | ENSG00000197933 | 0.003313705 | 0.087037977 | |
| NKAIN2 | | 154215 | ENSG00000188580 | 0.003358674 | 0.087037977 | Synapse cluster |
| TMEM239 | | 100288797 | ENSG00000198326 | 0.0034051 | 0.087037977 | |
| EHMT2 | | 10919 | ENSG00000204371 | 0.003469687 | 0.087037977 | Chromatin cluster |
| MAPK10 | | 5602 | ENSG00000109339 | 0.003470103 | 0.087037977 | Synapse cluster |
| ZBTB17 | | 7709 | ENSG00000116809 | 0.003514679 | 0.087037977 | Chromatin cluster |
| ADCY2 | | 108 | ENSG00000078295 | 0.00352257 | 0.087037977 | Synapse cluster |
| SSC5D | | 284297 | ENSG00000179954 | 0.003530341 | 0.087037977 | Synapse cluster |
| ATXN7L3 | | 56970 | ENSG00000087152 | 0.003545585 | 0.087037977 | Chromatin cluster |
| PTOV1 | | 53635 | ENSG00000104960 | 0.003552335 | 0.087037977 | Chromatin cluster |
| TAL1 | | 6886 | ENSG00000162367 | 0.003557867 | 0.087037977 | Synapse cluster |
| TRIM71 | | 131405 | ENSG00000206557 | 0.003628507 | 0.087037977 | |
| SBK3 | | 100130827 | ENSG00000231274 | 0.003630693 | 0.087037977 | |
| DMPK | | 1760 | ENSG00000104936 | 0.00364158 | 0.087037977 | Chromatin cluster |
| COQ5 | | 84274 | ENSG00000110871 | 0.003646053 | 0.087037977 | |
| ANKRD20A2 | | 441430 | ENSG00000183148 | 0.003655525 | 0.087037977 | Synapse cluster |
| CDC34 | | 997 | ENSG00000099804 | 0.003687144 | 0.087037977 | Chromatin cluster |
| TSPAN18 | | 90139 | ENSG00000157570 | 0.003714404 | 0.087037977 | |
| MADD | | 8567 | ENSG00000110514 | 0.003717658 | 0.087037977 | Chromatin cluster |
| SPG7 | | 6687 | ENSG00000197912 | 0.003724707 | 0.087037977 | Chromatin cluster |
| ADAM11 | | 4185 | ENSG00000073670 | 0.003730244 | 0.087037977 | Synapse cluster |
| ITPKA | | 3706 | ENSG00000137825 | 0.003756882 | 0.087037977 | Synapse cluster |
| NEUROD2 | | 4761 | ENSG00000171532 | 0.003767106 | 0.087037977 | Synapse cluster |
| HRH1 | | 3269 | ENSG00000196639 | 0.003790247 | 0.087037977 | |
| DTNA | | 1837 | ENSG00000134769 | 0.003799232 | 0.087037977 | Synapse cluster |
| PDE2A | | 5138 | ENSG00000186642 | 0.003801301 | 0.087037977 | |
| SCN3A | | 6328 | ENSG00000153253 | 0.003815814 | 0.087037977 | |
| TBX1 | | 6899 | ENSG00000184058 | 0.003846539 | 0.087037977 | Synapse cluster |
| HMG20B | | 10362 | ENSG00000064961 | 0.003847878 | 0.087037977 | Chromatin cluster |
| PBX1 | | 5087 | ENSG00000185630 | 0.003865201 | 0.087037977 | Synapse cluster |
| NAP1L6 | | 645996 | ENSG00000204118 | 0.003869016 | 0.087037977 | |
| JUND | | 3727 | ENSG00000130522 | 0.003876579 | 0.087037977 | Chromatin cluster |
| MAPK7 | | 5598 | ENSG00000166484 | 0.003888585 | 0.087037977 | Chromatin cluster |
| KLHL20 | | 27252 | ENSG00000076321 | 0.003906169 | 0.087037977 | |
| GNA14 | | 9630 | ENSG00000156049 | 0.003925569 | 0.087037977 | |
| ZNF71 | | 58491 | ENSG00000197951 | 0.003937023 | 0.087037977 | |
| KPTN | | 11133 | ENSG00000118162 | 0.003966773 | 0.087037977 | |
| TMEM215 | | 401498 | ENSG00000188133 | 0.003967819 | 0.087037977 | |
| CPXM1 | | 56265 | ENSG00000088882 | 0.003974122 | 0.087037977 | |
| UBE2R2 | | 54926 | ENSG00000107341 | 0.003986744 | 0.087037977 | Chromatin cluster |
| APLP1 | | 333 | ENSG00000105290 | 0.003989286 | 0.087037977 | Synapse cluster |
| NPR1 | | 4881 | ENSG00000169418 | 0.003992187 | 0.087037977 | Synapse cluster |
| KCNT1 | | 57582 | ENSG00000107147 | 0.003993482 | 0.087037977 | Synapse cluster |
| KRTAP5-2 | | 440021 | ENSG00000205867 | 0.003993717 | 0.087037977 | |
| FBXW7 | | 55294 | ENSG00000109670 | 0.00400332 | 0.087037977 | |
| MNX1 | | 3110 | ENSG00000130675 | 0.004005535 | 0.087037977 | |
| SMAGP | | 57228 | ENSG00000170545 | 0.004009655 | 0.087037977 | |
| ZFPM1 | | 161882 | ENSG00000179588 | 0.00401014 | 0.087037977 | Chromatin cluster |
| SARM1 | | 23098 | ENSG00000004139 | 0.004011238 | 0.087037977 | Synapse cluster |
| MBD3 | | 53615 | ENSG00000071655 | 0.004012178 | 0.087037977 | Chromatin cluster |
| RALGDS | | 5900 | ENSG00000160271 | 0.004015131 | 0.087037977 | Chromatin cluster |
| ZDHHC8 | | 29801 | ENSG00000099904 | 0.004027672 | 0.087037977 | Chromatin cluster |
| SRC | | 6714 | ENSG00000197122 | 0.004048676 | 0.087037977 | Synapse cluster |
| FAM227A | | 646851 | ENSG00000184949 | 0.004056183 | 0.087037977 | |
| PPARA | | 5465 | ENSG00000186951 | 0.004061345 | 0.087037977 | Synapse cluster |
| PSMB11 | | 122706 | ENSG00000222028 | 0.004074465 | 0.087037977 | |
| PLPPR5 | | 163404 | ENSG00000117598 | 0.004079554 | 0.087037977 | Synapse cluster |
| FIGN | | 55137 | ENSG00000182263 | 0.004081919 | 0.087037977 | |
| CACNA1A | | 773 | ENSG00000141837 | 0.004102498 | 0.087037977 | Synapse cluster |
| IL17RE | | 132014 | ENSG00000163701 | 0.004102705 | 0.087037977 | |
| SDHAF1 | | 644096 | ENSG00000205138 | 0.004104905 | 0.087037977 | Chromatin cluster |
| OPRL1 | | 4987 | ENSG00000125510 | 0.004110565 | 0.087037977 | Synapse cluster |
| SYMPK | | 8189 | ENSG00000125755 | 0.004138227 | 0.087037977 | Chromatin cluster |
| TP53TG3D | | 729264 | ENSG00000205456 | 0.004145885 | 0.087037977 | |
| VPS9D1 | | 9605 | ENSG00000075399 | 0.004147723 | 0.087037977 | Chromatin cluster |
| FUK | | 197258 | ENSG00000157353 | 0.004148971 | 0.087037977 | |
| NRP1 | | 8829 | ENSG00000099250 | 0.004163019 | 0.087037977 | Synapse cluster |
| PTPRO | | 5800 | ENSG00000151490 | 0.00418422 | 0.087037977 | Synapse cluster |
| DBX1 | | 120237 | ENSG00000109851 | 0.004189631 | 0.087037977 | |
| C9orf172 | | 389813 | ENSG00000232434 | 0.004199381 | 0.087037977 | |
| SMURF1 | | 57154 | ENSG00000198742 | 0.004206919 | 0.087037977 | Chromatin cluster |
| GPR155 | | 151556 | ENSG00000163328 | 0.0042299 | 0.087037977 | |
| KDM7A | | 80853 | ENSG00000006459 | 0.004245366 | 0.087037977 | |
| ABTB1 | | 80325 | ENSG00000114626 | 0.004247842 | 0.087037977 | Chromatin cluster |
| ODF3B | | 440836 | ENSG00000177989 | 0.004277698 | 0.087037977 | |
| PCGF3 | | 10336 | ENSG00000185619 | 0.004281484 | 0.087037977 | |
| ATN1 | | 1822 | ENSG00000111676 | 0.004296713 | 0.087037977 | Chromatin cluster |
| SLC35A4 | | 113829 | ENSG00000176087 | 0.004311524 | 0.087037977 | Chromatin cluster |
| SPACA5 | | 389852 | ENSG00000171489 | 0.004322342 | 0.087037977 | |
| PR5533 | | 260429 | ENSG00000103355 | 0.00432487 | 0.087037977 | Synapse cluster |
| ADORA1 | | 134 | ENSG00000163485 | 0.00435127 | 0.087037977 | Synapse cluster |
| CA10 | | 56934 | ENSG00000154975 | 0.004368778 | 0.087037977 | Synapse cluster |
| KCNMA1 | | 3778 | ENSG00000156113 | 0.004376723 | 0.087037977 | Synapse cluster |
| UBALD1 | | 124402 | ENSG00000153443 | 0.004393147 | 0.087037977 | Chromatin cluster |
| LGI1 | | 9211 | ENSG00000108231 | 0.00439841 | 0.087037977 | Synapse cluster |
| H3F3B | | 3021 | ENSG00000132475 | 0.004407621 | 0.087037977 | |
| UPB1 | | 51733 | ENSG00000100024 | 0.004425783 | 0.087037977 | |
| ATOH8 | | 84913 | ENSG00000168874 | 0.00445527 | 0.087037977 | Synapse cluster |
| LEFTY2 | | 7044 | ENSG00000143768 | 0.00448704 | 0.087037977 | |
| FAM83H | | 286077 | ENSG00000180921 | 0.004516011 | 0.087037977 | Chromatin cluster |
| CELSR2 | | 1952 | ENSG00000143126 | 0.004519953 | 0.087037977 | Chromatin cluster |
| MYO18A | | 399687 | ENSG00000196535 | 0.004533491 | 0.087037977 | Chromatin cluster |
| GRIN2A | | 2903 | ENSG00000183454 | 0.004568861 | 0.087037977 | Synapse cluster |
| NRN1L | | 123904 | ENSG00000188038 | 0.004574555 | 0.087037977 | |
| TAS2R31 | | 259290 | ENSG00000256436 | 0.004577289 | 0.087037977 | |
| KRTAP10-2 | | 386679 | ENSG00000205445 | 0.00458893 | 0.087037977 | |
| C19orf38 | | 255809 | ENSG00000214212 | 0.004589688 | 0.087037977 | |
| ELL | | 8178 | ENSG00000105656 | 0.004590445 | 0.087037977 | Chromatin cluster |
| ATP1A3 | | 478 | ENSG00000105409 | 0.004629419 | 0.087037977 | Synapse cluster |
| CHRD | | 8646 | ENSG00000090539 | 0.004629839 | 0.087037977 | Synapse cluster |
| PANX2 | | 56666 | ENSG00000073150 | 0.004637995 | 0.087037977 | Synapse cluster |
| DVL2 | | 1856 | ENSG00000004975 | 0.004641367 | 0.087037977 | Chromatin cluster |
| SOCS3 | | 9021 | ENSG00000184557 | 0.004645209 | 0.087037977 | Synapse cluster |
| CACHD1 | | 57685 | ENSG00000158966 | 0.004650121 | 0.087037977 | |
| CLOCK | | 9575 | ENSG00000134852 | 0.004657961 | 0.087037977 | Synapse cluster |
| LARGE1 | | 9215 | ENSG00000133424 | 0.004664198 | 0.087037977 | Synapse cluster |
| PLPPR4 | | 9890 | ENSG00000117600 | 0.004699519 | 0.087037977 | Synapse cluster |
| RRBP1 | | 6238 | ENSG00000125844 | 0.004716264 | 0.087037977 | Chromatin cluster |
| PTPN1 | | 5770 | ENSG00000196396 | 0.004724021 | 0.087037977 | Synapse cluster |
| | 11-Mar | 441061 | ENSG00000183654 | 0.004727654 | 0.087037977 | |

| | | | | | |
|---|---|---|---|---|---|
| PLEKHM2 | 23207 | ENSG00000116786 | 0.00473429 | 0.087037977 | Chromatin cluster |
| CADPS | 8618 | ENSG00000163618 | 0.004735347 | 0.087037977 | Synapse cluster |
| SMG6 | 23293 | ENSG00000070366 | 0.004749256 | 0.087037977 | Synapse cluster |
| LIMD1 | 8994 | ENSG00000144791 | 0.00475074 | 0.087037977 | Synapse cluster |
| CELF3 | 11189 | ENSG00000159409 | 0.004765586 | 0.087037977 | Synapse cluster |
| KLF12 | 11278 | ENSG00000118922 | 0.004775385 | 0.087037977 | Synapse cluster |
| CCDC166 | 100130274 | ENSG00000255181 | 0.004805471 | 0.087037977 | |
| APBB1 | 322 | ENSG00000166313 | 0.004818101 | 0.087037977 | Chromatin cluster |
| SLC6A2 | 6530 | ENSG00000103546 | 0.004823359 | 0.087037977 | Synapse cluster |
| TMEM219 | 124446 | ENSG00000149932 | 0.004835886 | 0.087037977 | Chromatin cluster |
| BFSP1 | 631 | ENSG00000125864 | 0.004849636 | 0.087037977 | |
| KCNA1 | 3736 | ENSG00000111262 | 0.004851678 | 0.087037977 | Synapse cluster |
| NUMA1 | 4926 | ENSG00000137497 | 0.004853663 | 0.087037977 | Chromatin cluster |
| RTN2 | 6253 | ENSG00000125744 | 0.004862879 | 0.087037977 | Chromatin cluster |
| MTRNR2L7 | 100288485 | ENSG00000256892 | 0.004868778 | 0.087037977 | |
| SEMA6B | 10501 | ENSG00000167680 | 0.004881902 | 0.087037977 | Synapse cluster |
| KCND2 | 3751 | ENSG00000184408 | 0.004902034 | 0.087037977 | Synapse cluster |
| SBK1 | 388228 | ENSG00000188322 | 0.004915877 | 0.087037977 | |
| KDM2A | 22992 | ENSG00000173120 | 0.004916383 | 0.087037977 | Chromatin cluster |
| ERBB4 | 2066 | ENSG00000178568 | 0.004935612 | 0.087037977 | Synapse cluster |
| BHLHA15 | 168620 | ENSG00000180535 | 0.004959064 | 0.087037977 | Synapse cluster |
| APPL2 | 55198 | ENSG00000136044 | 0.004962146 | 0.087037977 | |
| TMEM55B | 90809 | ENSG00000165782 | 0.004986773 | 0.087037977 | Chromatin cluster |
| DCLK1 | 9201 | ENSG00000133083 | 0.004989012 | 0.087037977 | Synapse cluster |
| MMP15 | 4324 | ENSG00000102996 | 0.005008156 | 0.087037977 | Chromatin cluster |

TABLE 5
Genes that Affect Drug Metabolism

| Medication | Gene (s) |
|---|---|
| abacavir | HLA-B |
| acenocoumarol | VKORC1, CYP2C9 |
| allopurinol | HLA-B |
| amitriptyline | CYP2C19, CYP2D6 |
| aripiprazole | CYP2D6 |
| atazanavir | UGT1A1 |
| atomoxetine | CYP2D6 |
| azathioprine | TPMT |
| capecitabine | DPYD |
| carbamazepine | HLA-A, HLA-B |
| carvedilol | CYP2D6 |
| cisplatin | TPMT |
| citalopram | CYP2C19 |
| clomipramine | CYP2C19, CYP2D6 |
| clopidogrel | CYP2C19 |
| clozapine | CYP2D6 |
| codeine | CYP2D6 |
| daunorubicin | RARG, SLC28A3, UGT1A6 |
| desflurane | CACNA1S, RYR1 |
| desipramine | CYP2D6 |
| doxepin | CYP2C19, CYP2D6 |
| doxorubicin | RARG, SLC28A3, UGT1A6 |
| duloxetine | CYP2D6 |
| enflurane | CACNA1S, RYR1 |
| escitalopram | CYP2C19 |
| esomeprazole | CYP2C19 |
| flecainide | CYP2D6 |
| fluorouracil | DPYD |
| flupenthixol | CYP2D6 |
| fluvoxamine | CYP2D6 |
| glibenclamide | CYP2C9 |
| gliclazide | CYP2C9 |
| glimepiride | CYP2C9 |
| haloperidol | CYP2D6 |
| halothane | CACNA1S, RYR1 |
| imipramine | CYP2C19, CYP2D6 |
| irinotecan | UGT1A1 |
| isoflurane | CACNA1S, RYR1 |
| ivacaftor | CFTR |
| lansoprazole | CYP2C19 |
| mercaptopurine | TPMT |
| methoxyflurane | CACNA1S, RYR1 |
| metoprolol | CYP2D6 |
| mirtazapine | CYP2D6 |
| moclobemide | CYP2C19 |
| nortriptyline | CYP2D6 |
| olanzapine | CYP2D6 |
| omeprazole | CYP2C19 |
| ondansetron | CYP2D6 |
| oxcarbazepine | HLA-B |
| oxycodone | CYP2D6 |
| pantoprazole | CYP2C19 |
| paroxetine | CYP2D6 |
| peginterferon alpha-2a | IFNL3 |
| peginterferon alpha-2b | IFNL3 |
| phenprocoumon | VKORC1, CYP2C9 |
| phenytoin | CYP2C9, HLA-B |
| propafenone | CYP2D6 |
| rabeprazole | CYP2C19 |
| rasburicase | G6PD |
| ribavirin | IFNL3, HLA-B |
| risperidone | CYP2D6 |
| sertraline | CYP2C19 |
| sevoflurane | CACNA1S, RYR1 |
| simvastin | SLCO1B1 |
| succinylcholine | CACNA1S, RYR1 |
| tacrolimus | CYP3A5 |
| tamoxifen | CYP2D6 |
| tegafur | DPYD |
| thioguanine | TPMT |
| tolbutamide | CYP2C9 |
| tramadol | CYP2D6 |
| trimipramine | CYP2C19, CYP2D6 |
| tropisetron | CYP2D6 |
| venlafaxine | CYP2D6 |
| voriconazole | CYP2C19 |
| warfarin | CYP2C9, CYP4F2, VKORC1 |
| zuclopenthixol | CYP2D6 |

1.-78. (canceled)

79. A method for evaluating genetic data to determine biochemical regulatory effects of variants, comprising:
training, using computer systems, a neural network computational model to yield a composite of biochemical regulatory effects,
wherein the biochemical regulatory effects are one of: effects on transcriptional regulation or effects on posttranscriptional regulation,
wherein the deep neural network computational model is trained utilizing a set of features of a regulatory effect profile, and
wherein the regulatory effect profile is one of: a chromatin regulatory effect profile and a RNA binding protein (RBP) and RNA element profile;
obtaining, using computer systems, genetic data of a collection of individuals;
identifying, using computer systems, a set of variants within the genetic data of the collection of individuals; and
determining, using computer systems and the trained neural network computational model, the biochemical regulatory effects of each variant of the set variants.

80. The method of claim 79, wherein the collection of individuals share a complex trait and each individual has been diagnosed as having the complex trait, or wherein the collection of individuals are unaffected and each individual has not been diagnosed as having the complex trait.

81. (canceled)

82. The method of claim 79, wherein the neural network is a deep neural network or a convolutional neural network.

83. (canceled)

84. The method of claim 79, wherein the regulatory profile is the chromatin regulatory effect profile, and wherein the set of features are cell-type specific.

85. The method of claim 79, wherein the regulatory profile is the chromatin regulatory effect profile, and wherein the set of features include at least one of: sites of chromatin accessibility, chromatin marks, and transcription factor binding sites.

86. The method of claim 85, wherein the chromatin regulatory effect profile is determined utilizing at least one epigenetic assay selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and a methyl array.

87. The method of claim 79, wherein the regulatory profile is the RBP and RNA element profile, wherein the set of features are cell-type specific, and wherein the set of features include RBP binding sites.

88. (canceled)

89. The method of claim 88, wherein the RBP and RNA element profile is determined utilizing at least one RNA-binding assays selected from a group consisting of: cross-linking immunoprecipitation sequencing (CLIP-seq) and RNA immunoprecipitation sequencing (RIP-seq).

90.-91. (canceled)

92. The method of claim 79, wherein the identified set of variants includes at least one de novo variant or at least one inherited variant.

93. (canceled)

94. The method of claim 79, further comprising performing a biochemical assay to further assess at least one variant of the set variants, wherein the biochemical assay assesses one of: transcription, RNA processing, translation, or cell function.

95. The method of claim 94, wherein the biochemical assay is selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis, qPCR, RNA hybridization, cross-linking immunoprecipitation sequencing (CLIP-seq), RNA immunoprecipitation sequencing (RIP-seq), RNA-seq, western blot, immunodetection, flow cytometry, enzyme-linked immunosorbent assay (ELISA), and mass spectrometry.

96. The method of claim 79, further comprising:
training, using computer systems, a linear regression model to yield a pathogenicity of a variant based on the variant's effect on biochemical regulation,
wherein the pathogenicity of the variant is based upon an aggregation of the effects upon the at least one biochemical regulatory process,
wherein the computational model is trained utilizing a set of known pathogenic variants and a set of null variants, and
wherein the effects on biochemical regulation has been determined for each variant of the set of pathogenic variants and of the set of null variants;
obtaining, using the computer systems, the set of identified variants,
wherein the effects on biochemical regulation has been determined for each variant of the set of variants by the trained neural network computational model; and
determining, using the computer systems and the trained linear regression model, the pathogenicity of at least one variant of the set of identified variants.

97.-98. (canceled)

99. The method of claim 96, wherein the linear regression model is L2 regularized.

100.-105. (canceled)

106. The method of claim 96 further comprising aggregating each obtained variant's pathogenicity to achieve a cumulative pathogenicity score for the set of obtained variants.

107.-108. (canceled)

109. The method of claim 96, further comprising performing a biochemical assay to further assess at least one variant of the set variants, wherein the biochemical assay assesses one of: transcription, RNA processing, translation, or cell function.

110. The method of claim 109, wherein the biochemical assay is selected from a group consisting of: chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis, qPCR, RNA hybridization, cross-linking immunoprecipitation sequencing (CLIP-seq), RNA immunoprecipitation sequencing (RIP-seq), RNA-seq, western blot, immunodetection, flow cytometry, enzyme-linked immunosorbent assay (ELISA), and mass spectrometry.

111. The method of claim 96, further comprising:
identifying a set of genomic loci, wherein each genetic locus of the set spans across at least one variant of a second set of variants,
wherein the second set of variants is at least a subset of the identified set of variants, and
wherein the second set of variants are selected based on their pathogenicity;
synthesizing a set of nucleic acid oligomers such that the set of nucleic acid oligomers can be utilized in a molecular assay to detect the presence of variants within the set of identified genomic loci.

112.-123. (canceled)

124. The method of claim 111, wherein the pathogenicity of each variant of the second set of variants is greater than a threshold.

125. The method of claim 111, wherein the molecular assay is capture sequencing and the set of nucleic acid oligomers is capable of hybridizing to the set of identified genomic loci.

126. The method of claim 111, wherein the molecular assay is a single nucleotide polymorphism (SNP) array and the set of nucleic acid oligomers is capable of hybridizing to the set of identified genomic loci.

127. The method of claim 111, wherein the molecular assay is a sequencing assay and the set of nucleic acid oligomers is capable of amplifying the set of identified genomic loci by polymerase chain reaction (PCR).

128.-146. (canceled)

147. The method of claim 96 further comprising:
introducing at least one pathogenic variant of the identified set of variants into the DNA of a biological cell.

148. The method of claim 96 further comprising:
identifying at least one pathogenic variant of the identified set of variants within the DNA of a biological cell; and
performing mutagenesis on the DNA at the site of the at least one pathogenic variant.
